SEQUENCE LISTING (49 sequences)

SEQ ID NO: 1 - protein, 4 aa, Homo sapiens
ETGE

SEQ ID NO: 2 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 3 - protein, 442 aa, Homo sapiens [sequence omitted]

SEQ ID NO: 4 - protein, 379 aa, Homo sapiens
MEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 5 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 6 - protein, 278 aa, Homo sapiens
MRLQHLLKMIFRIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 7 - protein, 278 aa, Homo sapiens; identical to SEQ ID NO: 6

SEQ ID NO: 8 - protein, 284 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIVYFPLL

SEQ ID NO: 9 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 10 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCVRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 11 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 12 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 13 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 14 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 15 - protein, 4857 aa, Homo sapiens
MVTGGGAAPPGTVTEPLPSVIVLSAGRKMAAAAAAASGPGCSSAAGAGAAGVSEWLVLRDGCMHCDADGLHSLSYHPALNAILAVTSRGTIKVIDGTSGATLQASALSAKPGGQVKCQYISAVDKVIFVDDYAVGCRKDLNGILLLDTALQTPVSKQDDVVQLELPVTEAQQLLSACLEKVDISSTEGYDLFITQLKDGLKNTSHETAANHKVAKWATVTFHLPHHVLKSIASAIVNELKKINQNVAALPVASSVMDRLSYLLPSARPELGVGPGRSVDRSLMYSEANRRETFTSWPHVGYRWAQPDPMAQAGFYHQPASSGDDRAMCFTCSVCLVCWEPTDEPWSEHERHSPNCPFVKGEHTQNVPLSVTLATSPAQFPCTDGTDRISCFGSGSCPHFLAAATKRGKICIWDVSKLMKVHLKFEINAYDPAIVQQLILSGDPSSGVDSRRPTLAWLEDSSSCSDIPKLEGDSDDLLEDSDSEEHSRSDSVTGHTSQKEAMEVSLDITALSILQQPEKLQWEIVANVLEDTVKDLEELGANPCLTNSKSEKTKEKHQEQHNIPFPCLLAGGLLTYKSPATSPISSNSHRSLDGLSRTQGESISEQGSTDNESCTNSELNSPLVRRTLPVLLLYSIKESDEKAGKIFSQMNNIMSKSLHDDGFTVPQIIEMELDSQEQLLLQDPPVTYIQQFADAAANLTSPDSEKWNSVFPKPGTLVQCLRLPKFAEEENLCIDSITPCADGIHLLVGLRTCPVESLSAINQVEALNNLNKLNSALCNRRKGELESNLAVVNGANISVIQHESPADVQTPLIIQPEQRNVSGGYLVLYKMNYATRIVTLEEEPIKIQHIKDPQDTITSLILLPPDILDNREDDCEEPIEDMQLTSKNGFEREKTSDISTLGHLVITTQGGYVKILDLSNFEILAKVEPPKKEGTEEQDTFVSVIYCSGTDRLCACTKGGELHFLQIGGTCDDIDEADILVDGSLSKGIEPSSEGSKPLSNPSSPGISGVDLLVDQPFTLEILTSLVELTRFETLTPRFSATVPPCWVEVQQEQQQRRHPQHLHQQHHGDAAQHTRTWKLQTDSNSWDEHVFELVLPKACMVGHVDFKFVLNSNITNIPQIQVTLLKNKAPGLGKVNALNIEVEQNGKPSLVDLNEEMQHMDVEESQCLRLCPFLEDHKEDILCGPVWLASGLDLSGHAGMLTLTSPKLVKGMAGGKYRSFLIHVKAVNERGTEEICNGGMRPVVRLPSLKHQSNKGYSLASLLAKVAAGKEKSSNVKNENTSGTRKSENLRGCDLLQEVSVTIRRFKKTSISKERVQRCAMLQFSEFHEKLVNTLCRKTDDGQITEHAQSLVLDTLCWLAGVHSNGPGSSKEGNENLLSKTRKFLSDIVRVCFFEAGRSIAHKCARFLALCISNGKCDPCQPAFGPVLLKALLDNMSFLPAATTGGSVYWYFVLLNYVKDEDLAGCSTACASLLTAVSRQLQDRLTPMEALLQTRYGLYSSPFDPVLFDLEMSGSSCKNVYNSSIGVQSDEIDLSDVLSGNGKVSSCTAAEGSFTSLTGLLEVEPLHFTCVSTSDGTRIERDDAMSSFGVTPAVGGLSSGTVGEASTALSSAAQVALQSLSHAMASAEQQLQVLQEKQQQLLKLQQQKAKLEAKLHQTTAAAAAAASAVGPVHNSVPSNPVAAPGFFIHPSDVIPPTPKTTPLFMTPPLTPPNEAVSVVINAELAQLFPGSVIDPPAVNLAAHNKNSNKSRMNPLGSGLALAISHASHFLQPPPHQSIIIERMHSGARRFVTLDFGRPILLTDVLIPTCGDLASLSIDIWTLGEEVDGRRLVVATDISTHSLILHDLIPPPVCRFMKITVIGRYGSTNARAKIPLGFYYGHTYILPWESELKLMHDPLKGEGESANQPEIDQHLAMMVALQEDIQCRYNLACHRLETLLQSIDLPPLNSANNAQYFLRKPDKAVEEDSRVFSAYQDCIQLQLQLNLAHNAVQRLKVALGASRKMLSETSNPEDLIQTSSTEQLRTIIRYLLDTLLSLLHASNGHSVPAVLQSTFHAQACEELFKHLCISGTPKIRLHTGLLLVQLCGGERWWGQFLSNVLQELYNSEQLLIFPQDRVFMLLSCIGQRSLSNSGVLESLLNLLDNLLSPLQPQLPMHRRTEGVLDIPMISWVVMLVSRLLDYVATVEDEAAAAKKPLNGNQWSFINNNLHTQSLNRSSKGSSSLDRLYSRKIRKQLVHHKQQLNLLKAKQKALVEQMEKEKIQSNKGSSYKLLVEQAKLKQATSKHFKDLIRLRRTAEWSRSNLDTEVTTAKESPEIEPLPFTLAHERCISVVQKLVLFLLSMDFTCHADLLLFVCKVLARIANATRPTIHLCEIVNEPQLERLLLLLVGTDFNRGDISWGGAWAQYSLTCMLQDILAGELLAPVAAEAMEEGTVGDDVGATAGDSDDSLQQSSVQLLETIDEPLTHDITGAPPLSSLEKDKEIDLELLQDLMEVDIDPLDIDLEKDPLAAKVFKPISSTWYDYWGADYGTYNYNPYIGGLGIPVAKPPANTEKNGSQTVSVSVSQALDARLEVGLEQQAELMLKMMSTLEADSILQALTNTSPTLSQSPTGTDDSLLGGLQAANQTSQLIIQLSSVPMLNVCFNKLFSMLQVHHVQLESLLQLWLTLSLNSSSTGNKENGADIFLYNANRIPVISLNQASITSFLTVLAWYPNTLLRTWCLVLHSLTLMTNMQLNSGSSSAIGTQESTAHLLVSDPNLIHVLVKFLSGTSPHGTNQHSPQVGPTATQAMQEFLTRLQVHLSSTCPQIFSEFLLKLIHILSTERGAFQTGQGPLDAQVKLLEFTLEQNFEVVSVSTISAVIESVTFLVHHYITCSDKVMSRSGSDSSVGARACFGGLFANLIRPGDAKAVCGEMTRDQLMFDLLKLVNILVQLPLSGNREYSARVSVTTNTTDSVSDEEKVSGGKDGNGSSTSVQGSPAYVADLVLANQQIMSQILSALGLCNSSAMAMIIGASGLHLTKHENFHGGLDAISVGDGLFTILTTLSKKASTVHMMLQPILTYMACGYMGRQGSLATCQLSEPLLWFILRVLDTSDALKAFHDMGGVQLICNNMVTSTRAIVNTARSMVSTIMKFLDSGPNKAVDSTLKTRILASEPDNAEGIHNFAPLGTITSSSPTAQPAEVLLQATPPHRRARSAAWSYIFLPEEAWCDLTIHLPAAVLLKEIHIQPHLASLATCPSSVSVEVSADGVNMLPLSTPVVTSGLTYIKIQLVKAEVASAVCLRLHRPRDASTLGLSQIKLLGLTAFGTTSSATVNNPFLPSEDQVSKTSIGWLRLLHHCLTHISDLEGMMASAAAPTANLLQTCAALLMSPYCGMHSPNIEVVLVKIGLQSTRIGLKLIDILLRNCAASGSDPTDLNSPLLFGRLNGLSSDSTIDILYQLGTTQDPGTKDRIQALLKWVSDSARVAAMKRSGRMNYMCPNSSTVEYGLLMPSPSHLHCVAAILWHSYELLVEYDLPALLDQELFELLFNWSMSLPCNMVLKKAVDSLLCSMCHVHPNYFSLLMGWMGITPPPVQCHHRLSMTDDSKKQDLSSSLTDDSKNAQAPLALTESHLATLASSSQSPEAIKQLLDSGLPSLLVRSLASFCFSHISSSESIAQSIDISQDKLRRHHVPQQCNKMPITADLVAPILRFLTEVGNSHIMKDWLGGSEVNPLWTALLFLLCHSGSTSGSHNLGAQQTSARSASLSSAATTGLTTQQRTAIENATVAFFLQCISCHPNNQKLMAQVLCELFQTSPQRGNLPTSGNISGFIRRLFLQLMLEDEKVTMFLQSPCPLYKGRINATSHVIQHPMYGAGHKFRTLHLPVSTTLSDVLDRVSDTPSITAKLISEQKDDKEKKNHEEKEKVKAENGFQDNYSVVVASGLKSQSKRAVSATPPRPPSRRGRTIPDKIGSTSGAEAANKIITVPVFHLFHKLLAGQPLPAEMTLAQLLTLLYDRKLPQGYRSIDLTVKLGSRVITDPSLSKTDSYKRLHPEKDHGDLLASCPEDEALTPGDECMDGILDESLLETCPIQSPLQVFAGMGGLALIAERLPMLYPEVIQQVSAPVVTSTTQEKPKDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 16 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 17 - protein, 236 aa, Homo sapiens
MTGYEARLITFGTWMYSVNKEQLARAGFYAIGQEDKVQCFHCGGGLANWKPKEDPWEQHAKWYPGCKYLLEEKGHEYINNIHLTRSLEGALVQTTKKTPSLTKRISDTIFPNPMLQEAIRMGFDFKDVKKIMEERIQTSGSNYKTLEVLVADLVSAQKDTTENELNQTSLQREISPEEPLRRLQEEKLCKICMDRHIAVVFIPCGHLVTCKQCAEAVDRCPMCSAVIDFKQRVFMS

SEQ ID NO: 18 - protein, 624 aa, Homo sapiens
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 19 - protein, 600 aa, Homo sapiens
MAPSSKSERNSGAGSGGGGPGGAGGKRAAGRRREHVLKQLERVKISGQLSPRLFRKLPPRVCVSLKNIVDEDFLYAGHIFLGFSKCGRYVLSYTSSSGDDDFSFYIYHLYWWEFNVHSKLKLVRQVRLFQDEEIYSDLYLTVCEWPSDASKVIVFGFNTRSANGMLMNMMMMSDENHRDIYVSTVAVPPPGRCAACQDASRAHPGDPNAQCLRHGFMLHTKYQVVYPFPTFQPAFQLKKDQVVLLNTSYSLVACAVSVHSAGDRSFCQILYDHSTCPLAPASPPEPQSPELPPALPSFCPEAAPARSSGSPEPSPAIAKAKEFVADIFRRAKEAKGGVPEEARPALCPGPSGSRCRAHSEPLALCGETAPRDSPPASEAPASEPGYVNYTKLYYVLESGEGTEPEDELEDDKISLPFVVTDLRGRNLRPMRERTAVQGQYLTVEQLTLDFEYVINEVIRHDATWGHQFCSFSDYDIVILEVCPETNQVLINIGLLLLAFPSPTEEGQLRPKTYHTSLKVAWDLNTGIFETVSVGDLTEVKGQTSGSVWSSYRKSCVDMVMKWLVPESSGRYVNRMTNEALHKGCSLKVLADSERYTWIVL

SEQ ID NO: 20 - protein, 190 aa, Homo sapiens
MSTRKRRGGAINSRQAQKRTREATSTPEISLEAEPIELVETAGDEIVDLTCESLEPVVVDLTHNDSVVIVDERRRPRRNARRLPQDHADSCVVSSDDEELSRDRDVYVTTHTPRNARDEGATGLRPSGTVSCPICMDGYSEIVQNGRLIVSTECGHVFCSQCLRDSLKNANTCPTCRKKINHKRYHPIYI

SEQ ID NO: 21 - protein, 113 aa, Homo sapiens
MSTRKRRGGAINSRQAQKRTREATSTPEISLEAEPIELVETAGDEIVDLTCESLEPVVVDLTHNDSVVIVDGPQVLSVVPSAWTDTQRSCRMDVSSFPQNAAMSSVASASVIP

SEQ ID NO: 22 - protein, 228 aa, Homo sapiens
MAAQQRDCGGAAQLAGPAAEADPLGRFTCPVCLEVYEKPVQVPCGHVFCSACLQECLKPKKPVCGVCRSALAPGVRAVELERQIESTETSCHGCRKNFFLSKIRSHVATCSKYQNYIMEGVKATIKDASLQPRNVPNRYTFPCPYCPEKNFDQEGLVEHCKLFHSTDTKSVVCPICASMPWGDPNYRSANFREHIQRRHRFSYDTFVDYDVDEEDMMNQVLQRSIIDQ

SEQ ID NO: 23 - protein, 191 aa, Homo sapiens
MAAQQRDCGGAAQLAGPAAEADPLGRFTCPVCLEVYEKPVQVPCGHVFCSACLQECLKPKKPVCGVCRSALAPGVRAVELERQIESTETSCHGCRKNFFLSKIRSHVATCSKYQNYIMEGVKATIKDASLQPRNVPNRYTFPCPYCPEKNFDQEGLVEHCKLFHSTDTKSVVSEQSPCLLSVSCYRASITY

SEQ ID NO: 24 - protein, 216 aa, Homo sapiens
MGPRNPSPDHLSESESEEEENISYLNESSGEEWDSSEEEDSMVPNLSPLESLAWQVKCLLKYSTTWKPLNPNSWLYHAKLLDPSTPVHILREIGLRLSHCSHCVPKLEPIPEWPPLASCGVPPFQKPLTSPSRLSRDHATLNGALQFATKQLSRTLSRATPIPEYLKQIPNSCVSGCCCGWLTKTVKETTRTEPINTTYSYTDFQKAVNKLLTASL

SEQ ID NO: 25 - protein, 848 aa, Homo sapiens
MNSSSANITYASRKRRKPVQKTVKPIPAEGIKSNPSKRHRDRLNTELDRLASLLPFPQDVINKLDKLSVLRLSVSYLRAKSFFDVALKSSPTERNGGQDNCRAANFREGLNLQEGEFLLQALNGFVLVVTTDALVFYASSTIQDYLGFQQSDVIHQSVYELIHTEDRAEFQRQLHWALNPSQCTESGQGIEEATGLPQTVVCYNPDQIPPENSPLMERCFICRLRCLLDNSSGFLAMNFQGKLKYLHGQKKKGKDGSILPPQLALFAIATPLQPPSILEIRTKNFIFRTKHKLDFTPIGCDAKGRIVLGYTEAELCTRGSGYQFIHAADMLYCAESHIRMIKTGESGMIVFRLLTKNNRWTWVQSNARLLYKNGRPDYIIVTQRPLTDEEGTEHLRKRNTKLPFMFTTGEAVLYEATNPFPAIMDPLPLRTKNGTSGKDSATTSTLSKDSLNPSSLLAAMMQQDESIYLYPASSTSSTAPFENNFFNESMNECRNWQDNTAPMGNDTILKHEQIDQPQDVNSFAGGHPGLFQDSKNSDLYSIMKNLGIDFEDIRHMQNEKFFRNDFSGEVDFRDIDLTDEILTYVQDSLSKSPFIPSDYQQQQSLALNSSCMVQEHLHLEQQQQHHQKQVVVEPQQQLCQKMKHMQVNGMFENWNSNQFVPFNCPQQDPQQYNVFTDLHGISQEFPYKSEMDSMPYTQNFISCNQPVLPQHSKCTELDYPMGSFEPSPYPTTSSLEDFVTCLQLPENQKHGLNPQSAIITPQTCYAGAVSMYQCQPEPQHTHVGQMQYNPVLPGQQAFLNKFQNGVLNETYPAELNNINNTQTTTHLQPLHHPSEARPFPDLTSSGFL

SEQ ID NO: 26 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 27 - protein, 1755 aa, Homo sapiens
MASELEPEVQAIDRSLLECSAEEIAGKWLQATDLTREVYQHLAHYVPKIYCRGPNPFPQKEDMLAQHVLLGPMEWYLCGEDPAFGFPKLEQANKPSHLCGRVFKVGEPTYSCRDCAVDPTCVLCMECFLGSIHRDHRYRMTTSGGGGFCDCGDTEAWKEGPYCQKHELNTSEIEEEEDPLVHLSEDVIARTYNIFAITFRYAVEILTWEKESELPADLEMVEKSDTYYCMLFNDEVHTYEQVIYTLQKAVNCTQKEAIGFATTVDRDGRRSVRYGDFQYCEQAKSVIVRNTSRQTKPLKVQVMHSSIVAHQNFGLKLLSWLGSIIGYSDGLRRILCQVGLQEGPDGENSSLVDRLMLSDSKLWKGARSVYHQLFMSSLLMDLKYKKLFAVRFAKNYQQLQRDFMEDDHERAVSVTALSVQFFTAPTLARMLITEENLMSIIIKTFMDHLRHRDAQGRFQFERYTALQAFKFRRVQSLILDLKYVLISKPTEWSDELRQKFLEGFDAFLELLKCMQGMDPITRQVGQHIEMEPEWEAAFTLQMKLTHVISMMQDWCASDEKVLIEAYKKCLAVLMQCHGGYTDGEQPITLSICGHSVETIRYCVSQEKVSIHLPVSRLLAGLHVLLSKSEVAYKFPELLPLSELSPPMLIEHPLRCLVLCAQVHAGMWRRNGFSLVNQIYYYHNVKCRREMFDKDVVMLQTGVSMMDPNHFLMIMLSRFELYQIFSTPDYGKRFSSEITHKDVVQQNNTLIEEMLYLIIMLVGERFSPGVGQVNATDEIKREIIHQLSIKPMAHSELVKSLPEDENKETGMESVIEAVAHFKKPGLTGRGMYELKPECAKEFNLYFYHFSRAEQSKAEEAQRKLKRQNREDTALPPPVLPPFCPLFASLVNILQSDVMLCIMGTILQWAVEHNGYAWSESMLQRVLHLIGMALQEEKQHLENVTEEHVVTFTFTQKISKPGEAPKNSPSILAMLETLQNAPYLEVHKDMIRWILKTFNAVKKMRESSPTSPVAETEGTIMEESSRDKDKAERKRKAEIARLRREKIMAQMSEMQRHFIDENKELFQQTLELDASTSAVLDHSPVASDMTLTALGPAQTQVPEQRQFVTCILCQEEQEVKVESRAMVLAAFVQRSTVLSKNRSKFIQDPEKYDPLFMHPDLSCGTHTSSCGHIMHAHCWQRYFDSVQAKEQRRQQRLRLHTSYDVENGEFLCPLCECLSNTVIPLLLPPRNIFNNRLNFSDQPNLTQWIRTISQQIKALQFLRKEESTPNNASTKNSENVDELQLPEGFRPDFRPKIPYSESIKEMLTTFGTATYKVGLKVHPNEEDPRVPIMCWGSCAYTIQSIERILSDEDKPLFGPLPCRLDDCLRSLTRFAAAHWTVASVSVVQGHFCKLFASLVPNDSHEELPCILDIDMFHLLVGLVLAFPALQCQDFSGISLGTGDLHIFHLVTMAHIIQILLTSCTEENGMDQENPPCEEESAVLALYKTLHQYTGSALKEIPSGWHLWRSVRAGIMPFLKCSALFFHYLNGVPSPPDIQVPGTSHFEHLCSYLSLPNNLICLFQENSEIMNSLIESWCRNSEVKRYLEGERDAIRYPRESNKLINLPEDYSSLINQASNFSCPKSGGDKSRAPTLCLVCGSLLCSQSYCCQTELEGEDVGACTAHTYSCGSGVGIFLRVRECQVLFLAGKTKGCFYSPPYLDDYGETDQGLRRGNPLHLCKERFKKIQKLWHQHSVTEEIGHAQEANQTLVGIDWQHL

SEQ ID NO: 28 - protein, 374 aa, Homo sapiens
MSRVPSPPPPAEMSSGPVAESWCYTQIKVVKFSYMWTINNFSFCREEMGEVIKSSTFSSGANDKLKWCLRVNPKGLDEESKDYLSLYLLLVSCPKSEVRAKFKFSILNAKGEETKAMESQRAYRFVQGKDWGFKKFIRRDFLLDEANGLLPDDKLTLFCEVSVVQDSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

SEQ ID NO: 29 - protein, 587 aa, Homo sapiens
MEGESVKLSSQTLIQAGDDEKNQRTITVNPAHMGKAFKVMNELRSKQLLCDVMIVAEDVEIEAHRVVLAACSPYFCAMFTGDMSESKAKKIEIKDVDGQTLSKLIDYIYTAEIEVTEENVQVLLPAASLLQLMDVRQNCCDFLQSQLHPTNCLGIRAFADVHTCTDLLQQANAYAEQHFPEVMLGEEFLSLSLDQVCSLISSDKLTVSSEEKVFEAVISWINYEKETRLEHMAKLMEHVRLPLLPRDYLVQTVEEEALIKNNNTCKDFLIEAMKYHLLPLDQRLLIKNPRTKPRTPVSLPKVMIVVGGQAPKAIRSVECYDFEEDRWDQIAELPSRRCRAGVVFMAGHVYAVGGFNGSLRVRTVDVYDGVKDQWTSIASMQERRSTLGAAVLNDLLYAVGGFDGSTGLASVEAYSYKTNEWFFVAPMNTRRSSVGVGVVEGKLYAVGGYDGASRQCLSTVEQYNPATNEWIYVADMSTRRSGAGVGVLSGQLYATGGHDGPLVRKSVEVYDPGTNTWKQVADMNMCRRNAGVCAVNGLLYVVGGDDGSCNLASVEYYNPVTDKWTLLPTNMSTGRSYAGVAVIHKSL

SEQ ID NO: 30 - protein, 568 aa, Homo sapiens
MGGIMAPKDIMTNTHAKSILNSMNSLRKSNTLCDVTLRVEQKDFPAHRIVLAACSDYFCAMFTSELSEKGKPYVDIQGLTASTMEILLDFVYTETVHVTVENVQELLPAACLLQLKGVKQACCEFLESQLDPSNCLGIRDFAETHNCVDLMQAAEVFSQKHFPEVVQHEEFILLSQGEVEKLIKCDEIQVDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASVSLHDRIYVIGGYDGRSRLSSVECLDYTADEDGVWYSVAPMNVRRGLAGATTLGDMIYVSGGFDGSRRHTSMERYDPNIDQWSMLGDMQTAREGAGLVVASGVIYCLGGYDGLNILNSVEKYDPHTGHWTNVTPMATKRSGAGVALLNDHIYVVGGFDGTAHLSSVEAYNIRTDSWTTVTSMTTPRCYVGATVLRGRLYAIAGYDGNSLLSSIECYDPIIDSWEVVTSMGTQRCDAGVCVLREK

SEQ ID NO: 31 - protein, 609 aa, Homo sapiens
MEGKPMRRCTNIRPGETGMDVTSRCTLGDPNKLPEGVPQPARMPYISDKHPRQTLEVINLLRKHRELCDVVLVVGAKKIYAHRVILSACSPYFRAMFTGELAESRQTEVVIRDIDERAMELLIDFAYTSQITVEEGNVQTLLPAACLLQLAEIQEACCEFLKRQLDPSNCLGIRAFADTHSCRELLRIADKFTQHNFQEVMESEEFMLLPANQLIDIISSDELNVRSEEQVFNAVMAWVKYSIQERRPQLPQVLQHVRLPLLSPKFLVGTVGSDPLIKSDEECRDLVDEAKNYLLLPQERPLMQGPRTRPRKPIRCGEVLFAVGGWCSGDAISSVERYDPQTNEWRMVASMSKRRCGVGVSVLDDLLYAVGGHDGSSYLNSVERYDPKTNQWSSDVAPTSTCRTSVGVAVLGGFLYAVGGQDGVSCLNIVERYDPKENKWTRVASMSTRRLGVAVAVLGGFLYAVGGSDGTSPLNTVERYNPQENRWHTIAPMGTRRKHLGCAVYQDMIYAVGGRDDTTELSSAERYNPRTNQWSPVVAMTSRRSGVGLAVVNGQLMAVGGFDGTTYLKTIEVFDPDANTWRLYGGMNYRRLGGGVGVIKMTHCESHIW

SEQ ID NO: 32 - protein, 406 aa, Homo sapiens
MADGNEDLRADDLPGPAFESYESMELACPAERSGHVAVSDGRHMFVWGGYKSNQVRGLYDFYLPREELWIYNMETGRWKKINTEGDVPPSMSGSCAVCVDRVLYLFGGHHSRGNTNKFYMLDSRSTDRVLQWERIDCQGIPPSSKDKLGVWVYKNKLIFFGGYGYLPEDKVLGTFEFDETSFWNSSHPRGWNDHVHILDTETFTWSQPITTGKAPSPRAAHACATVGNRGFVFGGRYRDARMNDLHYLNLDTWEWNELIPQGICPVGRSWHSLTPVSSDHLFLFGGFTTDKQPLSDAWTYCISKNEWIQFNHPYTEKPRLWHTACASDEGEVIVFGGCANNLLVHHRAAHSNEILIFSVQPKSLVRLSLEAVICFKEMLANSWNCLPKHLLHSVNQRFGSNNTSGS

SEQ ID NO: 33 - protein, 273 aa, Homo sapiens
MGQKVTGGIKTVDMRDPTYRPLKQELQGLDYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGRERLGEIHTLPLPASLKAYLLYQ

SEQ ID NO: 34 - protein, 263 aa, Homo sapiens
MGQTALAGGSSSTPTPQALYPDLSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERRAEPHSLLHLSRLCVRHNLGDTRLGQVSALPLPPAMKRYLLYQ

SEQ ID NO: 35 - protein, 273 aa, Homo sapiens
MGQKLSGSLKSVEVREPALRPAKRELRGAEPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNQPGVAYPAFLGPDEAFALPDSLLVVLDMDEGTLSFIVDGQYLGVAFRGLKGKKLYPVVSAVWGHCEVTMRYINGLDPEPLPLMDLCRRSIRSALGRQRLQDISSLPLPQSLKNYLQYQ

SEQ ID NO: 36 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 37 - protein, 535 aa, Homo sapiens
MKKISLKTLRKSFNLNKSKEETDFMVVQQPSLASDFGKDDSLFGSCYGKDMASCDINGEDEKGGKNRSKSESLMGTLKRRLSAKQKSKGKAGTPSGSSADEDTFSSSSAPIVFKDVRAQRPIRSTSLRSHHYSPAPWPLRPTNSEETCIKMEVRVKALVHSSSPSPALNGVRKDFHDLQSETTCQEQANSLKSSASHNGDLHLHLDEHVPVVIGLMPQDYIQYTVPLDEGMYPLEGSRSYCLDSSSPMEVSAVPPQVGGRAFPEDESQVDQDLVVAPEIFVDQSVNGLLIGTTGVMLQSPRAGHDDVPPLSPLLPPMQNNQIQRNFSGLTGTEAHVAESMRCHLNFDPNSAPGVARVYDSVQSSGPMVVTSLTEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGSATYPVRLTNPVSRFMQVRSLQYLCRFVIRQYTRIDLIQKLPLPNKMKDYLQEKHY

SEQ ID NO: 38 - protein, 387 aa, Homo sapiens
MAGSEPRSGTNSPPPPFSDWGRLEAAILSGWKTFWQSVSKERVARTTSREEVDEAASTLTRLPIDVQLYILSFLSPHDLCQLGSTNHYWNETVRDPILWRYFLLRDLPSWSSVDWKSLPDLEILKKPISEVTDGAFFDYMAVYRMCCPYTRRASKSSRPMYGAVTSFLHSLIIQNEPRFAMFGPGLEELNTSLVLSLMSSEELCPTAGLPQRQIDGIGSGVNFQLNNQHKFNILILYSTTRKERDRAREEHTSAVNKMFSRHNEGDDQQGSRYSVIPQIQKVCEVVDGFIYVANAEAHKRHEWQDEFSHIMAMTDPAFGSSGRPLLVLSCISQGDVKRMPCFYLAHELHLNLLNHPWLVQDTEAETLTGFLNGIEWILEEVESKRAR

SEQ ID NO: 39 - protein, 539 aa, Homo sapiens
MAVCARLCGVGPSRGCRRRQQRRGPAETAAADSEPDTDPEEERIEASAGVGGGLCAGPSPPPPRCSLLELPPELLVEIFASLPGTDLPSLAQVCTKFRRILHTDTIWRRRCREEYGVCENLRKLEITGVSCRDVYAKLLHRYRHILGLWQPDIGPYGGLLNVVVDGLFIIGWMYLPPHDPHVDDPMRFKPLFRIHLMERKAATVECMYGHKGPHHGHIQIVKKDEFSTKCNQTDHHRMSGGRQEEFRTWLREEWGRTLEDIFHEHMQELILMKFIYTSQYDNCLTYRRIYLPPSRPDDLIKPGLFKGTYGSHGLEIVMLSFHGRRARGTKITGDPNIPAGQQTVEIDLRHRIQLPDLENQRNFNELSRIVLEVRERVRQEQQEGGHEAGEGRGRQGPRESQPSPAQPRAEAPSKGPDGTPGEDGGEPGDAVAAAEQPAQCGQGQPFVLPVGVSSRNEDYPRTCRMCFYGTGLIAGHGFTSPERTPGVFILFDEDRFGFVWLELKSFSLYSRVQATFRNADAPSPQAFDEMLKNIQSLTS

SEQ ID NO: 40 - protein, 605 aa, Homo sapiens
MDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 41 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 42 - protein, 499 aa, Homo sapiens
MAQFAFESDLHSLLQLDAPIPNAPPARWQRKAKEAAGPAPSPMRAANRSHSAGRTPGRTPGKSSSKVQTTPSKPGGDRYIPHRSAAQMEVASFLLSKENQPENSQTPTKKEHQKAWALNLNGFDVEEAKILRLSGKPQNAPEGYQNRLKVLYSQKATPGSSRKTCRYIPSLPDRILDAPEIRNDYYLNLVDWSSGNVLAVALDNSVYLWSASSGDILQLLQMEQPGEYISSVAWIKEGNYLAVGTSSAEVQLWDVQQQKRLRNMTSHSARVGSLSWNSYILSSGSRSGHIHHHDVRVAEHHVATLSGHSQEVCGLRWAPDGRHLASGGNDNLVNVWPSAPGEGGWVPLQTFTQHQGAVKAVAWCPWQSNVLATGGGTSDRHIRIWNVCSGACLSAVDAHSQVCSILWSPHYKELISGHGFAQNQLVIWKYPTMAKVAELKGHTSRVLSLTMSPDGATVASAAADETLRLWRCFELDPARRREREKASAAKSSLIHQGIR

SEQ ID NO: 43 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 44 - protein, 882 aa, Homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNETQKISQLAAVNRESKFRVVIQPEAFFSIYSKAVSLEVGLQHFLSFLSSMRRPILACYKLWGPGLPNFFRALEDINRLWEFQEAISGFLAALPLIRERVPGASSFKLKNLAQTYLARNMSERSAMAAVLAMRDLCRLLEVSPGPQLAQHVYPFSSLQCFASLQPLVQAAVLPRAEARLLALHNVSFMELLSAHRRDRQGGLKKYSRYLSLQTTTLPPAQPAFNLQALGTYFEGLLEGPALARAEGVSTPLAGRGLAERASQQS

SEQ ID NO: 45 - protein, 475 aa, Homo sapiens [sequence omitted]

SEQ ID NO: 46 - protein, 1050 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQVMAQRQQVQRRPAPVGLPNPRMQGPIQQPSISHQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLK

SEQ ID NO: 47 - protein, 1127 aa, Homo sapiens
MAENKGGGEAESGGGGSGSAPVTAGAAGPAAQEAEPPLTAVLVEEEEEEGGRAGAEGGAAGPDDGGVAAASSGSAQAASSPAASVGTGVAGGAVSTPAPAPASAPAPGPSAGPPPGPPASLLDTCAVCQQSLQSRREAEPKLLPCLHSFCLRCLPEPERQLSVPIPGGSNGDIQQVGVIRCPVCRQECRQIDLVDNYFVKDTSEAPSSSDEKSEQVCTSCEDNASAVGFCVECGEWLCKTCIEAHQRVKFTKDHLIRKKEDVSESVGASGQRPVFCPVHKQEQLKLFCETCDRLTCRDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIASGSSTALLYSKRLITFQLRHILKARCDPVPAANGAIRFHCDPTFWAKNVVNLGNLVIESKPAPGYTPNVVVGQVPPGTNHISKTPGQINLAQLRLQHMQQQVYAQKHQQLQQMRMQQPPAPVPTTTTTTQQHPRQAAPQMLQQQPPRLISVQTMQRGNMNCGAFQAHQMRLAQNAARIPGIPRHSGPQYSMMQPHLQRQHSNPGHAGPFPVVSVHNTTINPTSPTTATMANANRGPTSPSVTAIELIPSVTNPENLPSLPDIPPIQLEDAGSSSLDNLLSRYISGSHLPPQPTSTMNPSPGPSALSPGSSGLSNSHTPVRPPSTSSTGSRGSCGSSGRTAEKTSLSFKSDQVKVKQEPGTEDEICSFSGGVKQEKTEDGRRSACMLSSPESSLTPPLSTNLHLESELDALASLENHVKIEPADMNESCKQSGLSSLVNGKSPIRSLMHRSARIGGDGNNKDDDPNEDWCAVCQNGGDLLCCEKCPKVFHLTCHVPTLLSFPSGDWICTFCRDIGKPEVEYDCDNLQHSKKGKTAQGLSPVDQRKCERLLLYLYCHELSIEFQEPVPASIPNYYKIIKKPMDLSTVKKKLQKKHSQHYQIPDDFVADVRLIFKNCERFNEMMKVVQVYADTQEINLKADSEVAQAGKAVALYFEDKLTEIYSDRTFAPLPEFEQEEDDGEVTEDSDEDFIQPRRKRLKSDERPVHIK

SEQ ID NO: 48 - protein, 300 aa, Homo sapiens
MCARGQVGRGTQLRTGRPCSQVPGSRWRPERLLRRQRAGGRPSRPHPARARPGLSLPATLLGSRAAAAVPLPLPPALAPGDPAMPVRTECPPPAGASAASAASLIPPPPINTQQPGVATSLLYSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHVPEHSAPIYEFR

SEQ ID NO: 49 - protein, 546 aa, Homo sapiens
MGSRNSSSAGSGSGDPSEGLPRRGAGLRRSEEEEEEDEDVDLAQVLAYLLRRGQVRLVQGGGAANLQFIQALLDSEEENDRAWDGRLGDRYNPPVDATPDTRELEFNEIKTQVELATGQLGLRRAAQKHSFPRMLHQRERGLCHRGSFSLGEQSRVISHFLPNDLGFTDSYSQKAFCGIYSKDGQIFMSACQDQTIRLYDCRYGRFRKFKSIKARDVGWSVLDVAFTPDGNHFLYSSWSDYIHICNIYGEGDTHTALDLRPDERRFAVFSIAVSSDGREVLGGANDGCLYVFDREQNRRTLQIESHEDDVNAVAFADISSQILFSGGDDAICKVWDRRTMREDDPKPVGALAGHQDGITFIDSKGDARYLISNSKDQTIKLWDIRRFSSREGMEASRQAATQQNWDYRWQQVPKKAWRKLKLPGDSSLMTYRGHGVLHTLIRCRFSPIHSTGQQFIYSGCSTGKVVVYDLLSGHIVKKLTNHKACVRDVSWHPFEEKIVSSSWDGNLRLWQYRQAEYFQDDMPESEECASAPAPVPQSSTPFSSPQ

(19) United States
(12) Patent Application Publication
(10) Pub. No.: US 2025/0037790 A1
(43) Pub. Date: Jan. 30, 2025

(54) DEGRON AND NEOSUBSTRATE IDENTIFICATION

(71) Applicant: MONTE ROSA THERAPEUTICS, INC., Boston, MA (US)

(72) Inventors: Pablo Gainza Cirauqui, Lausanne (CH); John Christopher Castle, Mainz (DE); Vladimiras Oleinikovas, Basel (CH); Sharon Townson, Somerville, MA (US); Richard David Bunker, Basel (CH)

(21) Appl. No.: 18/709,914

(22) PCT Filed: Nov. 17, 2022
(86) PCT No.: PCT/US2022/050242

§ 371 Date: May 14, 2024

Related U.S. Application Data
(60) Provisional application No. 63/280,508, filed on Nov. 17, 2021.
(60) Provisional application No. 63/419,550, filed on Oct. 26, 2022.

(51) Int. Cl. G16B 15/20; G16B 15/30; G16B 40/20

(52) U.S. Cl. CPC G16B 15/20; G16B 15/30; G16B 40/20

(57) ABSTRACT

Described herein are methods and systems useful, for example, for degron identification, and also, for example, for predicting, identifying, classifying, and selecting neosubstrates of E3 ligases.

### CLAIM OF PRIORITY

This application claims the benefit of U.S. Provisional Application Ser. No. 63/280,508, filed on Nov. 17, 2021, and U.S. Provisional Application Ser. No. 63/419,550, filed on Oct. 26, 2022. The entire contents of the foregoing are incorporated herein by reference.

### SEQUENCE LISTING

This application contains a Sequence Listing that has been submitted electronically as an XML file named 52271-0006WO1-SL_ST26.xml. The XML file, created on Nov. 16, 2022, is 71,488 bytes in size. The material in the XML file is hereby incorporated by reference in its entirety.

### TECHNICAL FIELD

Described herein are methods and systems useful, for example, for degron identification, and also, for example, for predicting, identifying, classifying, and selecting neosubstrates of E3 ligases.

### BACKGROUND

Protein biosynthesis and degradation is a dynamic process which sustains normal cell homeostasis. The ubiquitin-proteasome system is a master regulator of protein homeostasis, by which proteins are initially targeted for poly-ubiquitination by E3 ligases and then degraded into short peptides by the proteasome. Nature evolved diverse peptidic motifs, termed degrons, to signal substrates for degradation. A need exists for the development of methods that efficiently and accurately assess the structural basis of E3 ligase degron recognition and identify proteins capable of being targeted for degradation by the E3 ligase machinery.

### SUMMARY

The E3 ubiquitin ligase complex ubiquitinates many other proteins and can be manipulated with small molecules to trigger targeted degradation of specific substrate proteins of interest, including proteins that are not naturally targeted for degradation. Binding of substrate proteins with the E3 ubiquitin ligase complex is permitted if certain features, known as degrons, are present on the substrate proteins.

In some cases, binding of small molecules (e.g., molecular glues) to E3 ligase substrate receptors such as cereblon (CBRN) modulates the substrate selectivity of the complex, e.g., by changing the molecular surface of the E3 ligase substrate receptor protein, effectively hijacking the innate in vivo protein degradation system in order to degrade specific target proteins, e.g., for therapeutic effect (sometimes referred to as targeted protein degradation).

Molecular glues stabilize protein-protein interactions (e.g., between an E3 ligase substrate receptor protein and a neosubstrate), and, in cases where they lead to degradation of the neosubstrate, they are known as molecular glue degraders. Molecular glue degraders are a recently discovered therapeutic modality, with several clinically approved drugs (e.g. indisulam and lenalidomide), whose targets would have been otherwise considered undruggable. Molecular glue degraders have the potential to become the only modality capable of downregulating the large fraction of the proteome (>75%) considered undruggable using other approaches.

This raises the challenge of identifying neosubstrates and/or neosurfaces, in effect matching targets to particular E3 ligases, given a known or a yet unknown molecular glue. Thus, a critical need exists to identify neodegrons complementary to putative neosurfaces.

A need exists for alternative methods for the identification of target proteins (e.g., neosubstrates) capable of being targeted by E3 ligase machinery. Thus, described herein are, among other things, methods for the identification of target proteins capable of being targeted by E3 ligase machinery based on protein surface features.

Thus, described herein are, among other things, methods for the identification of substrate proteins capable of being targeted by E3 ligase machinery based on the protein molecular surface (quinary) representation of protein structure. The methods are useful, for example, in matching E3 ligases (e.g., an E3 ligase substrate receptor protein such as CRBN) to degrons (e.g., in target proteins), in the presence or absence of a molecular glue.

While degrons have been identified and described based on their primary and secondary structures (see, e.g., WO2022/153220), the use of surface features (the quinary protein structure) to identify degrons has not been performed in the art. The methods described herein provide, for the first time, the identification of degrons based on their surface features. The methods described herein are useful, for example, to identify degrons independently of their underlying primary sequence and secondary structure, based on how similar their molecular surface is to known degrons (degron mimicry) and/or their complementary to an E3 ligase substrate receptor protein surface or E3 ligase substrate receptor protein neosurface (e.g., induced by a molecular glue) (E3 complementarity).

The ability to identify degrons in this manner allows for the identification of degrons in completely unrelated proteins with no underlying structural similarity.

Thus, provided herein are methods for generating a degron similarity score for one or more protein(s), comprising: a) providing a first set of molecular surface features from a first set of one or more protein(s) comprising one or more known degron(s) of an E3 ligase substrate receptor and/or one or more predicted degron(s) of the E3 ligase substrate receptor; b) providing a second set of molecular surface features from a second set of one or more protein(s); and c) calculating a similarity score for the protein(s) of the second set by comparing the first and second sets of molecular surface features.

Also provided herein are methods for identifying a predicted neosubstrate of an E3 ligase, comprising: a) calculating a degron similarity score for one or more protein(s), according to any of the methods described herein; and b) based on the similarity score, identifying one or more of the protein(s) of the second set as a predicted neosubstrate(s) of the E3 ligase.

Also provided herein are methods for identifying a putative neosubstrate of an E3 ligase, comprising: a) identifying a predicted neosubstrate using any of the methods described herein; b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and c) if, based on said testing or having tested, the predicted neosubstrate is not determined to be a substrate of the E3 ligase, identifying the predicted neosubstrate as a putative neosubstrate of the E3 ligase.

Also provided herein are methods for classifying protein(s) as substrate(s) and/or putative neosubstrate(s) of an E3 ligase, comprising: a) calculating a degron similarity score for one or more protein(s) according to any of the methods described herein; b) based on the similarity score, identifying the protein(s) of the second set as a predicted neosubstrate of the E3 ligase or not; and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) i) if, based on said testing or having tested, the predicted neosubstrate is determined to be a substrate of the E3 ligase, classifying the predicted neosubstrate as a substrate of the E3 ligase; else ii) if, based on said testing or having tested, the predicted neosubstrate is not determined to be a substrate of the E3 ligase, classifying the predicted neosubstrate as a putative neosubstrate of the E3 ligase, thereby classifying protein(s) as substrate(s) and/or putative neosubstrate(s) of an E3 ligase.

Also provided herein are methods for selecting putative neosubstrate(s) of an E3 ligase from a set of potential neosubstrates, comprising: a) calculating a degron similarity score for one or more protein(s) according to any of the methods described herein; b) based on the similarity score, identifying a subset of the potential neosubstrates as predicted neosubstrate(s); and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates, thereby selecting putative neosubstrate(s) of an E3 ligase from a set of potential neosubstrates.

In some embodiments, the E3 ligase substrate detection assay is selected from the group consisting of a proximity assay, a binding assay, and a degradation assay.

In some embodiments: (i) the E3 ligase substrate detection assay is a proximity assay and the predicted neosubstrate is determined to be a substrate of the E3 ligase if an interaction between the putative neosubstrate and E3 ligase is detected; (ii) the E3 ligase substrate detection assay is a binding assay and the predicted neosubstrate is determined to be a substrate of the E3 ligase if binding of the neosubstrate and E3 ligase is detected; or (iii) the E3 ligase substrate detection assay is a degradation assay and the predicted neosubstrate is determined to be a substrate of the E3 ligase if degradation of the predicted neosubstrate is detected.

In some embodiments, the method comprises: testing or having tested a putative neosubstrate identified, classified, or selected by the method of any one of the methods described herein in an E3 ligase substrate detection assay with a binding modulator of the E3 ligase, and, if, based on said testing or having tested, the putative neosubstrate is determined to be a substrate of the E3 ligase in the presence of the E3 ligase binding modulator, identifying the putative neosubstrate as a neosubstrate of the E3 ligase. In some embodiments: (i) the E3 ligase substrate detection assay is a proximity assay and the putative neosubstrate is determined to be a substrate of the E3 ligase in the presence of the E3 ligase binding modulator if an interaction between the putative neosubstrate and E3 ligase is detected; (ii) the E3 ligase substrate detection assay is a binding assay and the putative neosubstrate is determined to be a substrate of the E3 ligase in the presence of the E3 ligase binding modulator if binding of the neosubstrate and E3 ligase is detected; or (iii) the E3 ligase substrate detection assay is a degradation assay and the putative neosubstrate is determined to be a substrate of the E3 ligase in the presence of the E3 ligase binding modulator if degradation of the predicted neosubstrate is detected.

In some embodiments, the one or more degron(s) is selected from the group consisting of N-degrons, C-degrons, phosphodegrons, oxygen-dependent degrons, G-loop degrons, and combinations thereof. In some embodiments, the degron(s) are N-degrons, C-degrons, phosphodegrons, oxygen-dependent degrons, or G-loop degrons. In some embodiments, the G-loop degron(s): (i) comprise or consist of the amino acid sequence X¹-X²-X³-X⁴-G-X⁶, wherein: each of X¹, X², X³, X⁴, and X⁶ are independently selected from any one of the natural occurring amino acids; and G (i.e. X⁵) is glycine; (ii) comprise or consists of the amino acid sequence X¹-X²-X³-X⁴-G-X⁶-X⁷, wherein: each of X¹, X², X³, X⁴, X⁶, and X⁷ are independently selected from any one of the natural occurring amino acids; and G (i.e. X⁵) is glycine; (iii) comprise or consists of the amino acid sequence X¹-X²-X³-X⁴-G-X⁶-X⁷-X⁸; wherein: each of X¹, X², X³, X⁴, X⁶, X⁷, and X⁸ are independently selected from any one of the natural occurring amino acids; and G (i.e. X⁵) is glycine; (iv) comprise or consists of the amino acid sequence X¹-X²-X³-X⁴-G-X⁶, wherein X¹ is selected from the group consisting of asparagine, aspartic acid, and cysteine; X² is selected from the group consisting of isoleucine, lysine, and asparagine; X³ is selected from the group consisting of threonine, lysine, and glutamine; X⁴ is selected from the group consisting of asparagine, serine, and cysteine; X⁵ is glycine; and X⁶ is selected from the group consisting of glutamic acid and glutamine; (v) comprise or consists of the amino acid sequence X¹-X²-X³-X⁴-G-X⁶, wherein X¹ is asparagine; X² is isoleucine; X³ is threonine; X⁴ is asparagine; X⁵ is glycine; and X⁶ is glutamic acid; (vi) comprise or consists of the amino acid sequence X¹-X²-X³-X⁴-G-X⁶, wherein X¹ is aspartic acid; X² is lysine; X³ is lysine; X⁴ is serine; X⁵ is glycine; and X⁶ is glutamic acid; and/or (vii) comprise or consists of the amino acid sequence X¹-X²-X³-X⁴-G-X⁶, wherein X¹ is cysteine; X² is asparagine; X³ is glutamine; X⁴ is cysteine; X⁵ is glycine; and X⁶ is glutamine.

In some embodiments, the degron(s): (i) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (ii) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid; (iii) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine; (iv) comprise or consists of the amino acid motif ETGE (SEQ ID NO: 1); (v) comprise or consists of the amino acid motif DLG; (vi) comprise or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine. In some cases the degron comprises or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; and/or (vii) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the E3 ligase comprises an E3 ligase substrate receptor protein selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

In some embodiments, the E3 ligase binding modulator is a compound shown in Table 1 or Table 2, or a pharmaceutically acceptable salt thereof, or a stereoisomer thereof.

In some embodiments, the second set of one or more protein(s) or set of potential neosubstrates comprises or consists of one or more of the proteins in Table 3.

In some embodiments: (i) the E3 ligase comprises the E3 ligase substrate receptor CRBN and the degron(s) are G-loop degron(s); (ii) the E3 ligase comprises the E3 ligase substrate receptor BTRC and the degron(s) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (iii) the E3 ligase comprises the E3 ligase substrate receptor KEAP1 and the degron(s) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid; (iv) the E3 ligase comprises the E3 ligase substrate receptor KEAP1 and the degron(s) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine; (v) the E3 ligase comprises the E3 ligase substrate receptor KEAP1 and the degron(s) comprise or consists of the amino acid motif ETGE ((SEQ ID NO: 1) and/or DLG; (vi) the E3 ligase comprises the E3 ligase substrate receptor MDM2 and the degron(s) comprise or consists of the amino acid motif X¹-X²-X³-X⁴-X⁵-X⁶-X⁷-X⁸, wherein X¹ is phenylalanine; X² is any one of the naturally occurring amino acids; X3 is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine; (vii) the E3 ligase comprises the E3 ligase substrate receptor MDM2 and the degron(s) comprise or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; or (viii) the E3 ligase comprises the E3 ligase substrate receptor VHL and the degron(s) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the molecular surface features comprise geometric and/or chemical features. In some embodiments, the geometric features are selected from the group consisting of shape index, distance-dependent curvature, geodesic polar coordinates, radial (angular) coordinates, and combinations thereof. In some embodiments, the chemical features are selected from the group consisting of hydropathy index, continuum electrostatics, location of free electrons, location of free proton donors, and combinations thereof. In some embodiments, the similarity score is calculated using a geometric deep learning model. In some embodiments, the geometric deep learning model is a neural network. In some embodiments, the neural network is trained on complementarity of E3 ligase surface(s) to known degron surface(s). In some embodiments, the neural network is trained on similarity to known and/or predicted degron surface(s).

In some embodiments, the second set of proteins comprises proteins that are not in the first set of proteins. In some embodiments, the second set of proteins does not include any proteins from the first set of proteins.

In some embodiments, the first set of molecular surface features consists of molecular surface features from one or more protein(s) comprising one or more known degron(s) of an E3 ligase substrate receptor. In some embodiments, the first set of molecular surface features consists of molecular surface features from one or more protein(s) comprising one or more predicted degron(s) of the E3 ligase substrate receptor. In some embodiments, the first set of molecular surface features consists of molecular surface features from one or more protein(s) comprising one or more known degron(s) of an E3 ligase substrate receptor and molecular surface feature(s) of one or more protein(s) comprising one or more predicted degron(s) of the E3 ligase substrate receptor.

In some embodiments, the known degron(s) of an E3 ligase substrate receptor are derived from a crystal structure.

Also provided herein are methods for generating a degron complementarity score for one or more protein(s), comprising: a) providing a first set of molecular surface features from a first set of one or more protein(s) comprising one or more E3 ligase substrate receptor proteins; b) providing a second set of molecular surface features from a second set of one or more protein(s); and c) calculating a complementarity score for the protein(s) of the second set by comparing the first and second sets of molecular surface features.

Also provided herein are methods for identifying a predicted neosubstrate of an E3 ligase, comprising: a) calculating a degron complementarity score for one or more protein(s) according to any one of the methods described herein; and b) based on the complementarity score, identifying one or more of the protein(s) of the second set as a predicted neosubstrate(s) of the E3 ligase.

Also provided herein are methods for identifying a putative neosubstrate of an E3 ligase, comprising: a) identifying a predicted neosubstrate according to any one of the methods described herein; b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and c) if, based on said testing or having tested, the predicted neosubstrate is not determined to be a substrate of the E3 ligase, identifying the predicted neosubstrate as a putative neosubstrate of the E3 ligase.

Also provided herein are methods for classifying protein(s) as substrate(s) and/or putative neosubstrate(s) of an E3 ligase, comprising: a) calculating a degron complementarity score for one or more protein(s) according to any one of the methods described herein; b) based on the complementarity score, identifying the protein(s) of the second set as a predicted neosubstrate of the E3 ligase or not; and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) i) if, based on said testing or having tested, the predicted neosubstrate is determined to be a substrate of the E3 ligase, classifying the predicted neosubstrate as a substrate of the E3 ligase; else ii) if, based on said testing or having tested, the predicted neosubstrate is not determined to be a substrate of the E3 ligase, classifying the predicted neosubstrate as a putative neosubstrate of the E3 ligase, thereby classifying protein(s) as substrate(s) and/or putative neosubstrate(s) of an E3 ligase.

Also provided herein are methods for selecting putative neosubstrate(s) of an E3 ligase from a set of potential neosubstrates, comprising: a) calculating a degron complementarity score for one or more protein(s) according to any one of the methods described herein; b) based on the complementarity score, identifying a subset of the potential neosubstrates as predicted neosubstrate(s); and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates, thereby selecting putative neosubstrate(s) of an E3 ligase from a set of potential neosubstrates.

In some embodiments, the E3 ligase substrate detection assay is selected from the group consisting of a proximity assay, a binding assay, and a degradation assay. In some embodiments: (i) the E3 ligase substrate detection assay is a proximity assay and the predicted neosubstrate is determined to be a substrate of the E3 ligase if an interaction between the putative neosubstrate and E3 ligase is detected; (ii) the E3 ligase substrate detection assay is a binding assay and the predicted neosubstrate is determined to be a substrate of the E3 ligase if binding of the neosubstrate and E3 ligase is detected; or (iii) the E3 ligase substrate detection assay is a degradation assay and the predicted neosubstrate is determined to be a substrate of the E3 ligase if degradation of the predicted neosubstrate is detected.

Also provided herein are methods of identifying a neosubstrate of an E3 ligase, comprising: testing or having tested a putative neosubstrate identified, classified, or selected by the method of any one of the methods described herein in an E3 ligase substrate detection assay with a binding modulator of the E3 ligase, and, if, based on said testing or having tested, the putative neosubstrate is determined to be a substrate of the E3 ligase in the presence of the E3 ligase binding modulator, identifying the putative neosubstrate as a neosubstrate of the E3 ligase.

In some embodiments: (i) the E3 ligase substrate detection assay is a proximity assay and the putative neosubstrate is determined to be a substrate of the E3 ligase in the presence of the E3 ligase binding modulator if an interaction between the putative neosubstrate and E3 ligase is detected; (ii) the E3 ligase substrate detection assay is a binding assay and the putative neosubstrate is determined to be a substrate of the E3 ligase in the presence of the E3 ligase binding modulator if binding of the neosubstrate and E3 ligase is detected; or (iii) the E3 ligase substrate detection assay is a degradation assay and the putative neosubstrate is determined to be a substrate of the E3 ligase in the presence of the E3 ligase binding modulator if degradation of the predicted neosubstrate is detected.

In some embodiments, the one or more degron(s) is selected from the group consisting of N-degrons, C-degrons, phosphodegrons, oxygen-dependent degrons, G-loop degrons, and combinations thereof. In some embodiments, the degron(s) are N-degrons, C-degrons, phosphodegrons, oxygen-dependent degrons, or G-loop degrons. In some embodiments, the G-loop degron(s): (i) comprise or consist of the amino acid sequence X¹-X²-X³-X⁴-G-X⁶, wherein: each of X¹, X², X³, X⁴, and X⁶ are independently selected from any one of the natural occurring amino acids; and G (i.e. X⁵) is glycine; (ii) comprise or consists of the amino acid sequence X¹-X²-X³-X⁴-G-X⁶-X⁷, wherein: each of X¹, X², X³, X⁴, X⁶, and X⁷ are independently selected from any one of the natural occurring amino acids; and G (i.e. X⁵) is glycine; (iii) comprise or consists of the amino acid sequence X¹-X²-X³-X⁴-G-X⁶-X⁷-X⁸; wherein: each of X¹, X², X³, X⁴, X⁶, X⁷, and X⁸ are independently selected from any one of the natural occurring amino acids; and G (i.e. X⁵) is glycine; (iv) comprise or consists of the amino acid sequence X¹-X²-X³-X⁴-G-X⁶, wherein X¹ is selected from the group consisting of asparagine, aspartic acid, and cysteine; X² is selected from the group consisting of isoleucine, lysine, and asparagine; X³ is selected from the group consisting of threonine, lysine, and glutamine; X⁴ is selected from the group consisting of asparagine, serine, and cysteine; X⁵ is glycine; and X⁶ is selected from the group consisting of glutamic acid and glutamine; (v) comprise or consists of the amino acid sequence X¹-X²-X³-X⁴-G-X⁶, wherein X¹ is asparagine; X² is isoleucine; X³ is threonine; X⁴ is asparagine; X⁵ is glycine; and X⁶ is glutamic acid; (vi) comprise or consists of the amino acid sequence X¹-X²-X³-X⁴-G-X⁶, wherein X¹ is aspartic acid; X² is lysine; X³ is lysine; X⁴ is serine; X⁵ is glycine; and X⁶ is glutamic acid; and/or (vii) comprise or consists of the amino acid sequence X¹-X²-X³-X⁴-G-X⁶, wherein X¹ is cysteine; X² is asparagine; X³ is glutamine; X⁴ is cysteine; X⁵ is glycine; and X⁶ is glutamine.

In some embodiments, the degron(s): (i) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (ii) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid; (iii) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine; (iv) comprise or consists of the amino acid motif ETGE (SEQ ID NO: 1); (v) comprise or consists of the amino acid motif DLG; (vi) comprise or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine. In some cases the degron comprises or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; and/or (vii) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the E3 ligase comprises an E3 ligase substrate receptor protein selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

In some embodiments, the E3 ligase binding modulator is a compound shown in Table 1 or Table 2, or a pharmaceutically acceptable salt thereof, or a stereoisomer thereof.

In some embodiments, the second set of one or more protein(s) or set of potential neosubstrates comprises or consists of one or more of the proteins in Table 3.

In some embodiments: (i) the E3 ligase comprises the E3 ligase substrate receptor CRBN and the degron(s) are G-loop degron(s); (ii) the E3 ligase comprises the E3 ligase substrate receptor BTRC and the degron(s) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (iii) the E3 ligase comprises the E3 ligase substrate receptor KEAP1 and the degron(s) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid;
(iv) the E3 ligase comprises the E3 ligase substrate receptor KEAP1 and the degron(s) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine;
(v) the E3 ligase comprises the E3 ligase substrate receptor KEAP1 and the degron(s) comprise or consists of the amino acid motif ETGE ((SEQ ID NO: 1) and/or DLG;
(vi) the E3 ligase comprises the E3 ligase substrate receptor MDM2 and the degron(s) comprise or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine; (vii) the E3 ligase comprises the E3 ligase substrate receptor MDM2 and the degron(s) comprise or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; or (viii) the E3 ligase comprises the E3 ligase substrate receptor VHL and the degron(s) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the molecular surface features comprise geometric and/or chemical features. In some embodiments, the geometric features are selected from the group consisting of shape index, distance-dependent curvature, geodesic polar coordinates, radial (angular) coordinates, and combinations thereof. In some embodiments, the chemical features are selected from the group consisting of hydropathy index, continuum electrostatics, location of free electrons, location of free proton donors, and combinations thereof. In some embodiments, the complementarity score is calculated using a geometric deep learning model. In some embodiments, the geometric deep learning model is a neural network. In some embodiments, the neural network is trained on complementarity of E3 ligase surface(s) to known degron surface(s). In some embodiments, the neural network is trained on similarity to known and/or predicted degron surface(s).

In some embodiments, the second set of proteins comprises proteins that are not in the first set of proteins. In some embodiments, the second set of proteins does not include any proteins from the first set of proteins.

Also provided herein are methods for generating a degron score for one or more protein(s), comprising: a) providing a set of molecular surface features from a set of one or more protein(s); and c) calculating a degron score for the protein(s) by comparing the molecular surface features to a reference set of molecular surface(s).

Also provided herein are methods for identifying a predicted neosubstrate of an E3 ligase, comprising: a) calculating a degron score for one or more protein(s) according to any one of the methods described herein; and b) based on the degron score, identifying one or more of the protein(s) of the second set as a predicted neosubstrate(s) of the E3 ligase.

Also provided herein are methods for identifying a putative neosubstrate of an E3 ligase, comprising: a) identifying a predicted neosubstrate according to any one of the methods described herein; b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and c) if, based on said testing or having tested, the predicted neosubstrate is not determined to be a substrate of the E3 ligase, identifying the predicted neosubstrate as a putative neosubstrate of the E3 ligase.

Also described herein are methods for classifying protein(s) as substrate(s) and/or putative neosubstrate(s) of an E3 ligase, comprising: a) calculating a degron score for one or more protein(s) according to any one of the methods described herein; b) based on the degron score, identifying the protein(s) of the second set as a predicted neosubstrate of the E3 ligase or not; and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) i) if, based on said testing or having tested, the predicted neosubstrate is determined to be a substrate of the E3 ligase, classifying the predicted neosubstrate as a substrate of the E3 ligase; else ii) if, based on said testing or having tested, the predicted neosubstrate is not determined to be a substrate of the E3 ligase, classifying the predicted neosubstrate as a putative neosubstrate of the E3 ligase, thereby classifying protein(s) as substrate(s) and/or putative neosubstrate(s) of an E3 ligase.

Also provided herein are methods for selecting putative neosubstrate(s) of an E3 ligase from a set of potential neosubstrates, comprising: a) calculating a degron score for one or more protein(s) according to any one of the methods described herein; b) based on the degron score, identifying a subset of the potential neosubstrates as predicted neosubstrate(s); and c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates, thereby selecting putative neosubstrate(s) of an E3 ligase from a set of potential neosubstrates.

In some embodiments, the E3 ligase substrate detection assay is selected from the group consisting of a proximity assay, a binding assay, and a degradation assay. In some embodiments: (i) the E3 ligase substrate detection assay is a proximity assay and the predicted neosubstrate is determined to be a substrate of the E3 ligase if an interaction between the putative neosubstrate and E3 ligase is detected; (ii) the E3 ligase substrate detection assay is a binding assay and the predicted neosubstrate is determined to be a substrate of the E3 ligase if binding of the neosubstrate and E3 ligase is detected; or (iii) the E3 ligase substrate detection assay is a degradation assay and the predicted neosubstrate is determined to be a substrate of the E3 ligase if degradation of the predicted neosubstrate is detected.

Also provided herein are methods of identifying a neosubstrate of an E3 ligase, comprising: testing or having tested a putative neosubstrate identified, classified, or selected by the method of any one of the methods described herein in an E3 ligase substrate detection assay with a binding modulator of the E3 ligase, and, if, based on said testing or having tested, the putative neosubstrate is determined to be a substrate of the E3 ligase in the presence of the E3 ligase binding modulator, identifying the putative neosubstrate as a neosubstrate of the E3 ligase.

In some embodiments: (i) the E3 ligase substrate detection assay is a proximity assay and the putative neosubstrate is determined to be a substrate of the E3 ligase in the presence of the E3 ligase binding modulator if an interaction between the putative neosubstrate and E3 ligase is detected; (ii) the E3 ligase substrate detection assay is a binding assay and the putative neosubstrate is determined to be a substrate of the E3 ligase in the presence of the E3 ligase binding modulator if binding of the neosubstrate and E3 ligase is detected; or (iii) the E3 ligase substrate detection assay is a degradation assay and the putative neosubstrate is determined to be a substrate of the E3 ligase in the presence of the E3 ligase binding modulator if degradation of the predicted neosubstrate is detected.

In some embodiments, the one or more degron(s) is selected from the group consisting of N-degrons, C-degrons, phosphodegrons, oxygen-dependent degrons, G-loop degrons, and combinations thereof. In some embodiments, the degron(s) are N-degrons, C-degrons, phosphodegrons, oxygen-dependent degrons, or G-loop degrons. In some embodiments, the G-loop degron(s): (i) comprise or consist of the amino acid sequence X¹-X²-X³-X⁴-G-X⁶, wherein: each of X¹, X², X³, X⁴, and X⁶ are independently selected from any one of the natural occurring amino acids; and G (i.e. X⁵) is glycine; (ii) comprise or consists of the amino acid sequence X¹-X²-X³-X⁴-G-X⁶-X⁷, wherein: each of X¹, X², X³, X⁴, X⁶, and X⁷ are independently selected from any one of the natural occurring amino acids; and G (i.e. X⁵) is glycine; (iii) comprise or consists of the amino acid sequence X¹-X²-X³-X⁴-G-X⁶-X⁷-X⁸; wherein: each of X¹, X², X³, X⁴, X⁶, X⁷, and X⁸ are independently selected from any one of the natural occurring amino acids; and G (i.e. X⁵) is glycine; (iv) comprise or consists of the amino acid sequence X¹-X²-X³-X⁴-G-X⁶, wherein X¹ is selected from the group consisting of asparagine, aspartic acid, and cysteine; X² is selected from the group consisting of isoleucine, lysine, and asparagine; X³ is selected from the group consisting of threonine, lysine, and glutamine; X⁴ is selected from the group consisting of asparagine, serine, and cysteine; X⁵ is glycine; and X⁶ is selected from the group consisting of glutamic acid and glutamine; (v) comprise or consists of the amino acid sequence X¹-X²-X³-X⁴-G-X⁶, wherein X¹ is asparagine; X² is isoleucine; X³ is threonine; X⁴ is asparagine; X⁵ is glycine; and X⁶ is glutamic acid; (vi) comprise or consists of the amino acid sequence X¹-X²-X³-X⁴-G-X⁶, wherein X¹ is aspartic acid; X² is lysine; X³ is lysine; X⁴ is serine; X⁵ is glycine; and X⁶ is glutamic acid; and/or (vii) comprise or consists of the amino acid sequence X¹-X²-X³-X⁴-G-X⁶, wherein X¹ is cysteine; X² is asparagine; X³ is glutamine; X⁴ is cysteine; X⁵ is glycine; and X⁶ is glutamine.

In some embodiments, the degron(s): (i) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (ii) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid; (iii) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine; (iv) comprise or consists of the amino acid motif ETGE (SEQ ID NO: 1); (v) comprise or consists of the amino acid motif DLG; (vi) comprise or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine. In some cases the degron comprises or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; and/or (vii) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the E3 ligase comprises an E3 ligase substrate receptor protein selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

In some embodiments, the E3 ligase binding modulator is a compound shown in Table 1 or Table 2, or a pharmaceutically acceptable salt thereof, or a stereoisomer thereof.

In some embodiments, the second set of one or more protein(s) or set of potential neosubstrates comprises or consists of one or more of the proteins in Table 3.

In some embodiments: (i) the E3 ligase comprises the E3 ligase substrate receptor CRBN and the degron(s) are G-loop degron(s); (ii) the E3 ligase comprises the E3 ligase substrate receptor BTRC and the degron(s) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid; (iii) the E3 ligase comprises the E3 ligase substrate receptor KEAP1 and the degron(s) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid; (iv) the E3 ligase comprises the E3 ligase substrate receptor KEAP1 and the degron(s) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine; (v) the E3 ligase comprises the E3 ligase substrate receptor KEAP1 and the degron(s) comprise or consists of the amino acid motif ETGE ((SEQ ID NO: 1) and/or DLG; (vi) the E3 ligase comprises the E3 ligase substrate receptor MDM2 and the degron(s) comprise or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine; (vii) the E3 ligase comprises the E3 ligase substrate receptor MDM2 and the degron(s) comprise or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; or (viii) the E3 ligase comprises the E3 ligase substrate receptor VHL and the degron(s) comprise or consists of the amino acid motif X¹-X²-X³-X⁴-X⁵-X⁶, wherein X is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some embodiments, the molecular surface features comprise geometric and/or chemical features. In some embodiments, the geometric features are selected from the group consisting of shape index, distance-dependent curvature, geodesic polar coordinates, radial (angular) coordinates, and combinations thereof. In some embodiments, the chemical features are selected from the group consisting of hydropathy index, continuum electrostatics, location of free electrons, location of free proton donors, and combinations thereof. In some embodiments, the degron score is calculated using a geometric deep learning model. In some embodiments, the geometric deep learning model is a neural network. In some embodiments, the neural network is trained on complementarity of E3 ligase surface(s) to known degron surface(s). In some embodiments, the neural network is trained on similarity to known and/or predicted degron surface(s).

In some embodiments, the second set of proteins comprises proteins that are not in the first set of proteins. In some embodiments, the second set of proteins does not include any proteins from the first set of proteins.

In some embodiments of any of the methods described herein, the E3 ligase is CRBN.

Throughout this application, various embodiments may be presented in a range format. It should be understood that the description in range format is merely for convenience and brevity and should not be construed as an inflexible limitation on the scope of the disclosure.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1C show an overview of the MaSIF conceptual framework, implementation and applications. FIG. 1A shows: Left, conceptual representation of a protein surface engraved with an interaction fingerprint, surface features that may reveal their potential biomolecular interactions. Right, surface segmentation into overlapping radial patches of a fixed geodesic radius used in MaSIF. FIG. 1B shows: Top, the patches comprise geometric and chemical features mapped on the protein surface; Bottom left: polar geodesic coordinates used to map the position of the features within the patch; Bottom right: MaSIF uses geometric deep learning tools to apply CNNs to the data. Fingerprint descriptors are computed for each patch using application-specific neural network architectures, which contain reusable building blocks (geodesic convolutional layers). FIG. 1C shows MaSIF applications.

FIGS. 2A-2E show an example of a method for prediction of protein-protein interactions (PPIs) based on surface fingerprints. FIG. 2A shows an overview of the MaSIF-search neural network optimization (Siamese architecture) to output fingerprint descriptors, such that the descriptors of interacting patches are similar, while those of non-interacting patches are dissimilar. The features of the target patch (with the exception of the hydropathy features) are inverted to enable the minimization of the fingerprint distance. FIG. 2B shows the distribution of fingerprint distances showing interacting and non-interacting patches for the test set (13338 positive pairs and 13338 negative pairs). MaSIF-search was trained and tested on both geometric and chemical features. FIG. 2C shows a comparison of the performance between different fingerprint features shown in ROC AUC (13338 positive pairs and 13338 negative pairs from test set). GIF: ROC AUC for GIF fingerprint descriptors; Geom: MaSIF-search trained with only geometric features; Chem: MaSIF-search only with chemical features; G+C: geometry and chemistry features. FIG. 2D shows a schematic of MaSIF-search workflow showing the 3 stages of the protocol (top) and MaSIF-search benchmarking by performing a large-scale docking of N binder proteins to N known targets with site information (bottom). FIG. 2E shows the results from the benchmarking shown in FIG. 2D: number of solved complexes for MaSIF and other competing methods for holo structures (top); number of solved complexes in apo structures (bottom).

FIG. 3 shows an example of training a degron identification system based on surface patches.

FIG. 4 shows an example of using an ultra-fast fingerprint search for similar surfaces, finding surface that mimic known degron surfaces.

FIG. 5 depicts a surface for an ultra-fast fingerprint search for complementary surfaces, such as for E3 ligase—neosubstrate matchmaking.

FIG. 6 depicts an example of a method for learning CRBN degron features from known degron surfaces. The algorithm classifies protein surfaces for the presence of degrons. The algorithm creates a feature-rich surface characterization and uses 3 layers of geodesic convolution with deep vertexes to classify input surfaces.

FIG. 7 depicts an example of a yeast-3-hybrid proximity assay. The assay identifies MGD-induced interactions between CRBN and cDNA library-derived targets. It maps degrons to individual domains.

FIG. 8 shows that 8 novel G-loops from 5 distinct domain classes, identified using yeast 3 hybrid experiments, match predictions made by a method for learning CRBN degron features from known degron surfaces.

FIG. 9 shows that a degron surface found and characterized using methods described herein has a unique G-loop surface; FIG. 10 shows that this enables selective MGD degradation.

FIG. 11 shows an example of encoding protein surfaces as fingerprints, which enables ultra-fast, proteome-wide searching for similar & complementary fingerprints for degron identification.

FIG. 12 shows an example of a multi-step pipeline.

FIG. 13 shows that the multi-step pipeline of FIG. 12 enables ultra-fast searching of, for example, proteome-wide queries of either complementary or similar surfaces to either E3 ligase surfaces or degron surfaces respectively.

FIG. 14 shows an example of proteome-wide fast matching of degron surface mimics by matching of surface fingerprints (and not, e.g., G-loops per se).

FIG. 15 shows an example of a novel degron identified by a mimicry search. The degron is a non-hairpin, non-canonical degron in an established oncology target.

FIG. 16 shows that NanoBRET confirmed the prediction and binding mode shown in FIG. 15.

FIG. 17 is an example of how the E3 ligase neosurface footprint can be used to find novel neosubstrates (as it defines the target-complementary surface).

FIG. 18 shows an example of a method for finding proteins complementary to E3 ligases. In this example, the E3 ligase footprint is encoded as a fingerprint for fast E3-target matchmaking.

FIG. 19 shows an example of how the methods described herein expand the target space to non-canonical degrons.

### DETAILED DESCRIPTION

Described herein are methods and compounds useful, for example, for predicting, identifying, classifying, and selecting neosubstrates of E3 ligases using, for example, molecular surface features of protein(s). The molecular surface is a higher-level representation of protein structure than protein structure or sequence and the methods described herein provide an improvement, for example, over methods utilizing lower level representation(s) of protein structure.

### E3 Ligases and E3 Ligase Substrate Receptors

E3 ligases recognize protein substrates and, when complexed with E2 conjugating enzymes loaded with ubiquitin, results in ubiquitination of the protein. E3 ligases and their substrate receptor proteins are known and described in the art, for example, in Ishida et al., “E3 Ligase Ligands for PROTACs: How They Were Found and How to Discover New Ones,” SLAS Discovery 26(4):484-502 (2021).

Cereblon (CRBN), for example, forms an E3 ubiquitin ligase complex with damaged DNA binding protein 1 (DDB1), Cullin-4A (CUL4A), and regulator of cullins 1 (ROC1).

In some cases, the E3 ligase substrate receptor protein is an E3 ligase substrate receptor protein selected from the group consisting of CRBN (e.g., UniProtKB Q96SW2), VHL (e.g., UniProtKB P40337), BIRC1 (e.g., UniProtKB Q13075), BIRC2 (e.g., UniProtKB Q13490), BIRC3 (e.g., UniProtKB Q13489), BIRC4 (e.g., UniProtKB P98170), BIRC5 (e.g., UniProtKB O15392), BIRC6 (e.g., UniProtKB Q9NR09), BIRC7 (e.g., UniProtKB Q96CA5), BIRC8 (e.g., UniProtKB Q96P09), KEAP1 (e.g., UniProtKB Q14145), DCAF15 (e.g., UniProtKB Q66K64), RNF4 (e.g., UniProtKB P78317) RNF4 isoform 2 (e.g., UniProtKB P78317-2), RNF114 (e.g., UniProtKB Q9Y508), RNF114 isoform 2 (e.g., UniProtKB Q9Y508-2), DCAF16 (e.g., UniProtKB Q9NXF7) AHR (e.g., UniProtKB P35869), MDM2 (e.g., UniProtKB Q00987), UBR2 (e.g., UniProtKB Q8IWV8), SPOP (e.g., UniProtKB Q43791), KLHL3 (e.g., UniProtKB Q9UH77), KLHL12 (e.g., UniProtKB Q53G59), KLHL20 (e.g., UniProtKB Q9Y2M5), KLHDC2 (e.g., UniProtKB Q9Y2U9), SPSB1 (e.g., UniProtKB Q96BD6), SPSB2 (e.g., UniProtKB Q99619), SBSB4 (e.g., UniProtKB Q96A44), SOCS2 (e.g., UniProtKB O14508), SOCS6 (e.g., UniProtKB O14544), FBXO4 (e.g., UniProtKB Q9UKT5), FBXO31 (e.g., UniProtKB Q5XUX0), BTRC (e.g., UniProtKB Q9Y297), FBW7 (e.g., UniProtKB Q969H0), CDC20 (e.g., UniProtKB Q12834), ITCH (e.g., UniProtKB Q96J02), PML (e.g., UniProtKB P29590), TRIM21 (e.g., UniProtKB P19474), TRIM24 (e.g., UniProtKB O15164), TRIM33 (e.g., UniProtKB Q9UPN9), GID4 (e.g., UniProtKB Q8IVV7), and DCAF11 (e.g., UniProtKB Q8TEB1).

In some cases, the E3 ligase is an E3 ligase selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

In some cases, the E3 ligase is at least 80%, e.g., at least 90%, at least 95%, or at least 99% identical to an E3 ligase selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

In some cases, the E3 ligase is an enzymatically active portion of an E3 ligase selected from the group consisting of CRBN (SEQ ID NO: 3), CRBN isoform 2 (SEQ ID NO: 2), VHL (SEQ ID NO: 9), BIRC1 (SEQ ID NO: 10), BIRC2 (SEQ ID NO: 11), BIRC3 (SEQ ID NO: 12), BIRC4 (SEQ ID NO: 13), BIRC5 (SEQ ID NO: 14), BIRC6 (SEQ ID NO: 15), BIRC7 (SEQ ID NO: 16), BIRC8 (SEQ ID NO: 17), KEAP1 (SEQ ID NO: 18), DCAF15 (SEQ ID NO: 19), RNF4 (SEQ ID NO: 20) RNF4 isoform 2 (SEQ ID NO: 21), RNF114 (SEQ ID NO: 22), RNF114 isoform 2 (SEQ ID NO: 23), DCAF16 (SEQ ID NO: 24) AHR (SEQ ID NO: 25), MDM2 (SEQ ID NO: 26), UBR2 (SEQ ID NO: 27), SPOP (SEQ ID NO: 28), KLHL3 (SEQ ID NO: 29), KLHL12 (SEQ ID NO: 30), KLHL20 (SEQ ID NO: 31), KLHDC2 (SEQ ID NO: 32), SPSB1 (SEQ ID NO: 33), SPSB2 (SEQ ID NO: 34), SBSB4 (SEQ ID NO: 35), SOCS2 (SEQ ID NO: 36), SOCS6 (SEQ ID NO: 37), FBXO4 (SEQ ID NO: 38), FBXO31 (SEQ ID NO: 39), BTRC (SEQ ID NO: 40), FBW7 (SEQ ID NO: 41), CDC20 (SEQ ID NO: 42), ITCH (SEQ ID NO: 43), PML (SEQ ID NO: 44), TRIM21 (SEQ ID NO: 45), TRIM24 (SEQ ID NO: 46), TRIM33 (SEQ ID NO: 47), GID4 (SEQ ID NO: 48), and DCAF11 (SEQ ID NO: 49).

### Cereblon

The cereblon protein, encoded by the gene CRBN, is the substrate recognition component of a DCX (DDB1-CUL4-X-box) E3 protein ligase complex that mediates the ubiquitination and subsequent proteasomal degradation of target proteins.

The hydrophobic tri-tryptophan cage is the canonical thalidomide-binding domain at the C-terminal end of CRBN. The glutarimide moiety of immunomodulatory imide drugs (IMiDs) such as thalidomide bind into this high conserved hydrophobic pocket, with the phthalamide ring exposed on the surface of the CRBN protein. See Chopra et al., “Protein Degradation for Drug Discovery,” Drug Discovery Today: Technologies 31:5-13 (2019).

The human cereblon protein (NCBI Gene ID 51185; UniProt ID Q96SW2) encodes the following transcripts and isoforms, of which NM_016302.4 (SEQ ID NO: 3, transcript 1) is the canonical transcript:

| Transcript | Length (nt) | Protein | Length (aa) | SEQ ID NO: | Isoform |
|---|---|---|---|---|---|
| XR_940448.3 | 2667 | | | | |
| XM_011533791.3 | 3586 | XP_011532093.1 | 398 | SEQ ID NO: 5 | X1 |
| XM_011533793.2 | 2927 | XP_011532095.1 | 278 | SEQ ID NO: 6 | X4 |
| XM_011533794.2 | 2798 | XP_011532096.1 | 278 | SEQ ID NO: 7 | X4 |
| NM_001173482.1 | 2593 | NP_001166953.1 | 441 | SEQ ID NO: 2 | 2 |
| XM_005265202.4 | 2472 | XP_005265259.1 | 379 | SEQ ID NO: 4 | X2 |
| NM_016302.4 | 2187 | NP_057386.2 | 442 | SEQ ID NO: 3 | 1 |
| XM_024453551.1 | 1458 | XP_024309319.1 | 284 | SEQ ID NO: 8 | X3 |

Isoform 1 of human CRBN (SEQ ID NO: 3) has the following features:

| Feature | Position(s) | Reference |
|---|---|---|
| Zinc binding | 323 | Chamberlain et al. Nat. Struct. Mol. |
| Zinc binding | 326 | Biol. 21: 803-9 (2014) |
| Zinc binding | 391 | |
| Zinc binding | 394 | |

Known mutants of human CRBN isoform 1 (SEQ ID NO: 3) have the following features:

| Feature | Posi- | | |
|---|---|---|---|
| key | tion(s) | Description | Reference(s) |
| Muta- | 384 | Y → A: Abolishes | Ito et al., Science |
| genesis | | thalidomide-binding without | 327: 1345-50 (2010) |
| | | affecting DCX protein ligase | |
| | | complex activity; when | |
| | | associated with A-386. | |
| Muta- | 386 | W → A: Abolishes | Ito et al., Science |
| genesis | | thalidomide-binding without | 327: 1345-50 (2010); |
| | | affecting DCX protein ligase | Chamberlain et al. |
| | | complex activity; when | Nat. Struct. Mol. |
| | | associated with A-384. | Biol. 21: 803-9 (2014) |
| | | Abolishes pomalidomide- | |
| | | induced change in substrate | |
| | | specificity and abolishes | |
| | | pomalidomide-induced | |
| | | decrease in cell viability that | |
| | | is brought about by increased | |
| | | degradation of MYC, IRF4 | |
| | | and IKZF3. | |
| Muta- | 419-442 | Missing: Fails to rescue | Choi et al., J. |
| genesis | | increased BK channel activity | Neurosci. 38: |
| | | and decreased probability of | 3571-83 (2018) |
| | | neurotransmission in a mouse | |
| | | hippocampal neuron model. | |

Isoform 1 of human CRBN (SEQ ID NO: 3) comprises a Lon N-terminal domain at positions 81-317, the canonical binding domain CULT (cereblon domain of unknown activity, binding cellular Ligands and; Thalomide) at positions 318-426, and canonical thalomide binding region at positions 378-386 (Chamberlain et al. Nat. Struct. Mol. Biol. 21:803-9 (2014)). The CULT domain binds thalidomide and related drugs, such as pomalidomide and lenalidomide. Drug binding leads to a change in substrate specificity of the human DCX (DDB1-CUL4-X-box) E3 protein ligase complex, while no such change is observed in rodents (Chamberlain et al. Nat. Struct. Mol. Biol. 21:803-9 (2014)).

In some cases, the cereblon protein is human cereblon protein. In some cases, the cereblon protein comprises or consists of SEQ ID NO: 2, SEQ ID NO: 3, SEQ ID NO: 4, SEQ ID NO: 5, SEQ ID NO: 6, SEQ ID NO: 7, or SEQ ID NO: 8. In some cases, the cerebelon protein is at least 80% identical to SEQ ID NO: 2, SEQ ID NO: 3, SEQ ID NO: 4, SEQ ID NO: 5, SEQ ID NO: 6, SEQ ID NO: 7, or SEQ ID NO: 8, e.g., at least 9000, at least 9500 or at least 99% identical to SEQ ID NO: 2, SEQ ID NO: 3, SEQ ID NO: 4, SEQ ID NO: 5, SEQ ID NO: 6, SEQ ID NO: 7, or SEQ ID NO: 8.

In some cases, the cereblon protein is human cereblon protein without the leading methionine (M). In some cases, the cereblon protein comprises or consists of SEQ ID NO: 2, SEQ ID NO: 3, SEQ ID NO: 4, SEQ ID NO: 5, SEQ ID NO: 6, SEQ ID NO: 7, or SEQ ID NO: 8 without the leading methionine (M). In some cases, the cerebelon protein is at least 800% identical to SEQ ID NO: 2, SEQ ID NO: 3, SEQ ID NO: 4, SEQ ID NO: 5, SEQ ID NO: 6, SEQ ID NO: 7, or SEQ ID NO: 8 without the leading methionine (M), e.g., at least 90%, at least 95% or at least 99% identical to SEQ ID NO: 2, SEQ ID NO: 3, SEQ ID NO: 4, SEQ ID NO: 5, SEQ ID NO: 6, SEQ ID NO: 7, or SEQ ID NO: 8 without the leading methionine (M).

In some cases, the cereblon protein is a mutant that is unable to bind compounds, e.g., an E3 ligase binding modulator, e.g., a cereblon binding modulator described herein, at a canonical binding site.

In some cases, the cereblon protein, e.g., a cereblon protein described herein, comprises point mutations at the positions corresponding to Y384 and/or W386 of SEQ ID NO: 3. In some cases, the cereblon protein, e.g., a cereblon protein described herein, comprises point mutations at the positions corresponding to Y384 and W386 of SEQ ID NO: 3. In some cases, the mutations are Y384A and/or W386A.

In some cases, the cereblon protein comprises or consists of SEQ ID NO: 3 with point mutations at Y384 and/or W386. In some cases, the cereblon protein comprises or consists of SEQ ID NO: 3 with point mutations at both Y384 and W386. In some cases, the mutations are Y384A and/or W386A.

### E3 Ligase Binding Modulators

The methods described herein are useful, for example, for identifying neosubstrates of E3 ligases. In some cases, the methods are used to validate and/or identify targets that selectively interact with, e.g., cereblon within the E3 ubiquitin ligase complex, in the presence of a compound, e.g., an E3 ligase binding modulator such as a molecular glue, e.g., a cereblon binding modulator such as a CRBN molecular glue.

E3 ligase binding modulators, e.g., cereblon binding modulators, are described, for example, in WO2021/069705, WO2021/053555, WO2022/152821, WO2022/219407, and WO2022219412, which are hereby incorporated by reference in their entirety.

In some cases, the E3 ligase binding modulator, e.g., cereblon binding modulator, is a compound shown in Table 1 or Table 2, or a pharmaceutically acceptable salt thereof, or a stereoisomer thereof.

TABLE 1
Cereblon Binding Modulators

| Compound | No. |
|---|---|
| 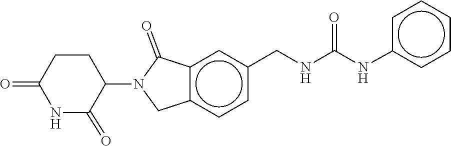 | 1 |
| 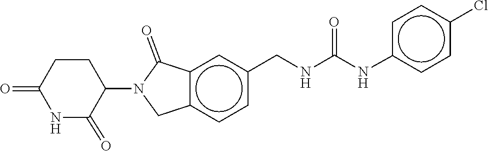 | 2 |
| 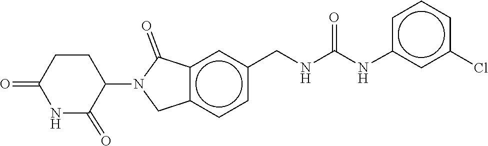 | 3 |
| 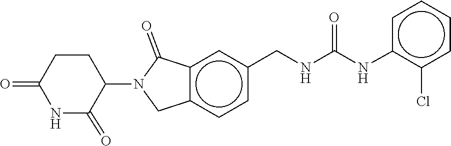 | 4 |
| 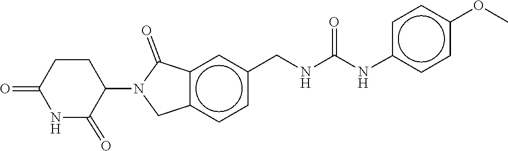 | 5 |
| 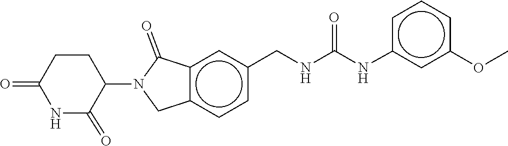 | 6 |
| 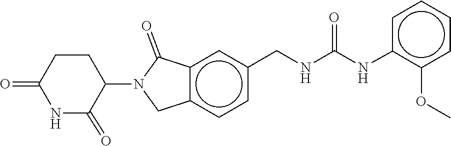 | 7 |
| 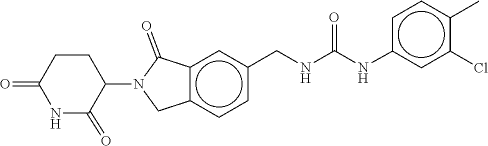 | 8 |
| 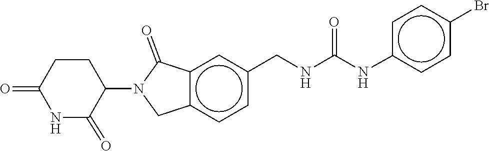 | 9 |
| 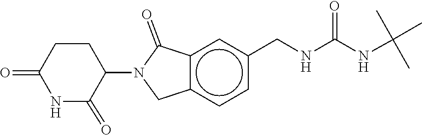 | 10 |
| 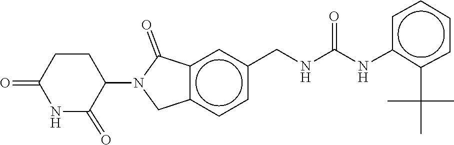 | 11 |
| 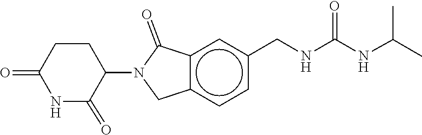 | 12 |
| 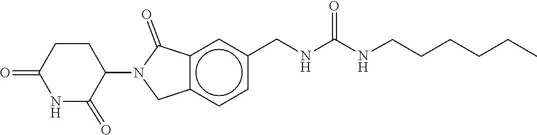 | 13 |
| 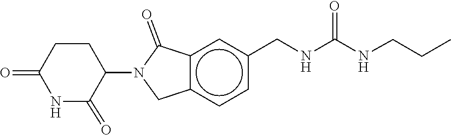 | 14 |
| 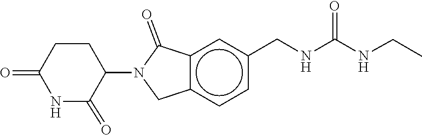 | 15 |
| 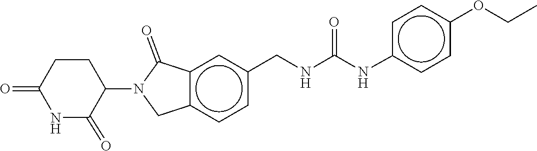 | 16 |
| 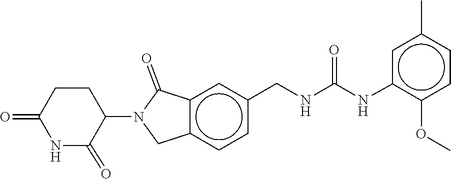 | 17 |
| 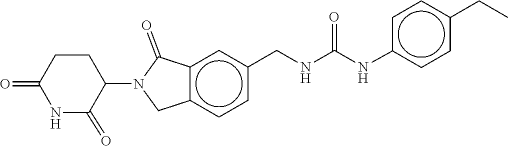 | 18 |
| 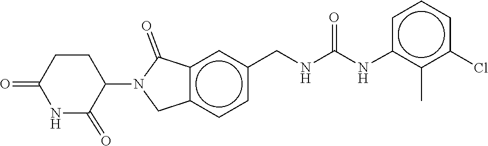 | 19 |
| 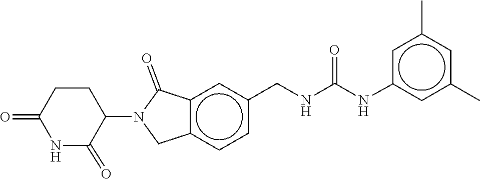 | 20 |
| 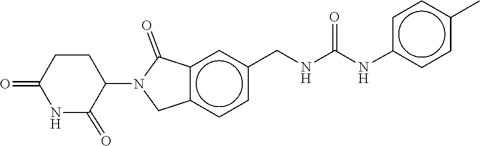 | 21 |
| 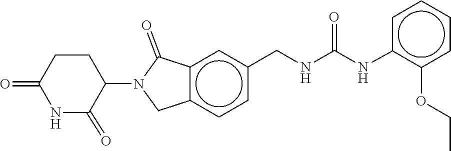 | 22 |
| 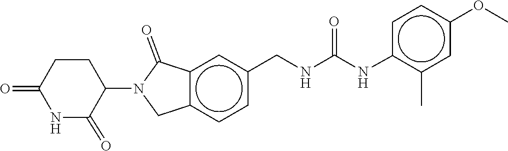 | 23 |
| 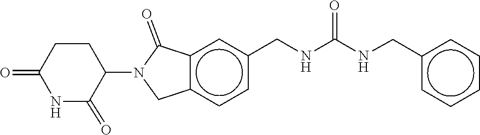 | 24 |
| 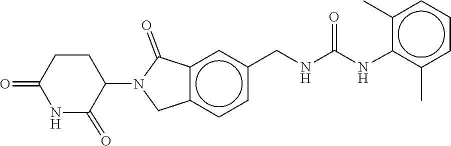 | 25 |
| 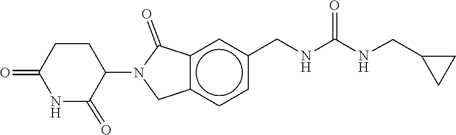 | 26 |
| 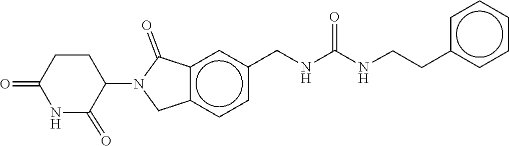 | 27 |
| 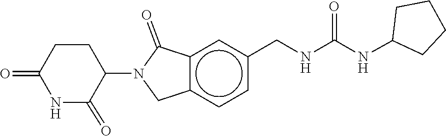 | 28 |
| 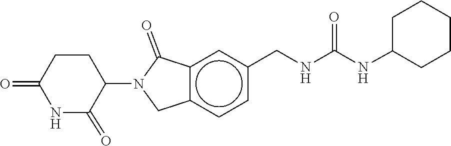 | 29 |
| 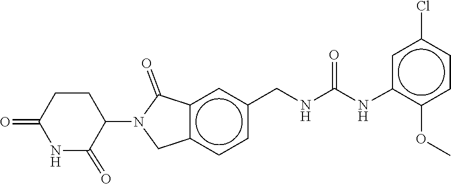 | 30 |
| 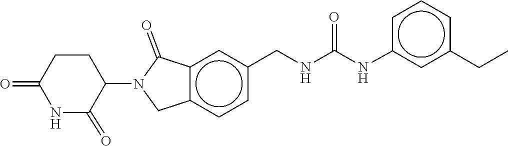 | 31 |
| 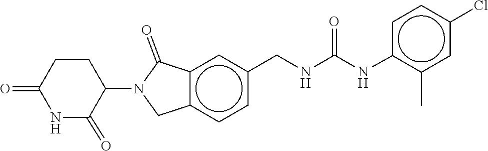 | 32 |
| 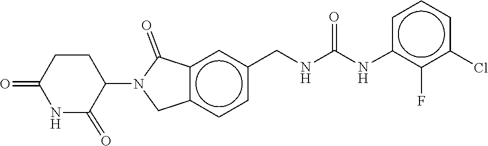 | 33 |
| 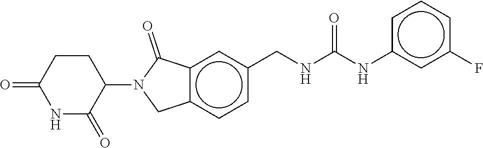 | 34 |
| 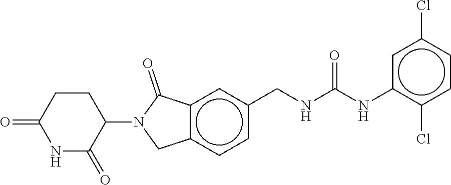 | 35 |
| 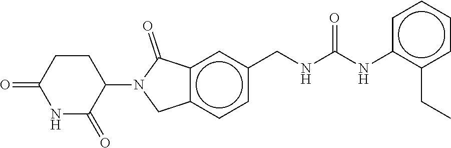 | 36 |
| 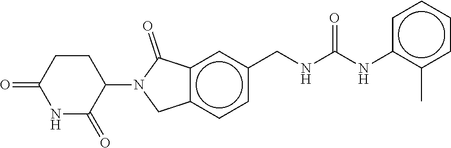 | 37 |
| 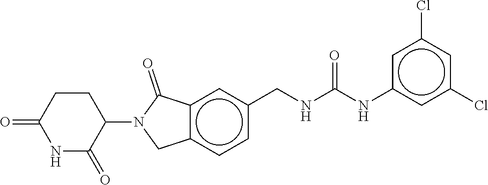 | 38 |
| 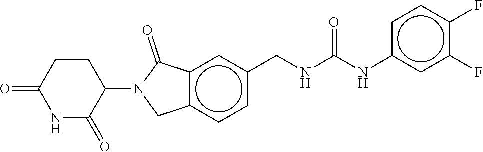 | 39 |
| 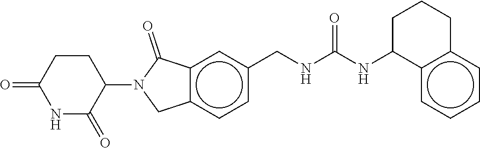 | 40 |
| 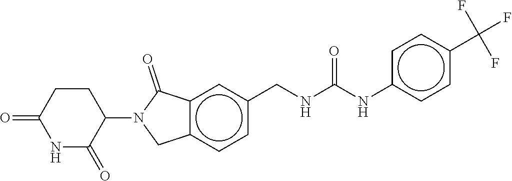 | 41 |
| 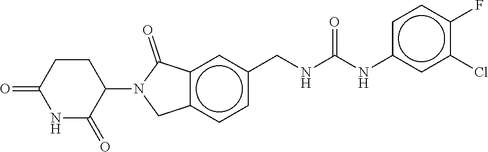 | 42 |
| 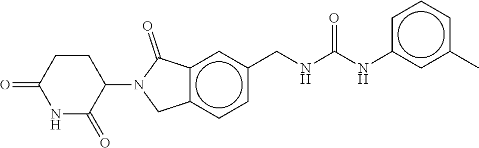 | 43 |
| 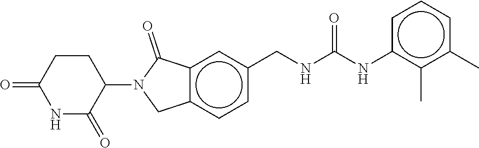 | 44 |
| 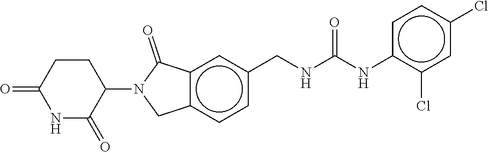 | 45 |
| 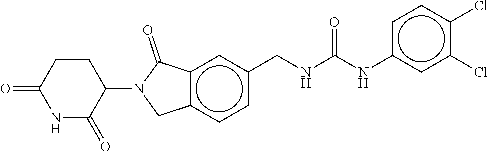 | 46 |
| 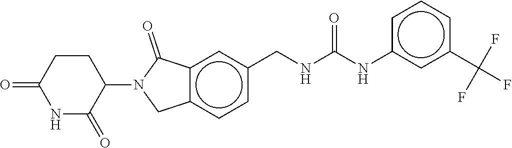 | 47 |
| 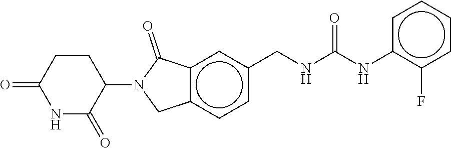 | 48 |
| 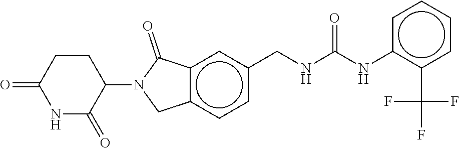 | 49 |
| 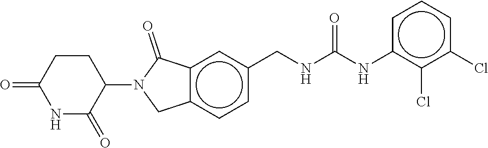 | 50 |
| 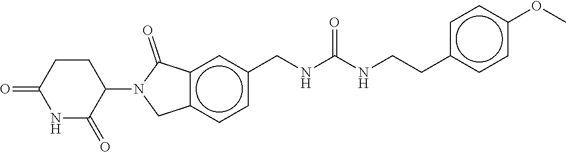 | 51 |
| 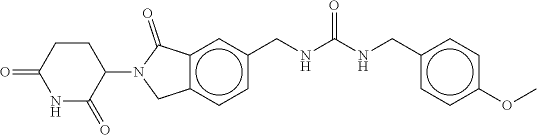 | 52 |
| 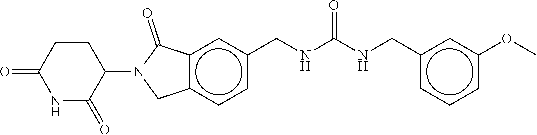 | 53 |
| 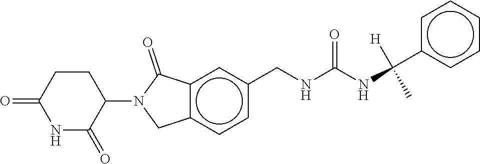 | 54 |
| 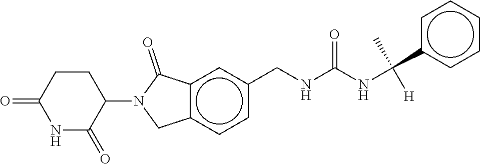 | 55 |
| 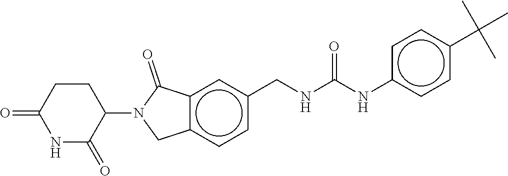 | 56 |
| 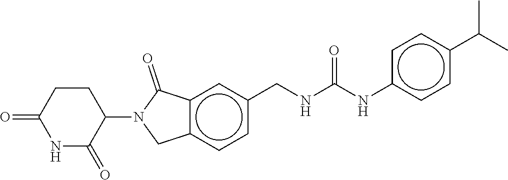 | 57 |
| 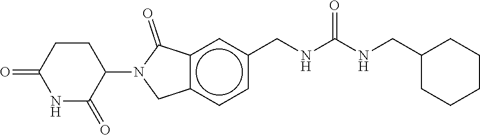 | 58 |
| 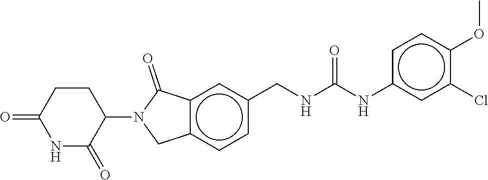 | 59 |
| 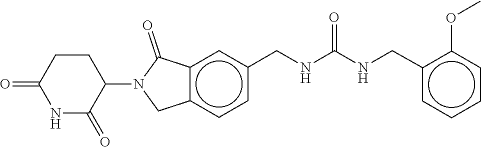 | 60 |
| 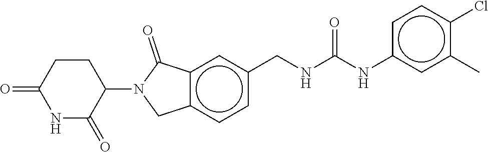 | 61 |
| 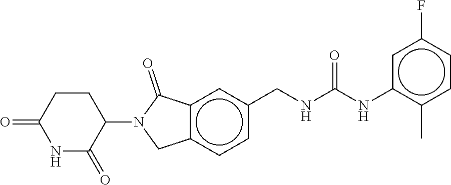 | 62 |
| 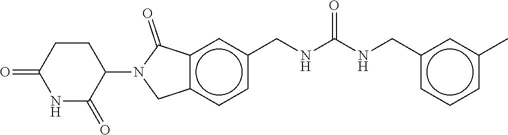 | 63 |
| 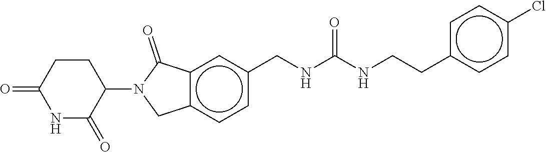 | 64 |
| 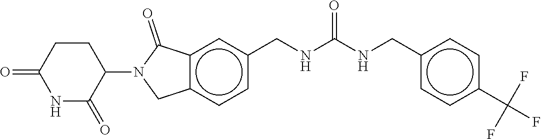 | 65 |
| 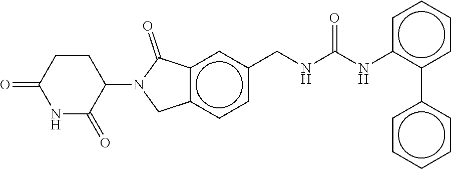 | 66 |
| 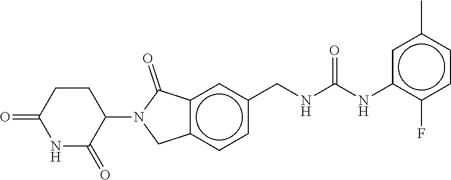 | 67 |
| 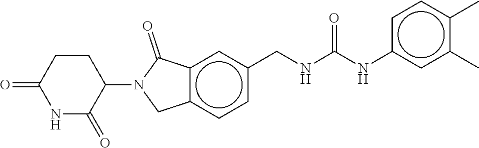 | 68 |
| 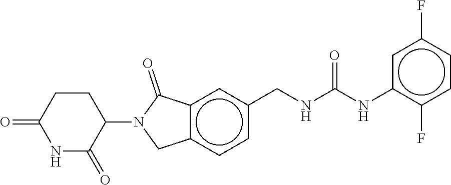 | 69 |
| 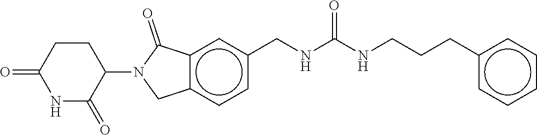 | 70 |
| 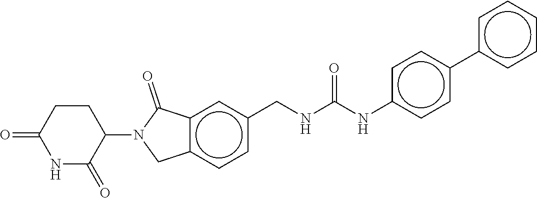 | 71 |
| 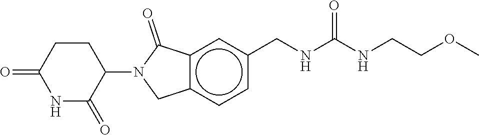 | 72 |
| 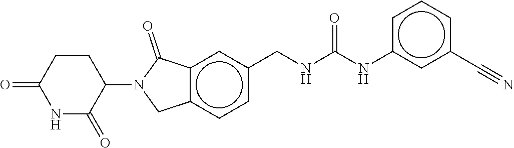 | 73 |
| 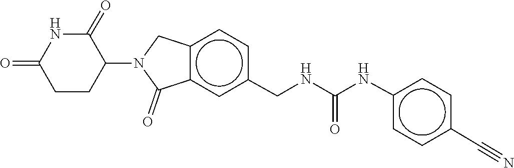 | 74 |
| 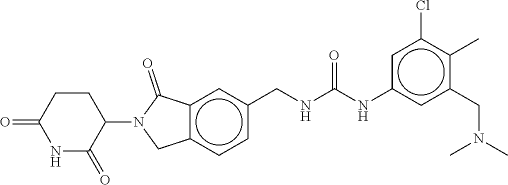 | 75 |
| 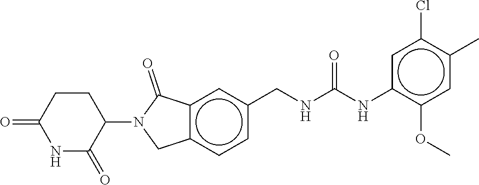 | 76 |
| 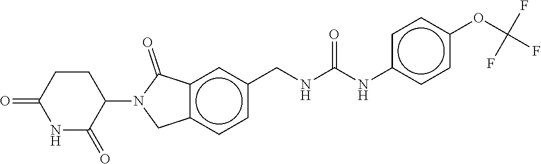 | 77 |
| 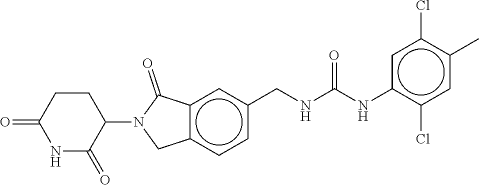 | 78 |
| 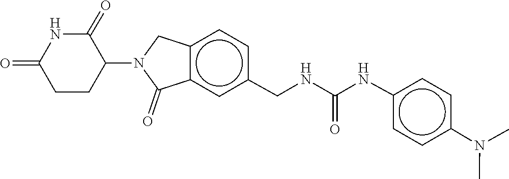 | 79 |
| 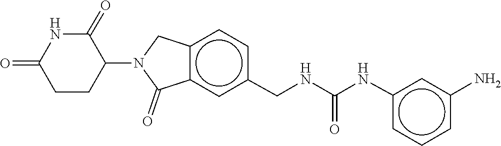 | 80 |
| 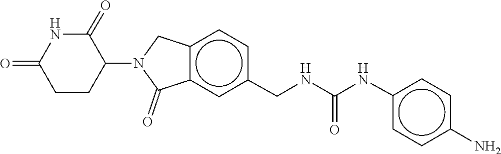 | 81 |
| 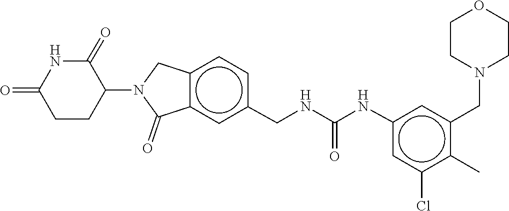 | 82 |
| 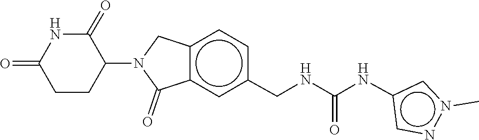 | 83 |
| 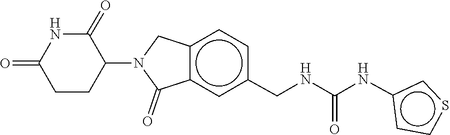 | 84 |
| 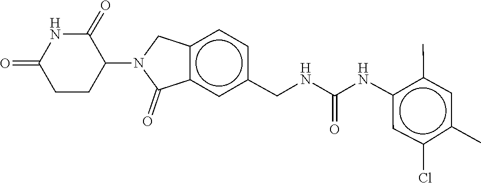 | 85 |
| 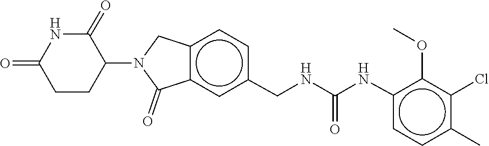 | 86 |
| 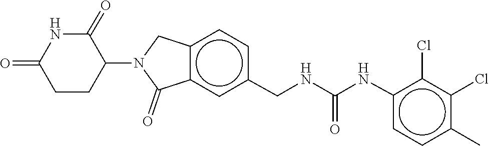 | 87 |
| 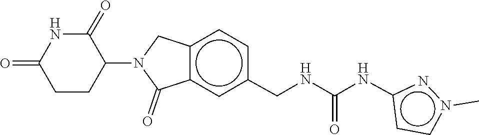 | 88 |
| 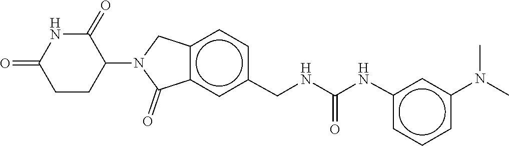 | 89 |
| 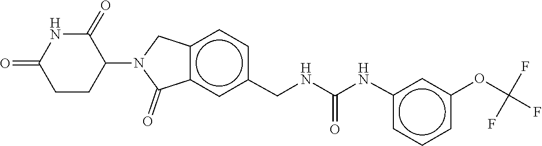 | 90 |
| 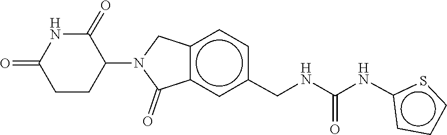 | 91 |
| 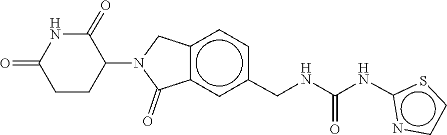 | 92 |
| 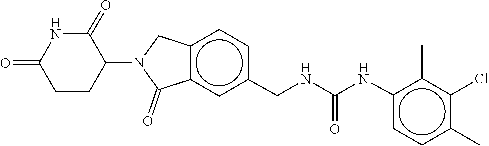 | 93 |
| 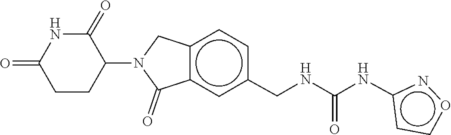 | 94 |
| 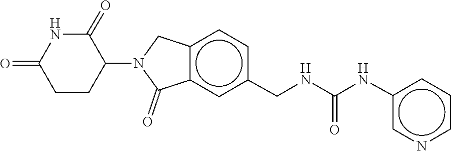 | 95 |
| 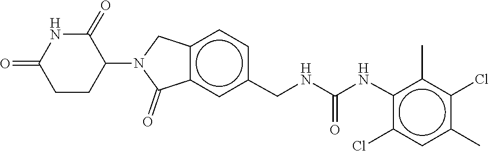 | 96 |
| 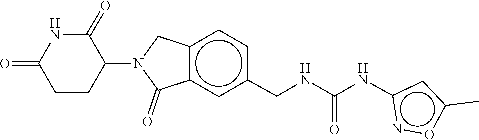 | 97 |
| 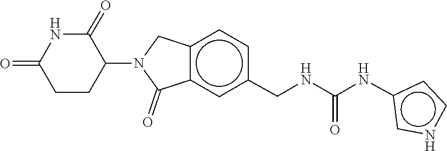 | 98 |
| 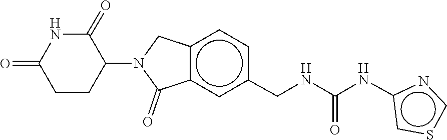 | 99 |
| 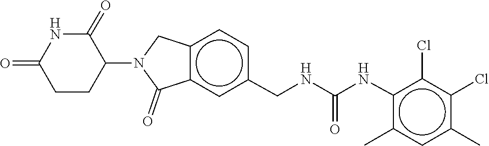 | 100 |
| 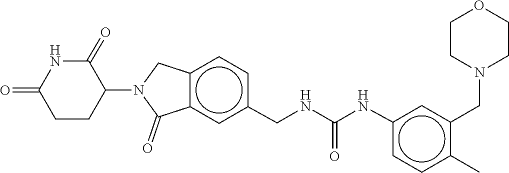 | 101 |
| 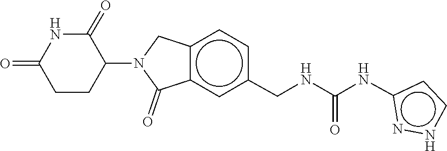 | 102 |
| 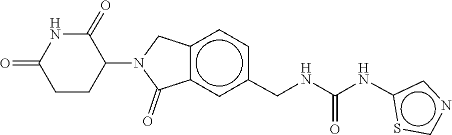 | 103 |
| 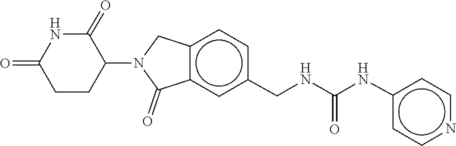 | 104 |
| 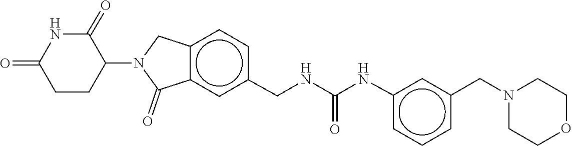 | 105 |
| 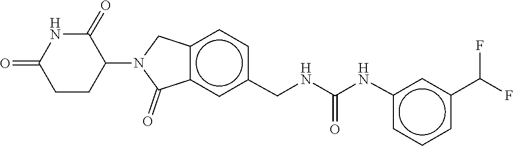 | 106 |
| 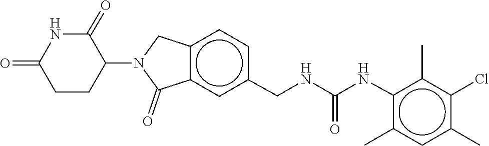 | 107 |
| 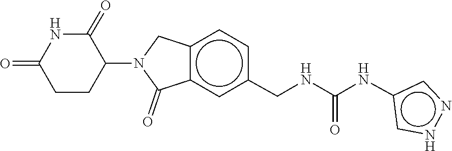 | 108 |
| 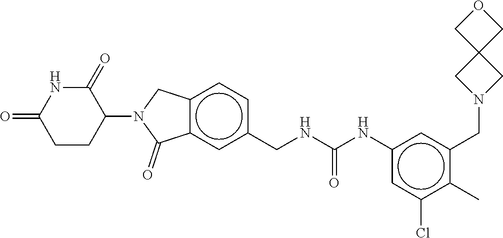 | 109 |
| 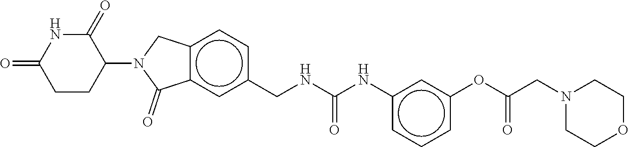 | 110 |
| 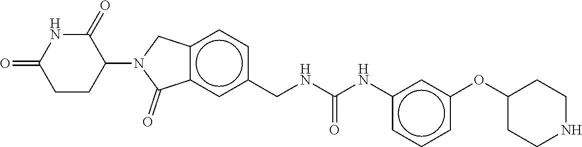 | 111 |
| 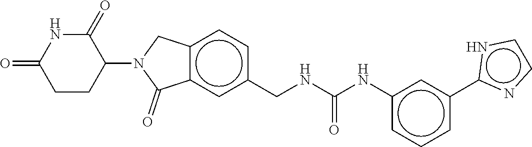 | 112 |
| 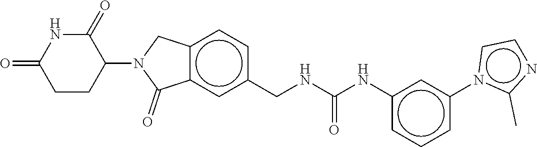 | 113 |
| 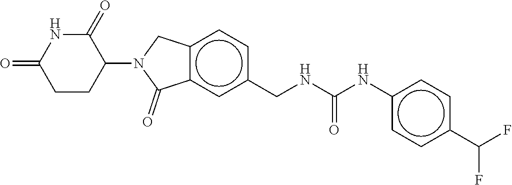 | 114 |
| 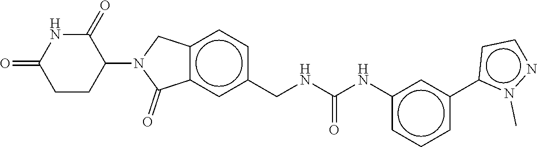 | 115 |
| 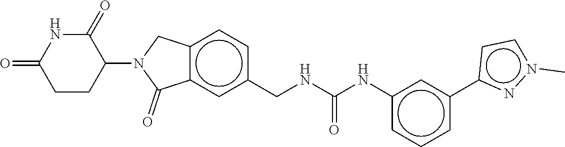 | 116 |
| 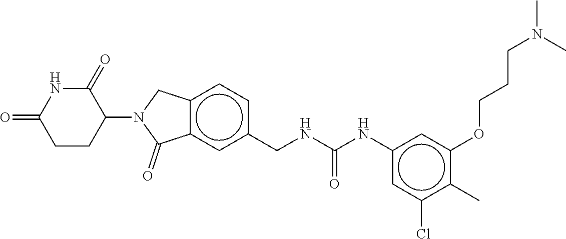 | 117 |
| 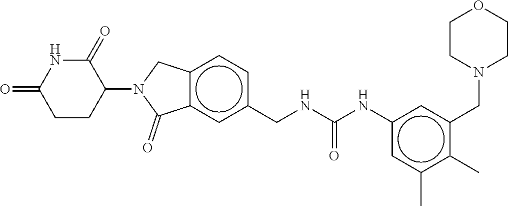 | 118 |
| 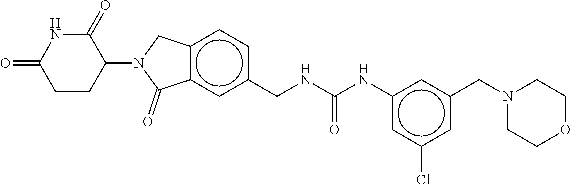 | 119 |
| 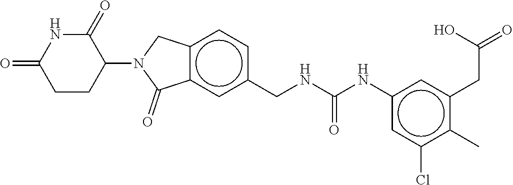 | 120 |
| 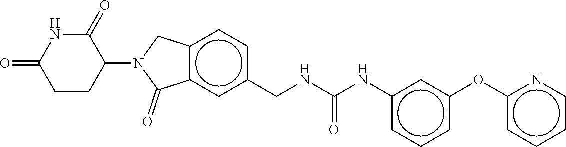 | 121 |
| 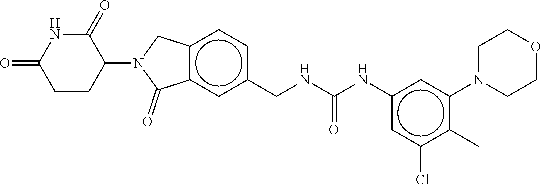 | 122 |
| 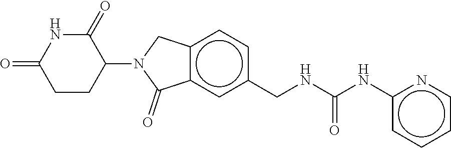 | 123 |
| 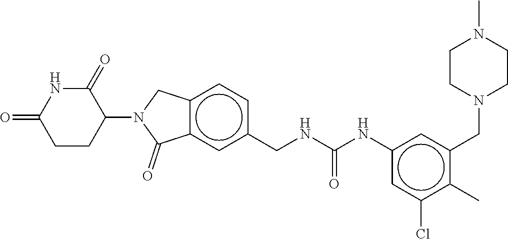 | 124 |
| 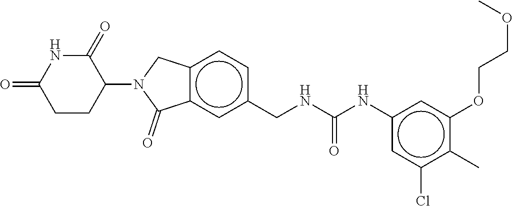 | 125 |
| 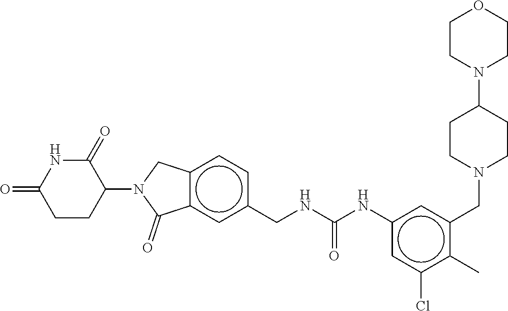 | 126 |
| 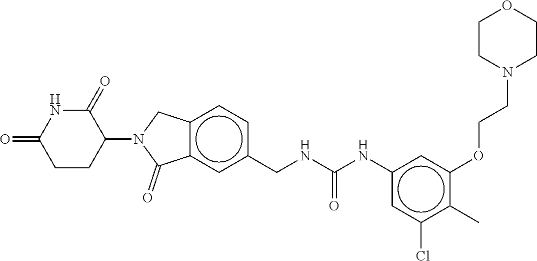 | 127 |
| 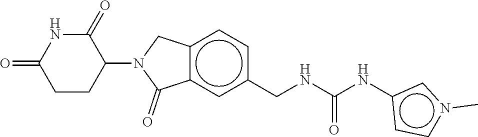 | 128 |
| 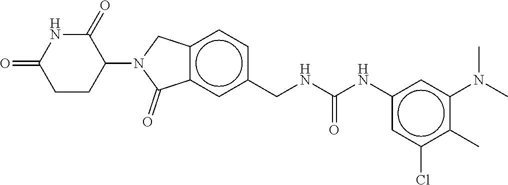 | 129 |
| 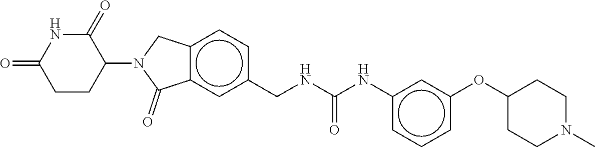 | 130 |
| 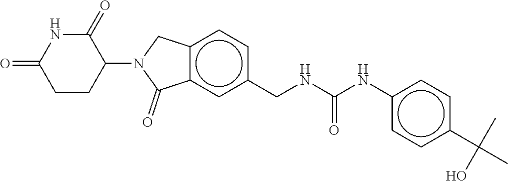 | 131 |
| 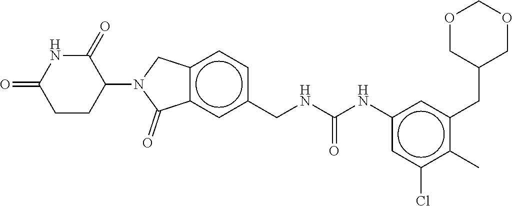 | 132 |
| 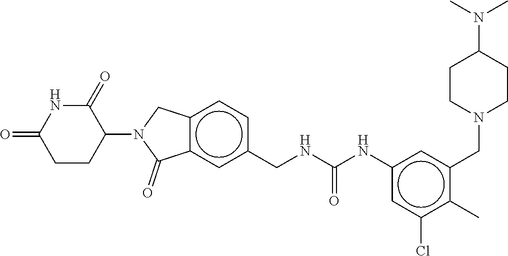 | 133 |
| 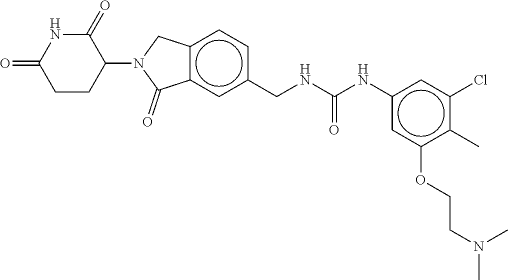 | 134 |
| 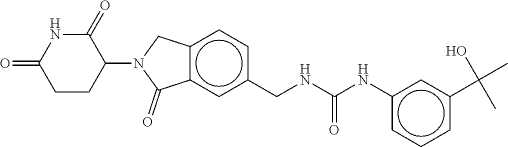 | 135 |
| 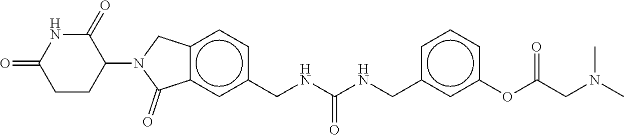 | 136 |
| 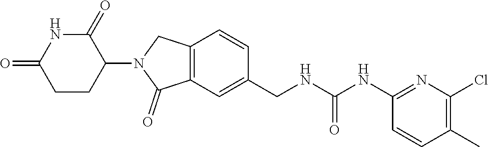 [SMILES: Cc1ccc(NC(=O)NCc2ccc3c(c2)C(=O)N(C2CCC(=O)NC2=O)C3)nc1Cl] | 137 |
| 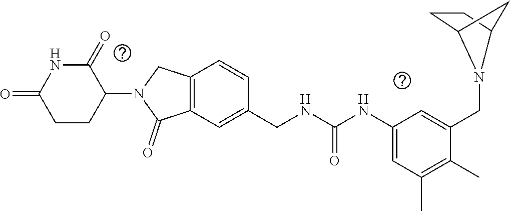 [SMILES: Cc1cc(NC(=O)NCc2ccc3c(c2)C(=O)N(C2CCC(=O)NC2=O)C3)cc(CN2C3CCC2C3)c1C] | 138 |
| 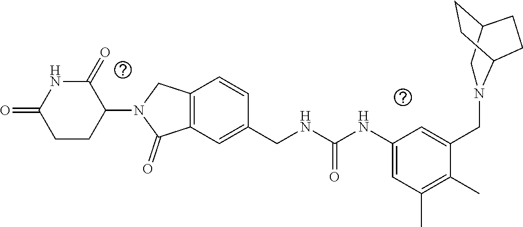 [SMILES: Cc1cc(NC(=O)NCc2ccc3c(c2)C(=O)N(C2CCC(=O)NC2=O)C3)cc(CN2CC3CCC2CC3)c1C] | 139 |
| 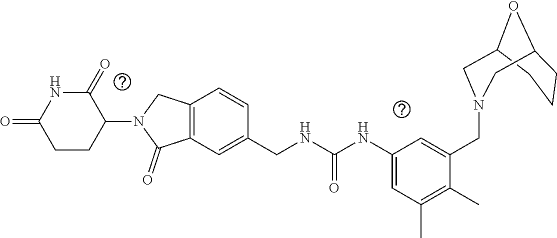 [SMILES: Cc1cc(NC(=O)NCc2ccc3c(c2)C(=O)N(C2CCC(=O)NC2=O)C3)cc(CN2CC3CCCC(C2)O3)c1C] | 140 |
| 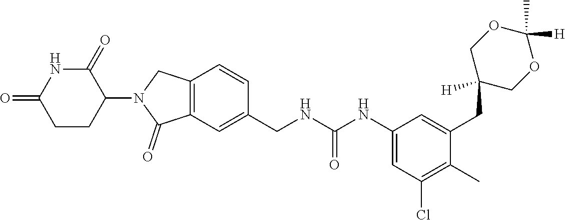 [SMILES: Cc1c(Cl)cc(NC(=O)NCc2ccc3c(c2)C(=O)N(C2CCC(=O)NC2=O)C3)cc1C[C@H]1CO[C@H](C)OC1] | 141 |
| 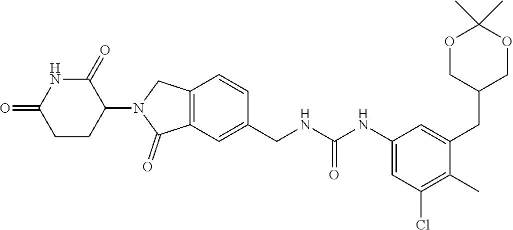 [SMILES: Cc1c(Cl)cc(NC(=O)NCc2ccc3c(c2)C(=O)N(C2CCC(=O)NC2=O)C3)cc1CC1COC(C)(C)OC1] | 142 |
| 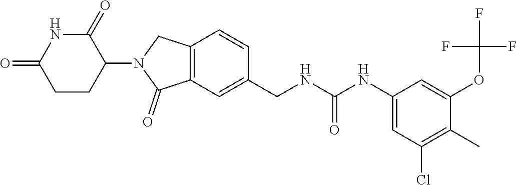 [SMILES: Cc1c(Cl)cc(NC(=O)NCc2ccc3c(c2)C(=O)N(C2CCC(=O)NC2=O)C3)cc1OC(F)(F)F] | 143 |
| 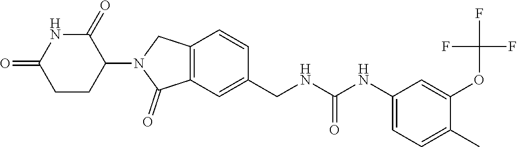 [SMILES: Cc1ccc(NC(=O)NCc2ccc3c(c2)C(=O)N(C2CCC(=O)NC2=O)C3)cc1OC(F)(F)F] | 144 |
| 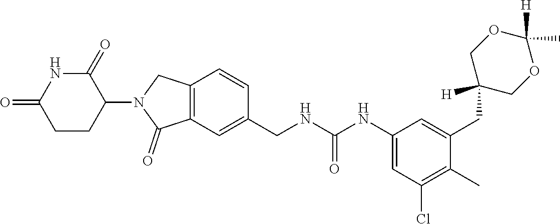 [SMILES: Cc1c(Cl)cc(NC(=O)NCc2ccc3c(c2)C(=O)N(C2CCC(=O)NC2=O)C3)cc1C[C@H]1CO[C@@H](C)OC1] | 145 |
| 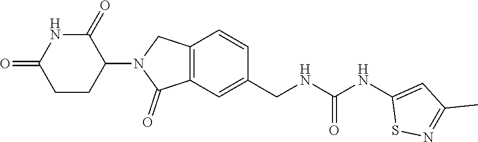 [SMILES: Cc1cc(NC(=O)NCc2ccc3c(c2)C(=O)N(C2CCC(=O)NC2=O)C3)sn1] | 146 |
| 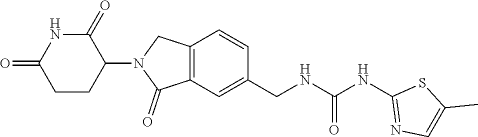 [SMILES: Cc1cnc(NC(=O)NCc2ccc3c(c2)C(=O)N(C2CCC(=O)NC2=O)C3)s1] | 147 |
| 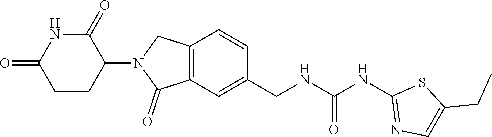 [SMILES: CCc1cnc(NC(=O)NCc2ccc3c(c2)C(=O)N(C2CCC(=O)NC2=O)C3)s1] | 148 |
| 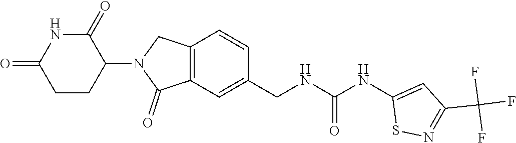 [SMILES: O=C1CCC(N2Cc3ccc(CNC(=O)Nc4cc(C(F)(F)F)ns4)cc3C2=O)C(=O)N1] | 149 |
| 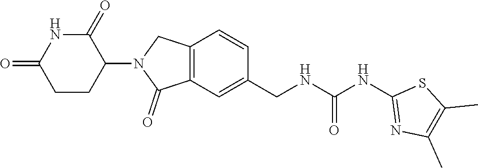 [SMILES: Cc1nc(NC(=O)NCc2ccc3c(c2)C(=O)N(C2CCC(=O)NC2=O)C3)sc1C] | 150 |
| 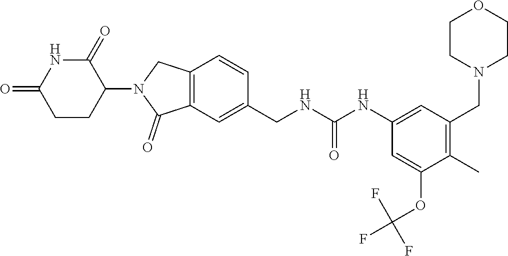 [SMILES: Cc1c(CN2CCOCC2)cc(NC(=O)NCc2ccc3c(c2)C(=O)N(C2CCC(=O)NC2=O)C3)cc1OC(F)(F)F] | 151 |
| 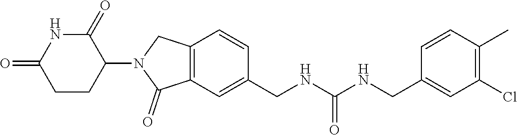 [SMILES: Cc1ccc(CNC(=O)NCc2ccc3c(c2)C(=O)N(C2CCC(=O)NC2=O)C3)cc1Cl] | 152 |
| 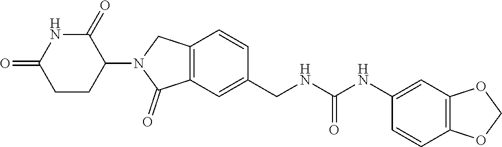 [SMILES: O=C1CCC(N2Cc3ccc(CNC(=O)Nc4ccc5c(c4)OCO5)cc3C2=O)C(=O)N1] | 153 |
| 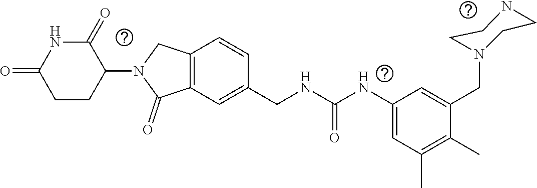 [SMILES: Cc1cc(NC(=O)NCc2ccc3c(c2)C(=O)N(C2CCC(=O)NC2=O)C3)cc(CN2CCNCC2)c1C] | 154 |
| 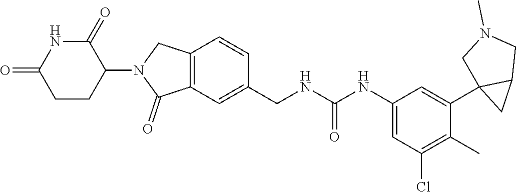 [SMILES: Cc1c(Cl)cc(NC(=O)NCc2ccc3c(c2)C(=O)N(C2CCC(=O)NC2=O)C3)cc1C12CC1CN(C)C2] | 155 |
| 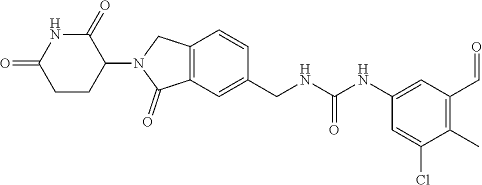 [SMILES: Cc1c(Cl)cc(NC(=O)NCc2ccc3c(c2)C(=O)N(C2CCC(=O)NC2=O)C3)cc1C=O] | 156 |
| 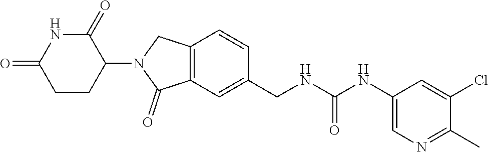 [SMILES: Cc1ncc(NC(=O)NCc2ccc3c(c2)C(=O)N(C2CCC(=O)NC2=O)C3)cc1Cl] | 157 |
| 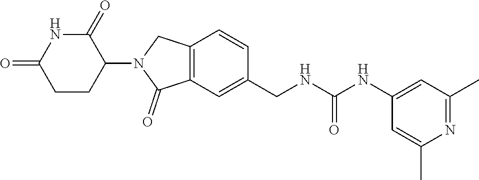 [SMILES: Cc1cc(NC(=O)NCc2ccc3c(c2)C(=O)N(C2CCC(=O)NC2=O)C3)cc(C)n1] | 158 |
| 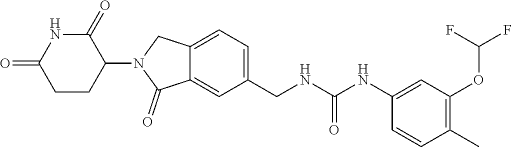 [SMILES: Cc1ccc(NC(=O)NCc2ccc3c(c2)C(=O)N(C2CCC(=O)NC2=O)C3)cc1OC(F)F] | 159 |
| 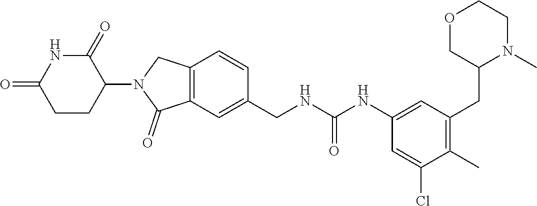 [SMILES: Cc1c(Cl)cc(NC(=O)NCc2ccc3c(c2)C(=O)N(C2CCC(=O)NC2=O)C3)cc1CC1COCCN1C] | 160 |
| 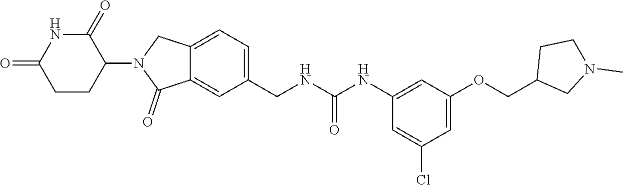 [SMILES: CN1CCC(COc2cc(Cl)cc(NC(=O)NCc3ccc4c(c3)C(=O)N(C3CCC(=O)NC3=O)C4)c2)C1] | 161 |
| 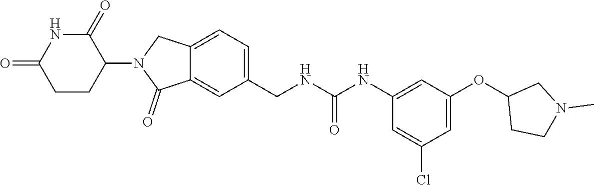 [SMILES: CN1CCC(Oc2cc(Cl)cc(NC(=O)NCc3ccc4c(c3)C(=O)N(C3CCC(=O)NC3=O)C4)c2)C1] | 162 |
| 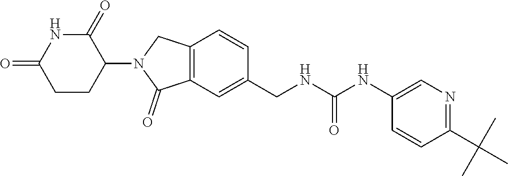 [SMILES: CC(C)(C)c1ccc(NC(=O)NCc2ccc3c(c2)C(=O)N(C2CCC(=O)NC2=O)C3)cn1] | 163 |
| 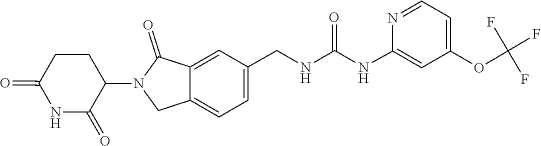 [SMILES: O=C1CCC(N2Cc3ccc(CNC(=O)Nc4cc(OC(F)(F)F)ccn4)cc3C2=O)C(=O)N1] | 164 |
| 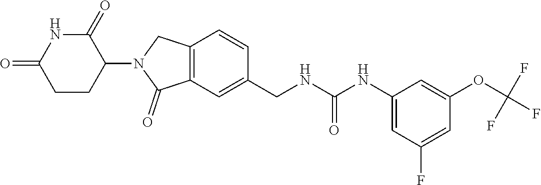 [SMILES: O=C1CCC(N2Cc3ccc(CNC(=O)Nc4cc(F)cc(OC(F)(F)F)c4)cc3C2=O)C(=O)N1] | 165 |

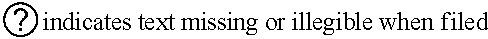

TABLE 2
Cereblon Binding Modulators

| Compound | | |
|---|---|---|
| No. | Structure | Compound Name |
| 1-1 | 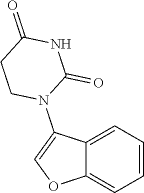 [SMILES: O=C1CCN(c2coc3ccccc23)C(=O)N1] | 1-(benzofuran-3- yl)dihydropyrimidine-2,4(1H,3H)- dione |
| 1-2 | 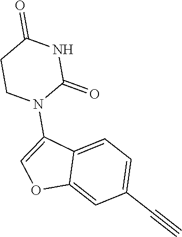 [SMILES: C#Cc1ccc2c(N3CCC(=O)NC3=O)coc2c1] | 1-(6-ethynylbenzofuran-3- yl)dihydropyrimidine-2,4(1H,3H)- dione |
| 1-3 | 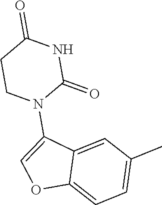 [SMILES: Cc1ccc2occ(N3CCC(=O)NC3=O)c2c1] | 1-(5-methylbenzofuran-3- yl)dihydropyrimidine-2,4(1H,3H)- dione |
| 1-4 | 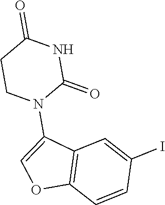 [SMILES: O=C1CCN(c2coc3ccc(I)cc23)C(=O)N1] | 1-(5-iodobenzofuran-3- yl)dihydropyrimidine-2,4(1H,3H)- dione |
| 1-5 | 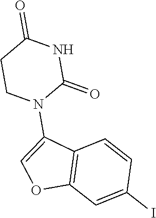 [SMILES: O=C1CCN(c2coc3cc(I)ccc23)C(=O)N1] | 1-(6-iodobenzofuran-3- yl)dihydropyrimidine-2,4(1H,3H)- dione |
| 1-6 | 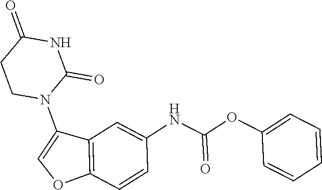 [SMILES: O=C1CCN(c2coc3ccc(NC(=O)Oc4ccccc4)cc23)C(=O)N1] | phenyl (3-(2,4- dioxotetrahydropyrimidin-1(2H)- yl)benzofuran-5-yl)carbamate |
| 1-7 | 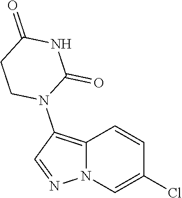 [SMILES: O=C1CCN(c2cnn3cc(Cl)ccc23)C(=O)N1] | 1-(6-chloropyrazolo[1,5-a]pyridin-3- yl)dihydropyrimidine-2,4(1H,3H)- dione |
| 1-8 | 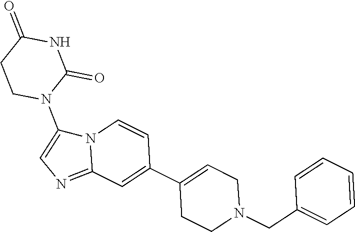 [SMILES: O=C1CCN(c2cnc3cc(C4=CCN(Cc5ccccc5)CC4)ccn23)C(=O)N1] | 1-(7-(1-benzyl-1,2,3,6- tetrahydropyridin-4-yl)imidazo[1,2- a]pyridin-3-yl)dihydropyrimidine- 2,4(1H,3H)-dione |
| 1-9 | 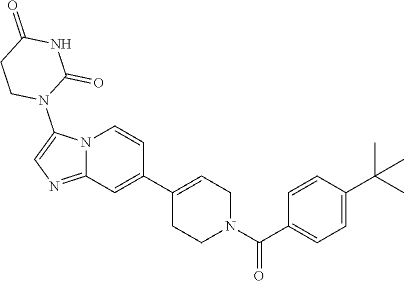 [SMILES: CC(C)(C)c1ccc(C(=O)N2CC=C(c3ccn4c(N5CCC(=O)NC5=O)cnc4c3)CC2)cc1] | 1-(7-(1-(4-(tert-butyl)benzoyl)- 1,2,3,6-tetrahydropyridin-4- yl)imidazo[1,2-a]pyridin-3- yl)dihydropyrimidine-2,4(1H,3H)- dione |
| 1-10 | 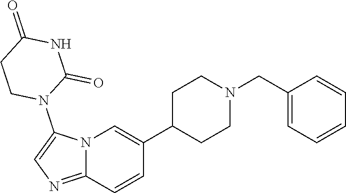 [SMILES: O=C1CCN(c2cnc3ccc(C4CCN(Cc5ccccc5)CC4)cn23)C(=O)N1] | 1-(6-(1-benzylpiperidin-4- yl)imidazo[1,2-a]pyridin-3- yl)dihydropyrimidine-2,4(1H,3H)- dione |
| 1-11 | 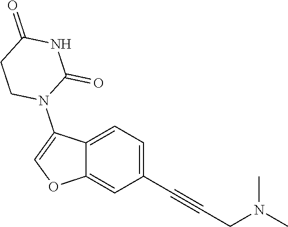 [SMILES: CN(C)CC#Cc1ccc2c(N3CCC(=O)NC3=O)coc2c1] | 1-(6-(3-(dimethylamino)prop-1-yn-1- yl)benzofuran-3- yl)dihydropyrimidine-2,4(1H,3H)- dione |
| 1-12 | 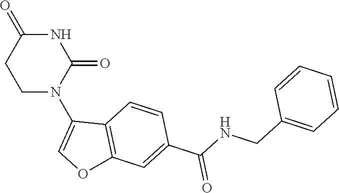 [SMILES: O=C1CCN(c2coc3cc(C(=O)NCc4ccccc4)ccc23)C(=O)N1] | N-benzyl-3-(2,4- dioxotetrahydropyrimidin-1(2H)- yl)benzofuran-6-carboxamide |
| 1-13 | 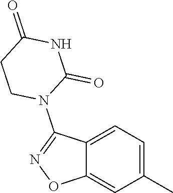 [SMILES: Cc1ccc2c(N3CCC(=O)NC3=O)noc2c1] | 1-(6-methylbenzo[d]isoxazol-3- yl)dihydropyrimidine-2,4(1H,3H)- dione |
| 1-14 | 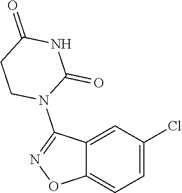 [SMILES: O=C1CCN(c2noc3ccc(Cl)cc23)C(=O)N1] | 1-(5-chlorobenzo[d]isoxazol-3- yl)dihydropyrimidine-2,4(1H,3H)- dione |
| 1-15 | 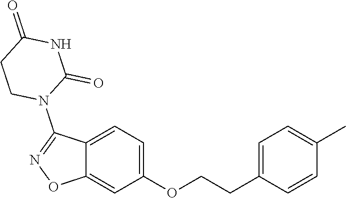 [SMILES: Cc1ccc(CCOc2ccc3c(N4CCC(=O)NC4=O)noc3c2)cc1] | 1-(6-(4- methylphenethoxy)benzo[d]isoxazol- 3-yl)dihydropyrimidine-2,4(1H,3H)- dione |
| I-16 | 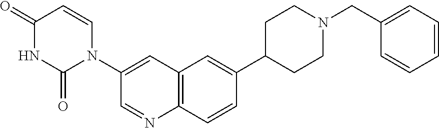 [SMILES: O=c1ccn(-c2cnc3ccc(C4CCN(Cc5ccccc5)CC4)cc3c2)c(=O)[nH]1] | 1-(6-(1-benzylpiperidin-4- yl)quinolin-3-yl)pyrimidine- 2,4(1H,3H)-dione |
| 1-17 | 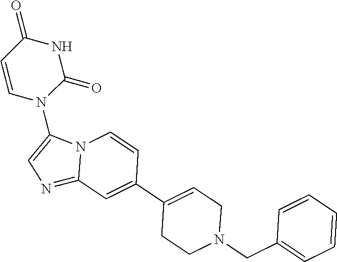 [SMILES: O=c1ccn(-c2cnc3cc(C4=CCN(Cc5ccccc5)CC4)ccn23)c(=O)[nH]1] | 1-(7-(1-benzyl-1,2,3,6- tetrahydropyridin-4-yl)imidazo[1,2- a]pyridin-3-yl)pyrimidine- 2,4(1H,3H)-dione |
| 1-18 | 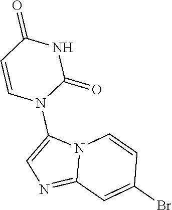 [SMILES: O=c1ccn(-c2cnc3cc(Br)ccn23)c(=O)[nH]1] | 1-(7-bromoimidazo[1,2-a]pyridin-3- yl)pyrimidine-2,4(1H,3H)-dione |

### Molecular Glues

In some cases, the E3 ligase binding modulator is a molecular glue.

A molecular glue is a small molecule that stabilizes the interaction of two or more biomolecules (e.g., proteins) at a protein-protein interaction (PPI) interface, e.g., by chemically inducing or strengthening surface interactions between the proteins. In some cases, the molecular glue stabilizes the interaction of an E3 ligase substrate receptor protein and one or more target protein(s).

In some cases, the molecular glue functions as a molecular glue drug by modulating (e.g., increasing or promoting) one or more of: the stability of protein-protein interaction(s), degradation of protein(s), sequestration of protein(s) (e.g., into specific regions of a cell), phosphorylation of protein(s), de-phosphorylation of protein(s), and stabilization of protein(s).

In some cases, the modulation is directly of the target protein (the “glued” target). In some cases, the modulation is indirect (e.g., of a target downstream of the “glued” target).

### Molecular Glue Degraders

Thalidomide and immunomodulatory imide drugs (IMiDs), such as lenalidomide, and pomalidomide, are examples of molecular glue drugs that induce degradation of normally unrecognized target proteins (sometimes referred to as “neosubstrates”) by generating an interaction between an E3 ligase substrate receptor (e.g., cereblon) and a target protein (e.g., IKZF1/3).

Molecular glue drugs, such as these, that induce the degradation of protein(s) are sometimes referred to as a molecular glue degraders. Molecular glue degraders are believed to create neosubstrate recognition interfaces on the surface of the E3 ligase substrate receptor protein that engage in induced protein-protein interactions with neosubstrates.

### Target Proteins

The compositions and methods describe herein are useful, for example, in identification and/or prediction of degrons on the surface of a protein, e.g., on the surface of a neosubstrate, potential neosubstrate, predicted neosubstrate and/or putative neosubstrate of an E3 ligase target protein and/or E3 ligase binding modulator target protein.

### Degrons

In the context of molecular glue degraders, for example, in some cases the target protein is the protein the protein that interfaces (e.g., binds) with the E3 ligase substrate receptor. In some cases, the target protein comprises a degron.

Degrons are structural features on the surface of a protein that mediate recruitment of and degradation by an E3 ligase complex, e.g., an E3 ligase complex described herein. Degrons are described, for example, in Lucas and Ciulli, “Recognition of Substrate Dependent Degrons by E3 Ubiquitin Ligases and Modulation by Small-Molecule Mimicry Strategies,” Current Opinion in Structural Biology 44:101-10 (2017). For CRBN, for example, a β-hairpin loop containing a glycine at a key position (G-loop) has been found as a degron based on the interaction of CK1a, GSPT1, and Zn-fingers with CRBN in their X-ray structures. See, e.g., Matyskiela et al., “A Novel Cereblon Modulator Recruits GSPT1 to the RL4 (CRBN) Ubiquitin Ligase, Nature 535(7611):252-7 (2016); Petzold et al. «Structural basis of lenalidomide-induced CK1α degradation by the CRL4CRBN ubiquitin ligase, “Nature, 532(7597), 127-130 (2016); Furihata et al., “Structural bases of IMiD selectivity that emerges by 5-hydroxythalidomide,” Nat Commun. 11(1):4578 (2020); Sievers et al., “Defining the human C2H2 zinc finger degrome targeted by thalidomide analogs through CRBN,” Science 362(6414):eaat0572 (2018); and Wang et al., “Acute pharmacological degradation of Helios destabilizes regulatory T cells,” Nat. Chem. Bio. 17(6):711-17 (2021).

Degrons have been described and/or identified based on their primary, secondary, or tertiary protein structures. In some cases, a degron is described and/or identified in terms of its quaternary structure (e.g., in complex). In some cases, a degron is described and/or identified in the context of a crystal structure (e.g., a PDB structure). For CRBN, for example, there are six known degrons in nine crystal structures (PDB ids: 6UML, 6H0G, 6H0F, 5FQD, 5HXB, 6XK9, 7LPS, 7BQU, and 7BQV).

In some cases, the degron is a small molecule dependent degron (i.e., is a structural feature on the surface of the protein that mediates recruitment of and degradation by an E3 ligase in the presence of an E3 ligase binding modulator, e.g., an E3 ligase binding modulator described herein). In some cases, the degron is a small molecule independent degron (i.e., is a structural feature on the surface of the protein that mediates recruitment of and degradation by an E3 ligase in the absence of an E3 ligase binding modulator, e.g., an E3 ligase binding modulator described herein).

Degrons may be present on the surface of the protein target as it is expressed or added to the protein target via a linker (e.g., a proteolysis targeting chimera (PROTAC), see, e.g., Pavia and Crews, “Targeted Protein Degradation: Elements of PROTAC Design,” Curr Opin Chem Biol 50:111-19 (2019).

Degrons include, e.g., N-degrons and C-degrons, which are known and described in the art. See, e.g., Lucas and Ciulli 2017; see also, e.g., Timms and Koren, “Typing up Loose Ends: the N-degron and C-degron Pathways of Protein Degradation,” Biochem Soc Trans 48(4):1557-67 (2020).

Degrons also include, e.g., phosphodegrons and oxygen-dependent degrons (ODDs), which are also known and described in the art. See, e.g., Lucas and Ciulli 2017. In some cases, the degron comprises or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid.

In some cases, the degron comprises or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid.

In some cases, the degron comprises or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine.

In some cases, the degron comprises or consists of the amino acid motif ETGE (SEQ ID NO: 1). In some cases, the degron comprises or consists of the amino acid motif DLG.

In some cases, the degron comprises or consists of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine. In some cases the degron comprises or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix.

In some cases, the degron comprises or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

Degrons also include, e.g., G-loop degrons. Thus, in some cases, the E3 ligase binding target is a protein comprising an E3 ligase-accessible loop, e.g., a cereblon-accessible loop, e.g., a G-loop.

In some cases, the G-loop degron comprises or consist of the amino acid sequence X¹-X²-X³-X⁴-G-X⁶, wherein: each of X¹, X², X³, X⁴, and X⁶ are independently selected from any one of the natural occurring amino acids; and G (i.e. X⁵) is glycine.

In some cases, the G-loop degron comprises or consists of the amino acid sequence X¹-X²-X³-X⁴-G-X⁶-X⁷, wherein: each of X¹, X², X³, X⁴, X⁶, and X⁷ are independently selected from any one of the natural occurring amino acids; and G (i.e. X⁵) is glycine.

In some cases, the G-loop degron comprises or consists of the amino acid sequence X¹-X²-X³-X⁴-G-X⁶-X⁷-X⁸; wherein: each of X¹, X², X³, X⁴, X⁶, X⁷, and X⁸ are independently selected from any one of the natural occurring amino acids; and G (i.e. X⁵) is glycine.

In some cases, a distance from X¹ to X⁴ is less than about 7 angstroms. In some cases, X¹ and X⁴ are the same. In some cases, X¹ is aspartic acid or asparagine and X⁴ is serine or threonine.

In some cases, the G-loop degron comprises or consists of the amino acid sequence X¹-X²-X³-X⁴-G-X⁶, wherein X¹ is selected from the group consisting of asparagine, aspartic acid, and cysteine; X² is selected from the group consisting of isoleucine, lysine, and asparagine; X³ is selected from the group consisting of threonine, lysine, and glutamine; X⁴ is selected from the group consisting of asparagine, serine, and cysteine; X⁵ is glycine; and X⁶ is selected from the group consisting of glutamic acid and glutamine.

In some cases, the G-loop degron comprises or consists of the amino acid sequence X¹-X²-X³-X⁴-G-X⁶, wherein X¹ is asparagine; X² is isoleucine; X³ is threonine; X⁴ is asparagine; X⁵ is glycine; and X⁶ is glutamic acid.

In some cases, the G-loop degron comprises or consists of the amino acid sequence X¹-X²-X³-X⁴-G-X⁶, wherein X¹ is aspartic acid; X² is lysine; X³ is lysine; X⁴ is serine; X⁵ is glycine; and X⁶ is glutamic acid.

In some cases, the G-loop degron comprises or consists of the amino acid sequence X¹-X²-X³-X⁴-G-X⁶, wherein X¹ is cysteine; X² is asparagine; X³ is glutamine; X⁴ is cysteine; X⁵ is glycine; and X⁶ is glutamine.

In some cases, the degron comprises or consists of an amino acid sequence of about 2 to about 15 amino acids in length. In some cases, the degron comprises or consists of an amino acid sequence of about 6 to about 12 amino acids in length. In some cases, the degron comprises or consists of at least about 6 amino acids. In some cases, the degron comprises or consists of at least about 7 amino acids. In some cases, the degron comprises or consists of at least about 8 amino acids. In some cases, the degron comprises or consists of at least about 9 amino acids. In some cases, the amino degron comprises or consists of at least about 10 amino acids. In some cases, the G-loop degron is 6, 7, or 8 amino acids long.

### Proteins

In some cases, the target protein is a protein listed in the table below or a variant, derivative, ortholog, or homolog thereof.

TABLE 3
Target Proteins

| Target | | |
|---|---|---|
| Protein | | |
| Symbol | Uniprot Name | Target Protein Name |
| A2M | A2MG_HUMAN | Alpha-2-macroglobulin |
| AADAT | AADAT_HUMAN | Kynurenine/alpha-aminoadipate aminotransferase, mitochondrial |
| AAKI | AAKI_HUMAN | AP2-associated protein kinase I |
| AAMDC | AAMDC_HUMAN | Mth938 domain-containing protein |
| AARS | SYAC_HUMAN | Alanine--tRNA ligase, cytoplasmic |
| AASDHPPT | ADPPT_HUMAN | L-aminoadipate-semialdehyde dehydrogenase-phosphopantetheiny |
| | | I transferase |
| AASS | AASS_HUMAN | Saccharopine dehydrogenase |
| ABLI | ABLI_HUMAN | Tyrosine-protein kinase ABL I |
| ABL2 | ABL2_HUMAN | Tyrosine-protein kinase ABL2 |
| ABLIM2 | ABLM2_HUMAN | Actin-binding LIM protein 2 |
| ACAAI | THIK_HUMAN | 3-ketoacyl-CoA thiolase, peroxisomal |
| ACAA2 | THIM_HUMAN | 3-ketoacyl-CoA thiolase, mitochondrial |
| ACACA | ACACA_HUMAN | Biotin carboxylase |
| ACACB | ACACB_HUMAN | Biotin carboxylase |
| ACADVL | ACADV_HUMAN | Very long-chain specific acyl-CoA dehydrogenase, mitochondrial |
| ACAPI | ACAPI_HUMAN | Arf-GAP with coiled-coil, ANK repeat and PH domain-containing |
| | | protein I |
| ACAP2 | ACAP2_HUMAN | Arf-GAP with coiled-coil, ANK repeat and PH domain-containing |
| | | protein 2 |
| ACAP3 | ACAP3_HUMAN | Arf-GAP with coiled-coil, ANK repeat and PH domain-containing |
| | | protein 3 |
| ACAT2 | THIC_HUMAN | Acety 1-CoA acety ltransferase, cytosolic |
| ACE | ACE_HUMAN | Angiotensin-converting enzyme, soluble form |
| ACHE | ACES_HUMAN | Acetylcholinesterase |
| ACLY | ACLY_HUMAN | ATP-citrate synthase |
| ACOI | ACOC_HUMAN | Cytoplasmic aconitate hydratase |
| ACOT12 | ACO12_HUMAN | Acetyl-coenzyme A thioesterase |
| ACOT13 | ACO13_HUMAN | Acyl-coenzyme A thioesterase 13, N-terminally processed |
| ACOT2 | ACOT2_HUMAN | Acyl-coenzyme A thioesterase 2, mitochondrial |
| ACOT4 | ACOT4_HUMAN | Peroxisomal succinyl-coenzyme A thioesterase |
| ACP5 | PPA5_HUMAN | Tartrate-resistant acid phosphatase type 5 |
| ACP6 | PPA6_HUMAN | Lysophosphatidic acid phosphatase type 6 |
| ACSM2A | ACS2A_HUMAN | Acyl-coenzyme A synthetase ACSM2A, mitochondrial |
| ACTB | ACTB_HUMAN | Actin, cytoplasmic 1, N-terminally processed |
| ACTGl | ACTG_HUMAN | Actin, cytoplasmic 2, N-terminally processed |
| ACVRl | ACVR1_HUMAN | Activin receptor type-1 |
| ACVRlB | ACV1B_HUMAN | Activin receptor type-1B |
| ACVR2A | AVR2A_HUMAN | Activin receptor type-2A |
| ACVR2B | AVR2B_HUMAN | Activin receptor type-2B |
| ACY1 | ACY1_HUMAN | Aminoacylase-1 |
| ADA2 | ADA2_HUMAN | Adenosine deaminase 2 |
| ADAM10 | ADA10_HUMAN | Disintegrin and metalloproteinase domain-containing protein 10 |
| ADAM17 | ADA17_HUMAN | Disintegrin and metalloproteinase domain-containing protein 17 |
| ADAP1 | ADAP1_HUMAN | Arf-GAP with dual PH domain-containing protein 1 |
| ADAP2 | ADAP2_HUMAN | Arf-GAP with dual PH domain-containing protein 2 |
| ADAR | DSRAD_HUMAN | Double-stranded RNA-specific adenosine deaminase |
| ADARB1 | RED1_HUMAN | Double-stranded RNA-specific editase 1 |
| ADCY10 | ADCYA_HUMAN | Adenylate cyclase type 10 |
| ADCYAP1R1 | PACR_HUMAN | Pituitary adenylate cyclase-activating polypeptide type I receptor |
| ADGRB3 | AGRB3_HUMAN | Adhesion G protein-coupled receptor B3 |
| ADGRL3 | AGRL3_HUMAN | Adhesion G protein-coupled receptor L3 |
| AD1POQ | AD1PO_HUMAN | Adiponectin |
| ADORA2A | AA2AR_HUMAN | Adenosine receptor A2a |
| ADRB2 | ADRB2_HUMAN | Beta-2 adrenergic receptor |
| ADRM1 | ADRM1_HUMAN | Proteasomal ubiquitin receptor ADRM1 |
| ADSS | PURA2_HUMAN | Adenylosuccinate synthetase isozyme 2 |
| AEBP2 | AEBP2_HUMAN | Zinc finger protein AEBP2 |
| AGA | ASPG_HUMAN | Glycosylasparaginase beta chain |
| AGAP2 | AGAP2_HUMAN | Arf-GAP with GTPase, ANK repeat and PH domain-containing |
| | | protein 2 |
| AGER | RAGE_HUMAN | Advanced glycosylation end product-specific receptor |
| AGFG1 | AGFG1_HUMAN | Arf-GAP domain and FG repeat-containing protein 1 |
| AGO1 | AGO1_HUMAN | Protein argonaute-1 |
| AGO2 | AGO2_HUMAN | Protein argonaute-2 |
| AGO3 | AGO3_HUMAN | Protein argonaute-3 |
| AGRP | AGRP_HUMAN | Agouti-related protein |
| AGTR2 | AGTR2_HUMAN | Type-2 angiotensin II receptor |
| AGXT | SPYA_HUMAN | Serine--pyruvate aminotransferase |
| AHCY | SAHH_HUMAN | Adenosylhomocysteinase |
| AHCYL1 | SAHH2_HUMAN | S-adenosylhomocysteine hydrolase-like protein 1 |
| AHCYL2 | SAHH3_HUMAN | Adenosylhomocysteinase 3 |
| A1FM1 | A1FM1_HUMAN | Apoptosis-inducing factor 1, mitochondrial |
| A1M2 | AIM2_HUMAN | Interferon-inducible protein A1M2 |
| A1MP1 | A1MP1_HUMAN | Endothelial monocyte-activating polypeptide 2 |
| A1P | A1P_HUMAN | AH receptor-interacting protein |
| A1RE | A1RE_HUMAN | Autoimmune regulator |
| AK2 | KAD2_HUMAN | Adenylate kinase 2, mitochondrial, N-terminally processed |
| AK3 | KAD3_HUMAN | GTP:AMP phosphotransferase AK3, mitochondrial |
| AK4 | KAD4_HUMAN | Adenylate kinase 4, mitochondrial |
| AKAP13 | AKP13_HUMAN | A-kinase anchor protein 13 |
| AKR1A1 | AK1A1_HUMAN | Aldo-keto reductase family 1 member A1 |
| AKR1B1 | ALDR_HUMAN | Aldo-keto reductase family 1 member B1 |
| AKR1C1 | AK1C1_HUMAN | Aldo-keto reductase family 1 member C1 |
| AKR1C2 | AK1C2_HUMAN | Aldo-keto reductase family 1 member C2 |
| AKR1C3 | AK1C3_HUMAN | Aldo-keto reductase family 1 member C3 |
| AKT1 | AKT1_HUMAN | RAC-alpha serine/threonine-protein kinase |
| AKT2 | AKT2_HUMAN | RAC-beta serine/threonine-protein kinase |
| AKT3 | AKT3_HUMAN | RAC-gamma serine/threonine-protein kinase |
| ALAS2 | HEM0_HUMAN | 5-aminolevulinate synthase, erythroid-specific, mitochondrial |
| ALCAM | CD166_HUMAN | CD 166 antigen |
| ALDH1A2 | AL1A2_HUMAN | Retinal dehydrogenase 2 |
| ALDH1L1 | AL1L1_HUMAN | Cytosolic 10-formyltetrahydrofolate dehydrogenase |
| ALDH2 | ALDH2_HUMAN | Aldehyde dehydrogenase, mitochondrial |
| ALDH5A1 | SSDH_HUMAN | Succinate-semialdehyde dehydrogenase, mitochondrial |
| ALDH7A1 | AL7A1_HUMAN | Alpha-aminoadipic semialdehyde dehydrogenase |
| ALDOB | ALDOB_HUMAN | Fructose-bisphosphate aldolase B |
| ALK | ALK_HUMAN | ALK tyrosine kinase receptor |
| ALKBH8 | ALKB8_HUMAN | Alkylated DNA repair protein alkB homolog 8 |
| ALOX12 | LOX12_HUMAN | Arachidonate 12-lipoxygenase, 12S-type |
| ALOX15B | LX15B_HUMAN | Arachidonate 15-lipoxygenase B |
| ALOX5 | LOX5_HUMAN | Arachidonate 5-lipoxygenase |
| AMBP | AMBP_HUMAN | Trypstatin |
| AMD1 | DCAM_HUMAN | S-adenosylmethionine decarboxylase beta chain |
| AMFR | AMFR_HUMAN | E3 ubiquitin-protein ligase AMFR |
| AMT | GCST_HUMAN | Aminomethyltransferase, mitochondrial |
| AMY1A\| | AMY1_HUMAN | Alpha-amylase 1 |
| AMY1B\| | | |
| AMY1C | | |
| AMY2A | AMYP_HUMAN | Pancreatic alpha-amylase |
| ANAPC1 | APC1_HUMAN | Anaphase-promoting complex subunit 1 |
| ANAPC4 | APC4_HUMAN | Anaphase-promoting complex subunit 4 |
| ANGPT1 | ANGP1_HUMAN | Angiopoietin-1 |
| ANGPT2 | ANGP2_HUMAN | Angiopoietin-2 |
| ANGPTL3 | ANGL3_HUMAN | ANGPTL3(17-224) |
| ANGPTL4 | ANGL4_HUMAN | ANGPTL4 C-terminal chain |
| ANK1 | ANK1_HUMAN | Ankyrin-1 |
| ANK2 | ANK2_HUMAN | Ankyrin-2 |
| ANKFY1 | ANFY1_HUMAN | Rabankyrin-5 |
| ANKMY1 | ANKY1_HUMAN | Ankyrin repeat and MYND domain-containing protein 1 |
| ANKMY2 | ANKY2_HUMAN | Ankyrin repeat and MYND domain-containing protein 2 |
| ANKRA2 | ANRA2_HUMAN | Ankyrin repeat family A protein 2 |
| ANKRD27 | ANR27_HUMAN | Ankyrin repeat domain-containing protein 27 |
| ANLN | ANLN_HUMAN | Anillin |
| ANO10 | ANO10_HUMAN | Anoctamin-10 |
| ANOS1 | KALM_HUMAN | Anosmin-1 |
| ANPEP | AMPN_HUMAN | Aminopeptidase N |
| ANTXR1 | ANTR1_HUMAN | Anthrax toxin receptor 1 |
| AOAH | AOAH_HUMAN | Acyloxyacyl hydrolase large subunit |
| AOC1 | AOC1_HUMAN | Amiloride-sensitive amine oxidase [copper containing] |
| AOC3 | AOC3_HUMAN | Membrane primary amine oxidase |
| AOX1 | AOXA_HUMAN | Aldehyde oxidase |
| AP1S3 | AP1S3_HUMAN | AP-1 complex subunit sigma-3 |
| AP2B1 | AP2B1_HUMAN | AP-2 complex subunit beta |
| AP4B1 | AP4B1_HUMAN | AP-4 complex subunit beta-1 |
| AP4M1 | AP4M1_HUMAN | AP-4 complex subunit mu-1 |
| APAF1 | APAF_HUMAN | Apoptotic protease-activating factor 1 |
| APBB1 | APBB1_HUMAN | Amyloid-beta A4 precursor protein-binding family B member 1 |
| APBB3 | APBB3_HUMAN | Amyloid-beta A4 precursor protein-binding family B member 3 |
| APCS | SAMP_HUMAN | Serum amyloid P-component(1-203) |
| APEX1 | APEX1_HUMAN | DNA-(apurinic or apyrimidinic site) lyase, mitochondrial |
| AP1P | MTNB_HUMAN | Methylthioribulose-1-phosphate dehydratase |
| APLF | APLF_HUMAN | Aprataxin and PNK-like factor |
| APLNR | APJ_HUMAN | Apelin receptor |
| APLP2 | APLP2_HUMAN | Amyloid-like protein 2 |
| APOBEC3A | ABC3A_HUMAN | DNA dC&minus;>dU-editing enzyme APOBEC-3A |
| APOD | APOD_HUMAN | Apolipoprotein D |
| APOH | APOH_HUMAN | Beta-2-glycoprotein 1 |
| APOM | APOM_HUMAN | Apolipoprotein M |
| APP | A4_HUMAN | C31 |
| APPL1 | DP13A_HUMAN | DCC-interacting protein 13-alpha |
| APRT | APT_HUMAN | Adenine phosphoribosyltransferase |
| APTX | APTX_HUMAN | Aprataxin |
| AQR | AQR_HUMAN | RNA helicase aquarius |
| AR | ANDR_HUMAN | Androgen receptor |
| ARAF | ARAF_HUMAN | Serine/threonine-protein kinase A-Raf |
| ARAP1 | ARAP1_HUMAN | Arf-GAP with Rho-GAP domain, ANK repeat and PH domain- |
| | | containing protein 1 |
| ARAP3 | ARAP3_HUMAN | Arf-GAP with Rho-GAP domain, ANK repeat and PH domain- |
| | | containing protein 3 |
| ARF1 | ARF1_HUMAN | ADP-ribosylation factor 1 |
| ARF6 | ARF6_HUMAN | ADP-ribosylation factor 6 |
| ARFGAP1 | ARFG1_HUMAN | ADP-ribosylation factor GTPase-activating protein 1 |
| ARFGAP2 | ARFG2_HUMAN | ADP-ribosylation factor GTPase-activating protein 2 |
| ARFGAP3 | ARFG3_HUMAN | ADP-ribosylation factor GTPase-activating protein 3 |
| ARHGAP10 | RHG10_HUMAN | Rho GTPase-activating protein 10 |
| ARHGAP11A | RHGBA_HUMAN | Rho GTPase-activating protein 11A |
| ARHGAP26 | RHG26_HUMAN | Rho GTPase-activating protein 26 |
| ARHGAP27 | RHG27_HUMAN | Rho GTPase-activating protein 27 |
| ARHGAP9 | RHG09_HUMAN | Rho GTPase-activating protein 9 |
| ARHGEF12 | ARHGC_HUMAN | Rho guanine nucleotide exchange factor 12 |
| ARHGEF16 | ARHGG_HUMAN | Rho guanine nucleotide exchange factor 16 |
| ARHGEF18 | ARHG1_HUMAN | Rho guanine nucleotide exchange factor 18 |
| ARHGEF2 | ARHG2_HUMAN | Rho guanine nucleotide exchange factor 2 |
| ARHGEF28 | ARG28_HUMAN | Rho guanine nucleotide exchange factor 28 |
| ARHGEF4 | ARHG4_HUMAN | Rho guanine nucleotide exchange factor 4 |
| AR1D4A | AR14A_HUMAN | AT-rich interactive domain-containing protein 4A |
| ARlH1 | ARl1_HUMAN | E3 ubiquitin-protein ligase ARlH1 |
| ARNT | ARNT_HUMAN | Aryl hydrocarbon receptor nuclear translocator |
| ARNTL2 | BMAL2_HUMAN | Ary I hydrocarbon receptor nuclear translocator like protein 2 |
| ARSB | ARSB_HUMAN | Arylsulfatase B |
| ASAH1 | ASAH1_HUMAN | Acid ceramidase subunit beta |
| ASAH2 | ASAH2_HUMAN | Neutral ceramidase soluble form |
| ASAP1 | ASAP1_HUMAN | Arf-GAP with SH3 domain, ANK repeat and PH domain-containing |
| | | protein 1 |
| ASAP3 | ASAP3_HUMAN | Arf-GAP with SH3 domain, ANK repeat and PH domain-containing |
| | | protein 3 |
| ASB11 | ASB11_HUMAN | Ankyrin repeat and SOCS box protein 11 |
| ASB9 | ASB9_HUMAN | Ankyrin repeat and SOCS box protein 9 |
| ASH1L | ASH1L_HUMAN | Histone-lysine N-methyltransferase ASH1L |
| ASH2L | ASH2L_HUMAN | Setl/Ash2 histone methyltransferase complex subunit ASH2 |
| ASPA | ACY2_HUMAN | Aspartoacylase |
| ASRGL1 | ASGL1_HUMAN | Isoaspartyl peptidase/L-asparaginase beta chain |
| ASS1 | ASSY_HUMAN | Argininosuccinate synthase |
| ASTN2 | ASTN2_HUMAN | Astrotactin-2 |
| ASXL1 | ASXL1_HUMAN | Putative Polycomb group protein ASXL1 |
| ASXL2 | ASXL2_HUMAN | Putative Polycomb group protein ASXL2 |
| ASXL3 | ASXL3_HUMAN | Putative Polycomb group protein ASXL3 |
| ATG101 | ATGA1_HUMAN | Autophagy-related protein 101 |
| ATG13 | ATG13_HUMAN | Autophagy-related protein 13 |
| ATG16L1 | Al6L1_HUMAN | Autophagy-related protein 16-1 |
| ATG5 | ATG5_HUMAN | Autophagy protein 5 |
| ATL1 | ATLA1_HUMAN | Atlastin-1 |
| ATL3 | ATLA3_HUMAN | Atlastin-3 |
| ATM | ATM_HUMAN | Serine-protein kinase ATM |
| ATP7A | ATP7A_HUMAN | Copper-transporting ATPase 1 |
| ATP7B | ATP7B_HUMAN | WND/140 kDa |
| ATR | ATR_HUMAN | Serine/threonine-protein kinase ATR |
| ATRX | ATRX_HUMAN | Transcriptional regulator ATRX |
| ATXN1 | ATX1_HUMAN | Ataxin-1 |
| AURKA | AURKA_HUMAN | Aurora kinase A |
| AXL | UFO_HUMAN | Tyrosine-protein kinase receptor UFO |
| AZGP1 | ZA2G_HUMAN | Zinc-alpha-2-glycoprotein |
| AZU1 | CAP7_HUMAN | Azurocidin |
| B2M | B2MG_HUMAN | Beta-2-microglobulin form pl 5.3 |
| B4GALT1 | B4GT1_HUMAN | Processed beta-1,4-galactosyltransferase 1 |
| BACE1 | BACE1_HUMAN | Beta-secretase 1 |
| BACE2 | BACE2_HUMAN | Beta-secretase 2 |
| BAK1 | BAK_HUMAN | Bcl-2 homologous antagonist/killer |
| BARD1 | BARD1_HUMAN | BRCA1-associated RING domain protein 1 |
| BAX | BAX_HUMAN | Apoptosis regulator BAX |
| BAZ2A | BAZ2A_HUMAN | Bromodomain adjacent to zinc finger domain protein 2A |
| BBS9 | PTHB1_HUMAN | Protein PTHB1 |
| BCAM | BCAM_HUMAN | Basal cell adhesion molecule |
| BCAT1 | BCAT1_HUMAN | Branched-chain-amino-acid aminotransferase, cytosolic |
| BCAT2 | BCAT2_HUMAN | Branched-chain-amino-acid aminotransferase, mitochondrial |
| BCHE | CHLE_HUMAN | Cholinesterase |
| BCL11A | BC11A_HUMAN | B-cell lymphoma/leukemia 11A |
| BCL11B | BC11B_HUMAN | B-cell lymphoma/leukemia 11B |
| BCL3 | BCL3_HUMAN | B-cell lymphoma 3 protein |
| BCL6 | BCL6_HUMAN | B-cell lymphoma 6 protein |
| BCL6B | BCL6B_HUMAN | B-cell CLL/lymphoma 6 member B protein |
| BCR | BCR_HUMAN | Breakpoint cluster region protein |
| BDNF | BDNF_HUMAN | Brain-derived neurotrophic factor |
| BECN1 | BECN1_HUMAN | Beclin-1-C 37 kDa |
| BHMT | BHMT1_HUMAN | Betaine--homocysteine S-methyltransferase 1 |
| BIRC2 | BIRC2_HUMAN | Baculoviral 1AP repeat-containing protein 2 |
| BIRC3 | BIRC3_HUMAN | Baculoviral 1AP repeat-containing protein 3 |
| BIRC6 | BIRC6_HUMAN | Baculoviral 1AP repeat-containing protein 6 |
| BIRC7 | BIRC7_HUMAN | Baculoviral 1AP repeat-containing protein 7 30 kDa subunit |
| BIRC8 | BIRC8_HUMAN | Baculoviral 1AP repeat-containing protein 8 |
| BLMH | BLMH_HUMAN | Bleomycin hydrolase |
| BM11 | BM11_HUMAN | Polycomb complex protein BMIl-1 |
| BMP2K | BMP2K_HUMAN | BMP-2-inducible protein kinase |
| BMPR1A | BMR1A_HUMAN | Bone morphogenetic protein receptor type-1A |
| BMPR1B | BMR1B_HUMAN | Bone morphogenetic protein receptor type-1B |
| BMPR2 | BMPR2_HUMAN | Bone morphogenetic protein receptor type-2 |
| BMX | BMX_HUMAN | Cytoplasmic tyrosine-protein kinase BMX |
| BNC2 | BNC2_HUMAN | Zinc finger protein basonuclin-2 |
| BOC | BOC_HUMAN | Brother of CDO |
| BOLA3 | BOLA3_HUMAN | BolA-like protein 3 |
| BP1 | BP1_HUMAN | Bactericidal permeability-increasing protein |
| BPIFA1 | BP1A1_HUMAN | BPI fold-containing family A member 1 |
| BRAF | BRAF_HUMAN | Serine/threonine-protein kinase B-raf |
| BRAP | BRAP_HUMAN | BRCA1-associated protein |
| BRD1 | BRD1_HUMAN | Bromodomain-containing protein 1 |
| BRF1 | TF3B_HUMAN | Transcription factor lllB 90 kDa subunit |
| BRF2 | BRF2_HUMAN | Transcription factor lllB 50 kDa subunit |
| BROX | BROX_HUMAN | BRO 1 domain-containing protein BROX |
| BSG | BAS1_HUMAN | Basigin |
| BSN | BSN_HUMAN | Protein bassoon |
| BSPRY | BSPRY_HUMAN | B box and SPRY domain-containing protein |
| BTBD2 | BTBD2_HUMAN | BTB/POZ domain-containing protein 2 |
| BTG2 | BTG2_HUMAN | Protein BTG2 |
| BTK | BTK_HUMAN | Tyrosine-protein kinase BTK |
| BTN3A1 | BT3A1_HUMAN | Butyrophilin subfamily 3 member A1 |
| BTN3A2 | BT3A2_HUMAN | Butyrophilin subfamily 3 member A2 |
| BTN3A3 | BT3A3_HUMAN | Butyrophilin subfamily 3 member A3 |
| BTRC | FBW1A_HUMAN | F-box/WD repeat-containing protein IA |
| BUD31 | BUD31_HUMAN | Protein BUD31 homolog |
| C11orf54 | CK054_HUMAN | Ester hydrolase C11orf54 |
| C11orf68 | CK068_HUMAN | UPF0696 protein C11orf68 |
| C1QA | C1QA_HUMAN | Complement C1q subcomponent subunit A |
| C1QB | C1QB_HUMAN | Complement C1q subcomponent subunit B |
| C1QBP | C1QBP_HUMAN | Complement component 1 Q subcomponent binding protein, |
| | | mitochondrial |
| C1QC | C1QC_HUMAN | Complement C1q subcomponent subunit C |
| C1QTNF5 | C1QT5_HUMAN | Complement C1q tumor necrosis factor-related protein 5 |
| C1R | C1R_HUMAN | Complement C1r subcomponent light chain |
| C1S | C1S_HUMAN | Complement C1s subcomponent light chain |
| C2 | CO2_HUMAN | Complement C2a fragment |
| C2CD2L | C2C2L_HUMAN | Phospholipid transfer protein C2CD2L |
| C3 | CO3_HUMAN | Complement C3c alpha′ chain fragment 2 |
| C4A | CO4A_HUMAN | Complement C4 gamma chain |
| C4B | CO4B_HUMAN | Complement C4 gamma chain |
| C4B_2 | | |
| C4BPA | C4BPA_HUMAN | C4b-binding protein alpha chain |
| C5 | CO5_HUMAN | Complement C5 alpha′ chain |
| C6 | CO6_HUMAN | Complement component C6 |
| C7 | CO7_HUMAN | Complement component C7 |
| CSA | CO8A_HUMAN | Complement component C8 alpha chain |
| C8B | CO8B_HUMAN | Complement component C8 beta chain |
| C8G | CO8G_HUMAN | Complement component C8 gamma chain |
| C9 | CO9_HUMAN | Complement component C9b |
| CA2 | CAH2_HUMAN | Carbonic anhydrase 2 |
| CA6 | CAH6_HUMAN | Carbonic anhydrase 6 |
| CABP1 | CABP1_HUMAN | Calcium-binding protein 1 |
| CACNG2 | CCG2_HUMAN | Voltage-dependent calcium channel gamma-2 subunit |
| CALCOCO2 | CACO2_HUMAN | Calcium-binding and coiled-coil domain containing protein 2 |
| CALM1 | CALM1_HUMAN | Calmodulin-1 |
| CALM2 | CALM2_HUMAN | Calmodulin-2 |
| CAMK1D | KCC1D_HUMAN | Calcium/calmodulin-dependent protein kinase type 1D |
| CAMK1G | KCC1G_HUMAN | Calcium/calmodulin-dependent protein kinase type 1G |
| CAMK2A | KCC2A_HUMAN | Calcium/calmodulin-dependent protein kinase type II subunit alpha |
| CAMK2B | KCC2B_HUMAN | Calcium/calmodulin-dependent protein kinase type II subunit beta |
| CAMK2D | KCC2D_HUMAN | Calcium/calmodulin-dependent protein kinase type II subunit delta |
| CAMKK1 | KKCC1_HUMAN | Calcium/calmodulin-dependent protein kinase kinase 1 |
| CAMKK2 | KKCC2_HUMAN | Calcium/calmodulin-dependent protein kinase kinase 2 |
| CANT1 | CANT1_HUMAN | Soluble calcium-activated nucleotidase 1 |
| CAPN15 | CAN15_HUMAN | Calpain-15 |
| CAPN2 | CAN2_HUMAN | Calpain-2 catalytic subunit |
| CAPN9 | CAN9_HUMAN | Calpain-9 |
| CAPNS1 | CPNS1_HUMAN | Calpain small subunit 1 |
| CAPR1N2 | CAPR2_HUMAN | Caprin-2 |
| CARHSP1 | CHSP1_HUMAN | Calcium-regulated heat-stable protein 1 |
| CARM1 | CARM1_HUMAN | Histone-arginine methyltransferase CARM1 |
| CASK | CSKP_HUMAN | Peripheral plasma membrane protein CASK |
| CASP1 | CASP1_HUMAN | Caspase-1 subunit p10 |
| CASP2 | CASP2_HUMAN | Caspase-2 subunit p12 |
| CASP3 | CASP3_HUMAN | Caspase-3 subunit p12 |
| CASP6 | CASP6_HUMAN | Caspase-6 subunit p11 |
| CASP7 | CASP7_HUMAN | Caspase-7 subunit p11 |
| CASP8 | CASP8_HUMAN | Caspase-8 subunit p10 |
| CASP9 | CASP9_HUMAN | Caspase-9 subunit p10 |
| CASR | CASR_HUMAN | Extracellular calcium-sensing receptor |
| CAT | CATA_HUMAN | Catalase |
| CBFA2T2 | MTG8R_HUMAN | Protein CBF A2T2 |
| CBFA2T3 | MTG16_HUMAN | Protein CBF A2T3 |
| CBFB | PEBB_HUMAN | Core-binding factor subunit beta |
| CBL | CBL_HUMAN | E3 ubiquitin-protein ligase CBL |
| CBLB | CBLB_HUMAN | E3 ubiquitin-protein ligase CBL-B |
| CBLC | CBLC_HUMAN | E3 ubiquitin-protein ligase CBL-C |
| CBLL1 | HAKA1_HUMAN | E3 ubiquitin-protein ligase Hakai |
| CBS | CBS_HUMAN | Cystathionine beta-synthase |
| CCL13 | CCL13_HUMAN | C-C motif chemokine 13, short chain |
| CCL14 | CCL14_HUMAN | HCC-1(9-74) |
| CCL17 | CCL17_HUMAN | C-C motif chemokine 17 |
| CCL18 | CCL18_HUMAN | CCL18(4-69) |
| CCL19 | CCL19_HUMAN | C-C motif chemokine 19 |
| CCL23 | CCL23_HUMAN | CCL23(30-99) |
| CCL24 | CCL24_HUMAN | C-C motif chemokine 24 |
| CCL26 | CCL26_HUMAN | C-C motif chemokine 26 |
| CCL8 | CCL8_HUMAN | MCP-2(6-76) |
| CCNB11P1 | C1P1_HUMAN | E3 ubiquitin-protein ligase CCNB11P1 |
| CCNT2 | CCNT2_HUMAN | Cyclin-T2 |
| CCR2 | CCR2_HUMAN | C-C chemokine receptor type 2 |
| CCR5 | CCR5_HUMAN | C-C chemokine receptor type 5 |
| CCS | CCS_HUMAN | Copper chaperone for superoxide dismutase |
| CCT5 | TCPE_HUMAN | T-complex protein 1 subunit epsilon |
| CD19 | CD19_HUMAN | B-lymphocyte antigen CD19 |
| CD1A | CD1A_HUMAN | T-cell surface glycoprotein CD1a |
| CD1B | CD1B_HUMAN | T-cell surface glycoprotein CD1b |
| CD1C | CD1C_HUMAN | T-cell surface glycoprotein CD1c |
| CD1D | CD1D_HUMAN | Antigen-presenting glycoprotein CD1d |
| CD1E | CD1E_HUMAN | T-cell surface glycoprotein CD1e, soluble |
| CD2 | CD2_HUMAN | T-cell surface antigen CD2 |
| CD207 | CLC4K_HUMAN | C-type lectin domain family 4 member K |
| CD22 | CD22_HUMAN | B-cell receptor CD22 |
| CD226 | CD226_HUMAN | CD226 antigen |
| CD2AP | CD2AP_HUMAN | CD2-associated protein |
| CD302 | CD302_HUMAN | CD302 antigen |
| CD320 | CD320_HUMAN | CD320 antigen |
| CD33 | CD33_HUMAN | Myeloid cell surface antigen CD33 |
| CD36 | CD36_HUMAN | Platelet glycoprotein 4 |
| CD4 | CD4_HUMAN | T-cell surface glycoprotein CD4 |
| CD44 | CD44_HUMAN | CD44 antigen |
| CD48 | CD48_HUMAN | CD48 antigen |
| CD5 | CD5_HUMAN | T-cell surface glycoprotein CD5 |
| CD55 | DAF_HUMAN | Complement decay-accelerating factor |
| CD58 | LFA3_HUMAN | Lymphocyte function-associated antigen 3 |
| CD74 | HG2A_HUMAN | HLA class II histocompatibility antigen gamma chain |
| CD86 | CD86_HUMAN | T-lymphocyte activation antigen CD86 |
| CD96 | TACT_HUMAN | T-cell surface protein tactile |
| CDA | CDD_HUMAN | Cytidine deaminase |
| CDC20 | CDC20_HUMAN | Cell division cycle protein 20 homolog |
| CDC40 | PRP17_HUMAN | Pre-mRNA-processing factor 17 |
| CDC42BPA | MRCKA_HUMAN | Serine/threonine-protein kinase MRCK alpha |
| CDC42BPB | MRCKB_HUMAN | Serine/threonine-protein kinase MRCK beta |
| CDC42BPG | MRCKG_HUMAN | Serine/threonine-protein kinase MRCK gamma |
| CDC45 | CDC45_HUMAN | Cell division control protein 45 homolog |
| CDH1 | CADH1_HUMAN | E-Cad/CTF3 |
| CDH13 | CAD13_HUMAN | Cadherin-13 |
| CDH23 | CAD23_HUMAN | Cadherin-23 |
| CDH3 | CADH3_HUMAN | Cadherin-3 |
| CDHR2 | CDHR2_HUMAN | Cadherin-related family member 2 |
| CDK1 | CDK1_HUMAN | Cyclin-dependent kinase 1 |
| CDK12 | CDK12_HUMAN | Cyclin-dependent kinase 12 |
| CDK13 | CDK13_HUMAN | Cyclin-dependent kinase 13 |
| CDK16 | CDK16_HUMAN | Cyclin-dependent kinase 16 |
| CDK2 | CDK2_HUMAN | Cyclin-dependent kinase 2 |
| CDK4 | CDK4_HUMAN | Cyclin-dependent kinase 4 |
| CDK5 | CDK5_HUMAN | Cyclin-dependent-like kinase 5 |
| CDK6 | CDK6_HUMAN | Cyclin-dependent kinase 6 |
| CDK7 | CDK7_HUMAN | Cyclin-dependent kinase 7 |
| CDK9 | CDK9_HUMAN | Cyclin-dependent kinase 9 |
| CDKL1 | CDKL1_HUMAN | Cyclin-dependent kinase-like 1 |
| CDKL2 | CDKL2_HUMAN | Cyclin-dependent kinase-like 2 |
| CDKL3 | CDKL3_HUMAN | Cyclin-dependent kinase-like 3 |
| CDKN2A | CDN2A_HUMAN | Cyclin-dependent kinase inhibitor 2A |
| CDKN2C | CDN2C_HUMAN | Cyclin-dependent kinase 4 inhibitor C |
| CDKN2D | CDN2D_HUMAN | Cyclin-dependent kinase 4 inhibitor D |
| CDO1 | CDO1_HUMAN | Cysteine dioxygenase type 1 |
| CDYL | CDYL_HUMAN | Chromodomain Y-like protein |
| CDYL2 | CDYL2_HUMAN | Chromodomain Y-like protein 2 |
| CEACAM5 | CEAM5_HUMAN | Carcinoembryonic antigen-related cell adhesion molecule 5 |
| CEACAM7 | CEAM7_HUMAN | Carcinoembryonic antigen-related cell adhesion molecule 7 |
| CEBPA | CEBPA_HUMAN | CCAAT/enhancer-binding protein alpha |
| CEL | CEL_HUMAN | Bile salt-activated lipase |
| CELF6 | CELF6_HUMAN | CUGBP Elav-like family member 6 |
| CEP104 | CE104_HUMAN | Centrosomal protein of 104 kDa |
| CEP170 | CE170_HUMAN | Centrosomal protein of 170 kDa |
| CES1 | ESTl_HUMAN | Liver carboxy lesterase 1 |
| CETP | CETP_HUMAN | Cholesteryl ester transfer protein |
| CFB | CFAB_HUMAN | Complement factor B Bb fragment |
| CFD | CFAD_HUMAN | Complement factor D |
| CFH | CFAH_HUMAN | Complement factor H |
| CFl | CFA1_HUMAN | Complement factor 1 light chain |
| CFP | PROP_HUMAN | Properdin |
| CFTR | CFTR_HUMAN | Cystic fibrosis transmembrane conductance regulator |
| CGA | GLHA_HUMAN | Glycoprotein hormones alpha chain |
| CHAMP1 | CHAP1_HUMAN | Chromosome alignment-maintaining phosphoprotein 1 |
| CHD1 | CHD1_HUMAN | Chromodomain-helicase-DNA-binding protein 1 |
| CHD4 | CHD4_HUMAN | Chromodomain-helicase-DNA-binding protein 4 |
| CHD6 | CHD6_HUMAN | Chromodomain-helicase-DNA-binding protein 6 |
| CHD7 | CHD7_HUMAN | Chromodomain-helicase-DNA-binding protein 7 |
| CHD8 | CHD8_HUMAN | Chromodomain-helicase-DNA-binding protein 8 |
| CHEK1 | CHK1_HUMAN | Serine/threonine-protein kinase Chk1 |
| CHFR | CHFR_HUMAN | E3 ubiquitin-protein ligase CHFR |
| CH1D1 | CH1D1_HUMAN | Chitinase domain-containing protein 1 |
| CHN1 | CH1N_HUMAN | N-chimaerin |
| CHN2 | CH1O_HUMAN | Beta-chimaerin |
| CHRM1 | ACM1_HUMAN | Muscarinic acetylcholine receptor M1 |
| CHRNA1 | ACHA_HUMAN | Acetylcholine receptor subunit alpha |
| CHRNA2 | ACHA2_HUMAN | Neuronal acetylcholine receptor subunit alpha-2 |
| CHRNA3 | ACHA3_HUMAN | Neuronal acetylcholine receptor subunit alpha-3 |
| CHRNA4 | ACHA4_HUMAN | Neuronal acetylcholine receptor subunit alpha-4 |
| CHRNA7 | ACHA7_HUMAN | Neuronal acetylcholine receptor subunit alpha-7 |
| CHRNA9 | ACHA9_HUMAN | Neuronal acetylcholine receptor subunit alpha-9 |
| CHRNB2 | ACHB2_HUMAN | Neuronal acetylcholine receptor subunit beta-2 |
| CHUK | IKKA_HUMAN | Inhibitor of nuclear factor kappa-B kinase subunit alpha |
| C1AO1 | C1AO1_HUMAN | Probable cytosolic iron-sulfur protein assembly protein C1AO1 |
| C1DEA | C1DEA_HUMAN | Cell death activator C1DE-A |
| C1DEB | C1DEB_HUMAN | Cell death activator C1DE-B |
| CKB | KCRB_HUMAN | Creatine kinase B-type |
| CKM | KCRM_HUMAN | Creatine kinase M-type |
| CKMTlA | KCRU_HUMAN | Creatine kinase U-type, mitochondrial |
| CKMTlB | | |
| CKMT2 | KCRS_HUMAN | Creatine kinase S-type, mitochondrial |
| CLDN2 | CLD2_HUMAN | Claudin-2 |
| CLDN4 | CLD4_HUMAN | Claudin-4 |
| CLEC2A | CLC2A_HUMAN | C-type lectin domain family 2 member A |
| CLEC2D | CLC2D_HUMAN | C-type lectin domain family 2 member D |
| CLEC4D | CLC4D_HUMAN | C-type lectin domain family 4 member D |
| CLEC4E | CLC4E_HUMAN | C-type lectin domain family 4 member E |
| CLEC4M | CLC4M_HUMAN | C-type lectin domain family 4 member M |
| CLEC6A | CLC6A_HUMAN | C-type lectin domain family 6 member A |
| CLEC9A | CLC9A_HUMAN | C-type lectin domain family 9 member A |
| CLK1 | CLK1_HUMAN | Dual specificity protein kinase CLK1 |
| CLK2 | CLK2_HUMAN | Dual specificity protein kinase CLK2 |
| CLK3 | CLK3_HUMAN | Dual specificity protein kinase CLK3 |
| CLPP | CLPP_HUMAN | ATP-dependent Clp protease proteolytic subunit, mitochondrial |
| CLPX | CLPX_HUMAN | ATP-dependent Clp protease ATP-binding subunit clpX-like, |
| | | mitochondrial |
| CLTC | CLH1_HUMAN | Clathrin heavy chain 1 |
| CMA1 | CMA1_HUMAN | Chymase |
| CNBP | CNBP_HUMAN | Cellular nucleic acid-binding protein |
| CNDP2 | CNDP2_HUMAN | Cytosolic non-specific dipeptidase |
| CNNM2 | CNNM2_HUMAN | Metal transporter CNNM2 |
| CNNM3 | CNNM3_HUMAN | Metal transporter CNNM3 |
| CNOT4 | CNOT4_HUMAN | CCR4-NOT transcription complex subunit 4 |
| CNOT7 | CNOT7_HUMAN | CCR4-NOT transcription complex subunit 7 |
| CNP | CN37_HUMAN | 2′,3′-cyclic-nucleotide 3′-phosphodiesterase |
| CNR2 | CNR2_HUMAN | Cannabinoid receptor 2 |
| CNTFR | CNTFR_HUMAN | Ciliary neurotrophic factor receptor subunit alpha |
| CNTN1 | CNTN1_HUMAN | Contactin-1 |
| CNTN2 | CNTN2_HUMAN | Contactin-2 |
| CNTN3 | CNTN3_HUMAN | Contactin-3 |
| CNTN5 | CNTN5_HUMAN | Contactin-5 |
| COL10A1 | COAA1_HUMAN | Collagen alpha- I(X) chain |
| COL1A1 | CO1A1_HUMAN | Collagen alpha-1(1) chain |
| COL20A1 | COKA1_HUMAN | Collagen alpha-1(XX) chain |
| COL3A1 | CO3A1_HUMAN | Collagen alpha-1(lll) chain |
| COL4A1 | CO4A1_HUMAN | Arresten |
| COL4A2 | CO4A2_HUMAN | Canstatin |
| COL4A3 | CO4A3_HUMAN | Tnmstatin |
| COL4A4 | CO4A4_HUMAN | Collagen alpha-4(1V) chain |
| COL4A5 | CO4A5_HUMAN | Collagen alpha-5(1V) chain |
| COLEC11 | COL11_HUMAN | Collectin-11 |
| COLEC12 | COL_12_HUMAN | Collectin-12 |
| COMP | COMP_HUMAN | Cartilage oligomeric matrix protein |
| COP1 | COP1_HUMAN | E3 ubiquitin-protein ligase COP1 |
| COPG1 | COPG1_HUMAN | Coatomer subunit gamma-1 |
| COPS3 | CSN3_HUMAN | COP9 signalosome complex subunit 3 |
| COPS4 | CSN4_HUMAN | COP9 signalosome complex subunit 4 |
| COQ8A | COQ8A_HUMAN | Atypical kinase COQ8A, mitochondrial |
| COX5B | COX5B_HUMAN | Cytochrome c oxidase subunit 5B, mitochondrial |
| CPA1 | CBPA1_HUMAN | Carboxypeptidase A1 |
| CPB1 | CBPB1_HUMAN | Carboxypeptidase B |
| CPD | CBPD_HUMAN | Carboxypeptidase D |
| CPM | CBPM_HUMAN | Carboxypeptidase M |
| CPN1 | CBPN_HUMAN | Carboxypeptidase N catalytic chain |
| CPOX | HEM6_HUMAN | Oxygen-dependent coproporphyrinogen-111 oxidase, mitochondrial |
| CPS1 | CPSM_HUMAN | Carbamoyl-phosphate synthase [ammonia], mitochondrial |
| CPSF1 | CPSF1_HUMAN | Cleavage and polyadenylation specificity factor subunit 1 |
| CPSF3 | CPSF3_HUMAN | Cleavage and polyadenylation specificity factor subunit 3 |
| CPSF4 | CPSF4_HUMAN | Cleavage and polyadenylation specificity factor subunit 4 |
| CPSF6 | CPSF6_HUMAN | Cleavage and polyadenylation specificity factor subunit 6 |
| CPSF7 | CPSF7_HUMAN | Cleavage and polyadenylation specificity factor subunit 7 |
| CR1 | CR1_HUMAN | Complement receptor type 1 |
| CR2 | CR2_HUMAN | Complement receptor type 2 |
| CRABP2 | RABP2_HUMAN | Cellular retinoic acid-binding protein 2 |
| CRBN | CRBN_HUMAN | Protein cereblon |
| CREBBP | CBP_HUMAN | CREB-binding protein |
| CRHR1 | CRFR1_HUMAN | Corticotropin-releasing factor receptor 1 |
| CRK | CRK_HUMAN | Adapter molecule erk |
| CRKL | CRKL_HUMAN | Crk-like protein |
| CRP | CRP_HUMAN | C-reactive protein(l-205) |
| CRTAM | CRTAM_HUMAN | Cytotoxic and regulatory T-cell molecule |
| CRYAB | CRYAB_HUMAN | Alpha-crystallin B chain |
| CRYM | CRYM_HUMAN | Ketimine reductase mu-crystallin |
| CS | C1SY_HUMAN | Citrate synthase, mitochondrial |
| CSAD | CSAD_HUMAN | Cysteine sulfinic acid decarboxylase |
| CSDE1 | CSDE1_HUMAN | Cold shock domain-containing protein E1 |
| CSF1R | CSF1R_HUMAN | Macrophage colony-stimulating factor 1 receptor |
| CSF3R | CSF3R_HUMAN | Granulocyte colony-stimulating factor receptor |
| CSK | CSK_HUMAN | Tyrosine-protein kinase CSK |
| CSNK1A1 | KC1A_HUMAN | Casein kinase 1 isoform alpha |
| CSNK1D | KC1D_HUMAN | Casein kinase 1 isoform delta |
| CSNK1E | KC1E_HUMAN | Casein kinase 1 isoform epsilon |
| CSNK1G3 | KC1G3_HUMAN | Casein kinase 1 isoform gamma-3 |
| CSRP3 | CSRP3_HUMAN | Cysteine and glycine-rich protein 3 |
| CST3 | CYTC_HUMAN | Cystatin-C |
| CSTF1 | CSTF1_HUMAN | Cleavage stimulation factor subunit 1 |
| CSTF2 | CSTF2_HUMAN | Cleavage stimulation factor subunit 2 |
| CTCF | CTCF_HUMAN | Transcriptional repressor CTCF |
| CTCFL | CTCFL_HUMAN | Transcriptional repressor CTCFL |
| CTLA4 | CTLA4_HUMAN | Cytotoxic T-lymphocyte protein 4 |
| CTPS1 | PYRG1_HUMAN | CTP synthase 1 |
| CTPS2 | PYRG2_HUMAN | CTP synthase 2 |
| CTRC | CTRC_HUMAN | Chymotrypsin-C |
| CTSA | PPGB_HUMAN | Lysosomal protective protein 20 kDa chain |
| CTSC | CATC_HUMAN | DipeptidyI peptidase 1 light chain |
| CTSD | CATD_HUMAN | Cathepsin D heavy chain |
| CTSE | CATE_HUMAN | Cathepsin E form 11 |
| CUL4B | CUL4B_HUMAN | Cullin-4B |
| CUL5 | CUL5_HUMAN | Cullin-5 |
| CUL7 | CUL7_HUMAN | Cullin-7 |
| CUL9 | CUL9_HUMAN | Cullin-9 |
| CUTC | CUTC_HUMAN | Copper homeostasis protein cutC homolog |
| CWC27 | CWC27_HUMAN | Spliceosome-associated protein CWC27 homolog |
| CWF19L2 | C19L2_HUMAN | CWF19-like protein 2 |
| CXADR | CXAR_HUMAN | Coxsackievirus and adenovirus receptor |
| CXCL10 | CXL10_HUMAN | CXCL 10(1-73) |
| CXCL2 | CXCL2_HUMAN | GRO-beta(5-73) |
| CXCL5 | CXCL5_HUMAN | EN A-78(9-78) |
| CXCL8 | 1L8_HUMAN | 1L-8(9-77) |
| CXCR4 | CXCR4_HUMAN | C-X-C chemokine receptor type 4 |
| CYC1 | CY1_HUMAN | Cytochrome cl, heme protein, mitochondrial |
| CYHR1 | CYHR1_HUMAN | Cysteine and histidine-rich protein 1 |
| CYLD | CYLD_HUMAN | Ubiquitin carboxyl-terminal hydrolase CYLD |
| CYP51A1 | CP51A_HUMAN | Lanosterol 14-alpha demethylase |
| CYP7A1 | CP7A1_HUMAN | Cholesterol 7-alpha-monooxygenase |
| CYTH3 | CYH3_HUMAN | Cytohesin-3 |
| CZ1B | CZ1B_HUMAN | CXXC motif containing zinc binding protein |
| DAG1 | DAG1_HUMAN | Beta-dystroglycan |
| DAPK1 | DAPK1_HUMAN | Death-associated protein kinase 1 |
| DAPK2 | DAPK2_HUMAN | Death-associated protein kinase 2 |
| DAPK3 | DAPK3_HUMAN | Death-associated protein kinase 3 |
| DARS2 | SYDM_HUMAN | Aspartate--tRNA ligase, mitochondrial |
| DAW1 | DAW1_HUMAN | Dynein assembly factor with WDR repeat domains 1 |
| DBH | DOPO_HUMAN | Soluble dopamine beta-hydroxylase |
| DBNL | DBNL_HUMAN | Drebrin-like protein |
| DCAF1 | DCAF1_HUMAN | DDB1- and CUL4-associated factor 1 |
| DCC | DCC_HUMAN | Netrin receptor DCC |
| DCDC2 | DCDC2_HUMAN | Doublecortin domain-containing protein 2 |
| DCLK1 | DCLK1_HUMAN | Serine/threonine-protein kinase DCLK1 |
| DCLRE1A | DCR1A_HUMAN | DNA cross-link repair 1A protein |
| DCLRE1B | DCR1B_HUMAN | 5′ exonuclease Apollo |
| DCTN1 | DCTN1_HUMAN | Dynactin subunit 1 |
| DCTN5 | DCTN5_HUMAN | Dynactin subunit 5 |
| DCUN1D1 | DCNL1_HUMAN | DCN1-like protein 1 |
| DCX | DCX_HUMAN | Neuronal migration protein doublecortin |
| DDAH1 | DDAH1_HUMAN | N(G),N(G)-dimethylarginine dimethylaminohydrolase 1 |
| DDB1 | DDB1_HUMAN | DNA damage-binding protein 1 |
| DDB2 | DDB2_HUMAN | DNA damage-binding protein 2 |
| DD11 | DD11_HUMAN | Protein DD11 homolog 1 |
| DD12 | DDl2_HUMAN | Protein DD11 homolog 2 |
| DDR1 | DDR1_HUMAN | Epithelial discoidin domain-containing receptor 1 |
| DDX1 | DDX1_HUMAN | ATP-dependent RNA helicase DDX1 |
| DDX39B | DX39B_HUMAN | Spliceosome RNA helicase DDX39B |
| DDX41 | DDX41_HUMAN | Probable ATP-dependent RNA helicase DDX41 |
| DDX58 | DDX58_HUMAN | Probable ATP-dependent RNA helicase DDX58 |
| DDX59 | DDX59_HUMAN | Probable ATP-dependent RNA helicase DDX59 |
| DEAF1 | DEAF1_HUMAN | Deformed epidermal autoregulatory factor 1 homolog |
| DEFA1\| | DEF1_HUMAN | Neutrophil defensin 2 |
| DEFA1B | | |
| DEFB4A\| | DFB4A_HUMAN | Beta-defensin 4A |
| DEFB4B | | |
| DES11 | DES11_HUMAN | Desumoylating isopeptidase 1 |
| DFFA | DFFA_HUMAN | DNA fragmentation factor subunit alpha |
| DFFB | DFFB_HUMAN | DNA fragmentation factor subunit beta |
| DGKE | DGKE_HUMAN | Diacylglycerol kinase epsilon |
| DGK1 | DGK1_HUMAN | Diacylglycerol kinase iota |
| DGKK | DGKK_HUMAN | Diacylglycerol kinase kappa |
| DGKQ | DGKQ_HUMAN | Diacylglycerol kinase theta |
| DGKZ | DGKZ_HUMAN | Diacylglycerol kinase zeta |
| DHFR | DYR_HUMAN | Dihydrofolate reductase |
| DHX16 | DHX16_HUMAN | Pre-mRNA-splicing factor ATP-dependent RNA helicase DHX16 |
| DHX58 | DHX58_HUMAN | Probable ATP-dependent RNA helicase DHX58 |
| DHX8 | DHX8_HUMAN | ATP-dependent RNA helicase DHX8 |
| DHX9 | DHX9_HUMAN | ATP-dependent RNA helicase A |
| DICER1 | DICER_HUMAN | Endoribonuclease Dicer |
| D1S3 | RRP44_HUMAN | Exosome complex exonuclease RRP44 |
| DIXDC1 | DIXC1_HUMAN | Dixin |
| DLAT | ODP2_HUMAN | Dihydrolipoyllysine-residue acetyltransferase component of pyruvate |
| | | dehydrogenase complex, mitochondrial |
| DLD | DLDH_HUMAN | DihydrolipoyI dehydrogenase, mitochondrial |
| DLG5 | DLG5_HUMAN | Disks large homolog 5 |
| DLL1 | DLL1_HUMAN | Delta-like protein 1 |
| DLL4 | DLL4_HUMAN | Delta-like protein 4 |
| DMC1 | DMC1_HUMAN | Meiotic recombination protein DMC1/LIM15 homolog |
| DMGDH | M2GD_HUMAN | Dimethylglycine dehydrogenase, mitochondrial |
| DMPK | DMPK_HUMAN | Myotonin-protein kinase |
| DNAJA1 | DNJA1_HUMAN | DnaJ homolog subfamily A member 1 |
| DNAJA3 | DNJA3_HUMANV | DnaJ homolog subfamily A member 3, mitochondrial |
| DNAJB1 | DNJB1_HUMAN | DnaJ homolog subfamily B member 1 |
| DNAJC24 | DJC24_HUMAN | DnaJ homolog subfamily C member 24 |
| DNLZ | DNLZ_HUMAN | DNL-type zinc finger protein |
| DNMT1 | DNMT1_HUMAN | DNA (cytosine-5)-methyltransferase 1 |
| DNMT3A | DNM3A_HUMAN | DNA (cytosine-5)-methyltransferase 3A |
| DNMT3B | DNM3B_HUMAN | DNA (cytosine-5)-methyltransferase 3B |
| DNMT3L | DNM3L_HUMAN | DNA (cytosine-5)-methyltransferase 3-like |
| DNPEP | DNPEP_HUMAN | AspartyI aminopeptidase |
| DOK2 | DOK2_HUMAN | Docking protein 2 |
| DPAGT1 | GPT_HUMAN | UDP-N-acetylglucosamine--dolichyl-phosphate N- |
| | | acetylglucosaminephosphotransferase |
| DPF1 | DPF1_HUMAN | Zinc finger protein neuro-d4 |
| DPF2 | REQU_HUMAN | Zinc finger protein ubi-d4 |
| DPF3 | DPF3_HUMAN | Zinc finger protein DPF3 |
| DPP10 | DPP10_HUMAN | Inactive dipeptidyI peptidase 10 |
| DPP3 | DPP3_HUMAN | DipeptidyI peptidase 3 |
| DPP4 | DPP4_HUMAN | Dipeptidyl peptidase 4 soluble form |
| DPP6 | DPP6_HUMAN | Dipeptidyl aminopeptidase-like protein 6 |
| DPP8 | DPP8_HUMAN | DipeptidyI peptidase 8 |
| DPP9 | DPP9_HUMAN | DipeptidyI peptidase 9 |
| DRD2 | DRD2_HUMAN | D(2) dopamine receptor |
| DRD3 | DRD3_HUMAN | D(3) dopamine receptor |
| DROSHA | RNC_HUMAN | Ribonuclease 3 |
| DSC1 | DSC1_HUMAN | Desmocollin-1 |
| DSC2 | DSC2_HUMAN | Desmocollin-2 |
| DSG2 | DSG2_HUMAN | Desmoglein-2 |
| DSG3 | DSG3_HUMAN | Desmoglein-3 |
| DSP | DESP_HUMAN | Desmoplakin |
| DTD1 | DTD1_HUMAN | D-aminoacy1-tRNA deacylase 1 |
| DTX3 | DTX3_HUMAN | Probable E3 ubiquitin-protein ligase DTX3 |
| DTX3L | DTX3L_HUMAN | E3 ubiquitin-protein ligase DTX3L |
| DUSP14 | DUS14_HUMAN | Dual specificity protein phosphatase 14 |
| DVL2 | DVL2_HUMAN | Segment polarity protein dishevelled homolog DVL-2 |
| DYNC1H1 | DYHC1_HUMAN | Cytoplasmic dynein 1 heavy chain 1 |
| DYNC112 | DC112_HUMAN | Cytoplasmic dynein 1 intermediate chain 2 |
| DYNC2H1 | DYHC2_HUMAN | Cytoplasmic dynein 2 heavy chain 1 |
| DYNLRB1 | DLRB1_HUMAN | Dynein light chain roadblock-type 1 |
| DYRK1A | DYR1A_HUMAN | Dual specificity tyrosine-phosphorylation regulated-kinase 1A |
| DYRK2 | DYRK2_HUMAN | Dual specificity tyrosine-phosphorylation-regulated kinase 2 |
| DYRK3 | DYRK3_HUMAN | Dual specificity tyrosine-phosphorylation-regulated kinase 3 |
| DYSF | DYSF_HUMAN | Dysferlin |
| DZANK1 | DZAN1_HUMAN | Double zinc ribbon and ankyrin repeat-containing protein 1 |
| E4F1 | E4F1_HUMAN | Transcription factor E4F1 |
| EBF1 | COE1_HUMAN | Transcription factor COE1 |
| ECE1 | ECE1_HUMAN | Endothelin-converting enzyme 1 |
| EC11 | EC11_HUMAN | Enoyl-CoA delta isomerase 1, mitochondrial |
| EDA | EDA_HUMAN | Ectodysplasin-A, secreted form |
| EDC3 | EDC3_HUMAN | Enhancer of mRNA-decapping protein 3 |
| EDNRB | EDNRB_HUMAN | Endothelin receptor type B |
| EEA1 | EEA1_HUMAN | Early endosome antigen 1 |
| EED | EED_HUMAN | Polycomb protein EED |
| EEF1G | EF1G_HUMAN | Elongation factor 1-gamma |
| EEFSEC | SELB_HUMAN | Selenocysteine-specific elongation factor |
| EFEMP2 | FBLN4_HUMAN | EGF-containing fibulin-like extracellular matrix protein 2 |
| EFL1 | EFL1_HUMAN | Elongation factor-like GTPase 1 |
| EFTUD2 | U5S1_HUMAN | 116 kDa U5 small nuclear ribonucleoprotein component |
| EGFR | EGFR_HUMAN | Epidermal growth factor receptor |
| EGLN1 | EGLN1_HUMAN | Egl nine homolog 1 |
| EGR1 | EGR1_HUMAN | Early growth response protein 1 |
| EGR2 | EGR2_HUMAN | E3 SUMO-protein ligase EGR2 |
| EGR3 | EGR3_HUMAN | Early growth response protein 3 |
| EGR4 | EGR4_HUMAN | Early growth response protein 4 |
| EHMT1 | EHMT1_HUMAN | Histone-lysine N-methyltransferase EHMT1 |
| EHMT2 | EHMT2_HUMAN | Histone-lysine N-methyltransferase EHMT2 |
| E1F1 | E1F1_HUMAN | Eukaryotic translation initiation factor 1 |
| E1F1AD | E1F1A_HUMAN | Probable RNA-binding protein E1F1AD |
| E1F2AK2 | E2AK2_HUMAN | Interferon-induced, double-stranded RNA-activated protein kinase |
| E1F2AK3 | E2AK3_HUMAN | Eukaryotic translation initiation factor 2-alpha kinase 3 |
| E1F2B1 | E12BA_HUMAN | Translation initiation factor e1F-2B subunit alpha |
| E1F2B2 | E12BB_HUMAN | Translation initiation factor e1F-2B subunit beta |
| E1F2B4 | E12BD_HUMAN | Translation initiation factor e1F-2B subunit delta |
| E1F2D | E1F2D_HUMAN | Eukaryotic translation initiation factor 2D |
| E1F2S1 | 1F2A_HUMAN | Eukaryotic translation initiation factor 2 subunit 1 |
| E1F3B | E1F3B_HUMAN | Eukaryotic translation initiation factor 3 subunit B |
| E1F3E | E1F3E_HUMAN | Eukaryotic translation initiation factor 3 subunit E |
| E1F3G | E1F3G_HUMAN | Eukaryotic translation initiation factor 3 subunit G |
| E1F4EBP2 | 4EBP2_HUMAN | Eukaryotic translation initiation factor 4E-binding protein 2 |
| E1F4G1 | IF4G1_HUMAN | Eukaryotic translation initiation factor 4 gamma 1 |
| E1F5 | IFS_HUMAN | Eukaryotic translation initiation factor 5 |
| E1F5A | 1F5A1_HUMAN | Eukaryotic translation initiation factor 5A-1 |
| ELAC1 | RNZ1_HUMAN | Zinc phosphodiesterase ELAC protein 1 |
| ELAVL1 | ELAV1_HUMAN | ELA V-like protein 1 |
| ELAVL4 | ELAV4_HUMAN | ELA V-like protein 4 |
| ELF5 | ELF5_HUMAN | ETS-related transcription factor Elf-5 |
| ELK1 | ELK1_HUMAN | ETS domain-containing protein Elk-1 |
| ELK4 | ELK4_HUMAN | ETS domain-containing protein Elk-4 |
| ELL | ELL_HUMAN | RNA polymerase II elongation factor ELL |
| ELOC | ELOC_HUMAN | Elongin-C |
| EMIL1N1 | EMIL1_HUMAN | EMILIN-1 |
| EML1 | EMAL1_HUMAN | Echinoderm rnicrotubule-associated protein-like 1 |
| ENO1 | ENOA_HUMAN | Alpha-enolase |
| ENO2 | ENOG_HUMAN | Gamma-enolase |
| ENO3 | ENOB_HUMAN | Beta-enolase |
| ENPEP | AMPE_HUMAN | Glutamyl arninopeptidase |
| EP300 | EP300_HUMAN | Histone acetyltransferase p300 |
| EPAS1 | EPAS1_HUMAN | Endothelial PAS domain-containing protein 1 |
| EPB41 | 41_HUMAN | Protein 4.1 |
| EPB41L3 | E41L3_HUMAN | Band 4.1-like protein 3, N-terminally processed |
| EPCAM | EPCAM_HUMAN | Epithelial cell adhesion molecule |
| EPDR1 | EPDR1_HUMAN | Mammalian ependymin-related protein 1 |
| EPHA2 | EPHA2_HUMAN | Ephrin type-A receptor 2 |
| EPHA3 | EPHA3_HUMAN | Ephrin type-A receptor 3 |
| EPHA4 | EPHA4_HUMAN | Ephrin type-A receptor 4 |
| EPHA5 | EPHA5_HUMAN | Ephrin type-A receptor 5 |
| EPHB4 | EPHB4_HUMAN | Ephrin type-B receptor 4 |
| EPM2A | EPM2A_HUMAN | Laforin |
| EPOR | EPOR_HUMAN | Erythropoietin receptor |
| EPRS | SYEP_HUMAN | Proline--tRNA ligase |
| EPS8L1 | ES8L1_HUMAN | Epidermal growth factor receptor kinase substrate 8-like protein 1 |
| EPS8L2 | ES8L2_HUMAN | Epidermal growth factor receptor kinase substrate 8-like protein 2 |
| EPS8L3 | ES8L3_HUMAN | Epidermal growth factor receptor kinase substrate 8-like protein 3 |
| ERAP1 | ERAP1_HUMAN | Endoplasmic reticulum aminopeptidase 1 |
| ERAP2 | ERAP2_HUMAN | Endoplasmic reticulum aminopeptidase 2 |
| ERBB2 | ERBB2_HUMAN | Receptor tyrosine-protein kinase erbB-2 |
| ERBB3 | ERBB3_HUMAN | Receptor tyrosine-protein kinase erbB-3 |
| ERCC6L2 | ER6L2_HUMAN | DNA excision repair protein ERCC-6-like 2 |
| ERCC8 | ERCC8_HUMAN | DNA excision repair protein ERCC-8 |
| ERG | ERG_HUMAN | Transcriptional regulator ERG |
| ERN1 | ERN1_HUMAN | Endoribonuclease |
| ERVK-10 | GAK10_HUMAN | Endogenous retrovirus group K member 10 Gag polyprotein |
| ERVK-19 | GAK19_HUMAN | Endogenous retrovirus group K member 19 Gag polyprotein |
| ERVK-21 | GAK21_HUMAN | Endogenous retrovirus group K member 21 Gag polyprotein |
| ERVK-24 | GAK24_HUMAN | Endogenous retrovirus group K member 24 Gag polyprotein |
| ERVK-5 | GAK5_HUMAN | Endogenous retrovirus group K member 5 Gag polyprotein |
| ERVK-6 | GAK5_HUMAN | Endogenous retrovirus group K member 6 Gag polyprotein |
| ERVK-7 | GAK7_HUMAN | Endogenous retrovirus group K member 7 Gag polyprotein |
| ERVK-8 | GAK8_HUMAN | Endogenous retrovirus group K member 8 Gag polyprotein |
| ERVK-9 | POK9_HUMAN | Reverse transcriptase/ribonuclease H |
| ERVK-9 | GAK9_HUMAN | Endogenous retrovirus group K member 9 Gag polyprotein |
| ESCO1 | ESCO1_HUMAN | N-acetyltransferase ESCO1 |
| ESCO2 | ESCO2_HUMAN | N-acetyltransferase ESCO2 |
| ESRRA | ERR1_HUMAN | Steroid hormone receptor ERR1 |
| ESRRB | ERR2_HUMAN | Steroid hormone receptor ERR2 |
| ESRRG | ERR3_HUMAN | Estrogen-related receptor gamma |
| ETF1 | ERF1_HUMAN | Eukaryotic peptide chain release factor subunit 1 |
| ETFB | ETFB_HUMAN | Electron transfer flavoprotein subunit beta |
| EVPL | EVPL_HUMAN | Envoplakin |
| EWSR1 | EWS_HUMAN | RNA-binding protein EWS |
| EXO1 | EXO1_HUMAN | Exonuclease 1 |
| EXOG | EXOG_HUMAN | Nuclease EXOG, mitochondrial |
| EXOSC2 | EXOS2_HUMAN | Exosome complex component RRP4 |
| EXOSC4 | EXOS4_HUMAN | Exosome complex component RRP41 |
| EXOSC5 | EXOS5_HUMAN | Exosome complex component RRP46 |
| EXOSC7 | EXOS7_HUMAN | Exosome complex component RRP42 |
| EXOSC9 | EXOS9_HUMAN | Exosome complex component RRP45 |
| EZH2 | EZH2_HUMAN | Histone-lysine N-methyltransferase EZH2 |
| EZR | EZR1_HUMAN | Ezrin |
| F10 | FA10_HUMAN | Activated factor Xa heavy chain |
| F11 | FA11_HUMAN | Coagulation factor X1a light chain |
| F11R | JAM1_HUMAN | Junctional adhesion molecule A |
| F12 | FA12_HUMAN | Coagulation factor Xlla light chain |
| F13A1 | Fl3A_HUMAN | Coagulation factor Xlll A chain |
| F2 | THRB_HUMAN | Thrombin heavy chain |
| F2R | PAR1_HUMAN | Proteinase-activated receptor 1 |
| F2RL1 | PAR2_HUMAN | Proteinase-activated receptor 2, alternate cleaved 2 |
| F3 | TF_HUMAN | Tissue factor |
| F5 | FA5_HUMAN | Coagulation factor V light chain |
| F7 | FA7_HUMAN | Factor Vll heavy chain |
| F8 | FA8_HUMAN | Factor VIIa light chain |
| F9 | FA9_HUMAN | Coagulation factor IXa heavy chain |
| FABP1 | FABPL_HUMAN | Fatty acid-binding protein, liver |
| FABP2 | FABPI_HUMAN | Fatty acid-binding protein, intestinal |
| FABP5 | FABP5_HUMAN | Fatty acid-binding protein 5 |
| FABP6 | FABP6_HUMAN | Gastrotropin |
| FAF1 | FAF1_HUMAN | FAS-associated factor 1 |
| FAIM | FAIM1_HUMAN | Fas apoptotic inhibitory molecule 1 |
| FAM3C | FAM3C_HUMAN | Protein FAM3C |
| FAM83A | FA83A_HUMAN | Protein FAM83A |
| FAM83B | FA83B_HUMAN | Protein FAM83B |
| FAN1 | FAN1_HUMAN | Fanconi-associated nuclease 1 |
| FANCF | FANCF_HUMAN | Fanconi anemia group F protein |
| FANCL | FANCL_HUMAN | E3 ubiquitin-protein ligase FANCL |
| FAP | SEPR_HUMAN | Antiplasmin-cleaving enzyme F AP, soluble form |
| FARSB | SYFB_HUMAN | Phenylalanine--tRNA ligase beta subunit |
| FASN | FAS_HUMAN | Oleoyl-[acyl-carrier-protein] hydrolase |
| FBL | FBRL_HUMAN | rRNA 2′-0-methyltransferase fibrillarin |
| FBN1 | FBN1_HUMAN | Asprosin |
| FBP1 | F16P1_HUMAN | Fmctose-1,6-bisphosphatase 1 |
| FBP2 | F16P2_HUMAN | Fmctose-1,6-bisphosphatase isozyme 2 |
| FBXL19 | FXL19_HUMAN | F-box/LRR-repeat protein 19 |
| FBX03 | FBX3_HUMAN | F-box only protein 3 |
| FBX031 | FBX31_HUMAN | F-box only protein 31 |
| FBX043 | FBX43_HUMAN | F-box only protein 43 |
| FBXW7 | FBXW7_HUMAN | F-box/WD repeat-containing protein 7 |
| FCER2 | FCER2_HUMAN | Low affinity immunoglobulin epsilon Fe receptor soluble form |
| FCGRT | FCGRN_HUMAN | IgG receptor FcRn large subunit p51 |
| FCHSD2 | FCSD2_HUMAN | F-BAR and double SH3 domains protein 2 |
| FCN1 | FCN1_HUMAN | Ficolin-1 |
| FCN3 | FCN3_HUMAN | Ficolin-3 |
| FDX1 | ADX_HUMAN | Adrenodoxin, mitochondrial |
| FDX2 | FDX2_HUMAN | Ferredoxin-2, mitochondrial |
| FEN1 | FEN1_HUMAN | Flap endonuclease 1 |
| FER | FER_HUMAN | Tyrosine-protein kinase Fer |
| FES | FES_HUMAN | Tyrosine-protein kinase Fes/Fps |
| FEV | FEV_HUMAN | Protein FEV |
| FEZF1 | FEZF1_HUMAN | Fez family zinc finger protein 1 |
| FEZF2 | FEZF2_HUMAN | Fez family zinc finger protein 2 |
| FFAR1 | FFAR1_HUMAN | Free fatty acid receptor 1 |
| FGA | FIBA_HUMAN | Fibrinogen alpha chain |
| FGB | FIBB_HUMAN | Fibrinogen beta chain |
| FGD1 | FGD1_HUMAN | FYVE, RhoGEF and PH domain-containing protein 1 |
| FGD2 | FGD2_HUMAN | FYVE, RhoGEF and PH domain-containing protein 2 |
| FGD3 | FGD3_HUMAN | FYVE, RhoGEF and PH domain-containing protein 3 |
| FGD4 | FGD4_HUMAN | FYVE, RhoGEF and PH domain-containing protein 4 |
| FGD5 | FGD5_HUMAN | FYVE, RhoGEF and PH domain-containing protein 5 |
| FGD6 | FGD6_HUMAN | FYVE, RhoGEF and PH domain-containing protein 6 |
| FGF1 | FGF1_HUMAN | Fibroblast growth factor 1 |
| FGF10 | FGF10_HUMAN | Fibroblast growth factor 10 |
| FGF12 | FGF12_HUMAN | Fibroblast growth factor 12 |
| FGF13 | FGF13_HUMAN | Fibroblast growth factor 13 |
| FGF18 | FGF18_HUMAN | Fibroblast growth factor 18 |
| FGF19 | FGF19_HUMAN | Fibroblast growth factor 19 |
| FGF2 | FGF2_HUMAN | Fibroblast growth factor 2 |
| FGF20 | FGF20_HUMAN | Fibroblast growth factor 20 |
| FGF23 | FGF23_HUMAN | Fibroblast growth factor 23 C-terminal peptide |
| FGF4 | FGF4_HUMAN | Fibroblast growth factor 4 |
| FGF8 | FGF8_HUMAN | Fibroblast growth factor 8 |
| FGF9 | FGF9_HUMAN | Fibroblast growth factor 9 |
| FGFR1 | FGFR1_HUMAN | Fibroblast growth factor receptor 1 |
| FGFR2 | FGFR2_HUMAN | Fibroblast growth factor receptor 2 |
| FGFR3 | FGFR3_HUMAN | Fibroblast growth factor receptor 3 |
| FGFR4 | FGFR4_HUMAN | Fibroblast growth factor receptor 4 |
| FGG | FIBG_HUMAN | Fibrinogen gamma chain |
| FH | FUMH_HUMAN | Fumarate hydratase, mitochondrial |
| FHL2 | FHL2_HUMAN | Four and a half LIM domains protein 2 |
| FHL3 | FHL3_HUMAN | Four and a half LIM domains protein 3 |
| FHOD1 | FHOD1_HUMAN | FH1/FH2 domain-containing protein 1 |
| FIBCD1 | FBCD1_HUMAN | Fibrinogen C domain-containing protein 1 |
| FIZ1 | FIZ1_HUMAN | Flt3-interacting zinc finger protein 1 |
| FKBP14 | FKB14_HUMAN | Peptidyl-prolyl cis-trans isomerase FKBP14 |
| FKBP1A | FKB1A_HUMAN | Peptidyl-prolyl cis-trans isomerase FKBP1A |
| FKBP3 | FKBP3_HUMAN | Peptidyl-prolyl cis-trans isomerase FKBP3 |
| FKBP4 | FKBP4_HUMAN | Peptidy1-prolyl cis-trans isomerase FKBP4, N-terminally processed |
| FKBP5 | FKBP5_HUMAN | Peptidyl-prolyl cis-trans isomerase FKBP5 |
| FKBP8 | FKBP8_HUMAN | Peptidyl-prolyl cis-trans isomerase FKBP8 |
| FLI1 | FLI1_HUMAN | Friend leukemia integration 1 transcription factor |
| FLNA | FLNA_HUMAN | Filamin-A |
| FLNB | FLNB_HUMAN | Filamin-B |
| FLNC | FLNC_HUMAN | Filamin-C |
| FLT1 | VGFR1_HUMAN | Vascular endothelial growth factor receptor 1 |
| FLT3 | FLT3_HUMAN | Receptor-type tyrosine-protein kinase FLT3 |
| FLT4 | VGFR3_HUMAN | Vascular endothelial growth factor receptor 3 |
| FLYWCH1 | FWCH1_HUMAN | FLYWCH-type zinc finger-containing protein 1 |
| FMR1 | FMR1_HUMAN | Synaptic functional regulator FMRI |
| FN1 | FINC_HUMAN | Ugl-Y3 |
| FNDC3A | FND3A_HUMAN | Fibronectin type-III domain-containing protein 3A |
| FNTB | FNTB_HUMAN | Protein famesyltransferase subunit beta |
| FOLH1 | FOLH1_HUMAN | Glutamate carboxypeptidase 2 |
| FOXO3 | FOXO3_HUMAN | Forkhead box protein O3 |
| FOXP2 | FOXP2_HUMAN | Forkhead box protein P2 |
| FOXP3 | FOXP3_HUMAN | Forkhead box protein P3 41 kDa form |
| FRS2 | FRS2_HUMAN | Fibroblast growth factor receptor substrate 2 |
| FRS3 | FRS3_HUMAN | Fibroblast growth factor receptor substrate 3 |
| FSCN1 | FSCN1_HUMAN | Fascin |
| FST | FST_HUMAN | Follistatin |
| FSTL3 | FSTL3_HUMAN | Follistatin-related protein 3 |
| FTO | FTO_HUMAN | Alpha-ketoglutarate-dependent dioxygenase FTO |
| FURIN | FURIN_HUMAN | Furin |
| FUS | FUS_HUMAN | RNA-binding protein FUS |
| FUT8 | FUT8_HUMAN | Alpha-(1,6)-fucosy ltransferase |
| FXN | FRDA_HUMAN | Frataxin mature form |
| FXR1 | FXR1_HUMAN | Fragile X mental retardation syndrome-related protein 1 |
| FXR2 | FXR2_HUMAN | Fragile X mental retardation syndrome-related protein 2 |
| FYB1 | FYB1_HUMAN | FYN-binding protein 1 |
| FYCO1 | FYCO1_HUMAN | FYVE and coiled-coil domain-containing protein 1 |
| FYN | FYN_HUMAN | Tyrosine-protein kinase Fyn |
| FZD4 | FZD4_HUMAN | Frizzled-4 |
| FZR1 | FZR1_HUMAN | Fizzy-related protein homolog |
| G2E3 | G2E3_HUMAN | G2/M phase-specific E3 ubiquitin-protein ligase |
| G3BP1 | G3BP1_HUMAN | Ras GTPase-activating protein-binding protein 1 |
| GAA | LYAG_HUMAN | 70 kDa lysosomal alpha-glucosidase |
| GABBR1 | GABR1_HUMAN | Gamma-aminobutyric acid type B receptor subunit 1 |
| GABRA1 | GBRA1_HUMAN | Gamma-aminobutyric acid receptor subunit alpha-1 |
| GABRA5 | GBRA5_HUMAN | Gamma-aminobutyric acid receptor subunit alpha-5 |
| GABRB2 | GBRB2_HUMAN | Gamma-aminobutyric acid receptor subunit beta-2 |
| GABRB3 | GBRB3_HUMAN | Gamma-aminobutyric acid receptor subunit beta-3 |
| GABRG2 | GBRG2_HUMAN | Gamma-aminobutyric acid receptor subunit gamma-2 |
| GAD1 | DCE1_HUMAN | Glutamate decarboxylase 1 |
| GAD2 | DCE2_HUMAN | Glutamate decarboxylase 2 |
| GAK | GAK_HUMAN | Cyclin-G-associated kinase |
| GALM | GALM_HUMAN | Aldose 1-epimerase |
| GALNS | GALNS_HUMAN | N-acetylgalactosamine-6-sulfatase |
| GALNT10 | GLT10_HUMAN | Polypeptide N-acetylgalactosaminyltransferase 10 |
| GALNT4 | GALT4_HUMAN | Polypeptide N-acetylgalactosaminyltransferase 4 |
| GALNT7 | GALT7_HUMAN | N-acetylgalactosaminyltransferase 7 |
| GALT | GALT_HUMAN | Galactose-1-phosphate uridylyltransferase |
| GARS | GARS_HUMAN | Glycine--tRNA Iigase |
| GART | PUR2_HUMAN | Phosphoribosylglycinamide formyltransferase |
| GAS7 | GAS7_HUMAN | Growth arrest-specific protein 7 |
| GATA1 | GATA1_HUMAN | Erythroid transcription factor |
| GATA2 | GATA2_HUMAN | Endothelial transcription factor GATA-2 |
| GATA3 | GATA3_HUMAN | Trans-acting T-cell-specific transcription factor GATA-3 |
| GATA4 | GATA4_HUMAN | Transcription factor GATA-4 |
| GATA5 | GATA5_HUMAN | Transcription factor GATA-5 |
| GATA6 | GATA6_HUMAN | Transcription factor GATA-6 |
| GBA | GLCM_HUMAN | Lysosomal acid glucosylceramidase |
| GBA3 | GBA3_HUMAN | Cytosolic beta-glucosidase |
| GBE1 | GLGB_HUMAN | 1,4-alpha-glucan-branching enzyme |
| GCA | GRAN_HUMAN | Grancalcin |
| GCGR | GLR_HUMAN | Glucagon receptor |
| GCK | HXK4_HUMAN | Glucokinase |
| GDF15 | GDF15_HUMAN | Growth/differentiation factor 15 |
| GDF2 | GDF2_HUMAN | Growth/differentiation factor 2 |
| GEMIN5 | GEM15_HUMAN | Gem-associated protein 5 |
| GEMIN7 | GEM17_HUMAN | Gem-associated protein 7 |
| GFI1 | GFI1_HUMAN | Zinc finger protein Gfi-1 |
| GFI1B | GFI1B_HUMAN | Zinc finger protein Gfi-Ib |
| GFM1 | EFGM_HUMAN | Elongation factor G, mitochondrial |
| GFRA3 | GFRA3_HUMAN | GDNF family receptor alpha-3 |
| GGCT | GGCT_HUMAN | Gamma-glutamyIcyclotransferase |
| GGT1 | GGT1_HUMAN | Glutathione hydrolase 1 light chain |
| GHR | GHR_HUMAN | Growth hormone-binding protein |
| GINS2 | PSF2_HUMAN | DNA replication complex GINS protein PSF2 |
| GIPC2 | GIPC2_HUMAN | PDZ domain-containing protein GIPC2 |
| GLDN | GLDN_HUMAN | Gliomedin shedded ectodomain |
| GLI4 | GLI4_HUMAN | Zinc finger protein GLI4 |
| GLIPR2 | GAPR1_HUMAN | Golgi-associated plant pathogenesis-related protein 1 |
| GLIS2 | GLIS2_HUMAN | Zinc finger protein GLIS2 |
| GLO1 | LGUL_HUMAN | Lactoylglutathione Iyase |
| GLOD4 | GLOD4_HUMAN | Glyoxalase domain-containing protein 4 |
| GLP1R | GLP1R_HUMAN | Glucagon-like peptide 1 receptor |
| GLRA1 | GLRA1_HUMAN | Glycine receptor subunit alpha-I |
| GLRA3 | GLRA3_HUMAN | Glycine receptor subunit alpha-3 |
| GLS | GLSK_HUMAN | Glutaminase kidney isoform, mitochondrial |
| GLS2 | GLSL_HUMAN | Glutaminase liver isoform, mitochondrial |
| GLUD1 | DHE3_HUMAN | Glutamate dehydrogenase 1, mitochondrial |
| GMDS | GMDS_HUMAN | GDP-mannose 4,6 dehydratase |
| GMFG | GMFG_HUMAN | Glia maturation factor gamma |
| GNB1 | GBB1_HUMAN | Guanine nucleotide-binding protein G(I)/G(S)/G(T) subunit beta-I |
| GNE | GLCNE_HUMAN | N-acetylmannosamine kinase |
| GNPDA1 | GNPI1_HUMAN | Glucosamine-6-phosphate isomerase 1 |
| GNPNAT1 | GNA1_HUMAN | Glucosamine 6-phosphate N-acetyltransferase |
| GOT1 | AATC_HUMAN | Aspartate aminotransferase, cytoplasmic |
| GOT2 | AATM_HUMAN | Aspartate aminotransferase, mitochondrial |
| GPD1 | GPDA_HUMAN | Glycerol-3-phosphate dehydrogenase [NAD(+)], cytoplasmic |
| GPD1L | GPD1L_HUMAN | Glycerol-3-phosphate dehydrogenase I-like protein |
| GPI | G6PI_HUMAN | Glucose-6-phosphate isomerase |
| GPIHBP1 | HDBP1_HUMAN | Glycosylphosphatidy !inositol-anchored high density lipoprotein- |
| | | binding protein 1 |
| GPT2 | ALAT2_HUMAN | Alanine aminotransferase 2 |
| GPX1 | GPX1_HUMAN | Glutathione peroxidase 1 |
| GPX2 | GPX2_HUMAN | Glutathione peroxidase 2 |
| GPX4 | GPX4_HUMAN | Phospholipid hydroperoxide glutathione peroxidase |
| GPX7 | GPX7_HUMAN | Glutathione peroxidase 7 |
| GPX8 | GPX8_HUMAN | Probable glutathione peroxidase 8 |
| GRAP2 | GRAP2_HUMAN | GRB2-related adapter protein 2 |
| GRB10 | GRB10_HUMAN | Growth factor receptor-bound protein 10 |
| GRB14 | GRB14_HUMAN | Growth factor receptor-bound protein 14 |
| GRB2 | GRB2_HUMAN | Growth factor receptor-bound protein 2 |
| GRB7 | GRB7_HUMAN | Growth factor receptor-bound protein 7 |
| GRIA2 | GRIA2_HUMAN | Glutamate receptor 2 |
| GRIK1 | GRIK1_HUMAN | Glutamate receptor ionotropic, kainate 1 |
| GRIK2 | GRIK2_HUMAN | Glutamate receptor ionotropic, kainate 2 |
| GRIN2A | NMDE1_HUMAN | Glutamate receptor ionotropic, NMDA 2A |
| GRK2 | ARBK1_HUMAN | Beta-adrenergic receptor kinase 1 |
| GRK4 | GRK4_HUMAN | G protein-coupled receptor kinase 4 |
| GRK5 | GRK5_HUMAN | G protein-coupled receptor kinase 5 |
| GRK6 | GRK6_HUMAN | G protein-coupled receptor kinase 6 |
| GRM1 | GRM1_HUMAN | Metabotropic glutamate receptor 1 |
| GRM2 | GRM2_HUMAN | Metabotropic glutamate receptor 2 |
| GRM3 | GRM3_HUMAN | Metabotropic glutamate receptor 3 |
| GRM5 | GRM5_HUMAN | Metabotropic glutamate receptor 5 |
| GRM7 | GRM7_HUMAN | Metabotropic glutamate receptor 7 |
| GRM8 | GRM8_HUMAN | Metabotropic glutamate receptor 8 |
| GRN | GRN_HUMAN | Granulin-7 |
| GSK3B | GSK3B_HUMAN | Glycogen synthase kinase-3 beta |
| GSN | GELS_HUMAN | Gelsolin |
| GSPT1 | ERF3A_HUMAN | Eukaryotic peptide chain release factor GTP-binding subunit ERF3A |
| GSR | GSHR_HUMAN | Glutathione reductase, mitochondrial |
| GSTOl | GSTO1_HUMAN | Glutathione S-transferase omega-1 |
| GTF2B | TF2B_HUMAN | Transcription initiation factor IIB |
| GTF2E1 | T2EA_HUMAN | General transcription factor IIE subunit 1 |
| GTF2F1 | T2FA_HUMAN | General transcription factor IIF subunit 1 |
| GTF2H1 | TF2H1_HUMAN | General transcription factor IIH subunit 1 |
| GTF3A | TF3A_HUMAN | Transcription factor IIIA |
| GUSB | BGLR_HUMAN | Beta-glucuronidase |
| GZF1 | GZF1_HUMAN | GDNF-inducible zinc finger protein 1 |
| GZMB | GRAB_HUMAN | Granzyme B |
| GZMM | GRAM_HUMAN | Granzyme M |
| H2AFY | H2AY_HUMAN | Core histone macro-H2A.1 |
| H2AFY2 | H2AW_HUMAN | Core histone macro-H2A.2 |
| HADHA | ECHA_HUMAN | Long chain 3-hydroxyacyl-CoA dehydrogenase |
| HASPIN | HASP_HUMAN | Serine/threonine-protein kinase haspin |
| HAT1 | HAT1_HUMAN | Histone acetyltransferase type B catalytic subunit |
| HBP1 | HBP1_HUMAN | HMG box-containing protein 1 |
| HCFC1 | HCFC1_HUMAN | HCF C-terminal chain 6 |
| HCK | HCK_HUMAN | Tyrosine-protein kinase HCK |
| HDAC4 | HDAC4_HUMAN | Histone deacetylase 4 |
| HDAC6 | HDAC6_HUMAN | Histone deacetylase 6 |
| HDAC7 | HDAC7_HUMAN | Histone deacetylase 7 |
| HDHD2 | HDHD2_HUMAN | Haloacid dehalogenase-like hydrolase domain containing protein 2 |
| HECTD1 | HECD1_HUMAN | E3 ubiquitin-protein ligase HECTD1 |
| HECW1 | HECW1_HUMAN | E3 ubiquitin-protein ligase HECW1 |
| HECW2 | HECW2_HUMAN | E3 ubiquitin-protein ligase HECW2 |
| HERC1 | HERCI_HUMAN | Probable E3 ubiquitin-protein ligase HERC1 |
| HERC2 | HERC2_HUMAN | E3 ubiquitin-protein ligase HERC2 |
| HERVK 113 | GA113_HUMAN | Endogenous retrovirus group K member 113 Gag polyprotein |
| HEXA | HEXA_HUMAN | Beta-hexosaminidase subunit alpha |
| HEXB | HEXB_HUMAN | Beta-hexosaminidase subunit beta chain A |
| HFE | HFE_HUMAN | Hereditary hemochromatosis protein |
| HGD | HGD_HUMAN | Homogentisate 1,2-dioxygenase |
| HGS | HGS_HUMAN | Hepatocyte growth factor-regulated tyrosine kinase substrate |
| HHIP | HHIP_HUMAN | Hedgehog-interacting protein |
| HIC1 | HIC1_HUMAN | Hypermethylated in cancer 1 protein |
| HIC2 | HIC2_HUMAN | Hypermethylated in cancer 2 protein |
| HIF1A | HIF1A_HUMAN | Hypoxia-inducible factor 1-alpha |
| HIF3A | HIF3A_HUMAN | Hypoxia-inducible factor 3-alpha |
| HINFP | HINFP_HUMAN | Histone H4 transcription factor |
| HIRA | HIRA_HUMAN | Protein HIRA |
| HIVEPl | ZEP1_HUMAN | Zinc finger protein 40 |
| HIVEP2 | ZEP2_HUMAN | Transcription factor HIVEP2 |
| HIVEP3 | ZEP3_HUMAN | Transcription factor HIVEP3 |
| HMCES | HMCES_HUMAN | Abasic site processing protein HMCES |
| HMGCL | HMGCL_HUMAN | Hydroxymethylglutary 1-CoA lyase, mitochondrial |
| HNF4A | HNF4A_HUMAN | Hepatocyte nuclear factor 4-alpha |
| HNF4G | HNF4G_HUMAN | Hepatocyte nuclear factor 4-gamma |
| HNRNPA1 | ROA1_HUMAN | Heterogeneous nuclear ribonucleoprotein A1, N-terminally processed |
| HNRNPA2B1 | ROA2_HUMAN | Heterogeneous nuclear ribonucleoproteins A2/B1 |
| HNRNPAB | ROAA_HUMAN | Heterogeneous nuclear ribonucleoprotein A/B |
| HNRNPD | HNRPD_HUMAN | Heterogeneous nuclear ribonucleoprotein D0 |
| HNRNPH2 | HNRH2_HUMAN | Heterogeneous nuclear ribonucleoprotein H2, N-terminally processed |
| HPD | HPPD_HUMAN | 4-hydroxyphenylpymvate dioxygenase |
| HPN | HEPS_HUMAN | Serine protease hepsin catalytic chain |
| HRH1 | HRH1_HUMAN | Histamine H1 receptor |
| HS3ST1 | HS3S1_HUMAN | Heparan sulfate glucosamine 3-O-sulfotransferase 1 |
| HS3ST3A1 | HS3SA_HUMAN | Heparan sulfate glucosamine 3-O-sulfotransferase 3A1 |
| HS3ST5 | HS3S5_HUMAN | Heparan sulfate glucosamine 3-O-sulfotransferase 5 |
| HSCB | HSC20_HUMAN | Iron-sulfur cluster co-chaperone protein HscB, mitochondrial |
| HSD17B10 | HCD2_HUMAN | 3-hydroxyacyl-CoA dehydrogenase type-2 |
| HSD17B4 | DHB4_HUMAN | Enoyl-CoA hydratase 2 |
| HSPA1A | HS71A_HUMAN | Heat shock 70 kDa protein 1A |
| HSPA5 | BIP_HUMAN | Endoplasmic reticulum chaperone BiP |
| HSPA8 | HSP7C_HUMAN | Heat shock cognate 71 kDa protein |
| HSPA9 | GRP75_HUMAN | Stress-70 protein, mitochondrial |
| HSPB1 | HSPB1_HUMAN | Heat shock protein beta-1 |
| HSPB2 | HSPB2_HUMAN | Heat shock protein beta-2 |
| HSPB6 | HSPB6_HUMAN | Heat shock protein beta-6 |
| HSPDl | CH60_HUMAN | 60 kDa heat shock protein, mitochondrial |
| HSPG2 | PGBM_HUMAN | LG3 peptide |
| HTRA1 | HTRA1_HUMAN | Serine protease HTRA1 |
| HTRA2 | HTRA2_HUMAN | Serine protease HTRA2, mitochondrial |
| HTRA3 | HTRA3_HUMAN | Serine protease HTRA3 |
| HTT | HD_HUMAN | Huntingtin |
| HUS1 | HUS1_HUMAN | Checkpoint protein HUS1 |
| HUWE1 | HUWE1_HUMAN | E3 ubiquitin-protein ligase HUWE1 |
| HYAL1 | HYAL1_HUMAN | Hyaluronidase-1 |
| HYDIN | HYDIN_HUMAN | Hydrocephalus-inducing protein homolog |
| ICAM1 | ICAM1_HUMAN | Intercellular adhesion molecule 1 |
| IDE | IDE_HUMAN | Insulin-degrading enzyme |
| IDH3G | IDH3G_HUMAN | Isocitrate dehydrogenase [NAD] subunit gamma, mitochondrial |
| IDO1 | 123O1_HUMAN | Indoleamine 2,3-dioxygenase 1 |
| IDS | IDS_HUMAN | Iduronate 2-sulfatase 14 kDa chain |
| IDUA | IDUA_HUMAN | Alpha-L-iduronidase |
| IFI16 | IF16_HUMAN | Gamma-interferon-inducible protein 16 |
| IFNAR1 | INARI_HUMAN | Interferon alpha/beta receptor 1 |
| IFNGR1 | INGR1_HUMAN | Interferon gamma receptor 1 |
| IFNGR2 | INGR2_HUMAN | Interferon gamma receptor 2 |
| IFNLR1 | INLR1_HUMAN | Interferon lambda receptor 1 |
| IGF1R | IGF1R_HUMAN | Insulin-like growth factor 1 receptor beta chain |
| IGF2R | MPRI_HUMAN | Cation-independent mannose-6-phosphate receptor |
| IGFBP1 | IBP1_HUMAN | Insulin-like growth factor-binding protein 1 |
| IGFBP4 | IBP4_HUMAN | Insulin-like growth factor-binding protein 4 |
| IGFBP6 | IBP6_HUMAN | Insulin-like growth factor-binding protein 6 |
| IGHA1 | IGHA1_HUMAN | Immunoglobulin heavy constant alpha 1 |
| IGHE | IGHE_HUMAN | Immunoglobulin heavy constant epsilon |
| IGHG1 | IGHG1_HUMAN | Immunoglobulin heavy constant gamma 1 |
| IGHG4 | IGHG4_HUMAN | Immunoglobulin heavy constant gamma 4 |
| IGHM | IGHM_HUMAN | Immunoglobulin heavy constant mu |
| IGHV3-23 | HV323_HUMAN | Immunoglobulin heavy variable 3-23 |
| IGHV3-33 | HV333_HUMAN | Immunoglobulin heavy variable 3-33 |
| IGHV4-59 | HV459_HUMAN | Immunoglobulin heavy variable 4-59 |
| IGKC | IGKC_HUMAN | Immunoglobulin kappa constant |
| IGKV1-33 | KV133_HUMAN | Immunoglobulin kappa variable 1-33 |
| IKBKB | IKKB_HUMAN | Inhibitor of nuclear factor kappa-B kinase subunit beta |
| IKZF1 | IKZF1_HUMAN | DNA-binding protein Ikaros |
| IKZF2 | IKZF2_HUMAN | Zinc finger protein Helios |
| IKZF3 | IKZF3_HUMAN | Zinc finger protein Aiolos |
| IKZF4 | IKZF4_HUMAN | Zinc finger protein Eos |
| IKZF5 | IKZF5_HUMAN | Zinc finger protein Pegasus |
| IL12B | IL12B_HUMAN | Interleukin-12 subunit beta |
| IL13RA2 | 113R2_HUMAN | Interleukin-13 receptor subunit alpha-2 |
| IL17A | IL17_HUMAN | Interleukin-17A |
| IL17F | IL17F_HUMAN | Interleukin-17F |
| IL17RA | IL7RA_HUMAN | Interleukin-17 receptor A |
| IL18R1 | IL8R_HUMAN | Interleukin-18 receptor 1 |
| IL18RAP | IL8RA_HUMAN | Interleukin-18 receptor accessory protein |
| IL1F10 | IL1FA_HUMAN | Interleukin-I family member 10 |
| IL1RAP | IL1AP_HUMAN | Interleukin-I receptor accessory protein |
| IL20RB | I20RB_HUMAN | Interleukin-20 receptor subunit beta |
| IL22RA1 | I22R1_HUMAN | Interleukin-22 receptor subunit alpha-1 |
| IL23R | IL23R_HUMAN | Interleukin-23 receptor |
| IL4R | IL4RA_HUMAN | Soluble interleukin-4 receptor subunit alpha |
| IL5RA | IL5RA_HUMAN | Interleukin-5 receptor subunit alpha |
| IL6R | IL6RA_HUMAN | Interleukin-6 receptor subunit alpha |
| IL6ST | IL6RB_HUMAN | Interleukin-6 receptor subunit beta |
| ILK | ILK_HUMAN | Integrin-linked protein kinase |
| IMPAl | IMPA1_HUMAN | Inositol monophosphatase 1 |
| INHBA | INHBA_HUMAN | Inhibin beta A chain |
| INKAl | INKA1_HUMAN | P AK4-inhibitor INKAl |
| INO80B | IN80B_HUMAN | INO80 complex subunit B |
| INPPL1 | SHIP2_HUMAN | Phosphatidylinositol 3,4,5-trisphosphate 5-phosphatase 2 |
| INSM1 | INSM1_HUMAN | Insulinoma-associated protein 1 |
| INSM2 | INSM2_HUMAN | Insulinoma-associated protein 2 |
| INSR | INSR_HUMAN | Insulin receptor subunit beta |
| INTS11 | INT11_HUMAN | Integrator complex subunit 11 |
| IPMK | IPMK_HUMAN | Inositol polyphosphate multikinase |
| IQGAP1 | IQGA1_HUMAN | Ras GTPase-activating-like protein IQGAP1 |
| IQGAP2 | IQGA2_HUMAN | Ras GTPase-activating-like protein IQGAP2 |
| IQGAP3 | IQGA3_HUMAN | Ras GTPase-activating-like protein IQGAP3 |
| IQUB | IQUB_HUMAN | IQ and ubiquitin-like domain-containing protein |
| IRAKl | IRAKl_HUMAN | Interleukin-1 receptor-associated kinase 1 |
| IRAK4 | IRAK4_HUMAN | Interleukin-1 receptor-associated kinase 4 |
| ISCU | ISCU_HUMAN | Iron-sulfur cluster assembly enzyme ISCU, mitochondrial |
| ISG15 | ISG15_HUMAN | Ubiquitin-like protein ISG15 |
| ISG20 | ISG20_HUMAN | Interferon-stimulated gene 20 kDa protein |
| ITCH | ITCH_HUMAN | E3 ubiquitin-protein ligase Itchy homolog |
| ITGA2B | ITA2B_HUMAN | Integrin alpha-IIb light chain, form 2 |
| ITGA4 | ITA4_HUMAN | Integrin alpha-4 |
| ITGA5 | ITA5_HUMAN | Integrin alpha-5 light chain |
| ITGAL | ITAL_HUMAN | Integrin alpha-L |
| ITGAV | ITAV_HUMAN | Integrin alpha-V light chain |
| ITGAX | ITAX_HUMAN | Integrin alpha-X |
| ITGB1 | ITB1_HUMAN | Integrin beta-1 |
| ITGBlBPl | ITBP1_HUMAN | Integrin beta-1-binding protein 1 |
| ITGB2 | ITB2_HUMAN | Integrin beta-2 |
| ITGB3 | ITB3_HUMAN | Integrin beta-3 |
| ITGB4 | ITB4_HUMAN | Integrin beta-4 |
| ITGB6 | ITB6_HUMAN | Integrin beta-6 |
| ITIHl | ITIH1_HUMAN | Inter-alpha-trypsin inhibitor heavy chain Hl |
| ITK | ITK_HUMAN | Tyrosine-protein kinase ITK/TSK |
| ITLNl | ITLN1_HUMAN | Intelectin-1 |
| ITPA | ITPA_HUMAN | Inosine triphosphate pyrophosphatase |
| ITPKl | ITPKl_HUMAN | Inositol-tetrakisphosphate 1-kinase |
| ITPKA | IP3KA_HUMAN | Inositol-trisphosphate 3-kinase A |
| ITPKC | IP3KC_HUMAN | Inositol-trisphosphate 3-kinase C |
| ITSNl | ITSNl_HUMAN | Intersectin-1 |
| ITSN2 | ITSN2_HUMAN | Intersectin-2 |
| IYD | IYD1_HUMAN | lodotyrosine deiodinase 1 |
| JAG1 | JAGl_HUMAN | Protein jagged-1 |
| JAG2 | JAG2_HUMAN | Protein jagged-2 |
| JAKl | JAKl_HUMAN | Tyrosine-protein kinase JAKl |
| JAK2 | JAK2_HUMAN | Tyrosine-protein kinase JAK2 |
| JAK3 | JAK3_HUMAN | Tyrosine-protein kinase JAK3 |
| JMJDlC | JHD2C_HUMAN | Probable JmjC domain-containing histone demethylation protein 2C |
| JMJD6 | JMJD6_HUMAN | Bifunctional arginine demethylase and lysyl-hydroxylase JMJD6 |
| JMJD7 | JMJD7_HUMAN | Bifunctional peptidase and (3S)-lysyl hydroxylase JMJD7 |
| KANKl | KANKl_HUMAN | KN motif and ankyrin repeat domain-containing protein 1 |
| KANK2 | KANK2_HUMAN | KN motif and ankyrin repeat domain-containing protein 2 |
| KARS | SYK_HUMAN | Lysine--tRNA ligase |
| KAT2A | KAT2A_HUMAN | Histone acetyltransferase KAT2A |
| KAT2B | KAT2B_HUMAN | Histone acetyltransferase KAT2B |
| KAT6A | KAT6A_HUMAN | Histone acetyltransferase KAT6A |
| KAT6B | KAT6B_HUMAN | Histone acetyltransferase KAT6B |
| KCMFl | KCMFl_HUMAN | E3 ubiquitin-protein ligase KCMFI |
| KCNAB2 | KCAB2_HUMAN | Voltage-gated potassium channel subunit beta-2 |
| KCNH2 | KCNH2_HUMAN | Potassium voltage-gated channel subfamily H member 2 |
| KCNJ11 | KCJ11_HUMAN | ATP-sensitive inward rectifier potassium channel 11 |
| KCTD10 | BACD3_HUMAN | BTB/POZ domain-containing adapter for CUL3-mediated RhoA |
| | | degradation protein 3 |
| KCTD13 | BACDl_HUMAN | BTB/POZ domain-containing adapter for CUL3-mediated RhoA |
| | | degradation protein 1 |
| KCTD16 | KCD16_HUMAN | BTB/POZ domain-containing protein KCTD 16 |
| KCTD17 | KCD17_HUMAN | BTB/POZ domain-containing protein KCTD 17 |
| KCTD5 | KCTD5_HUMAN | BTB/POZ domain-containing protein KCTD5 |
| KCTD9 | KCTD9_HUMAN | BTB/POZ domain-containing protein KCTD9 |
| KDMlA | KDMlA_HUMAN | Lysine-specific histone demethylase 1A |
| KDMlB | KDMlB_HUMAN | Lysine-specific histone demethylase 1B |
| KDM2A | KDM2A_HUMAN | Lysine-specific demethylase 2A |
| KDM2B | KDM2B_HUMAN | Lysine-specific demethylase 2B |
| KDM3A | KDM3A_HUMAN | Lysine-specific demethylase 3A |
| KDM3B | KDM3B_HUMAN | Lysine-specific demethylase 3B |
| KDM4A | KDM4A_HUMAN | Lysine-specific demethylase 4A |
| KDM4B | KDM4B_HUMAN | Lysine-specific demethylase 4B |
| KDM4C | KDM4C_HUMAN | Lysine-specific demethylase 4C |
| KDM5A | KDM5A_HUMAN | Lysine-specific demethylase 5A |
| KDM5B | KDM5B_HUMAN | Lysine-specific demethylase 5B |
| KDR | VGFR2_HUMAN | Vascular endothelial growth factor receptor 2 |
| KEAP1 | KEAP1_HUMAN | Kelch-like ECH-associated protein 1 |
| KHDC4 | KHDC4_HUMAN | KH homology domain-containing protein 4 |
| KHK | KHK_HUMAN | Ketohexokinase |
| KIAA0391 | MRPP3_HUMAN | Mitochondrial ribonuclease P catalytic subunit |
| KIF11 | KIF11_HUMAN | Kinesin-like protein KIF11 |
| K1Fl3B | K113B_HUMAN | Kinesin-like protein KIF13B |
| KIFI5 | KIFI5_HUMAN | Kinesin-like protein KIFI5 |
| KIFI8A | Kll8A_HUMAN | Kinesin-like protein KIFI8A |
| KIFIA | KIFIA_HUMAN | Kinesin-like protein KIF IA |
| KIFlB | KIFIB_HUMAN | Kinesin-like protein KIF1B |
| KIFIC | KIFIC_HUMAN | Kinesin-like protein KIF1C |
| KIF22 | KIF22_HUMAN | Kinesin-like protein KIF22 |
| KIF23 | KIF23_HUMAN | Kinesin-like protein KIF23 |
| KIF2C | KIF2C_HUMAN | Kinesin-like protein KIF2C |
| KIF3B | KIF3B_HUMAN | Kinesin-like protein KIF3B, N-terminally processed |
| KIF3C | KIF3C_HUMAN | Kinesin-like protein KIF3C |
| KIF7 | KIF7_HUMAN | Kinesin-like protein KIF7 |
| KIF9 | KIF9_HUMAN | Kinesin-like protein KIF9 |
| KIFC1 | KIFC1_HUMAN | Kinesin-like protein KIFC1 |
| KIFC3 | KIFC3_HUMAN | Kinesin-like protein KIFC3 |
| KIN | KINI7_HUMAN | DNA/RNA-binding protein KINI7 |
| KIR2DS4 | K12S4_HUMAN | Killer cell immunoglobulin-like receptor 2DS4 |
| KIRREL3 | KIRR3_HUMAN | Processed kin of IRRE-like protein 3 |
| KIT | KIT_HUMAN | Mast/stem cell growth factor receptor Kit |
| KLB | KLOTB_HUMAN | Beta-klotho |
| KLFl | KLFl_HUMAN | Krueppel-like factor 1 |
| KLF10 | KLF10_HUMAN | Krueppel-like factor 10 |
| KLHDC2 | KLDC2_HUMAN | Kelch domain-containing protein 2 |
| KLHLll | KLH11_HUMAN | Kelch-like protein 11 |
| KLHL12 | KLH12_HUMAN | Kelch-like protein 12 |
| KLHL17 | KLH17_HUMAN | Kelch-like protein 17 |
| KLHL40 | KLH40_HUMAN | Kelch-like protein 40 |
| KLHL7 | KLHL7_HUMAN | Kelch-like protein 7 |
| KLK4 | KLK4_HUMAN | Kallikrein-4 |
| KLK6 | KLK6_HUMAN | Kallikrein-6 |
| KLKBl | KLKB1_HUMAN | Plasma kallikrein light chain |
| KLRDl | KLRD1_HUMAN | Natural killer cells antigen CD94 |
| KLRGl | KLRG1_HUMAN | Killer cell lectin-like receptor subfamily G member 1 |
| KLRG2 | KLRG2_HUMAN | Killer cell lectin-like receptor subfamily G member 2 |
| KLRKl | NKG2D_HUMAN | NKG2-D type II integral membrane protein |
| KMO | KMO_HUMAN | Kynurenine 3-monooxygenase |
| KMT2A | KMT2A_HUMAN | MLL cleavage product C 180 |
| KMT2B | KMT2B_HUMAN | Histone-lysine N-methyltransferase 2B |
| KMT2C | KMT2C_HUMAN | Histone-lysine N-methyltransferase 2C |
| KMT2D | KMT2D_HUMAN | Histone-lysine N-methyltransferase 2D |
| KMT2E | KMT2E_HUMAN | Inactive histone-lysine N-methyltransferase 2E |
| KMT5A | KMT5A_HUMAN | N-lysine methyltransferase KMT5A |
| KREMEN1 | KREMl_HUMAN | Kremen protein 1 |
| KRlTl | KRlTl_HUMAN | Krev interaction trapped protein 1 |
| KSR2 | KSR2_HUMAN | Kinase suppressor of Ras 2 |
| KYAT1 | KAT1_HUMAN | Kynurenine--oxoglutarate transaminase 1 |
| KYNU | KYNU_HUMAN | Kynureninase |
| L3MBTL2 | LMBL2_HUMAN | Lethal(3)malignant brain tumor-like protein 2 |
| LAMA5 | LAMA5_HUMAN | Laminin subunit alpha-5 |
| LAMP3 | LAMP3_HUMAN | Lysosome-associated membrane glycoprotein 3 |
| LAMTOR2 | LTOR2_HUMAN | Ragulator complex protein LAMTOR2 |
| LAMTOR3 | LTOR3_HUMAN | Ragulator complex protein LAMTOR3 |
| LAMTOR5 | LTOR5_HUMAN | Ragulator complex protein LAMTOR5 |
| LANCLl | LANCI_HUMAN | Glutathione S-transferase LANCLl |
| LARP7 | LARP7_HUMAN | La-related protein 7 |
| LARS | SYLC_HUMAN | Leucine--tRNA ligase, cytoplasmic |
| LASPl | LASP1_HUMAN | LIM and SH3 domain protein 1 |
| LBR | LBR_HUMAN | Delta(14)-sterol reductase |
| LCAT | LCAT_HUMAN | Phosphatidylcholine-sterol acyltransferase |
| LCK | LCK_HUMAN | Tyrosine-protein kinase Lek |
| LCNl | LCNl_HUMAN | Lipocalin-1 |
| LCNl5 | LCN15_HUMAN | Lipocalin-15 |
| LCN2 | NGAL_HUMAN | Neutrophil gelatinase-associated lipocalin |
| LDLR | LDLR_HUMAN | Low-density lipoprotein receptor |
| LEOl | LEO1_HUMAN | RNA polymerase-associated protein LEOl |
| LEPR | LEPR_HUMAN | Leptin receptor |
| LGALS1 | LEGl_HUMAN | Galectin-1 |
| LGALS2 | LEG2_HUMAN | Galectin-2 |
| LGALS3 | LEG3_HUMAN | Galectin-3 |
| LGALS4 | LEG4_HUMAN | Galectin-4 |
| LGALS7\| | LEG7_HUMAN | Galectin-7 |
| LGALS7B | | |
| LGALS8 | LEG8_HUMAN | Galectin-8 |
| LGALS9 | LEG9_HUMAN | Galectin-9 |
| LG11 | LG11_HUMAN | Leucine-rich glioma-inactivated protein 1 |
| LGMN | LGMN_HUMAN | Legumain |
| LGR4 | LGR4_HUMAN | Leucine-rich repeat-containing G-protein coupled receptor 4 |
| LIFR | LIFR_HUMAN | Leukemia inhibitory factor receptor |
| LIGl | DNL11_HUMAN | DNA ligase 1 |
| LIG3 | DNL13_HUMAN | DNA ligase 3 |
| LIG4 | DNL14_HUMAN | DNA ligase 4 |
| LILRA5 | LIRA5_HUMAN | Leukocyte immunoglobulin-like receptor subfamily A member 5 |
| LILRB4 | LIRB4_HUMAN | Leukocyte immunoglobulin-like receptor subfamily B member 4 |
| LIMKl | LIMKl_HUMAN | LIM domain kinase 1 |
| LIMK2 | LIMK2_HUMAN | LIM domain kinase 2 |
| LIMSI | LIMSl_HUMAN | LIM and senescent cell antigen-like-containing domain protein 1 |
| LIN28A | LN28A_HUMAN | Protein lin-28 homolog A |
| LIN28B | LN28B_HUMAN | Protein lin-28 homolog B |
| LINGOI | LIGOI_HUMAN | Leucine-rich repeat and immunoglobulin-like domain-containing nogo |
| | | receptor-interacting protein 1 |
| LIPP | LIPG_HUMAN | Gastric triacylglycerol lipase |
| LMNBl | LMNBl_HUMAN | Lamin-Bl |
| LMO2 | RBTN2_HUMAN | Rhombotin-2 |
| LMO4 | LMO4_HUMAN | LIM domain transcription factor LM04 |
| LNPEP | LCAP_HUMAN | Leucyl-cystinyl aminopeptidase, pregnancy serum form |
| LNXl | LNXl_HUMAN | E3 ubiquitin-protein ligase LNX |
| LNX2 | LNX2_HUMAN | Ligand of Numb protein X 2 |
| LONPl | LONM_HUMAN | Lon protease homolog, mitochondrial |
| LONRF3 | LONF3_HUMAN | LON peptidase N-terminal domain and RING finger protein 3 |
| LRBA | LRBA_HUMAN | Lipopolysaccharide-responsive and beige-like anchor protein |
| LRFN5 | LRFN5_HUMAN | Leucine-rich repeat and fibronectin type-III domain-containing protein |
| | | 5 |
| LR1Gl | LR1Gl_HUMAN | Leucine-rich repeats and immunoglobulin-like domains protein 1 |
| LRPl | LRPl_HUMAN | Low-density lipoprotein receptor-related protein 1 intracellular domain |
| LRP6 | LRP6_HUMAN | Low-density lipoprotein receptor-related protein 6 |
| LRP8 | LRP8_HUMAN | Low-density lipoprotein receptor-related protein 8 |
| LRRC32 | LRC32_HUMAN | Transforming growth factor beta activator LRRC32 |
| LRRC4 | LRRC4_HUMAN | Leucine-rich repeat-containing protein 4 |
| LRRC4C | LRC4C_HUMAN | Leucine-rich repeat-containing protein 4C |
| LRRK2 | LRRK2_HUMAN | Leucine-rich repeat serine/threonine-protein kinase 2 |
| LSM4 | LSM4_HUMAN | U6 snRNA-associated Sm-like protein LSm4 |
| LSM6 | LSM6_HUMAN | U6 snRNA-associated Sm-like protein LSm6 |
| LSM7 | LSM7_HUMAN | U6 snRNA-associated Sm-like protein LSm7 |
| LSM8 | LSM8_HUMAN | U6 snRNA-associated Sm-like protein LSm8 |
| LSS | ERG7_HUMAN | Lanosterol synthase |
| LTF | TRFL_HUMAN | Lactoferroxin-C |
| LXN | LXN_HUMAN | Latexin |
| LY86 | LY86_HUMAN | Lymphocyte antigen 86 |
| LYAR | LYAR_HUMAN | Cell growth-regulating nucleolar protein |
| LYPD6 | LYPD6_HUMAN | Ly6/PLAUR domain-containing protein 6 |
| LYZ | LYSC_HUMAN | Lysozyme C |
| MAD2L1 | MD2L1_HUMAN | Mitotic spindle assembly checkpoint protein MAD2A |
| MAGll | MAG11_HUMAN | Membrane-associated guanylate kinase, WW and PDZ domain- |
| | | containing protein 1 |
| MAGOH | MGN_HUMAN | Protein mago nashi homolog |
| MAGOHB | MGN2_HUMAN | Protein mago nashi homolog 2 |
| MALTl | MALTl_HUMAN | Mucosa-associated lymphoid tissue lymphoma |
| | | translocation protein 1 |
| MANlBl | MAlBl_HUMAN | Endoplasmic reticulum mannosy 1-oligosaccharide 1,2-alpha- |
| | | mannosidase |
| MAP2Kl | MP2Kl_HUMAN | Dual specificity mitogen-activated protein kinase kinase 1 |
| MAP2K2 | MP2K2_HUMAN | Dual specificity mitogen-activated protein kinase kinase 2 |
| MAP2K4 | MP2K4_HUMAN | Dual specificity mitogen-activated protein kinase kinase 4 |
| MAP2K5 | MP2K5_HUMAN | Dual specificity mitogen-activated protein kinase kinase 5 |
| MAP2K6 | MP2K6_HUMAN | Dual specificity mitogen-activated protein kinase kinase 6 |
| MAP2K7 | MP2K7_HUMAN | Dual specificity mitogen-activated protein kinase kinase 7 |
| MAP3K10 | M3K10_HUMAN | Mitogen-activated protein kinase kinase kinase 10 |
| MAP3K11 | M3K11_HUMAN | Mitogen-activated protein kinase kinase kinase 11 |
| MAP3K12 | M3K12_HUMAN | Mitogen-activated protein kinase kinase kinase 12 |
| MAP3K14 | M3K14_HUMAN | Mitogen-activated protein kinase kinase kinase 14 |
| MAP3K20 | M3K20_HUMAN | Mitogen-activated protein kinase kinase kinase 20 |
| MAP3K5 | M3K5_HUMAN | Mitogen-activated protein kinase kinase kinase 5 |
| MAP3K7 | M3K7_HUMAN | Mitogen-activated protein kinase kinase kinase 7 |
| MAP3K9 | M3K9_HUMAN | Mitogen-activated protein kinase kinase kinase 9 |
| MAP4K1 | M4K1_HUMAN | Mitogen-activated protein kinase kinase kinase kinase 1 |
| MAP4K3 | M4K3_HUMAN | Mitogen-activated protein kinase kinase kinase kinase 3 |
| MAP4K4 | M4K4_HUMAN | Mitogen-activated protein kinase kinase kinase kinase 4 |
| MAPK1 | MK01_HUMAN | Mitogen-activated protein kinase 1 |
| MAPK10 | MK10_HUMAN | Mitogen-activated protein kinase 10 |
| MAPK12 | MK12_HUMAN | Mitogen-activated protein kinase 12 |
| MAPK13 | MK13_HUMAN | Mitogen-activated protein kinase 13 |
| MAPK14 | MK14_HUMAN | Mitogen-activated protein kinase 14 |
| MAPK3 | MK03_HUMAN | Mitogen-activated protein kinase 3 |
| MAPK7 | MK07_HUMAN | Mitogen-activated protein kinase 7 |
| MAPK8 | MK08_HUMAN | Mitogen-activated protein kinase 8 |
| MAPK9 | MK09_HUMAN | Mitogen-activated protein kinase 9 |
| MAPKAPK2 | MAPK2_HUMAN | MAP kinase-activated protein kinase 2 |
| MAPKAPK3 | MAPK3_HUMAN | MAP kinase-activated protein kinase 3 |
| MARCI | MARCI_HUMAN | Mitochondrial amidoxime-reducing component 1 |
| MARK1 | MARK1_HUMAN | Serine/threonine-protein kinase MARK1 |
| MARK2 | MARK2_HUMAN | Serine/threonine-protein kinase MARK2 |
| MARK3 | MARK3_HUMAN | MAP/microtubule affinity-regulating kinase 3 |
| MARK4 | MARK4_HUMAN | MAP/microtubule affinity-regulating kinase 4 |
| MARS | SYMC_HUMAN | Methionine -- tRNA ligase, cytoplasmic |
| MASP1 | MASP1_HUMAN | Mannan-binding lectin serine protease 1 light chain |
| MASP2 | MASP2_HUMAN | Mannan-binding lectin serine protease 2 B chain |
| MASTL | GWL_HUMAN | Serine/threonine-protein kinase greatwall |
| MATK | MATK_HUMAN | Megakaryocyte-associated tyrosine-protein kinase |
| MAZ | MAZ_HUMAN | Myc-associated zinc finger protein |
| MBD1 | MBD1_HUMAN | Methyl-CpG-binding domain protein 1 |
| MBD2 | MBD2_HUMAN | Methyl-CpG-binding domain protein 2 |
| MBD3 | MBD3_HUMAN | Methyl-CpG-binding domain protein 3 |
| MBD4 | MBD4_HUMAN | Methyl-CpG-binding domain protein 4 |
| MBL2 | MBL2_HUMAN | Mannose-binding protein C |
| MBLAC1 | MBLC1_HUMAN | Metallo-beta-lactamase domain-containing protein 1 |
| MBTD1 | MBTD1_HUMAN | MBT domain-containing protein 1 |
| MCAT | FABD_HUMAN | Malonyl-CoA-acyl carrier protein transacylase, mitochondrial |
| MCEE | MCEE_HUMAN | Methylmalony 1-CoA epimerase, mitochondrial |
| MCOLN1 | MCLN1_HUMAN | Mucolipin-1 |
| MCTS1 | MCTS1_HUMAN | Malignant T-cell-amplified sequence 1 |
| MCU | MCU_HUMAN | Calcium uniporter protein, mitochondrial |
| MDM2 | MDM2_HUMAN | E3 ubiquitin-protein ligase Mdm2 |
| MDP1 | MGDP1_HUMAN | Magnesium-dependent phosphatase 1 |
| ME1 | MAOX_HUMAN | NADP-dependent malic enzyme |
| ME2 | MAOM_HUMAN | NAD-dependent malic enzyme, mitochondrial |
| MECOM | MECOM_HUMAN | Histone-lysine N-methyltransferase MECOM |
| MECP2 | MECP2_HUMAN | Methyl-CpG-binding protein 2 |
| MEFV | MEFV_HUMAN | Pyrin |
| MELK | MELK_HUMAN | Maternal embryonic leucine zipper kinase |
| MEN1 | MEN1_HUMAN | Menin |
| MEPlB | MEP1B_HUMAN | Meprin A subunit beta |
| MERTK | MERTK_HUMAN | Tyrosine-protein kinase Mer |
| MET | MET_HUMAN | Hepatocyte growth factor receptor |
| METAP2 | MAP2_HUMAN | Methionine aminopeptidase 2 |
| METTL16 | MET16_HUMAN | RNA N6-adenosine-methyltransferase METTL16 |
| METTL18 | MET18_HUMAN | Histidine protein methyltransferase 1 homolog |
| MEX3C | MEX3C_HUMAN | RNA-binding E3 ubiquitin-protein ligase MEX3C |
| MGAM | MGA_HUMAN | Glucoamylase |
| MGLL | MGLL_HUMAN | Monoglyceride lipase |
| MGMT | MGMT_HUMAN | Methylated-DNA -- protein-cysteine methyltransferase |
| M1A | M1A_HUMAN | Melanoma-derived growth regulatory protein |
| M1Bl | M1Bl_HUMAN | E3 ubiquitin-protein ligase MIB1 |
| M1B2 | M1B2_HUMAN | E3 ubiquitin-protein ligase MIB2 |
| MICAL1 | M1CA1_HUMAN | [F-actin]-monooxygenase MICAL1 |
| MICU1 | M1CU1_HUMAN | Calcium uptake protein 1, mitochondrial |
| MINDY1 | M1NY1_HUMAN | Ubiquitin carboxyl-terminal hydro lase MINDY-1 |
| MKNK1 | MKNK1_HUMAN | MAP kinase-interacting serine/threonine-protein kinase 1 |
| MLH1 | MLH1_HUMAN | DNA mismatch repair protein Mlhl |
| MLLT1 | ENL_HUMAN | Protein ENL |
| MLLT10 | AF10_HUMAN | Protein AF-10 |
| MLLT3 | AF9_HUMAN | Protein AF -9 |
| MLLT6 | AF17_HUMAN | Protein AF -17 |
| MLPH | MELPH_HUMAN | Melanophilin |
| MLST8 | LST8_HUMAN | Target of rapamycin complex subunit LST8 |
| MMAB | MMAB_HUMAN | Corrinoid adenosyltransferase |
| MMADHC | MMAD_HUMAN | Methylmalonic aciduria and homocystinuria type D protein, |
| | | mitochondrial |
| MME | NEP_HUMAN | Neprilysin |
| MMP1 | MMP1_HUMAN | 27 kDa interstitial collagenase |
| MMP13 | MMP13_HUMAN | Collagenase 3 |
| MMP14 | MMP14_HUMAN | Matrix metalloproteinase-14 |
| MMP2 | MMP2_HUMAN | PEX |
| MMUT | MUTA_HUMAN | Methylmalonyl-CoA mutase, mitochondrial |
| MNAT1 | MAT1_HUMAN | CDK-activating kinase assembly factor MATl |
| MPG | 3MG_HUMAN | DNA-3-methyladenine glycosylase |
| MPP7 | MPP7_HUMAN | MAGUK p55 subfamily member 7 |
| MPST | THTM_HUMAN | 3-mercaptopyruvate sulfurtransferase |
| MR1 | HMR1_HUMAN | Major histocompatibility complex class I-related gene protein |
| MRC1 | MRC1_HUMAN | Macrophage mannose receptor 1 |
| MRC2 | MRC2_HUMAN | C-type mannose receptor 2 |
| MR11 | MTNA_HUMAN | Methylthioribose-1-phosphate isomerase |
| MRPL13 | RM13_HUMAN | 39S ribosomal protein Ll3, mitochondrial |
| MRPL18 | RM18_HUMAN | 39S ribosomal protein Ll8, mitochondrial |
| MRPL24 | RM24_HUMAN | 39S ribosomal protein L24, mitochondrial |
| MRPL28 | RM28_HUMAN | 39S ribosomal protein L28, mitochondrial |
| MRPL3 | RM03_HUMAN | 39S ribosomal protein L3, mitochondrial |
| MRPL30 | RM30_HUMAN | 39S ribosomal protein L30, mitochondrial |
| MRPL32 | RM32_HUMAN | 39S ribosomal protein L32, mitochondrial |
| MRPL35 | RM35_HUMAN | 39S ribosomal protein L35, mitochondrial |
| MRPL43 | RM43_HUMAN | 39S ribosomal protein L43, mitochondrial |
| MRPL45 | RM45_HUMAN | 39S ribosomal protein L45, mitochondrial |
| MRPL46 | RM46_HUMAN | 39S ribosomal protein L46, mitochondrial |
| MRPL47 | RM47_HUMAN | 39S ribosomal protein L47, mitochondrial |
| MRPL49 | RM49_HUMAN | 39S ribosomal protein L49, mitochondrial |
| MRPL53 | RM53_HUMAN | 39S ribosomal protein L53, mitochondrial |
| MRPL55 | RM55_HUMAN | 39S ribosomal protein L55, mitochondrial |
| MRPS18A | RT18A_HUMAN | 39S ribosomal protein S18a, mitochondrial |
| MSH2 | MSH2_HUMAN | DNA mismatch repair protein Msh2 |
| MSH3 | MSH3_HUMAN | DNA mismatch repair protein Msh3 |
| MSH6 | MSH6_HUMAN | DNA mismatch repair protein Msh6 |
| MSL2 | MSL2_HUMAN | E3 ubiquitin-protein ligase MSL2 |
| MSL3 | MS3L1_HUMAN | Male-specific lethal 3 homolog |
| MSMB | MSMB_HUMAN | Beta-microseminoprotein |
| MSN | MOES_HUMAN | Moesin |
| MSRB1 | MSRB1_HUMAN | Methionine-R-sulfoxide reductase Bl |
| MST1R | RON_HUMAN | Macrophage-stimulating protein receptor beta chain |
| MSTN | GDF8_HUMAN | Growth/differentiation factor 8 |
| MT-CO2 | COX2_HUMAN | Cytochrome c oxidase subunit 2 |
| MTERF4 | MTEF4_HUMAN | mTERF domain-containing protein 2 processed |
| MTF1 | MTF1_HUMAN | Metal regulatory transcription factor 1 |
| MTF2 | MTF2_HUMAN | Metal-response element-binding transcription factor 2 |
| MTHFR | MTHR_HUMAN | Methylenetetrahydrofolate reductase |
| MTHFS | MTHFS_HUMAN | 5-formyltetrahydrofolate cyclo-ligase |
| MT1F3 | IF3M_HUMAN | Translation initiation factor IF-3, mitochondrial |
| MTMR1 | MTMR1_HUMAN | Myotubularin-related protein 1 |
| MTMR2 | MTMR2_HUMAN | Myotubularin-related protein 2 |
| MTMR3 | MTMR3_HUMAN | Myotubularin-related protein 3 |
| MTMR4 | MTMR4_HUMAN | Myotubularin-related protein 4 |
| MTOR | MTOR_HUMAN | Serine/threonine-protein kinase mTOR |
| MTPAP | PAPD1_HUMAN | Poly(A) RNA polymerase, mitochondrial |
| MTR | METH_HUMAN | Methionine synthase |
| MVK | KIME_HUMAN | Mevalonate kinase |
| MYBPC3 | MYPC3_HUMAN | Myosin-binding protein C, cardiac-type |
| MYCBP2 | MYCB2_HUMAN | E3 ubiquitin-protein ligase MYCBP2 |
| MYH10 | MYH10_HUMAN | Myosin-10 |
| MYH14 | MYH14_HUMAN | Myosin-14 |
| MYH7 | MYH7_HUMAN | Myosin-7 |
| MYL3 | MYL3_HUMAN | Myosin light chain 3 |
| MYL6B | MYL6B_HUMAN | Myosin light chain 6B |
| MYLIP | MYLIP_HUMAN | E3 ubiquitin-protein ligase MYL1P |
| MYLK4 | MYLK4_HUMAN | Myosin light chain kinase family member 4 |
| MYNN | MYNN_HUMAN | Myoneurin |
| MYOl0 | MYOl0_HUMAN | Unconventional myosin-X |
| MYO1C | MYOlC_HUMAN | Unconventional myosin-lc |
| MYO5C | MYO5C_HUMAN | Unconventional myosin-Vc |
| MYO7A | MYO7A_HUMAN | Unconventional myosin-Vlla |
| MYO7B | MYO7B_HUMAN | Unconventional myosin-Vllb |
| MYOC | MYOC_HUMAN | Myocilin, C-terminal fragment |
| MYOF | MYOF_HUMAN | Myoferlin |
| MYOM1 | MYOM1_HUMAN | Myomesin-1 |
| MYOT | MYOT1_HUMAN | Myotilin |
| MYRF | MYRF_HUMAN | Myelin regulatory factor, C-terminal |
| MYZAP | MYZAP_HUMAN | Myocardial zonula adherens protein |
| MZF1 | MZF1_HUMAN | Myeloid zinc finger 1 |
| NAA10 | NAA10_HUMAN | N-alpha-acetyltransferase 10 |
| NAAA | NAAA_HUMAN | N-acylethanolamine-hydrolyzing acid amidase subunit beta |
| NAALADL1 | NALDL_HUMAN | Aminopeptidase NAALADL1 |
| NABP2 | SOSB1_HUMAN | SOSS complex subunit B1 |
| NAE1 | ULA1_HUMAN | NEDD8-activating enzyme El regulatory subunit |
| NAGA | NAGAB_HUMAN | Alpha-N-acety lgalactosaminidase |
| NAGK | NAGK_HUMAN | N-acetyl-D-glucosamine kinase |
| NA1P | B1RC1_HUMAN | Baculoviral 1AP repeat-containing protein 1 |
| NAMPT | NAMPT_HUMAN | Nicotinamide phosphoribosyltransferase |
| NANOS1 | NANO1_HUMAN | Nanos homolog 1 |
| NANOS2 | NANO2_HUMAN | Nanos homolog 2 |
| NANOS3 | NANO3_HUMAN | Nanos homolog 3 |
| NARS | SYNC_HUMAN | Asparagine--tRNA ligase, cytoplasmic |
| NCAM1 | NCAM1_HUMAN | Neural cell adhesion molecule 1 |
| NCAM2 | NCAM2_HUMAN | Neural cell adhesion molecule 2 |
| NCF4 | NCF4_HUMAN | Neutrophil cytosol factor 4 |
| NCK1 | NCK1_HUMAN | Cytoplasmic protein NCK1 |
| NCK2 | NCK2_HUMAN | Cytoplasmic protein NCK2 |
| NCL | NUCL_HUMAN | Nucleolin |
| NCOA1 | NCOA1_HUMAN | Nuclear receptor coactivator 1 |
| NCR2 | NCTR2_HUMAN | Natural cytotoxicity triggering receptor 2 |
| NCR3 | NCTR3_HUMAN | Natural cytotoxicity triggering receptor 3 |
| NCR3LG1 | NR3L1_HUMAN | Natural cytotoxicity triggering receptor 3 ligand 1 |
| NDP | NDP_HUMAN | Norrin |
| NDRG2 | NDRG2_HUMAN | Protein NDRG2 |
| NDSTl | NDSTl_HUMAN | Heparan sulfate N-sulfotransferase 1 |
| NDUFA2 | NDUA2_HUMAN | NADH dehydrogenase [ubiquinone] 1 alpha subcomplex subunit 2 |
| NDUFS1 | NDUSl_HUMAN | NADH-ubiquinone oxidoreductase 75 kDa subunit, mitochondrial |
| NDUFS4 | NDUS4_HUMAN | NADH dehydrogenase [ubiquinone] iron-sulfur protein 4, |
| | | mitochondrial |
| NDUFS6 | NDUS6_HUMAN | NADH dehydrogenase [ubiquinone] iron-sulfur protein 6, |
| | | mitochondrial |
| NDUFVl | NDUVl_HUMAN | NADH dehydrogenase [ubiquinone] flavoprotein 1, mitochondrial |
| NEB | NEBU_HUMAN | Nebulin |
| NEBL | NEBL_HUMAN | Nebulette |
| NECTIN1 | NECT1_HUMAN | Nectin-1 |
| NECTIN2 | NECT2_HUMAN | Nectin-2 |
| NECTIN3 | NECT3_HUMAN | Nectin-3 |
| NECTIN4 | NECT4_HUMAN | Processed poliovirus receptor-related protein 4 |
| NEDD4 | NEDD4_HUMAN | E3 ubiquitin-protein ligase NEDD4 |
| NEDD4L | NED4L_HUMAN | E3 ubiquitin-protein ligase NEDD4-like |
| NEDD8 | NEDD8_HUMAN | NEDD8 |
| NEIL1 | NEIL1_HUMAN | Endonuclease 8-like 1 |
| NEK1 | NEK1_HUMAN | Serine/threonine-protein kinase Nekl |
| NEK2 | NEK2_HUMAN | Serine/threonine-protein kinase Nek2 |
| NEK7 | NEK7_HUMAN | Serine/threonine-protein kinase Nek7 |
| NEO1 | NEO1_HUMAN | Neogenin |
| NET1 | ARHG8_HUMAN | Neuroepithelial cell-transforming gene 1 protein |
| NEU2 | NEUR2_HUMAN | Sialidase-2 |
| NEURL1 | NEUL1_HUMAN | E3 ubiquitin-protein ligase NEURL1 |
| NEURL1B | NEU1B_HUMAN | E3 ubiquitin-protein ligase NEURL1B |
| NEURL4 | NEUL4_HUMAN | Neuralized-like protein 4 |
| NF1 | NF1_HUMAN | Neurofibromin truncated |
| NF2 | MERL_HUMAN | Merlin |
| NFASC | NFASC_HUMAN | Neurofascin |
| NFATC1 | NFAC1_HUMAN | Nuclear factor of activated T-cells, cytoplasmic 1 |
| NFATC2 | NFAC2_HUMAN | Nuclear factor of activated T-cells, cytoplasmic 2 |
| NFE2L2 | NF2L2_HUMAN | Nuclear factor erythroid 2-related factor 2 |
| NFKB1 | NFKB1_HUMAN | Nuclear factor NF-kappa-B p50 subunit |
| NFKB2 | NFKB2_HUMAN | Nuclear factor NF-kappa-B p52 subunit |
| NFKBlA | IKBA_HUMAN | NF-kappa-B inhibitor alpha |
| NFS1 | NFS1_HUMAN | Cysteine desulfurase, mitochondrial |
| NGF | NGF_HUMAN | Beta-nerve growth factor |
| NHLRC2 | NHLC2_HUMAN | NHL repeat-containing protein 2 |
| NKTR | NKTR_HUMAN | NK-tumor recognition protein |
| NLGN1 | NLGN1_HUMAN | Neuroligin-1 |
| NLGN2 | NLGN2_HUMAN | Neuroligin-2 |
| NLGN4X | NLGNX_HUMAN | Neuroligin-4, X-linked |
| NLN | NEUL_HUMAN | Neurolysin, mitochondrial |
| NMRK1 | NRK1_HUMAN | Nicotinamide riboside kinase 1 |
| NMTl | NMT1_HUMAN | Glycylpeptide N-tetradecanoyltransferase 1 |
| NNMT | NNMT_HUMAN | Nicotinamide N-methyltransferase |
| NOBl | NOBl_HUMAN | RNA-binding protein NOB1 |
| NOCT | NOCT_HUMAN | Nocturnin |
| NONO | NONO_HUMAN | Non-POU domain-containing octamer-binding protein |
| NOSl | NOSl_HUMAN | Nitric oxide synthase, brain |
| NOS2 | NOS2_HUMAN | Nitric oxide synthase, inducible |
| NOS3 | NOS3_HUMAN | Nitric oxide synthase, endothelial |
| NOTCH1 | NOTCl_HUMAN | Notch 1 intracellular domain |
| NOTUM | NOTUM_HUMAN | Palmitoleoyl-protein carboxylesterase NOTUM |
| NPC1 | NPCl_HUMAN | NPC intracellular cholesterol transporter 1 |
| NPHP1 | NPHPl_HUMAN | Nephrocystin-1 |
| NPM1 | NPM_HUMAN | Nucleophosmin |
| NPR1 | ANPRA_HUMAN | Atrial natriuretic peptide receptor 1 |
| NPR2 | ANPRB_HUMAN | Atrial natriuretic peptide receptor 2 |
| NPR3 | ANPRC_HUMAN | Atrial natriuretic peptide receptor 3 |
| NPRL2 | NPRL2_HUMAN | GATOR complex protein NPRL2 |
| NPTN | NPTN_HUMAN | Neuroplastin |
| NPY1R | NPY1R_HUMAN | Neuropeptide Y receptor type 1 |
| NR1Dl | NR1D1_HUMAN | Nuclear receptor subfamily 1 group D member 1 |
| NR1D2 | NR1D2_HUMAN | Nuclear receptor subfamily 1 group D member 2 |
| NR1H2 | NR1H2_HUMAN | Oxysterols receptor LXR-beta |
| NR1H3 | NR1H3_HUMAN | Oxysterols receptor LXR-alpha |
| NR1H4 | NR1H4_HUMAN | Bile acid receptor |
| NR112 | NR112_HUMAN | Nuclear receptor subfamily 1 group 1 member 2 |
| NR113 | NR113_HUMAN | Nuclear receptor subfamily 1 group 1 member 3 |
| NR2Cl | NR2Cl_HUMAN | Nuclear receptor subfamily 2 group C member 1 |
| NR2C2 | NR2C2_HUMAN | Nuclear receptor subfamily 2 group C member 2 |
| NR2El | NR2El_HUMAN | Nuclear receptor subfamily 2 group E member 1 |
| NR2E3 | NR2E3_HUMAN | Photoreceptor-specific nuclear receptor |
| NR2Fl | COT1_HUMAN | COUP transcription factor 1 |
| NR2F2 | COT2_HUMAN | COUP transcription factor 2 |
| NR2F6 | NR2F6_HUMAN | Nuclear receptor subfamily 2 group F member 6 |
| NR3Cl | GCR_HUMAN | Glucocorticoid receptor |
| NR3C2 | MCR_HUMAN | Mineralocorticoid receptor |
| NR4Al | NR4Al_HUMAN | Nuclear receptor subfamily 4 group A member 1 |
| NR4A2 | NR4A2_HUMAN | Nuclear receptor subfamily 4 group A member 2 |
| NR4A3 | NR4A3_HUMAN | Nuclear receptor subfamily 4 group A member 3 |
| NR5Al | STFl_HUMAN | Steroidogenic factor 1 |
| NR5A2 | NR5A2_HUMAN | Nuclear receptor subfamily 5 group A member 2 |
| NR6Al | NR6Al_HUMAN | Nuclear receptor subfamily 6 group A member 1 |
| NRCAM | NRCAM_HUMAN | Neuronal cell adhesion molecule |
| NSDl | NSDl_HUMAN | Histone-lysine N-methyltransferase, H3 lysine-36 and H4 lysine-20 |
| | | specific |
| NSD2 | NSD2_HUMAN | Histone-lysine N-methyltransferase NSD2 |
| NSD3 | NSD3_HUMAN | Histone-lysine N-methyltransferase NSD3 |
| NSFL1C | NSF1C_HUMAN | NSFL1 cofactor p47 |
| NSMCE1 | NSEl_HUMAN | Non-structural maintenance of chromosomes element 1 homolog |
| NSMCE2 | NSE2_HUMAN | E3 SUMO-protein ligase NSE2 |
| NT5C2 | 5NTC_HUMAN | Cytosolic purine 5′-nucleotidase |
| NT5E | 5NTD_HUMAN | 5′-nucleotidase |
| NTF3 | NTF3_HUMAN | Neurotrophin-3 |
| NTF4 | NTF4_HUMAN | Neurotrophin-4 |
| NTN1 | NET1_HUMAN | Netrin-1 |
| NTNG1 | NTNG1_HUMAN | Netrin-Gl |
| NTNG2 | NTNG2_HUMAN | Netrin-G2 |
| NTPCR | NTPCR_HUMAN | Cancer-related nucleoside-triphosphatase |
| NTRK1 | NTRKl_HUMAN | High affinity nerve growth factor receptor |
| NTRK2 | NTRK2_HUMAN | BDNF/NT-3 growth factors receptor |
| NTRK3 | NTRK3_HUMAN | NT-3 growth factor receptor |
| NUDT1 | 8ODP_HUMAN | 7,8-dihydro-8-oxoguanine triphosphatase |
| NUDT14 | NUD14_HUMAN | Uridine diphosphate glucose pyrophosphatase |
| NUDT16 | NUD16_HUMAN | U8 snoRNA-decapping enzyme |
| NUDT4 | NUDT4_HUMAN | Diphosphoinositol polyphosphate phosphohydrolase 2 |
| NUDT5 | NUDT5_HUMAN | ADP-sugar pyrophosphatase |
| NUDT6 | NUDT6_HUMAN | Nucleoside diphosphate-linked moiety X motif 6 |
| NUDT7 | NUDT7_HUMAN | Peroxisomal coenzyme A diphosphatase NUDT7 |
| NUDT9 | NUDT9_HUMAN | ADP-ribose pyrophosphatase, mitochondrial |
| NUMB | NUMB_HUMAN | Protein numb homolog |
| NUP133 | NU133_HUMAN | Nuclear pore complex protein Nupl33 |
| NUP155 | NU155_HUMAN | Nuclear pore complex protein Nupl55 |
| NUP160 | NU160_HUMAN | Nuclear pore complex protein Nupl60 |
| NUP214 | NU214_HUMAN | Nuclear pore complex protein Nup2 1 4 |
| NUP37 | NUP37_HUMAN | Nucleoporin Nup37 |
| NUP43 | NUP43_HUMAN | Nucleoporin Nup43 |
| NUP50 | NUP50_HUMAN | Nuclear pore complex protein Nup50 |
| NUP54 | NUP54_HUMAN | Nucleoporin p54 |
| NUP98 | NUP98_HUMAN | Nuclear pore complex protein Nup96 |
| NXF1 | NXF1_HUMAN | Nuclear RNA export factor 1 |
| OAS1 | OAS1_HUMAN | 2′-5′-oligoadenylate synthase 1 |
| OASL | OASL_HUMAN | 2′-5′-oligoadenylate synthase-like protein |
| OAT | OAT_HUMAN | Ornithine aminotransferase, renal form |
| OBP2A | OBP2A_HUMAN | Odorant-binding protein 2a |
| OBSCN | OBSCN_HUMAN | Obscurin |
| OBSL1 | OBSL1_HUMAN | Obscurin-like protein 1 |
| OLFM1 | NOE1_HUMAN | Noelin |
| OPCML | OPCM_HUMAN | Opioid-binding protein/cell adhesion molecule |
| OPRK1 | OPRK_HUMAN | Kappa-type opioid receptor |
| OPTN | OPTN_HUMAN | Optineurin |
| ORC2 | ORC2_HUMAN | Origin recognition complex subunit 2 |
| ORM1 | A1AG1_HUMAN | Alpha- I-acid glycoprotein 1 |
| ORM2 | AlAG2_HUMAN | Alpha- I-acid glycoprotein 2 |
| OS9 | OS9_HUMAN | Protein OS-9 |
| OSBPL11 | OSB11_HUMAN | Oxysterol-binding protein-related protein 11 |
| OSBPL1A | OSBL1_HUMAN | Oxysterol-binding protein-related protein 1 |
| OSBPL2 | OSBL2_HUMAN | Oxysterol-binding protein-related protein 2 |
| OSBPL8 | OSBL8_HUMAN | Oxysterol-binding protein-related protein 8 |
| OSR1 | OSRl_HUMAN | Protein odd-skipped-related 1 |
| OSR2 | OSR2_HUMAN | Protein odd-skipped-related 2 |
| OSTF1 | OSTFl_HUMAN | Osteoclast-stimulating factor 1 |
| OTUD1 | OTUDl_HUMAN | OTU domain-containing protein 1 |
| OVOL1 | OVOLl_HUMAN | Putative transcription factor Ovo-like 1 |
| OVOL2 | OVOL2_HUMAN | Transcription factor Ovo-like 2 |
| OVOL3 | OVOL3_HUMAN | Putative transcription factor ovo-like protein 3 |
| OXCT1 | SCOTl_HUMAN | Succinyl-CoA:3-ketoacid coenzyme A transferase 1, mitochondrial |
| OXSM | OXSM_HUMAN | 3-oxoacy 1-[acyl-carrier-protein] synthase, mitochondrial |
| OXSR1 | OXSR1_HUMAN | Serine/threonine-protein kinase OSR1 |
| P2RX3 | P2RX3_HUMAN | P2X purinoceptor 3 |
| P2RY1 | P2RY1_HUMAN | P2Y purinoceptor 1 |
| PABPCl | PABP1_HUMAN | Polyadeny late-binding protein 1 |
| PACSlN1 | PACN1_HUMAN | Protein kinase C and casein kinase substrate in neurons protein 1 |
| PACS1N2 | PACN2_HUMAN | Protein kinase C and casein kinase substrate in neurons protein 2 |
| PAD12 | PAD12_HUMAN | Protein-arginine deiminase type-2 |
| PAD14 | PAD14_HUMAN | Protein-arginine deiminase type-4 |
| PAFl | PAF1_HUMAN | RNA polymerase II-associated factor 1 homolog |
| PAlP1 | PAlPl_HUMAN | Polyadenylate-binding protein-interacting protein 1 |
| PAKl | PAK1_HUMAN | Serine/threonine-protein kinase PAK 1 |
| PAK2 | PAK2_HUMAN | PAK-2p34 |
| PAK3 | PAK3_HUMAN | Serine/threonine-protein kinase PAK 3 |
| PAK4 | PAK4_HUMAN | Serine/threonine-protein kinase PAK 4 |
| PAK5 | PAK5_HUMAN | Serine/threonine-protein kinase PAK 5 |
| PAK6 | PAK6_HUMAN | Serine/threonine-protein kinase PAK 6 |
| PALB2 | PALB2_HUMAN | Partner and localizer of BRCA2 |
| PALLD | PALLD_HUMAN | Palladin |
| PANK1 | PANK1_HUMAN | Pantothenate kinase 1 |
| PANK2 | PANK2_HUMAN | Pantothenate kinase 2, mitochondrial |
| PANK3 | PANK3_HUMAN | Pantothenate kinase 3 |
| PAPSS1 | PAPS1_HUMAN | Adenyly-sulfate kinase |
| PARD3 | PARD3_HUMAN | Partitioning defective 3 homolog |
| PARD6A | PAR6A_HUMAN | Partitioning defective 6 homolog alpha |
| PARP1 | PARP1_HUMAN | Poly [ADP-ribose] polymerase 1 |
| PARP10 | PAR10_HUMAN | Protein mono-ADP-ribosyltransferase PARP10 |
| PARP11 | PAR11_HUMAN | Protein mono-ADP-ribosyltransferase PARP11 |
| PARP14 | PAR14_HUMAN | Protein mono-ADP-ribosyltransferase PARP14 |
| PARP15 | PAR15_HUMAN | Protein mono-ADP-ribosyltransferase PARP15 |
| PASK | PASK_HUMAN | PAS domain-containing serine/threonine-protein ckinase |
| PATJ | INADL_HUMAN | lnaD-like protein |
| PATZ1 | PATZ1_HUMAN | POZ-, AT hook-, and zinc finger-containing protein 1 |
| PAX5 | PAX5_HUMAN | Paired box protein Pax-5 |
| PAX6 | PAX6_HUMAN | Paired box protein Pax-6 |
| PBRM1 | PB1_HUMAN | Protein polybromo-1 |
| PC | PYC_HUMAN | Pyruvate carboxylase, mitochondrial |
| PCBD2 | PHS2_HUMAN | Pterin-4-alpha-carbinolamine dehydratase 2 |
| PCDH1 | PCDH1_HUMAN | Protocadherin-1 |
| PCDH15 | PCD15_HUMAN | Protocadherin-15 |
| PCDH7 | PCDH7_HUMAN | Protocadherin-7 |
| PCDH9 | PCDH9_HUMAN | Protocadherin-9 |
| PCDHGB3 | PCDGF_HUMAN | Protocadherin gamma-B3 |
| PCGF2 | PCGF2_HUMAN | Polycomb group RING finger protein 2 |
| PCGF5 | PCGF5_HUMAN | Polycomb group RING finger protein 5 |
| PCK1 | PCKGC_HUMAN | Phosphoenolpymvate carboxykinase, cytosolic [GTP] |
| PCMT1 | PIMT_HUMAN | Protein-L-isoaspartate(D-aspartate) 0-methy Itransferase |
| PCNA | PCNA_HUMAN | Proliferating cell nuclear antigen |
| PCOLCE | PCOC1_HUMAN | Procollagen C-endopeptidase enhancer 1 |
| PCSK9 | PCSK9_HUMAN | Proprotein convertase subtilisin/kexin type 9 |
| PCTP | PPCT_HUMAN | Phosphatidylcholine transfer protein |
| PDCD1 | PDCD1_HUMAN | Programmed cell death protein 1 |
| PDCD11 | RRP5_HUMAN | Protein RRP5 homolog |
| PDCD2 | PDCD2_HUMAN | Programmed cell death protein 2 |
| PDCD6 | PDCD6_HUMAN | Programmed cell death protein 6 |
| PDE4B | PDE4B_HUMAN | CAMP-specific 3′,5′-cyclic phosphodiesterase 4B |
| PDE4D | PDE4D_HUMAN | CAMP-specific 3′,5′-cyclic phosphodiesterase 4D |
| PDE5A | PDE5A_HUMAN | cGMP-specific 3′,5′-cyclic phosphodiesterase |
| PDE6D | PDE6D_HUMAN | Retinal rod rhodopsin-sensitive cGMP 3′,5′-cyclic phosphodiesterase |
| | | subunit delta |
| PDF | DEFM_HUMAN | Peptide deformylase, mitochondrial |
| PDGFRB | PGFRB_HUMAN | Platelet-derived growth factor receptor beta |
| PD1A3 | PD1A3_HUMAN | Protein disulfide-isomerase A3 |
| PDK2 | PDK2_HUMAN | [Pymvate dehydrogenase (acetyl-transferring)] kinase isozyme 2, |
| | | mitochondrial |
| PDK4 | PDK4_HUMAN | [Pymvate dehydrogenase (acetyl-transferring)] kinase isozyme 4, |
| | | mitochondrial |
| PDL1Ml | PDLI1_HUMAN | PDZ and LIM domain protein 1 |
| PDXK | PDXK_HUMAN | Pyridoxal kinase |
| PDZD3 | NHRF4_HUMAN | Na(+)/H(+) exchange regulatory cofactor NHERF4 |
| PDZRN3 | PZRN3_HUMAN | E3 ubiquitin-protein ligase PDZRN3 |
| PDZRN4 | PZRN4_HUMAN | PDZ domain-containing RING finger protein 4 |
| PEG10 | PEG10_HUMAN | Retrotransposon-derived protein PEG 10 |
| PEG3 | PEG3_HUMAN | Paternally-expressed gene 3 protein |
| PEL12 | PELl2_HUMAN | E3 ubiquitin-protein ligase pellino homolog 2 |
| PEPD | PEPD_HUMAN | Xaa-Pro dipeptidase |
| PEX2 | PEX2_HUMAN | Peroxisome biogenesis factor 2 |
| PEX5 | PEX5_HUMAN | Peroxisomal targeting signal 1 receptor |
| PF4 | PLF4_HUMAN | Platelet factor 4, short form |
| PF4Vl | PF4V_HUMAN | Platelet factor 4 variant( 6-7 4) |
| PFKFBl | F261_HUMAN | Fmctose-2,6-bisphosphatase |
| PGA4 | PEPA4_HUMAN | PepsinA-4 |
| PGAMS | PGAM5_HUMAN | Serine/threonine-protein phosphatase PGAM5, mitochondrial |
| PGC | PEPC_HUMAN | Gastricsin |
| PGD | 6PGD_HUMAN | 6-phosphogluconate dehydrogenase, decarboxylating |
| PGK1 | PGK1_HUMAN | Phosphoglycerate kinase 1 |
| PGLYRP3 | PGRP3_HUMAN | Peptidoglycan recognition protein 3 |
| PGLYRP4 | PGRP4_HUMAN | Peptidoglycan recognition protein 4 |
| PGM1 | PGM1_HUMAN | Phosphoglucomutase-1 |
| PGR | PRGR_HUMAN | Progesterone receptor |
| PHC1 | PHC1_HUMAN | Polyhomeotic-like protein 1 |
| PHC2 | PHC2_HUMAN | Polyhomeotic-like protein 2 |
| PHC3 | PHC3_HUMAN | Polyhomeotic-like protein 3 |
| PHF1 | PHF1_HUMAN | PHD finger protein 1 |
| PHF14 | PHF14_HUMAN | PHD finger protein 14 |
| PHF19 | PHF19_HUMAN | PHD finger protein 19 |
| PHF20 | PHF20_HUMAN | PHD finger protein 20 |
| PHF20L1 | P20L1_HUMAN | PHD finger protein 20-like protein 1 |
| PHF23 | PHF23_HUMAN | PHD finger protein 23 |
| PHF5A | PHF5A_HUMAN | PHD finger-like domain-containing protein 5A |
| PHF6 | PHF6_HUMAN | PHD finger protein 6 |
| PHF7 | PHF7_HUMAN | PHD finger protein 7 |
| PHKG2 | PHKG2_HUMAN | Phosphorylase b kinase gamma catalytic chain, liver/testis isoform |
| PHRF1 | PHRF1_HUMAN | PHD and RING finger domain-containing protein 1 |
| Pl4K2A | P4K2A_HUMAN | Phosphatidylinositol 4-kinase type 2-alpha |
| Pl4K2B | P4K2B_HUMAN | Phosphatidylinositol 4-kinase type 2-beta |
| Pl4KA | P14KA_HUMAN | Phosphatidylinositol 4-kinase alpha |
| Pl4KB | Pl4KB_HUMAN | Phosphatidylinositol 4-kinase beta |
| PIAS3 | PIAS3_HUMAN | E3 SUMO-protein ligase PIAS3 |
| PIFl | PIFl_HUMAN | ATP-dependent DNA helicase PIFl |
| PIGR | PIGR_HUMAN | Secretory component |
| PIHlDl | PIHDl_HUMAN | PIH1 domain-containing protein 1 |
| PIK3C3 | PK3C3_HUMAN | Phosphatidylinositol 3-kinase catalytic subunit type 3 |
| PIK3CA | PK3CA_HUMAN | Phosphatidylinositol 4,5-bisphosphate 3-kinase catalytic subunit alpha |
| | | isoform |
| PIK3CD | PK3CD_HUMAN | Phosphatidylinositol 4,5-bisphosphate 3-kinase catalytic subunit delta |
| | | isoform |
| PIK3CG | PK3CG_HUMAN | Phosphatidylinositol 4,5-bisphosphate 3-kinase catalytic subunit |
| | | gamma isoform |
| PIK3R1 | P85A_HUMAN | Phosphatidylinositol 3-kinase regulatory subunit alpha |
| PIKFYVE | FYV1_HUMAN | 1-phosphatidylinositol 3-phosphate 5-kinase |
| PILRA | PILRA_HUMAN | Paired immunoglobulin-like type 2 receptor alpha |
| PILRB | PILRB_HUMAN | Paired immunoglobulin-like type 2 receptor beta |
| PIM1 | PIM1_HUMAN | Serine/threonine-protein kinase pim-1 |
| PIM2 | PIM2_HUMAN | Serine/threonine-protein kinase pim-2 |
| PIN1 | PIN1_HUMAN | Peptidyl-prolyl cis-trans isomerase NIMA-interacting 1 |
| PIN4 | PIN4_HUMAN | Peptidy1-prolyl cis-trans isomerase NIMA-interacting 4 |
| PIP4K2B | Pl42B_HUMAN | Phosphatidylinositol 5-phosphate 4-kinase type-2 beta |
| PIR | PIR_HUMAN | Pirin |
| PITPNA | PIPNA_HUMAN | Phosphatidylinositol transfer protein alpha isoform |
| PlTRM1 | PREP_HUMAN | Presequence protease, mitochondrial |
| PlWlL1 | PlWL1_HUMAN | Piwi-like protein 1 |
| PlWlL2 | PlWL2_HUMAN | Piwi-like protein 2 |
| PKD1 | PKD1_HUMAN | Polycystin-1 |
| PKD2 | PKD2_HUMAN | Polycystin-2 |
| PKD2Ll | PK2Ll_HUMAN | Polycystic kidney disease 2-like 1 protein |
| PKLR | KPYR_HUMAN | Pymvate kinase PKLR |
| PKM | KPYM_HUMAN | Pymvate kinase PKM |
| PKMYT1 | PMYT1_HUMAN | Membrane-associated tyrosine- and threonine-specific cdc2-inhibitory |
| | | kinase |
| PKN1 | PKN1_HUMAN | Serine/threonine-protein kinase Nl |
| PKN2 | PKN2_HUMAN | Serine/threonine-protein kinase N2 |
| PLA2G2E | PA2GE_HUMAN | Group IIE secretory phospholipase A2 |
| PLA2G4A | PA24A_HUMAN | Lysophospholipase |
| PLA2G4D | PA24D_HUMAN | Cytosolic phospholipase A2 delta |
| PLAA | PLAP_HUMAN | Phospholipase A-2-activating protein |
| PLAG1 | PLAG1_HUMAN | Zinc finger protein PLAG1 |
| PLAGL1 | PLAL1_HUMAN | Zinc finger protein PLAGL1 |
| PLAGL2 | PLAL2_HUMAN | Zinc finger protein PLAGL2 |
| PLAU | UROK_HUMAN | Urokinase-type plasminogen activator chain B |
| PLAUR | UPAR_HUMAN | Urokinase plasminogen activator surface receptor |
| PLCG1 | PLCG1_HUMAN | 1-phosphatidy linositol 4,5-bisphosphate phosphodiesterase gamma-I |
| PLCG2 | PLCG2_HUMAN | 1-phosphatidy linositol 4,5-bisphosphate phosphodiesterase gamma-2 |
| PLEC | PLEC_HUMAN | Plectin |
| PLEKHB2 | PKHB2_HUMAN | Pleckstrin homology domain-containing family B member 2 |
| PLEKHF1 | PKHF1_HUMAN | Pleckstrin homology domain-containing family F member 1 |
| PLEKHF2 | PKHF2_HUMAN | Pleckstrin homology domain-containing family F member 2 |
| PLEKHM3 | PKHM3_HUMAN | Pleckstrin homology domain-containing family M member 3 |
| PLG | PLMN_HUMAN | Plasmin light chain B |
| PLK1 | PLK1_HUMAN | Serine/threonine-protein kinase PLK1 |
| PLK2 | PLK2_HUMAN | Serine/threonine-protein kinase PLK2 |
| PLK3 | PLK3_HUMAN | Serine/threonine-protein kinase PLK3 |
| PLK4 | PLK4_HUMAN | Serine/threonine-protein kinase PLK4 |
| PLRG1 | PLRG1_HUMAN | Pleiotropic regulator 1 |
| PLXNA4 | PLXA4_HUMAN | Plexin-A4 |
| PLXNB1 | PLXB1_HUMAN | Plexin-B1 |
| PLXNB2 | PLXB2_HUMAN | Plexin-B2 |
| PLXNC1 | PLXC1_HUMAN | Plexin-Cl |
| PLXND1 | PLXD1_HUMAN | Plexin-Dl |
| PMS2 | PMS2_HUMAN | Mismatch repair endonuclease PMS2 |
| PNLIP | LIPP_HUMAN | Pancreatic triacylglycerol lipase |
| PNLIPRP1 | LIPR1_HUMAN | Inactive pancreatic lipase-related protein 1 |
| PNLIPRP2 | LIPR2_HUMAN | Pancreatic lipase-related protein 2 |
| PNMA3 | PNMA3_HUMAN | Paraneoplastic antigen Ma3 |
| PNPO | PNPO_HUMAN | Pyridoxine-5′-phosphate oxidase |
| PNPT1 | PNPT1_HUMAN | Polyribonucleotide nucleotidy ltransferase 1, mitochondrial |
| POGLUT2 | PLGT2_HUMAN | Protein O-glucosy ltransferase 2 |
| POLA1 | DPOLA_HUMAN | DNA polymerase alpha catalytic subunit |
| POLB | DPOLB_HUMAN | DNA polymerase beta |
| POLE2 | DPOE2_HUMAN | DNA polymerase epsilon subunit 2 |
| POLG | DPOG1_HUMAN | DNA polymerase subunit gamma-1 |
| POLG2 | DPOG2_HUMAN | DNA polymerase subunit gamma-2, mitochondrial |
| POLH | POLH_HUMAN | DNA polymerase eta |
| POLL | DPOLL_HUMAN | DNA polymerase lambda |
| POLM | DPOLM_HUMAN | DNA-directed DNA/RNA polymerase mu |
| POLN | DPOLN_HUMAN | DNA polymerase nu |
| POLQ | DPOLQ_HUMAN | DNA polymerase theta |
| POLR1B | RPA2_HUMAN | DNA-directed RNA polymerase I subunit RPA2 |
| POLR2A | RPB1_HUMAN | DNA-directed RNA polymerase II subunit RPB1 |
| POLR2B | RPB2_HUMAN | DNA-directed RNA polymerase II subunit RPB2 |
| POLR2E | RPAB1_HUMAN | DNA-directed RNA polymerases 1, II, and Ill subunit RPABC1 |
| POLR2G | RPB7_HUMAN | DNA-directed RNA polymerase II subunit RPB7 |
| POLR21 | RPB9_HUMAN | DNA-directed RNA polymerase II subunit RPB9 |
| POLR2K | RPAB4_HUMAN | DNA-directed RNA polymerases 1, II, and Ill subunit RPABC4 |
| POLR2L | RPAB5_HUMAN | DNA-directed RNA polymerases 1, II, and Ill subunit RPABC5 |
| POLR3B | RPC2_HUMAN | DNA-directed RNA polymerase Ill subunit RPC2 |
| POLR3C | RPC3_HUMAN | DNA-directed RNA polymerase Ill subunit RPC3 |
| POLR3K | RPC10_HUMAN | DNA-directed RNA polymerase Ill subunit RPC10 |
| POLRMT | RPOM_HUMAN | DNA-directed RNA polymerase, mitochondrial |
| POMGNT1 | PMGT1_HUMAN | Protein O-linked-mannose beta-1,2-Nacetylglucosaminyltransferase 1 |
| POP1 | POPI_HUMAN | Ribonucleases P/MRP protein subunit POP1 |
| POP5 | POP5_HUMAN | Ribonuclease P/MRP protein subunit POP5 |
| POR | NCPR_HUMAN | NADPH -- cytochrome P450 reductase |
| POSTN | POSTN_HUMAN | Periostin |
| POT1 | POTE1_HUMAN | Protection of telomeres protein 1 |
| PPA1 | IPYR_HUMAN | Inorganic pyrophosphatase |
| PPARA | PPARA_HUMAN | Peroxisome proliferator-activated receptor alpha |
| PPARD | PPARD_HUMAN | Peroxisome proliferator-activated receptor delta |
| PPARG | PPARG_HUMAN | Peroxisome proliferator-activated receptor gamma |
| PPBP | CXCL7_HUMAN | Neutrophil-activating peptide 2(1-63) |
| PPIA | PP1A_HUMAN | Peptidyl-prolyl cis-trans isomerase A, N-terminally processed |
| PPIE | PPIE_HUMAN | Peptidyl-prolyl cis-trans isomerase E |
| PPIL1 | PPILl_HUMAN | Peptidy1-prolyl cis-trans isomerase-like 1 |
| PPIL3 | PPIL3_HUMAN | Peptidyl-prolyl cis-trans isomerase-like 3 |
| PPL | PEPL_HUMAN | Periplakin |
| PPM1K | PPM1K_HUMAN | Protein phosphatase lK, mitochondrial |
| PPME1 | PPME1_HUMAN | Protein phosphatase methylesterase 1 |
| PPOX | PPOX_HUMAN | Protoporphyrinogen oxidase |
| PPP1Rl3L | IASPP_HUMAN | RelA-associated inhibitor |
| PPP2R2A | 2ABA_HUMAN | Serine/threonine-protein phosphatase 2A 55 kDa regulatory subunit B |
| | | alpha isoform |
| PPP3CA | PP2BA_HUMAN | Serine/threonine-protein phosphatase 2B catalytic subunit alpha |
| | | isoform |
| PPP3CB | PP2BB_HUMAN | Serine/threonine-protein phosphatase 2B catalytic subunit beta isoform |
| PRDM1 | PRDM1_HUMAN | PR domain zinc finger protein 1 |
| PRDM10 | PRD10_HUMAN | PR domain zinc finger protein 10 |
| PRDM11 | PRD11_HUMAN | PR domain-containing protein 11 |
| PRDM12 | PRD12_HUMAN | PR domain zinc finger protein 12 |
| PRDM13 | PRD13_HUMAN | PR domain zinc finger protein 13 |
| PRDM14 | PRD14_HUMAN | PR domain zinc finger protein 14 |
| PRDM15 | PRD15_HUMAN | PR domain zinc finger protein 15 |
| PRDM16 | PRD16_HUMAN | Histone-lysine N-methyltransferase PRDM16 |
| PRDM2 | PRDM2_HUMAN | PR domain zinc finger protein 2 |
| PRDM5 | PRDM5_HUMAN | PR domain zinc finger protein 5 |
| PRDM6 | PRDM6_HUMAN | Putative histone-lysine N-methyltransferase PRDM6 |
| PRDM9 | PRDM9_HUMAN | Histone-lysine N-methyltransferase PRDM9 |
| PRDX1 | PRDX1_HUMAN | Peroxiredoxin-1 |
| PRDX2 | PRDX2_HUMAN | Peroxiredoxin-2 |
| PRDX3 | PRDX3_HUMAN | Thioredoxin-dependent peroxide reductase, mitochondrial |
| PRDX4 | PRDX4_HUMAN | Peroxiredoxin-4 |
| PRDX5 | PRDX5_HUMAN | Peroxiredoxin-5, mitochondrial |
| PRDX6 | PRDX6_HUMAN | Peroxiredoxin-6 |
| PREB | PREB_HUMAN | Prolactin regulatory element-binding protein |
| PREP | PPCE_HUMAN | Prolyl endopeptidase |
| PREX2 | PREX2_HUMAN | Phosphatidylinositol 3,4,5-trisphosphate-dependent Rae exchanger 2 |
| | | protein |
| PRG2 | PRG2_HUMAN | Eosinophil granule major basic protein |
| PRIM1 | PRI1_HUMAN | DNA primase small subunit |
| PR1MPOL | PR1PO_HUMAN | DNA-directed primase/polymerase protein |
| PRKAA1 | AAPK1_HUMAN | 5′-AMP-activated protein kinase catalytic subunit alpha-1 |
| PRKAA2 | AAPK2_HUMAN | 5′-AMP-activated protein kinase catalytic subunit alpha-2 |
| PRKAB1 | AAKB1_HUMAN | 5′-AMP-activated protein kinase subunit beta-1 |
| PRKAB2 | AAKB2_HUMAN | 5′-AMP-activated protein kinase subunit beta-2 |
| PRKACA | KAPCA_HUMAN | cAMP-dependent protein kinase catalytic subunit alpha |
| PRKAG1 | AAKG1_HUMAN | 5′-AMP-activated protein kinase subunit gamma-1 |
| PRKCA | KPCA_HUMAN | Protein kinase C alpha type |
| PRKCB | KPCB_HUMAN | Protein kinase C beta type |
| PRKCD | KPCD_HUMAN | Protein kinase C delta type catalytic subunit |
| PRKCE | KPCE_HUMAN | Protein kinase C epsilon type |
| PRKCG | KPCG_HUMAN | Protein kinase C gamma type |
| PRKCH | KPCL_HUMAN | Protein kinase C eta type |
| PRKC1 | KPC1_HUMAN | Protein kinase C iota type |
| PRKCQ | KPCT_HUMAN | Protein kinase C iota type |
| PRKD1 | KPCD1_HUMAN | Serine/threonine-protein kinase DI |
| PRKD2 | KPCD2_HUMAN | Serine/threonine-protein kinase D2 |
| PRKD3 | KPCD3_HUMAN | Serine/threonine-protein kinase D3 |
| PRKDC | PRKDC_HUMAN | DNA-dependent protein kinase catalytic subunit |
| PRKG1 | KGP1_HUMAN | cGMP-dependent protein kinase 1 |
| PRKN | PRKN_HUMAN | E3 ubiquitin-protein ligase parkin |
| PRLR | PRLR_HUMAN | Prolactin receptor |
| PRMT5 | ANM5_HUMAN | Protein arginine N-methyltransferase 5, N-terminally processed |
| PRNP | PR10_HUMAN | Major prion protein |
| PROS1 | PROS_HUMAN | Vitamin K-dependent protein S |
| PROZ | PROZ_HUMAN | Vitamin K-dependent protein Z |
| PRPF19 | PRP19_HUMAN | Pre-mRNA-processing factor 19 |
| PRPF38A | PR38A_HUMAN | Pre-mRNA-splicing factor 38A |
| PRPF4 | PRP4_HUMAN | U4/U6 small nuclear ribonucleoprotein Prp4 |
| PRPF40A | PR40A_HUMAN | Pre-mRNA-processing factor 40 homolog A |
| PRPF8 | PRP8_HUMAN | Pre-mRNA-processing-splicing factor 8 |
| PRPSAP1 | KPRA_HUMAN | Phosphoribosyl pyrophosphate synthase-associated protein 1 |
| PSAT1 | SERC_HUMAN | Phosphoserine aminotransferase |
| PSMA1 | PSA1_HUMAN | Proteasome subunit alpha type-1 |
| PSMA2 | PSA2_HUMAN | Proteasome subunit alpha type-2 |
| PSMA3 | PSA3_HUMAN | Proteasome subunit alpha type-3 |
| PSMA4 | PSA4_HUMAN | Proteasome subunit alpha type-4 |
| PSMA5 | PSA5_HUMAN | Proteasome subunit alpha type-5 |
| PSMA6 | PSA6_HUMAN | Proteasome subunit alpha type-6 |
| PSMA7 | PSA7_HUMAN | Proteasome subunit alpha type-7 |
| PSMB1 | PSB1_HUMAN | Proteasome subunit beta type-1 |
| PSMB10 | PSB10_HUMAN | Proteasome subunit beta type-10 |
| PSMB2 | PSB2_HUMAN | Proteasome subunit beta type-2 |
| PSMB3 | PSB3_HUMAN | Proteasome subunit beta type-3 |
| PSMB4 | PSB4_HUMAN | Proteasome subunit beta type-4 |
| PSMB5 | PSB5_HUMAN | Proteasome subunit beta type-5 |
| PSMB6 | PSB6_HUMAN | Proteasome subunit beta type-6 |
| PSMB7 | PSB7_HUMAN | Proteasome subunit beta type-7 |
| PSMB8 | PSB8_HUMAN | Proteasome subunit beta type-8 |
| PSMB9 | PSB9_HUMAN | Proteasome subunit beta type-9 |
| PSMC1 | PRS4_HUMAN | 26S proteasome regulatory subunit 4 |
| PSMC4 | PRS6B_HUMAN | 26S proteasome regulatory subunit 6B |
| PSMC5 | PRS8_HUMAN | 26S proteasome regulatory subunit 8 |
| PSMC6 | PRS10_HUMAN | 26S proteasome regulatory subunit 10B |
| PSMD1 | PSMD1_HUMAN | 26S proteasome non-ATPase regulatory subunit 1 |
| PSMD10 | PSD10_HUMAN | 26S proteasome non-ATPase regulatory subunit 10 |
| PSMD11 | PSD11_HUMAN | 26S proteasome non-ATPase regulatory subunit 11 |
| PSMD12 | PSD12_HUMAN | 26S proteasome non-ATPase regulatory subunit 12 |
| PSMD14 | PSDE_HUMAN | 26S proteasome non-ATPase regulatory subunit 14 |
| PSMD3 | PSMD3_HUMAN | 26S proteasome non-ATPase regulatory subunit 3 |
| PSPC1 | PSPC1_HUMAN | Paraspeckle component 1 |
| PTCRA | PTCRA_HUMAN | Pre T-cell antigen receptor alpha |
| PTGDS | PTGDS_HUMAN | Prostaglandin-H2 D-isomerase |
| PTGER3 | PE2R3_HUMAN | Prostaglandin E2 receptor EP3 subtype |
| PTGS2 | PGH2_HUMAN | Prostaglandin G/H synthase 2 |
| PTK2 | FAK1_HUMAN | Focal adhesion kinase 1 |
| PTK2B | FAK2_HUMAN | Protein-tyrosine kinase 2-beta |
| PTK6 | PTK6_HUMAN | Protein-tyrosine kinase 6 |
| PTPN11 | PTN11_HUMAN | Tyrosine-protein phosphatase non-receptor type 11 |
| PTPN12 | PTN12_HUMAN | Tyrosine-protein phosphatase non-receptor type 12 |
| PTPN13 | PTN13_HUMAN | Tyrosine-protein phosphatase non-receptor type 13 |
| PTPN14 | PTN14_HUMAN | Tyrosine-protein phosphatase non-receptor type 14 |
| PTPN2 | PTN2_HUMAN | Tyrosine-protein phosphatase non-receptor type 2 |
| PTPN23 | PTN23_HUMAN | Tyrosine-protein phosphatase non-receptor type 23 |
| PTPN3 | PTN3_HUMAN | Tyrosine-protein phosphatase non-receptor type 3 |
| PTPN5 | PTN5_HUMAN | Tyrosine-protein phosphatase non-receptor type 5 |
| PTPN6 | PTN6_HUMAN | Tyrosine-protein phosphatase non-receptor type 6 |
| PTPN7 | PTN7_HUMAN | Tyrosine-protein phosphatase non-receptor type 7 |
| PTPRD | PTPRD_HUMAN | Receptor-type tyrosine-protein phosphatase delta |
| PTPRF | PTPRF_HUMAN | Receptor-type tyrosine-protein phosphatase F |
| PTPRM | PTPRM_HUMAN | Receptor-type tyrosine-protein phosphatase mu |
| PTPRR | PTPRR_HUMAN | Receptor-type tyrosine-protein phosphatase R |
| PTPRS | PTPRS_HUMAN | Receptor-type tyrosine-protein phosphatase S |
| PTPRZ1 | PTPRZ_HUMAN | Receptor-type tyrosine-protein phosphatase zeta |
| PTS | PTPS_HUMAN | 6-pymvoyl tetrahydrobiopterin synthase |
| PUF60 | PUF60_HUMAN | Poly(U)-binding-splicing factor PUF60 |
| PUS7 | PUS7_HUMAN | Pseudouridylate synthase 7 homolog |
| PVR | PVR_HUMAN | Poliovirus receptor |
| PWWP2B | PWP2B_HUMAN | PWWP domain-containing protein 2B |
| PYGL | PYGL_HUMAN | Glycogen phosphorylase, liver form |
| QARS | SYQ_HUMAN | Glutamine--tRNA ligase |
| QPCT | QPCT_HUMAN | Glutaminyl-peptide cyclotransferase |
| QSOX1 | QSOX1_HUMAN | Sulfhydryl oxidase 1 |
| QTRT1 | TGT_HUMAN | Queuine tRNA-ribosyltransferase catalytic subunit |
| RAB3IP | RAB31_HUMAN | Rab-3A-interacting protein |
| RABIF | MSS4_HUMAN | Guanine nucleotide exchange factor MSS4 |
| RAC1 | RAC1_HUMAN | Ras-related C3 botulinum toxin substrate 1 |
| RACGAP1 | RGAP1_HUMAN | Rae GTPase-activating protein 1 |
| RACKI | RACK1_HUMAN | Receptor of activated protein C kinase 1, N-terminally processed |
| RAD1 | RAD1_HUMAN | Cell cycle checkpoint protein RAD1 |
| RAD18 | RAD18_HUMAN | E3 ubiquitin-protein ligase RAD18 |
| RAD51 | RAD51_HUMAN | DNA repair protein RAD51 homolog 1 |
| RAD52 | RAD52_HUMAN | DNA repair protein RAD52 homolog |
| RAE1 | RAE1L_HUMAN | mRNA export factor |
| RAET1L | ULBP6_HUMAN | UL16-binding protein 6 |
| RAF1 | RAF1_HUMAN | RAF proto-oncogene serine/threonine-protein kinase |
| RALGDS | GNDS_HUMAN | Ral guanine nucleotide dissociation stimulator |
| RAN | RAN_HUMAN | GTP-binding nuclear protein Ran |
| RANBP1 | RANG_HUMAN | Ran-specific GTPase-activating protein |
| RANBP2 | RBP2_HUMAN | E3 SUMO-protein ligase RanBP2 |
| RANBP3 | RANB3_HUMAN | Ran-binding protein 3 |
| RANBP9 | RANB9_HUMAN | Ran-binding protein 9 |
| RAP1GAP | RPGP1_HUMAN | Rap1 GTPase-activating protein 1 |
| RAPGEF5 | RPGF5_HUMAN | Rap guanine nucleotide exchange factor 5 |
| RAPGEFL1 | RPGFL_HUMAN | Rap guanine nucleotide exchange factor-like 1 |
| RAPH1 | RAPH1_HUMAN | Ras-associated and pleckstrin homology domains-containing protein 1 |
| RAPSN | RAPSN_HUMAN | 43 kDa receptor-associated protein of the synapse |
| RARA | RARA_HUMAN | Retinoic acid receptor alpha |
| RARB | RARB_HUMAN | Retinoic acid receptor beta |
| RARG | RARG_HUMAN | Retinoic acid receptor gamma |
| RARS | SYRC_HUMAN | Arginine--tRNA ligase, cytoplasmic |
| RASA1 | RASA1_HUMAN | Ras GTPase-activating protein 1 |
| RASGRP1 | GRP1_HUMAN | RAS guanyl-releasing protein 1 |
| RASGRP2 | GRP2_HUMAN | RAS guanyl-releasing protein 2 |
| RASGRP3 | GRP3_HUMAN | Ras guanyl-releasing protein 3 |
| RASGRP4 | GRP4_HUMAN | RAS guany1-releasing protein 4 |
| RASSF1 | RASF1_HUMAN | Ras association domain-containing protein 1 |
| RASSF5 | RASF5_HUMAN | Ras association domain-containing protein 5 |
| RAVER1 | RAVR1_HUMAN | Ribonucleoprotein PTB-binding 1 |
| RBAK | RBAK_HUMAN | RB-associated KRAB zinc finger protein |
| RBBP4 | RBBP4_HUMAN | Histone-binding protein RBBP4 |
| RBBP6 | RBBP6_HUMAN | E3 ubiquitin-protein ligase RBBP6 |
| RBBP8 | CT1P_HUMAN | DNA endonuclease RBBP8 |
| RBKS | RBSK_HUMAN | Ribokinase |
| RBM10 | RBMl10_HUMAN | RNA-binding protein 10 |
| RBM11 | RBM11_HUMAN | Splicing regulator RBM11 |
| RBM22 | RBM22_HUMAN | Pre-mRNA-splicing factor RBM22 |
| RBM23 | RBM23_HUMAN | Probable RNA-binding protein 23 |
| RBM38 | RBM38_HUMAN | RNA-binding protein 38 |
| RBM39 | RBM39_HUMAN | RNA-binding protein 39 |
| RBM4 | RBM4_HUMAN | RNA-binding protein 4 |
| RBM4B | RBM4B_HUMAN | RNA-binding protein 4B |
| RBM5 | RBM5_HUMAN | RNA-binding protein 5 |
| RBM7 | RBM7_HUMAN | RNA-binding protein 7 |
| RBM8A | RBM8A_HUMAN | RNA-binding protein 8A |
| RBMX2 | RBMX2_HUMAN | RNA-binding motif protein, X-linked 2 |
| RBP4 | RET4_HUMAN | Plasma retinol-binding protein(1-176) |
| RBP5 | RET5_HUMAN | Retinol-binding protein 5 |
| RBPJ | SUH_HUMAN | Recombining binding protein suppressor of hairless |
| RBSN | RBNS5_HUMAN | Rabenosyn-5 |
| RCC1 | RCC1_HUMAN | Regulator of chromosome condensation |
| RCC1L | RCC1L_HUMAN | RCC1-like G exchanging factor-like protein |
| RCC2 | RCC2_HUMAN | Protein RCC2 |
| RCHY1 | ZN363_HUMAN | RING finger and CHY zinc finger domain-containing protein 1 |
| RECQL4 | RECQ4_HUMAN | ATP-dependent DNA helicase Q4 |
| REN | REN1_HUMAN | Renin |
| REP1N1 | REP11_HUMAN | Replication initiator 1 |
| REST | REST_HUMAN | RE1-silencing transcription factor |
| RET | RET_HUMAN | Extracellular cell-membrane anchored RET cadherin 120 kDa |
| | | fragment |
| RFFL | RFFL_HUMAN | E3 ubiquitin-protein ligase rififylin |
| RFK | RIFK_HUMAN | Riboflavin kinase |
| RFPL4A | RFPLA_HUMAN | Ret finger protein-like 4A |
| RFWD3 | RFWD3_HUMAN | E3 ubiquitin-protein ligase RFWD3 |
| RFXANK | RFXK_HUMAN | DNA-binding protein RFXANK |
| RGCC | RFXK_HUMAN | Regulator of cell cycle RGCC |
| RGMB | RGMB_HUMAN | RGM domain family member B |
| RGN | RGN_HUMAN | Regucalcin |
| RHEB | RHEB_HUMAN | GTP-binding protein Rheb |
| RHO | OPSD_HUMAN | Rhodopsin |
| R1DA | RIDA_HUMAN | 2-iminobutanoate/2-iminopropanoate deaminase |
| RIMBP2 | RIMB2_HUMAN | RIMS-binding protein 2 |
| RIMBP3 | RIM3A_HUMAN | RIMS-binding protein 3A |
| RIMS1 | RlMS1_HUMAN | Regulating synaptic membrane exocytosis protein 1 |
| RIMS2 | RlMS2_HUMAN | Regulating synaptic membrane exocytosis protein 2 |
| RIOK1 | RIOK1_HUMAN | Serine/threonine-protein kinase RIO1 |
| RIOK2 | RIOK2_HUMAN | Serine/threonine-protein kinase RlO2 |
| RIPK1 | RIPK1_HUMAN | Receptor-interacting serine/threonine-protein kinase 1 |
| RIPK2 | RIPK2_HUMAN | Receptor-interacting serine/threonine-protein kinase 2 |
| RLBP1 | RLBP1_HUMAN | Retinaldehyde-binding protein 1 |
| RM12 | RM12_HUMAN | RecQ-mediated genome instability protein 2 |
| RNASE4 | RNAS4_HUMAN | Ribonuclease 4 |
| RNASEH2B | RNH2B_HUMAN | Ribonuclease H2 subunit B |
| RNASEH2C | RNH2C_HUMAN | Ribonuclease H2 subunit C |
| RNASEL | RN5A_HUMAN | 2-5A-dependent ribonuclease |
| RNF121 | RN121_HUMAN | RING finger protein 121 |
| RNF123 | RN123_HUMAN | E3 ubiquitin-protein ligase RNF123 |
| RNF125 | RN125_HUMAN | E3 ubiquitin-protein ligase RNF125 |
| RNF14 | RNF14_HUMAN | E3 ubiquitin-protein ligase RNF14 |
| RNF166 | RN166_HUMAN | RING finger protein 166 |
| RNF17 | RNF17_HUMAN | RING finger protein 17 |
| RNF170 | RN170_HUMAN | E3 ubiquitin-protein ligase RNFl 70 |
| RNF175 | RN175_HUMAN | RING finger protein 175 |
| RNF19A | RN19A_HUMAN | E3 ubiquitin-protein ligase RNF19A |
| RNF19B | RN19B_HUMAN | E3 ubiquitin-protein ligase RNF19B |
| RNF2 | RlNG2_HUMAN | E3 ubiquitin-protein ligase RING2 |
| RNF207 | RN207_HUMAN | RING finger protein 207 |
| RNF208 | RN208_HUMAN | RING finger protein 208 |
| RNF212B | R212B_HUMAN | RING finger protein 212B |
| RNF216 | RN216_HUMAN | E3 ubiquitin-protein ligase RNF216 |
| RNF31 | RNF31_HUMAN | E3 ubiquitin-protein ligase RNF3 1 |
| RNF34 | RNF34_HUMAN | E3 ubiquitin-protein ligase RNF34 |
| RNF39 | RNF39_HUMAN | RING finger protein 39 |
| RNF4 | RNF4_HUMAN | E3 ubiquitin-protein ligase RNF4 |
| RNF8 | RNF8_HUMAN | E3 ubiquitin-protein ligase RNF8 |
| RNGTT | MCEl_HUMAN | mRN A guany ly ltransferase |
| ROBOl | ROBOl_HUMAN | Roundabout homolog 1 |
| ROBO2 | ROBO2_HUMAN | Roundabout homolog 2 |
| ROCKl | ROCK1_HUMAN | Rho-associated protein kinase 1 |
| ROCK2 | ROCK2_HUMAN | Rho-associated protein kinase 2 |
| ROR2 | ROR2_HUMAN | Tyrosine-protein kinase transmembrane receptor |
| | | ROR2 |
| RORA | RORA_HUMAN | Nuclear receptor ROR-alpha |
| RORB | RORB_HUMAN | Nuclear receptor ROR-beta |
| RORC | RORG_HUMAN | Nuclear receptor ROR-gamma |
| RPAl | RFAl_HUMAN | Replication protein A 70 kDa DNA-binding |
| | | subunit, N-terminally processed |
| RPA3 | RFA3_HUMAN | Replication protein A 14 kDa subunit |
| RPGR | RPGR_HUMAN | X-linked retinitis pigmentosa GTPase regulator |
| RPH3A | RP3A_HUMAN | Rabphilin-3A |
| RPH3AL | RPH3L_HUMAN | Rab effector Noc2 |
| RPLll | RLll_HUMAN | 60S ribosomal protein L1 1 |
| RPL37 | RL37_HUMAN | 60S ribosomal protein L37 |
| RPL37A | RL37A_HUMAN | 60S ribosomal protein L37a |
| RPL37AP8 | RL37L_HUMAN | Putative 60S ribosomal protein L37a-like protein |
| RPS12 | RS12_HUMAN | 40S ribosomal protein S 12 |
| RPS15A | RS15A_HUMAN | 40S ribosomal protein Sl5a |
| RPS18 | RS18_HUMAN | 40S ribosomal protein Sl8 |
| RPS19 | RS19_HUMAN | 40S ribosomal protein Sl9 |
| RPS21 | RS21_HUMAN | 40S ribosomal protein S21 |
| RPS23 | RS23_HUMAN | 40S ribosomal protein S23 |
| RPS24 | RS24_HUMAN | 40S ribosomal protein S24 |
| RPS27A | RS27A_HUMAN | 40S ribosomal protein S27a |
| RPS3A | RS3A_HUMAN | 40S ribosomal protein S3a |
| RPS4X | RS4X_HUMAN | 40S ribosomal protein S4, X isoform |
| RPS4YI | RS4YI_HUMAN | 40S ribosomal protein S4, Y isoform I |
| RPS6 | RS6_HUMAN | 40S ribosomal protein S6 |
| RPS6KAI | KS6AI_HUMAN | Ribosomal protein S6 kinase alpha-I |
| RPS6KA3 | KS6A3_HUMAN | Ribosomal protein S6 kinase alpha-3 |
| RPS6KA5 | KS6A5_HUMAN | Ribosomal protein S6 kinase alpha-5 |
| RPS6KBI | KS6BI_HUMAN | Ribosomal protein S6 kinase beta-I |
| RPS7 | RS7_HUMAN | 40S ribosomal protein S7 |
| RPS8 | RS8_HUMAN | 40S ribosomal protein S8 |
| RPSA | RSSA_HUMAN | 40S ribosomal protein SA |
| RPTOR | RPTOR_HUMAN | Regulatory-associated protein ofmTOR |
| RREBI | RREBI_HUMAN | Ras-responsive element-binding protein I |
| RRMI | RlRI_HUMAN | Ribonucleoside-diphosphate reductase large |
| | | subunit |

The molecular surface is a higher-level representation of protein structure than protein structure or sequence. It models a protein as a continuous shape with geometric and chemical features. See Richards et al., “Ann. Rev. Biophysics Bioeng. 6:151-76 (2003).

The molecular surface is useful for the methods described herein, for example, for identifying proteins with similar and/or complementary surface features, predicting molecular interactions between an E3 ligase and a target protein and/or binding modulator. Thus, in some cases, the methods described herein comprise providing molecular surface feature(s) of one or more protein(s). Molecular surface features that are useful for the methods described herein include, for example, geometric features and/or chemical features.

In some cases, the molecular surface features are extracted from a crystal structure. In some cases, the crystal structure is a ligand bound (i.e. holo). In some cases, the crystal structure is unbound (i.e. apo). In some cases, the molecular surface features are extracted from a computer modeled structure. In some cases, the computer modeled structure is ligand bound. In some cases, the computer modeled structure is unbound.

In some cases, the molecular surface features are obtained from a database. For example, the Protein Data Bank (PDB, rcsb.org) or the AlphaFold Protein Structure Database (alphafold.ebi.ac.uk).

PDB is a database for the three-dimensional structural data of large biological molecules, such as proteins and nucleic acids (Nucleic Acids Res. 2019 Jan. 8; 47(D1):D520-D528. doi: 10.1093/nar/gky949). The data is submitted by biologists and biochemists from around the world, are freely accessible on the Internet via the websites of its member organizations (e.g. PDBe—pdbe.org, PDBj—pdbj.org, RCSB—rcsb.org/pdb, and BMRB—bmrb.wisc.edu). The PDB is overseen by an organization called the Worldwide Protein Data Bank—wwPDB—.

In some embodiments, providing molecular surface feature(s) comprises determining a three-dimensional structure experimentally, e.g., using X-ray crystallyography, nuclear magnetic resonance (NMR spectroscopy), cry-electron microscropy (cryoEM), small-angle X-ray scattering (SAXS), small-angle neutron scattering (SANS), or combinations thereof.

In some embodiments, providing molecular surface feature(s) comprises modeling of the three-dimensional structural context, e.g., if the three-dimensional structure of the identified protein is not known.

In some cases, modeling of the three-dimensional structural context is carried out using computer modeling. In some cases, the computer modeling is carried out using an artificial intelligence program, e.g., according to the methods described in Jumper et al., “Highly Accurate Protein Structure Prediction with AlphaFold,” Nature 596:583-89 (2021) or Evans et al., “Protein Complex Prediction with AlphaFold-Multimer,” bioRxiv doi.org/10.1101/2021.10.04.463034 (2021).

The molecular surface feature(s) can be provided together or separately. In some cases, the structure of one or more of the proteins is a ligand bound (i.e. holo) structure. In some cases, the structure of one or more of the proteins is unbound (i.e. apo).

In some cases, the molecular surface features(s) are based on the three-dimensional structure of a region of a protein, e.g., the interface region of the protein that participates in (or is hypothesized to participate in) a PPI.

In some cases, for example, where the three-dimensional structures are unbound, starting structure(s) are built by superimposing the three-dimensional structures onto a reference structure.

In some cases, the molecular surface feature (s) are provided as parameters in digital format, e.g., in a MasIF data file, for use in the methods described herein. Thus, in some cases, the methods described herein comprise providing data defining the molecular surface feature(s) of two or more proteins (or fragments thereof).

In some cases, the molecular surface feature(s) are geometric feature(s) and/or chemical feature(s).

### Geometric Features

In some cases, the surface feature(s) are geometric feature(s). In some cases, the geometric feature(s) are selected from the group consisting of a shape index (Koenderink et al., “Surface Shape and Curvature Scales,” Image Vis. Comput. 10:557-64 (1992), which is hereby incorporated by reference in its entirety), distance-dependent curvature (Yin et al., “Fast Screening of Protein Surfaces using Geometric Invariant Fingerprints” Proc. Natl. Acad. Sci. USA 106:16622-26 (2009), which is hereby incorporated by reference in its entirety), geodesic polar coordinate(s), radial (angular) coordinate(s), and combinations thereof. In other cases, the geometric features are learned directly from the underlying tertiary structure of the protein and its atomic arrangements.

### Chemical Features

In some cases, the surface feature(s) are chemical feature(s). In some cases, the chemical feature(s) are selected from the group consisting of hydropathy index (Kyte et al., “A Simple Method for Displaying the Hydropathic Character of a Protein” J. Mol. Biol. 157:105-32 (1982)), continuum electrostatics (Jurrus et al. “Improvements to the APBS Biomolecular Solvation Software Suite,” Protein Sci. 27:112-28 (2018), which is hereby incorporated by reference in its entirety), location of free electrons (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), location of free proton donors (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), and combinations thereof. In other cases, the chemical feature are learned directly from the underlying tertiary structure of the protein and its atomic arrangements.

### Identification and Characterization of Degrons, Substrates, and Neosubstrates

Provided herein are compositions and methods for identification, classification, and/or selection of substrates and/or neosubstrates of E3 ligase(s), e.g., E3 ligase(s) described herein.

In some cases, the methods described herein comprise providing a set of molecular surface features, e.g., as described herein, of one or more protein(s). In some cases, the set of molecular surface features describes a protein surface. In some cases, the set of molecular surface features describes a space complementary to a protein surface.

In some cases, the methods described herein comprise providing a set of molecular surface features (e.g., molecular surface features described herein) of E3 ligase substrate receptor protein(s). In some cases, the molecular surface features of the E3 ligase substrate receptor protein is in an unbound state (e.g., an E3 ligase “surface”). In some cases, the molecular surface features of the E3 ligase substrate receptor protein is in a bound state (e.g., an E3 ligase “neosurface”).

In some cases, the methods described herein comprise providing a first set of molecular surface features, e.g., molecular surface features described herein, derived from a set of proteins having degron(s) of an E3 ligase (e.g., an E3 ligase substrate receptor protein) and/or predicted to have degron(s) of the E3 ligase (e.g., the E3 ligase substrate receptor protein), e.g., degron(s) described herein.

In some cases, the E3 ligase substrate receptor protein is Cereblon (CRBN; e.g., human CRBN), or a variant, derivative, ortholog, or homolog thereof, e.g., an enzymatically active variant, derivative, ortholog, or homolog thereof, e.g., as described herein, and the degron is a G-loop degron, e.g., as described herein.

In some cases, the E3 ligase substrate receptor protein is BTRC (e.g., human BTRC, e.g., SEQ ID NO: 40), or a variant, derivative, ortholog, or homolog thereof, e.g., an enzymatically active variant, derivative, ortholog, or homolog thereof, and the degron comprises or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid.

In some cases, the E3 ligase substrate receptor protein is KEAP1 (e.g., human KEAP1, e.g., SEQ ID NO: 18), or a variant, derivative, ortholog, or homolog thereof, e.g., an enzymatically active variant, derivative, ortholog, or homolog thereof, and the degron comprises or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid.

In some cases, the E3 ligase substrate receptor protein is KEAP1 (e.g., human KEAP1, e.g., SEQ ID NO: 18), or a variant, derivative, ortholog, or homolog thereof, e.g., an enzymatically active variant, derivative, ortholog, or homolog thereof, and the degron comprises or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine.

In some cases, the E3 ligase substrate receptor protein is KEAP1 (e.g., human KEAP1, e.g., SEQ ID NO: 18), or a variant, derivative, ortholog, or homolog thereof, e.g., an enzymatically active variant, derivative, ortholog, or homolog thereof, and the degron comprises or consists of the amino acid motif ETGE ((SEQ ID NO: 1) and/or DLG.

In some cases, the E3 ligase substrate receptor protein is MDM2 (e.g., human MDM2, e.g., SEQ ID NO: 26), or a variant, derivative, ortholog, or homolog thereof, e.g., an enzymatically active variant, derivative, ortholog, or homolog thereof, and the degron comprises or consists of the amino acid motif X¹-X²-X³-X⁴-X⁵-X⁶-X⁷-X⁸, wherein X¹ is phenylalanine; X² is any one of the naturally occurring amino acids; X3 is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine.

In some cases, the E3 ligase substrate receptor protein is MDM2 (e.g., human MDM2, e.g., SEQ ID NO: 26), or a variant, derivative, ortholog, or homolog thereof, e.g., an enzymatically active variant, derivative, ortholog, or homolog thereof, and the degron comprises or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix.

In some cases, the E3 ligase substrate receptor protein is VHL (e.g., human VHL, e.g., SEQ ID NO: 9), or a variant, derivative, ortholog, or homolog thereof, e.g., an enzymatically active variant, derivative, ortholog, or homolog thereof, and the degron comprises or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

In some cases, the methods described herein include providing a second set of molecular surface features derived from a second set of one or more proteins. In some cases, the one or more proteins comprise or consist of human proteins. In some cases, the one or more proteins are selected from the proteins in Table 3. In some cases, the first and second sets of proteins are mutually exclusive. In some cases, the first and second sets of proteins overlap by one or more proteins.

In some cases, the methods described herein include calculating a similarity and/or complementary score for protein(s) of the second set. In some cases, calculating the similarity score includes comparing first and second sets of molecular surface features, e.g., the molecular surface features described herein.

In some cases, providing a first set of molecular surface features, providing a second set of molecular surface features, calculating a similarity score, and/or calculating a complementarity score is carried out using a pipeline that exploits geometric deep learning to process the molecular surface data which lies in a non-euclidean domain.

In some cases, the methods described herein comprise identifying predicted neosubstrate(s) of E3 ligase(s) based on a similarity and/or complementarity score, e.g., as described herein, using a geometric deep learning model trained on a set of protein-protein interactions to produce embeddings that are similar for surface patches that are similar or (e.g., an interaction fingerprint).

In some cases, the methods described herein comprise identifying predicted neosubstrate(s) of E3 ligase(s) based on a similarity and/or complementarity score, e.g., as described herein, using interaction fingerprints produced by a geometric deep learning model trained on a set of degron and/or putative degron molecular surface feature(s)).

In some cases, the methods described herein comprise identifying predicted degron(s) of neosubstrate(s) of E3 ligase(s) based on similarity to a set of degrons that comprises predicted degrons identified based on interaction fingerprints produced by a geometric deep learning model trained on a set of molecular surface features complementary to the E3 ligase (e.g., an interaction fingerprint).

In some cases, the methods described herein comprise testing or having tested protein(s), e.g., predicted neosubstrate(s) in an E3 ligase substrate detection assay. In some cases, the assay is carried out in the absence of a binding modulator of the E3 ligase. In some cases, the assay is carried out in the presence of a binding modulator of the E3 ligase.

E3 ligase substrate detection assays are described, for example, in Liu et al., “Assays and Technologies for Developing Proteolysis Targeting Chimera Degraders,” Future Medicinal Chemistry 12(12):1155-79 (2020).

E3 ligase substrate detection assays include, for example, binding/ternary binding affinities and ternary complex formation assays used to profile, for example, ternary complex formation, population, stability, binding affinities, cooperative or kinetics such as fluorescence polarization (FP) assay, an amplified luminescent proximity homogenous assay (ALPHA), time-resolved fluorescence energy transfer assay (TR-FRET), isothermal titration calorimetry (ITC), surface plasma resonance (SPR), bio-layer interferometry (BLI), nano-bioluminescence resonance energy transfer (nano-BRET), size exclusive chromatography (SEC), crystallography, co-immunoprecipitation (Co-IP), mass spectrometry (MS), and protein-fragment complementation (e.g., NanoBiT®). See, e.g., Liu et al., 2020.

E3 ligase substrate detection assays include, for example, protein ubiquitination assays. See, e.g., Liu et al., 2020.

E3 ligase substrate detection assays include, for example, target degradation assays such as immunoassays, reporter assays, mass spectrometry (MS), protein degradation-based phenotypic screening such as amplified luminescent proximity homogenous assay (ALPHA), bio-layer interferometry (BLI), cellular thermal shift assay (CETSA), co-immunoprecipitation (Co-IP), cryogenic electron microscopy (Cryo-EM), differential scanning fluorimetry (DSF), fluorescence polarization (FP), isothermal titration calorimetry (ITC), microscale thermophoresis (MST), NanoLuc binary technology (Nano-BiT), nano-bioluminescence resonance energy transfer (BRET), surface plasma resonance (SPR), time-resolved fluorescence energy transfer (TR-FRET), tandem ubiquitin-binding entities-amplified luminescent proximity homogenous and enzyme-linked immunosorbent assay (TUBE-ALPHALISA), and tandem ubiquitin-binding entities-dissociation-enhanced lanthanide fluorescent immunoassay (TUBE-DELFIA). See, e.g., Liu et al., 2020.

In some cases, the E3 ligase substrate detection assay is a proximity assay. In some cases, the E3 ligase substrate detection assay is a binding assay. In some cases, the E3 ligase substrate detection assay is a degradation assay.

In some cases, the proximity assay is a homogeneous time resolved fluorescence (HTRF) assay. In some cases, the proximity assay is a quantitative proteomics assay. In some cases, the proximity assay is a biotinylation assay, e.g., a promiscuous biotinylation assay.

In some cases, the degradation assay is a High efficiency Binary Technology (HiBiT) assay.

In some cases, the degradation assay is a quantitative proteomics assay.

In some cases, the E3 ligase substrate detection assay is a yeast-2-hybrid system. See, e.g., Kohalmi et al., “Identification and Characterization of Protein Interactions Using the Yeast-2-Hybrid System,” In: Gelvin S. B., Schilperoort R. A. (eds) Plant Molecular Biology Manual. Springer, Dordrecht (1998). In some cases, the E3 ligase substrate detection assay is a yeast-3-hybrid system. See, e.g., Glass et al., “The Yeast Three-Hybrid System for Protein Interactions,” Methods Mol. Biol 1794:195-205 (2018).

In some cases, the E3 ligase substrate detection assay is a genomic construct based method, e.g., as described in Sievers et al., “Defining the Human C2H2 Zinc Finger Degrome Targeted by Thalidomide Analogs through CRBN,” Science 362(6414):eaat0572 (2018).

In some cases, the E3 ligase substrate detection assay is an indirect screen, e.g., to detect changes in gene and/or protein expression.

### Sequences, Mutants, and Variants

The polypeptide and nucleic acid sequences described herein are described using their IUPAC ambiguity codes (Table 4), unless otherwise noted.

TABLE 4
IUPAC ambiguity codes

| Nucleotide Code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | Gap |

In some cases, the polypeptide or nucleic acid sequences described herein have at least 80%, e.g., at least 85%, 90%, 95%, 98%, or 100% identity to a polypeptide or nucleic acid sequence provided herein, e.g., has differences at up to 1%, 2%, 5%, 10%, 15%, or 20% of the residues of the sequence provided herein replaced, e.g., with conservative mutations, e.g., including or in addition to the mutations described herein.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### EXAMPLES

The following examples are included for illustrative purposes only and are not intended to limit the scope of the invention.

### Example 1: MaSIF—A Computational Framework to Study Protein Surface Properties

A high-level representation of protein structure, the molecular surface, displays patterns of chemical and geometric features that fingerprint a protein's modes of interactions with other biomolecules. Proteins performing similar interactions may share common fingerprints, independent of their evolutionary history. Fingerprints may be difficult to grasp by visual analysis but could be learned from large-scale datasets. MaSIF (Molecular Surface Interaction Fingerprinting) (P. Gainza et al., Deciphering interaction fingerprints from protein molecular surfaces using geometric deep learning. Nat Methods 17, 184-192 (2020)) is a conceptual framework based on a geometric deep learning (GDL) method (M. M. Bronstein, J. Bruna, Y. LeCun, A. Szlam, P. Vandergheynst, Geometric Deep Learning: Going beyond Euclidean data. IEEE Signal Processing Magazine 34, 18-42 (2017)) to capture fingerprints that drive specific biomolecular interactions.

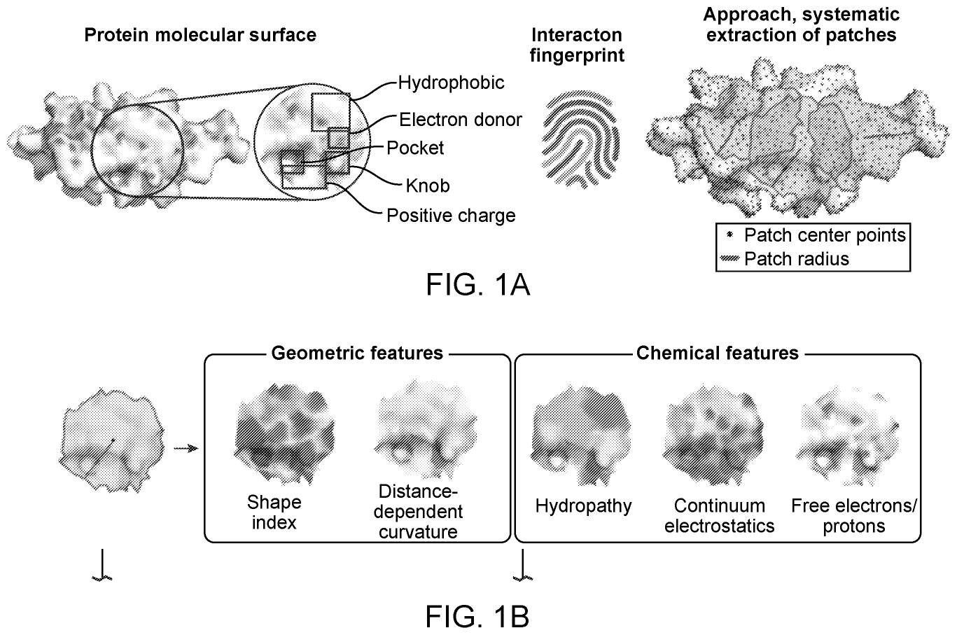

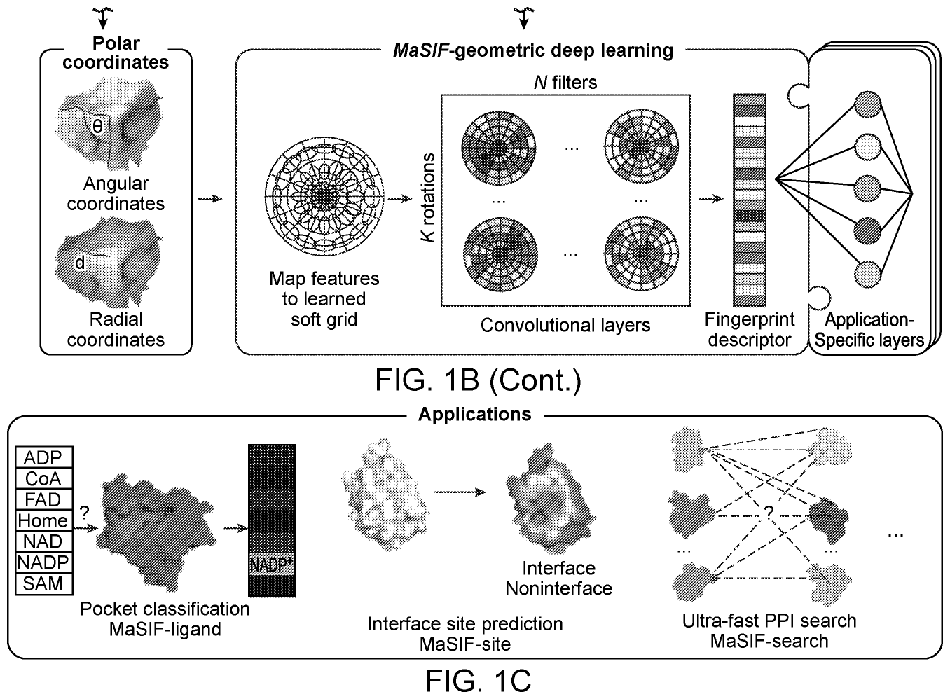

MaSIF exploits GDL to learn interaction fingerprints in protein molecular surfaces. First, MaSIF decomposes a surface into overlapping radial patches with a fixed geodesic radius (FIG. 1A). Each point within a patch is assigned an array of geometric and chemical input features (FIG. 1B top). MaSIF then learns to embed the surface patch's input features into a numerical vector descriptor (FIG. 1B, bottom). Each descriptor is further processed with application-dependent neural network layers. MaSIF was showcased with three proof-of-concept applications (FIG. 1C): a) ligand pocket similarity comparison (MaSIF-ligand) where MaSIF performed on par with other algorithms; b) protein-protein interaction (PPI) site prediction in protein surfaces (MaSIF-site), where MaSIF was clearly the top performer; c) ultrafast scanning of surfaces, exploiting surface fingerprints to predict the structural configuration of protein-protein complexes (MaSIF-search) where MaSIF shows an acceleration of several orders of magnitude in computational runtimes compared to other methods.

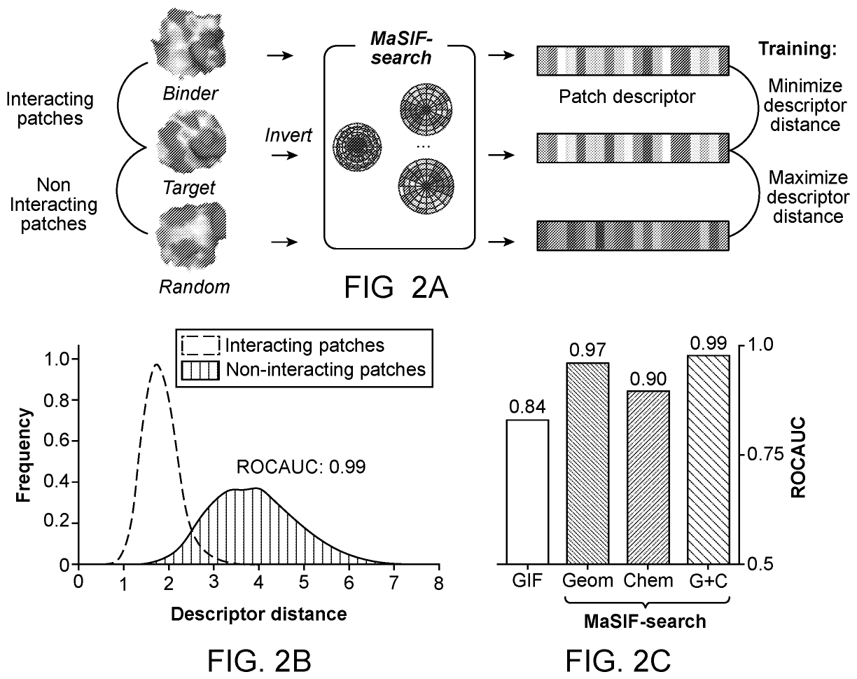

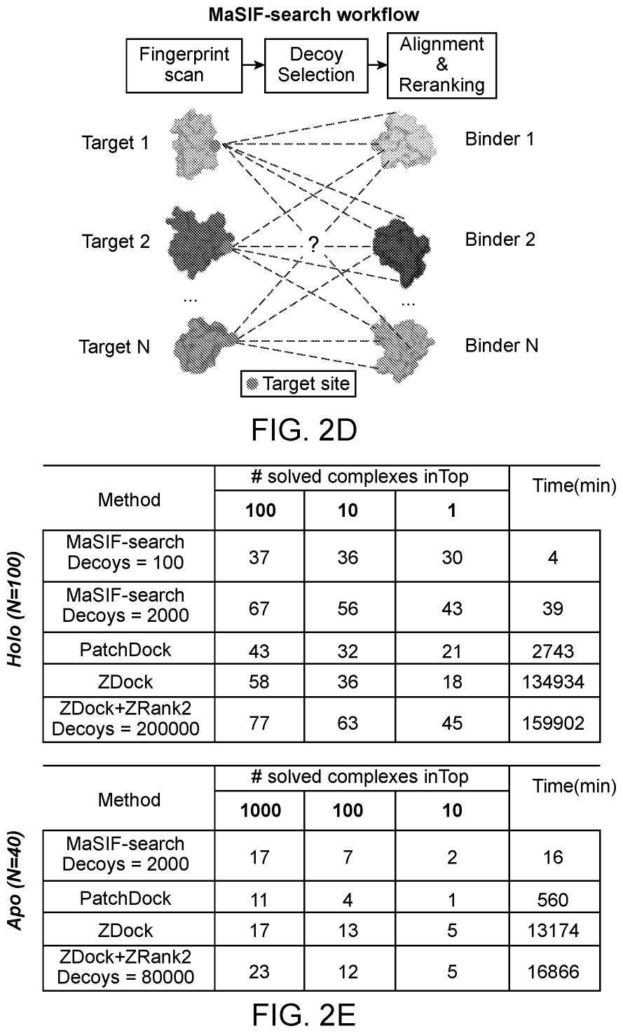

Within the MaSIF framework, MaSIF-search was developed (FIG. 2A) which learns patterns in interacting pairs of surface patches. PPIs occur through surface patches with some degree of complementary geometric and chemical features. To formalize this observation, MaSIF-search inverts the numerical features of one protein partner (multiplied by −1), with the exception of hydropathy. Although the models of complementarity are not perfect, the network may be able to learn different levels of complementarity. After performing the inversion on one patch, the Euclidean distance between the fingerprint descriptors of two complementary surface patches should be close to 0. Within this framework, MaSIF-search will produce similar descriptors for pairs of interacting patches (low Euclidean distances between fingerprint descriptors), and dissimilar descriptors for non-interacting patches (larger Euclidean distances between fingerprint descriptors) (FIG. 2A). Thus, identifying potential binding partners is reduced to a comparison of numerical vectors.

To test this concept, a database with >100K pairs of interacting protein surface patches with high shape complementarity, as well as a set of randomly chosen surface patches, to be used as non-interacting patches, was developed. A trio of protein surface patches with the labels, binder, target, and random patches were fed into the MaSIF-search network (FIG. 2A). The neural network was trained to simultaneously minimize the Euclidean distance between the fingerprint descriptors of binders vs targets, while maximizing the Euclidean distance between targets vs random, commonly referred to as a Siamese architecture in the machine learning literature.

Performance on the test set shows that the descriptor Euclidean distances for interacting surface patches is much lower than that of non-interacting patches, resulting in a ROC AUC of 0.99 (FIG. 2B; FIG. 2C).

Next, MaSIF-search was used to predict the structure of known protein-protein complexes. Ideally, one would be able to predict whether two proteins interact simply by comparing their respective fingerprints, avoiding a time-consuming, systematic exploration of the 3D docking space. It was found that fingerprint descriptors can provide an initial and fast evaluation of candidate binding partners. However, a better performance can be achieved by including a subsequent stage where candidate patches (referred to as decoys) selected by the Euclidean fingerprint distance of the patches center points to the target patch are rescored using fingerprints of neighboring points within the patch. Specifically, the MaSIF-search workflow entails two stages (FIG. 2D): I) scanning a large database of descriptors of potential binders and selecting the top decoys by descriptor similarity; and II) three-dimensional alignment of the complexes exploiting fingerprint descriptors of multiple points within the patch, coupled to a reranking of the predictions with a separate neural network.

To benchmark MaSIF-search a scenario was simulated where the binding site of a target protein is known, and one attempts to recapitulate the true binder of a protein among many other binders. Specifically, MaSIF-search was benchmarked in 100 bound protein complexes randomly selected from the testing set (disjoint from the training set). For each complex, the center of the interface in the target protein was selected, and then an attempt was made to recover the bound complex within the 100 binder proteins comprising the test set (FIG. 2D). A successful prediction means that a predicted complex with an interface Root Mean Square Deviation (iRMSD) of less than 5 Å relative to the known complex is found in a shortlist of the top 100, top 10, or top 1 results. For comparison, the same task was performed using: PatchDock (D. Duhovny, R. Nussinov, H. J. Wolfson. (Springer Berlin Heidelberg, Berlin, Heidelberg, 2002), pp. 185-200); Zdock (M. F. Lensink, S. Velankar, S. J. Wodak, Modeling protein-protein and protein-peptide complexes: CAPRI 6th edition. Proteins 85, 359-377 (2017); B. G. Pierce, Y. Hourai, Z. Weng, Accelerating protein docking in ZDOCK using an advanced 3D convolution library. PLoS One 6, e24657 (2011)); and ZDock in combination with the scoring application ZRank2 (B. Pierce, Z. Weng, A combination of rescoring and refinement significantly improves protein docking performance. Proteins 72, 270-279 (2008)) (ZDock+ZRank2). For each program runtime performance and number of recovered complexes were compared (FIG. 2E). Among the baseline tools, PatchDock showed the fastest performance, while ZDock+ZRank2 showed the best performance. MaSIF-search with only 100 decoys per target shows performances similar to PatchDock, but the entire benchmark is performed in just 4 CPU minutes, compared to 2743 CPU minutes for PatchDock. If MaSIF-search's decoys were expanded to 2000, it achieved similar performances to ZDock+ZRank2 with much faster runtimes (˜4000-fold).

Even though MaSIF was trained only on co-crystallized protein complexes, the method was also tested in a benchmark set of 40 proteins crystallized in the unbound (apo) state. Since unbound docking is significantly more challenging, the success criteria were changed to finding the correct complex within the top-1000, top-100, and top-10, for all methods (FIG. 2E). Here the performance of all tools deteriorates, with slightly better accuracy for ZDock and ZDock+ZRank2. Although MaSIF-search can recover many of the complexes within the top 1000 results, the scoring neural network, which was trained on holo structures, does not rank these into the top 10. These results pointed to the need of training MaSIF on apo structures, perhaps by augmenting datasets with simulated unbound states.

### Example 2: An Atlas of Degron Fingerprints Across the Structurally Characterized Proteome (fAIceit-Mimicry)

In order to utilize molecular surface features for the identification of degron fingerprints, a first-in-kind method was developed for identifying putative degrons based on the similarity of molecular surface features (patches).

Unlike previous approaches using molecular surface representations (see, e.g., Yin et al., “Fast Screening of Protein Surfaces Using Geometric Invariant Fingerprints,” PNAS 106(39):1662-26 (2009)), the machine learning approach does not rely on ‘handcrafted’ descriptors that are manually optimized vectors that describe protein surface features. Such approaches are limited in their usefulness and application, as it is difficult to determine a prior the right set of features for a given prediction task. See, e.g., Gainza et al., “Deciphering Interaction Fingerprints from Protein Molecular Surfaces Using Geometric Deep Learning,” Nature Methods 17:184-92 (2020).

Furthermore, one of the challenges of performing machine learning on CRBN degrons is how little data is available. There are only 9 publicly available structures of 6 known degrons (IKZF1, IKZF2, SALL4, CK1a, GSPT1, ZNF692), which represents a very important challenge in terms of learning using any deep learning tool. Where the number of data points for training is limited, the usefulness of a machine learning algorithm trained on those data points, in order to identify similar data points, will be limited.

Here, a database of all protein surface patches recognized by E3 ligases was constructed using a modification of the MaSIF framework. The method was originally trained to minimize the Euclidian distance between the fingerprint descriptors of a binder and target, and to maximize the distance between the descriptors of target and random (i.e., trained on complementarity rather than similarity), to identify complementary surfaces (i.e., predicted protein-protein interactions). To avoid and overcome the difficulties noted above in training an algorithm to search for degrons based on similarity, the MaSIF model was not re-trained.

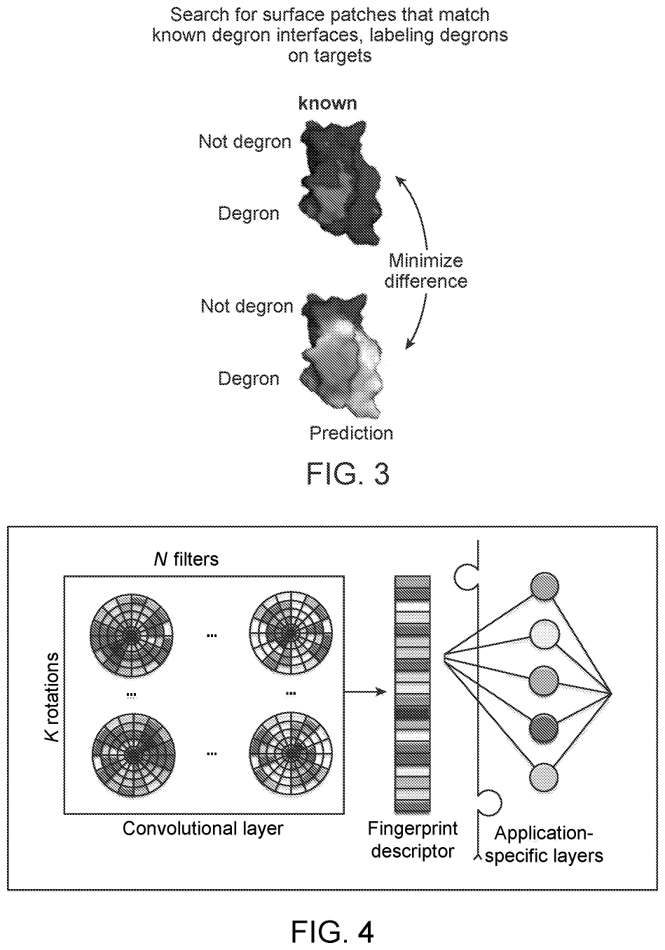

Rather, the algorithm was modified to perform matching of surface patches recognized by E3 ligases (that is, MaSIF was modified to search for similarity rather than complementarity), as depicted in FIG. 3 and FIG. 4.

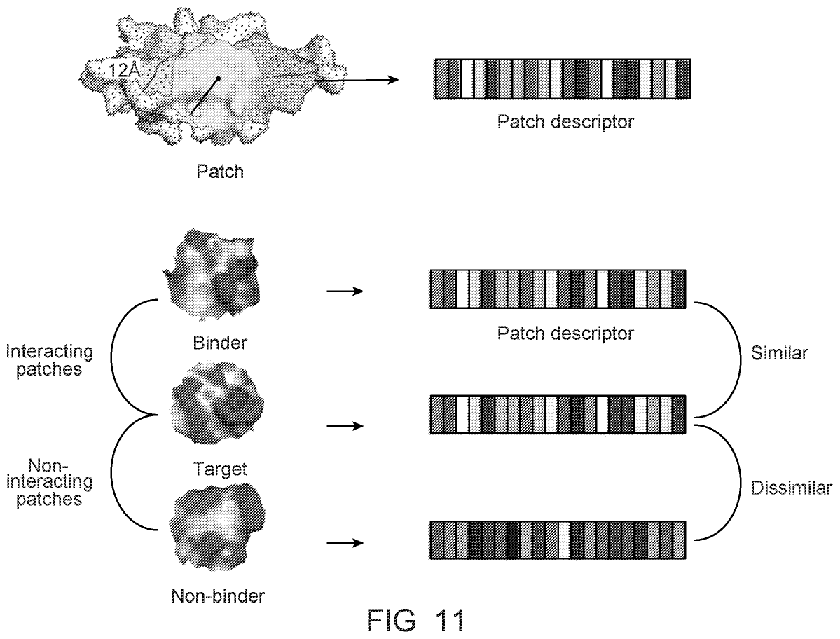

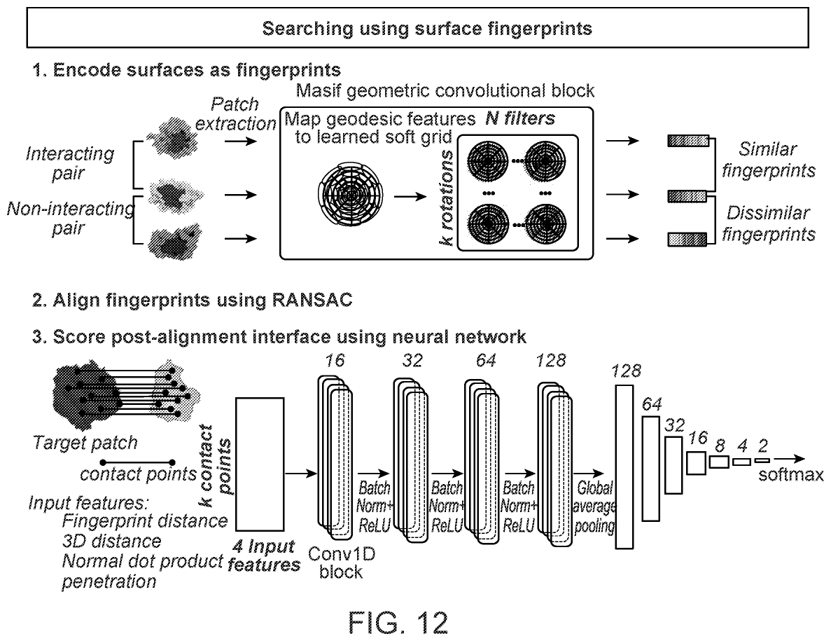

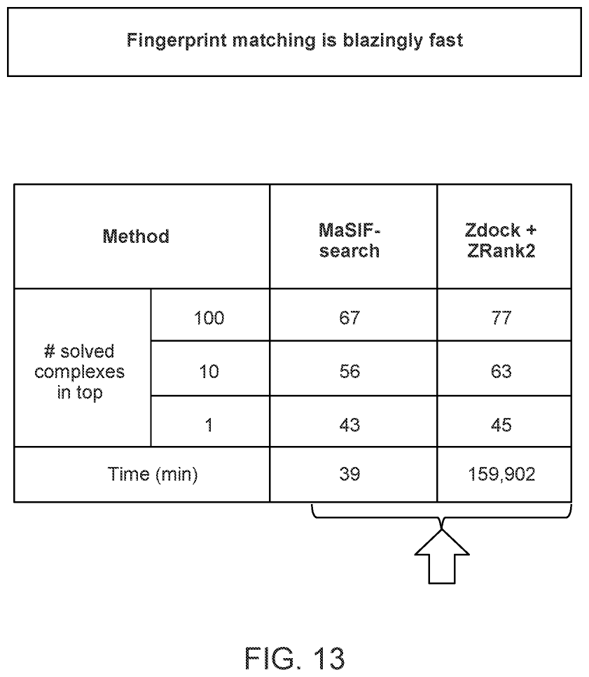

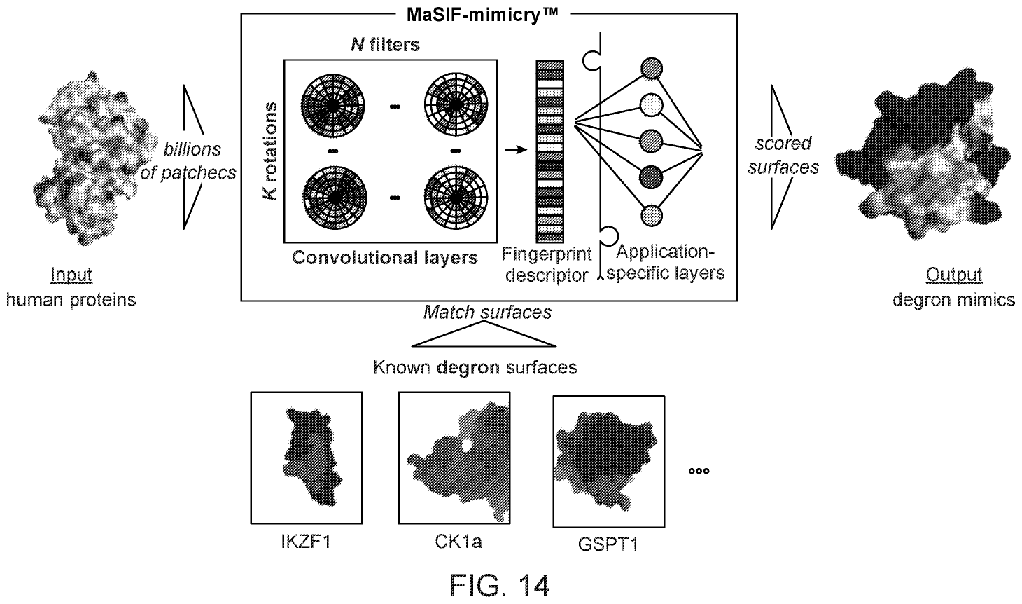

During the matching stage the different patches were clustered in an unsupervised fashion, providing cluster/families of proteins that display similar surface fingerprints and that can potentially engage (the same) E3 ligases, as shown in FIG. 11, FIG. 12, FIG. 13, and FIG. 14.

The structurally characterized proteome was searched for similar surface patches. A target list of potential E3 substrates was assembled based on the presence of similar surface patch(es).

As a final embodiment of the fingerprint matching, structural complexes between E3 ligases and predicted substrates were docked in three-dimensional space. These docked complexes were used for the search of chemical compounds to facilitate the formation of ternary complexes.

### Example 3: Degron Feature Identification (fAIceit-Degron)

A first-in-kind machine learning based approach is presented to learn features of degrons directly from the molecular surface of degron containing proteins. Unlike the method described in Example 2, this method is trained on degron data.

As noted in Example 2, one of the challenges of performing machine learning on CRBN degrons is how little data is available. The surface-based approach described in Example 1, however, was found to be remarkably capable of learning from a small number of examples, if the training examples are increased using data augmentation, as described herein.

In this method, a protein surface, with per-vertex features (shape index, distance dependent curvature, APBS electrostatics, hydrophobicity, and free/proton electrons), as well as a system of geodesic polar coordinates (angular and radial) for each decomposed patch from the surface was used as input. The output was the same protein surface, but where each vertex has assigned a single value, which is the predicted score for that surface vertex as a degron. This score was represented by a regression score from 0 to 1.

To augment the training data set, the 6 known degrons in 9 crystal structures (PDB ids: 6UML, 6H0G, 6H0F, 5FQD, 5HXB, 6XK9, 7LPS, 7BQU, 7BQV) were used as input to identify similar surfaces, as described in Example 2, and added to the training set. For each of the input structures (either known or augmented), the structure was placed in complex with CRBN, forming a complex between the input structure and CRBN. Then, a surface was computed for both the input structure and for CRBN. The points in the surface of the input structure that belong to the buried surface area of the interface with CRBN were labeled as the degron. Points outside this buried surface area of the interface were labeled as non-degron.

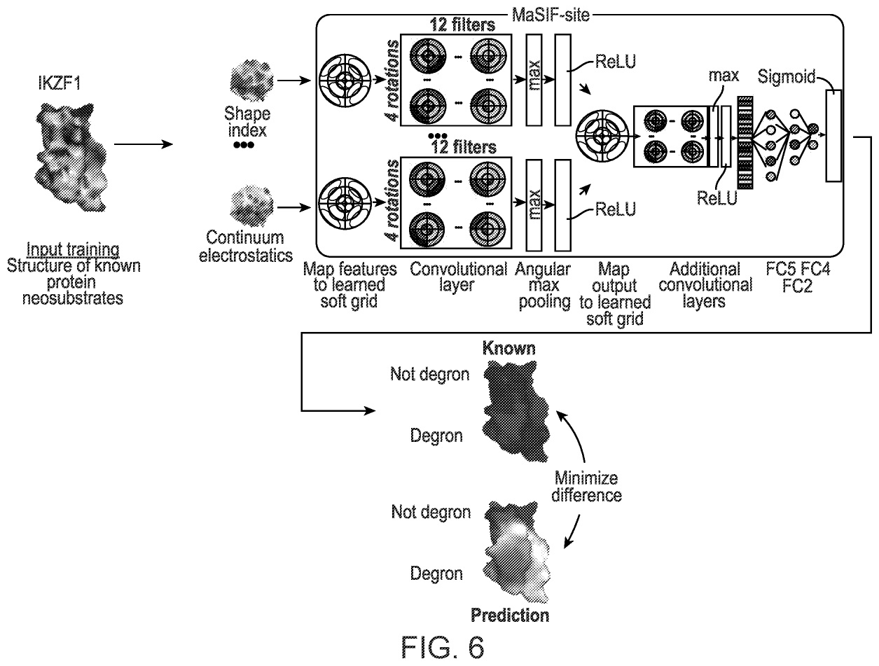

The neural network was then trained using these labeled input structure examples (known or augmented). The input during training was a protein surface, with per-vertex features (shape index, distance dependent curvature, APBS electrostatics, hydrophobicity, and free/proton electrons), as well as a system of geodesic polar coordinates (angular and radial) for each decomposed patch from the surface. In the forward pass, the surface passed over three layers of geodesic convolution, and the output layer was a sigmoid activation function (details of the architecture are shown in FIG. 6). As a loss function, a binary cross entropy loss function was used to minimize the difference between the ground truth degron of the training neosubstrate, and the predicted degron surface. In the backward pass, the weights of the neural network were optimized using an Adam optimizer.

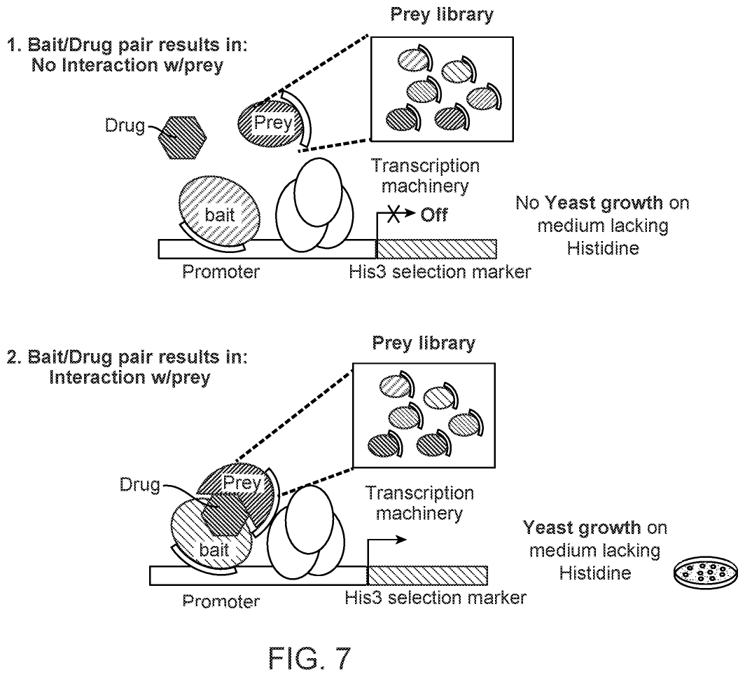

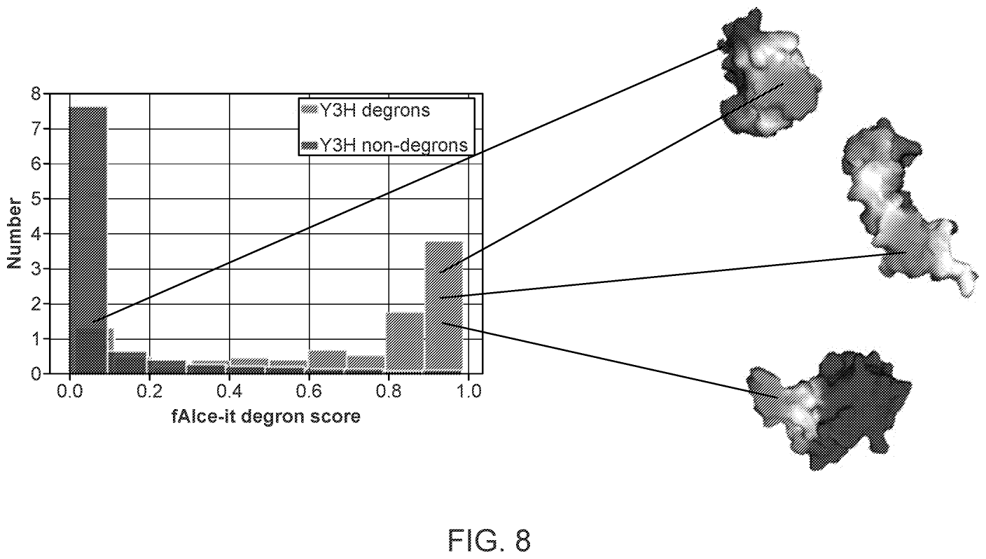

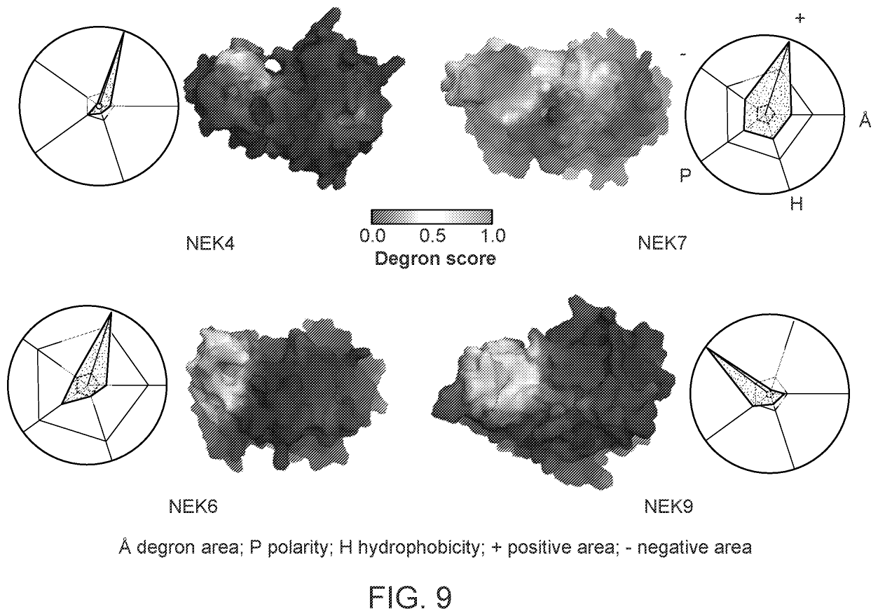

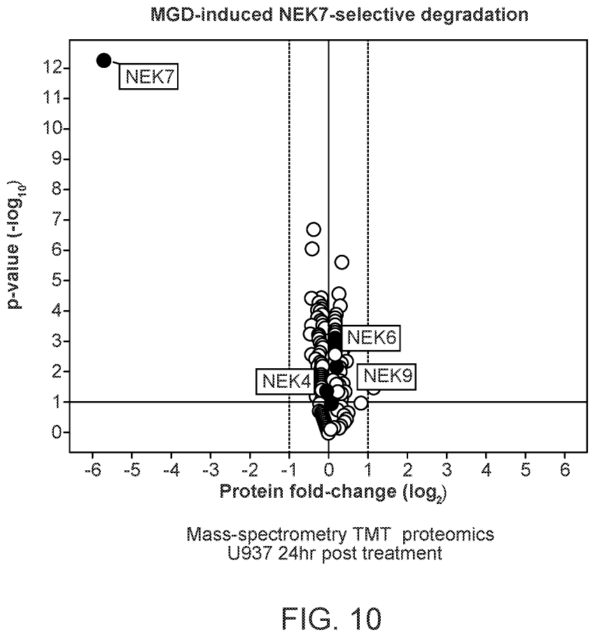

The neural network was validated in multiple ways. First, multiple examples from the training set were separated into a testing set to validate the learning. In addition, several proteins identified from a yeast-3-hybrid assay (FIG. 7) were used as positive examples of validated degrons, and their ground truth degron was compared to the one predicted by fAIceit-degron (FIG. 8). fAIceit-degron was also used to validate degrons for functionally identified targets. In one specific example (FIG. 9), multiple structures of members of the NIMA-related kinase (NEK) family were ran to compute the degron. NEK7 is a target of CRBN which seems to have a higher propensity to engage CRBN than other members of the family. In all cases, fAIceit-degron correctly identified the region where the corresponding degron should be with very high confidence (FIG. 9). Moreover, the strength of the prediction for NEK7 is much higher than all other NEK family members.

Overall, fAIceit-degron is transformative for several reasons. First, it is capable of learning from a very small number of examples. Second, it can learn from the surface which is the best representation of structural degrons, as it is the shape of the protein that is recognized by CRBN. Finally, fAIceit-degron is generalizable to other applications and degron types.

A database of CRBN degrons was constructed using this method, although, as noted above, it can be generalized to other applications and degron types as well.

### Example 4: E3 Ligase (CRBN) Target Finder (fAIceit-Complementarity)

A first-in-kind method was developed for identifying putative neosubstrates through proteome-wide searches of surface complementarity to E3 ligase substrate receptors. This method allows, for the first time, an efficient method for scanning vast databases of proteins for neosubstrates complementary to a neosurface (e.g., of a molecular glue bound E3 ligase substrate receptor such as CRBN). The method performs up to 4000× faster than traditional docking tools.

Structural complexes between E3 ligases and predicted substrates were docked in three-dimensional space and these docked complexes were used for the search of chemical compounds to facilitate the formation of ternary complexes, as follows.

### Potential Neosubstrate (Degron)

Surface fingerprints for a set of potential neosubstrates were prepared for binding to an E3 ligase substrate receptor based on complementarity using a modification of the MasIF framework described in Example 1. Briefly, all structures available for a given gene (PDB and AlphaFold2) were processed by computing chemical features and output with extracted chains and surface features. Then MasIF input was generated and geodesic and radial (angular) coordinates were computed for each patch. Geometric features for each patch were computed and the chemical features which were previously read as input were assigned to each vertex in the patch. MasIF was then used to compute the interface propensity for each patch in the protein, and a fingerprint describing each patch. The fingerprint was used to compare to E3 ligase surfaces (and, in this case, neosurfaces).

### E3 Ligase Substrate Receptor Neosurface

Neosurface features of E3 ligase substrate receptors (including CRBN) were generated for a set of binary complexes of E3 ligase substrate receptors and small molecules, in this example, CRBN in complex with a series of molecular glues. MasIF was modified to receive the neosurface (protein+small molecule) and generate fingerprints and angular/geodesic coordinates as for the potential neosubstrates.

Some of the neosurface fingerprints were extracted from crystal structures (in this case PDB entries) of CRBN bound to a particular molecular glue (PDB ids: 6UML, 6H0G, 6H0F, 5HXB, 6XK9, 7LPS, 7BQU, 7BQV). Some of the neosurface fingerprints were generated by docking molecular glues to CRBN in silico.

MaSIF, as originally implemented, is unable to generate molecular surface fingerprints for these small molecules or binary complexes. To overcome this deficiency, new code was developed to process this type of biomolecule to compute the features of the entire neosurface, making no distinction between protein and small molecule, and assigning all small molecules the hydrophobicity of Tyrosine. Neosurfaces were then processed by computing chemical features, as for neosubstrates, and MasIF input was generated as described above and fingerprints were generated and compared to neosubstrate surfaces.

The fAIceit-complementarity method allows, for the first time, proteome-wide searches of surface complementary, e.g., to E3 ligase substrate receptor proteins such as CRBN, and for the scanning of vast databases of proteins for neosubstrates complementary to a neosurface.

### Matching of Degrons and Neosurfaces

The fingerprints describing the E3 ligase neosurfaces were matched to the neosubstrate surfaces and, for those under a threshold Euclidian distance, a plurality of alignments was generated and scored and filtered to identify potential degrons.

### Example 5: E3 Ligase (CRBN) Target Finder

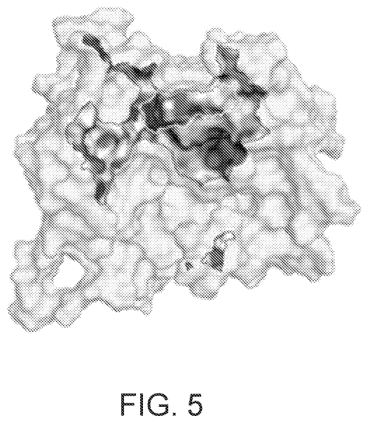

Global docking using MaSIF_search using apo-CRBN (i.e., CRBN without a small molecule bound) or holo-CRBN (i.e., CRBN with a small molecule bound) was carried out against the structurally characterized proteome to identify potential targets for an E3 Ligase Complex. An example of a protein surface is depicted in FIG. 5. Global docking using MaSIF_search of apo-CRBN (drug unbound) was carried out against the structurally characterized proteome. The fast-docking algorithm MaSIF_search was used, followed by a neural network to evaluate the quality of the complexes generated by surface alignment. Optionally, additional steps of filtering and refinement were performed. Predicted complexes of potential targets docked to apo-E3 ligase were identified.

Global docking using MaSIF_search of holo-CRBN was carried out against the structurally characterized proteome. To generate a holo-CRBN for use in this method, a small molecule E3 ligase binding modulator was parameterized and included in the E3 ligase structures. Predicted complexes of potential targets docked to holo-E3 ligase were identified.

### Example 6: MaSIF-Ligand

Testing distinct ligand descriptors based on geometry, chemistry and different structural representations was carried out. Generic training/test sets for small molecule-protein interactions were created and/or identified (e.g., PDBbind database) and processed for compatibility with MaSIF.

Training MaSIF-ligand for the identification of complementary ligands in drug-receptors was carried out. Structural descriptors and learning approaches for capturing the interactions of the small molecules with the proteins' surface patches was identified. The performance of MaSIF-ligand was evaluated by the ability of identifying the correct ligands or ligand fragments for their respective pockets.

A generative pipeline of ligands for E3-substrate-compound ternary complexes was created, stemming only from the surface signature of a given target. Approaches like variational autoencoders can be used. MaSIF-ligand was explicitly tested with E3 ligase ternary pairs to score existing ligands and to generate ligands.

Predicted E3 ligase target ligands were identified.

### Example 7: Identification and Validation of Neosubstrates

Putative neosubstrates of CRBN were identified using the methods described in Examples 2-4.

Yeast three hybrid experiments were carried out to identify molecular glue induced interactions between CRBN and cDNA library-derived targets, as depicted in FIG. 7, which allowed mapping degrons to individual protein domains. The experiments identified 8 novel G-loops from 5 distinct domain classes, which agreed with predictions generated using the methods described in Example 2, as shown in FIG. 8.

As shown in FIG. 9, a unique G-loop surface was identified for NEK7, which allows selective MGD degradation, as shown in FIG. 10.

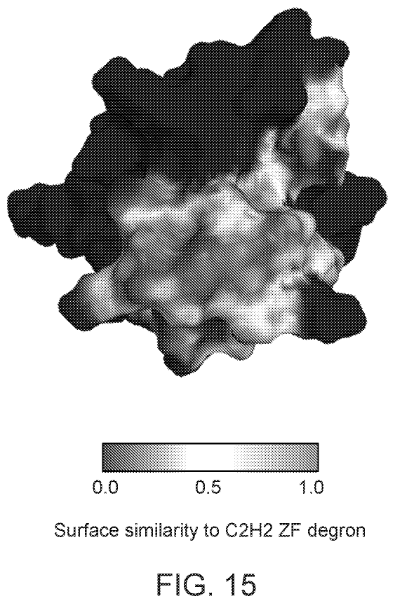

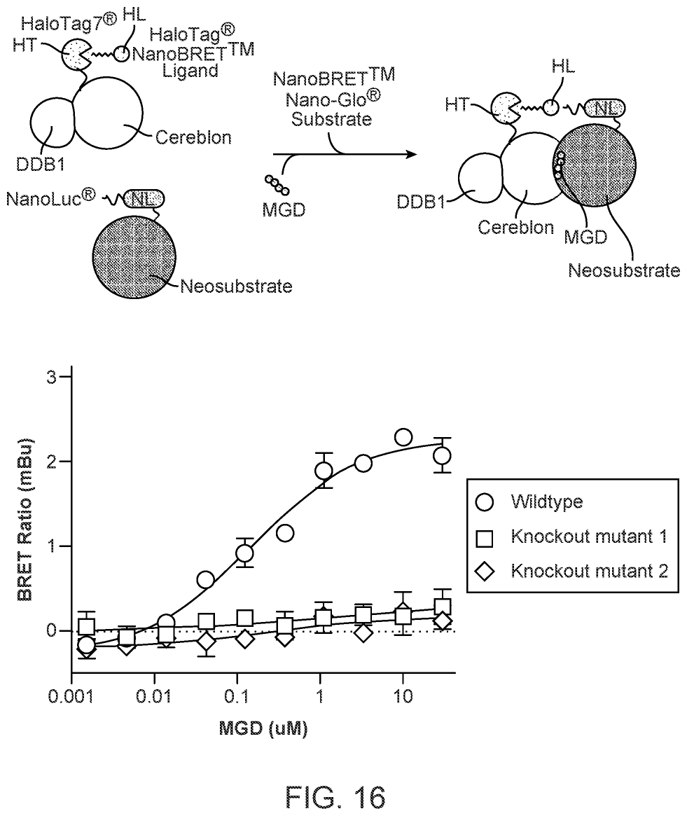

As shown in FIG. 15, a novel non-hairpin, non-canonical degron in an established oncology target (with surface similarity to C2H2 ZF degron), was identified by proteome-wide fast matching of degron surface mimics (i.e., surface fingerprint matching as opposed to G-loop identification)—as described in Example 2). As shown in FIG. 16, NanoBRET confirmed the prediction and binding mode.

### Example 8: Identification and Validation of Neosubstrates

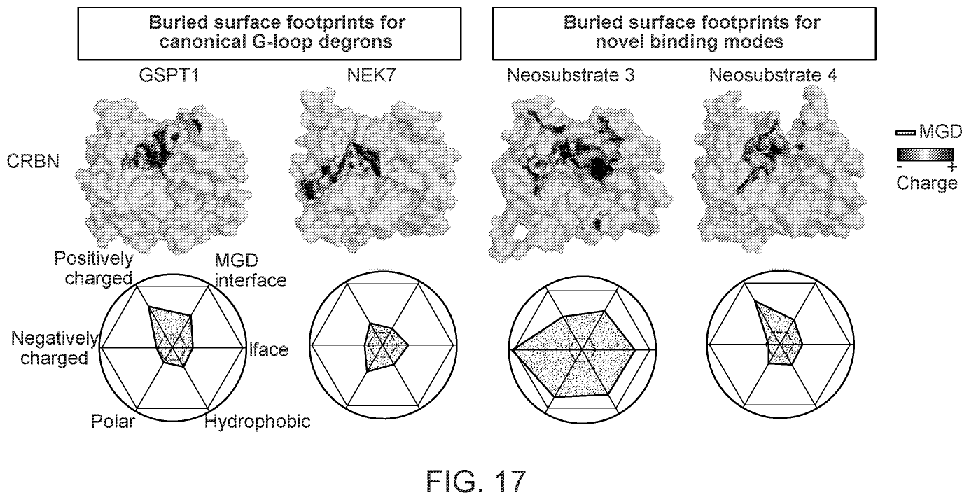

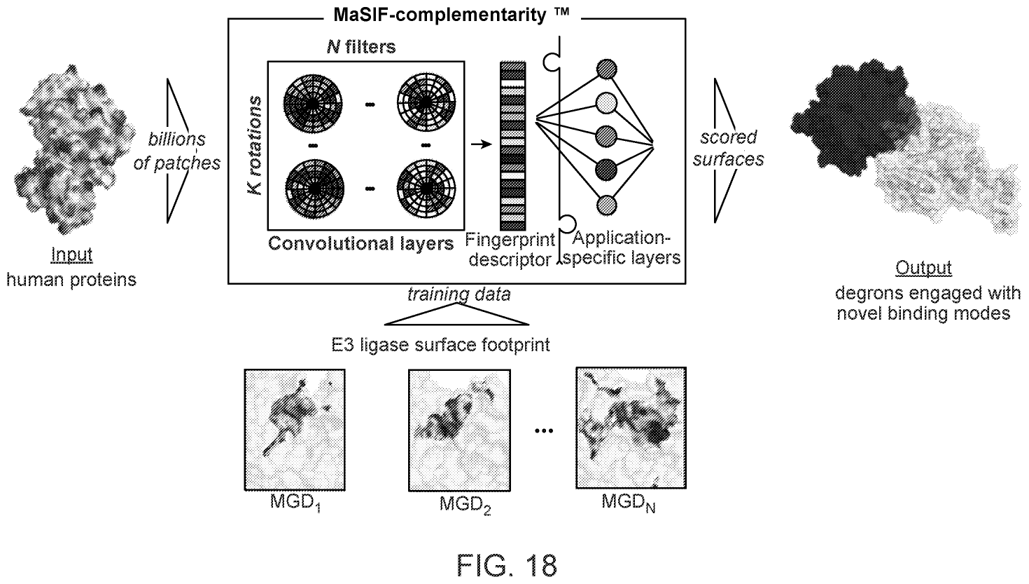

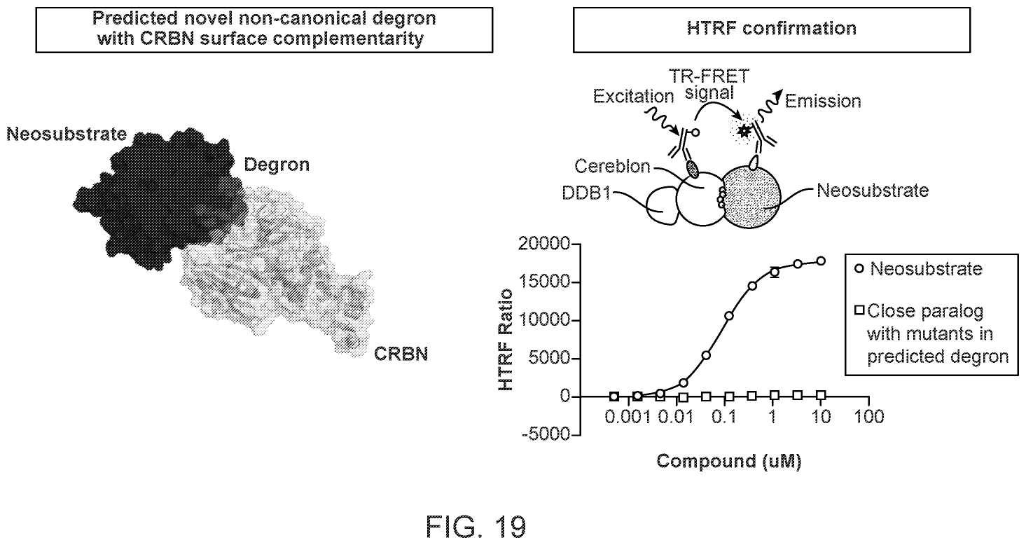

Putative neosubstrates of CRBN were identified using the methods described in Example 3. The CRBN neosurface was used to find novel substrates (e.g., as depicted in FIG. 17 and FIG. 18), and validated in an HTRF assay (e.g., as depicted in FIG. 19).

SEQUENCES
NP_001166953.1
>NP_001166953.1 CRBN [organism = Homo sapiens]
[GeneID = 51185][isoform = 2]
SEQ ID NO: 2
MAGEGDQQDAAHNMGNHLPLLPESEEEDEMEVEDQDSKEAKKPNI
INFDTSLPTSHTYLGADMEEFHGRTLHDDDSCQVIPVLPQVMMIL
IPGQTLPLQLFHPQEVSMVRNLIQKDRTFAVLAYSNVQEREAQFG
TTAEIYAYREEQDFGIEIVKVKAIGRQRFKVLELRTQSDGIQQAK
VQILPECVLPSTMSAVQLESLNKCQIFPSKPVSREDQCSYKWWQK
YQKRKFHCANLTSWPRWLYSLYDAETLMDRIKKQLREWDENLKDD
SLPSNPIDESYRVAACLPIDDVLRIQLLKIGSAIQRLRCELDIMN
KCTSLCCKQCQETEITTKNEIFSLSLCGPMAAYVNPHGYVHETLT
VYKACNLNLIGRPSTEHSWFPGYAWTVAQCKICASHIGWKFTATK
KDMSPQKFWGLTRSALLPTIPDTEDEISPDKVILCL
NP_057386.2
>NP_057386.2 CRBN [organism = Homo sapiens]
[GeneID = 51185][isoform = 1]
SEQ ID NO: 3
MAGEGDQQDAAHNMGNHLPLLPAESEEEDEMEVEDQDSKEAKKPN
IINFDTSLPTSHTYLGADMEEFHGRTLHDDDSCQVIPVLPQVMMI
LIPGQTLPLQLFHPQEVSMVRNLIQKDRTFAVLAYSNVQEREAQF
GTTAEIYAYREEQDFGIEIVKVKAIGRQRFKVLELRTQSDGIQQA
KVQILPECVLPSTMSAVQLESLNKCQIFPSKPVSREDQCSYKWWQ
KYQKRKFHCANLTSWPRWLYSLYDAETLMDRIKKQLREWDENLKD
DSLPSNPIDESYRVAACLPIDDVLRIQLLKIGSAIQRLRCELDIM
NKCTSLCCKQCQETEITTKNEIFSLSLCGPMAAYVNPHGYVHETL
TVYKACNLNLIGRPSTEHSWFPGYAWTVAQCKICASHIGWKFTAT
KKDMSPQKFWGLTRSALLPTIPDTEDEISPDKVILCL
XP_005265259.1
>XP_005265259. 1 CRBN [organism = Homo sapiens]
[GeneID = 51185][isoform = X2]
SEQ ID NO: 4
MEEFHGRTLHDDDSCQVIPVLPQVMMILIPGQTLPLQLFHPQEVS
MVRNLIQKDRTFAVLAYSNVQEREAQFGTTAEIYAYREEQDFGIE
IVKVKAIGRQRFKVLELRTQSDGIQQAKVQILPECVLPSTMSAVQ
LESLNKCQIFPSKPVSREDQCSYKWWQKYQKRKFHCANLTSWPRW
LYSLYDAETLMDRIKKQLREWDENLKDDSLPSNPIDESYRVAACL
PIDDVLRIQLLKIGSAIQRLRCELDIMNKCTSLCCKQCQETEITT
KNEIFSLSLCGPMAAYVNPHGYVHETLTVYKACNLNLIGRPSTEH
SWFPGYAWTVAQCKICASHIGWKFTATKKDMSPQKFWGLTRSALL
PTIPDTEDEISPDKVILCL
XP_011532093.1
>XP_011532093.1 CRBN [organism = Homo sapiens]
[GeneID = 51185][isoform = X1]
SEQ ID NO: 5
MAGEGDQQDAAHNMGNHLPLLPAESEEEDEMEVEDQDSKEAKKPN
IINFDTSLPTSHTYLGADMEEFHGRTLHDDDSCQVIPVLPQVMMI
LIPGQTLPLQLFHPQEVSMVRNLIQKDRTFAVLAYSNVQEREAQF
GTTAEIYAYREEQDFGIEIVKVKAIGRQRFKVLELRTQSDGIQQA
KVQILPECVLPSTMSAVQLESLNKCQIFPSKPVSREDQCSYKWWQ
KYQKRKFHCANLTSWPRWLYSLYDAETLMDRIKKQLREWDENLKD
DSLPSNPIDESYRVAACLPIDDVLRIQLLKIGSAIQRLRCELDIM
NKCTSLCCKQCQETEITTKNEIFRYAWTVAQCKICASHIGWKFTA
TKKDMSPQKFWGLTRSALLPTIPDTEDEISPDKVILCL
XP_011532095.1
>XP_011532095. 1 CRBN [organism = Homo sapiens]
[GeneID = 51185][isoform = x4]
SEQ ID NO: 6
MRLQHLLKMIFRIQQAKVQILPECVLPSTMSAVQLESLNKCQIFP
SKPVSREDQCSYKWWQKYQKRKFHCANLTSWPRWLYSLYDAETLM
DRIKKQLREWDENLKDDSLPSNPIDESYRVAACLPIDDVLRIQLL
KIGSAIQRLRCELDIMNKCTSLCCKQCQETEITTKNEIFSLSLCG
PMAAYVNPHGYVHETLTVYKACNLNLIGRPSTEHSWFPGYAWTVA
QCKICASHIGWKFTATKKDMSPQKFWGLTRSALLPTIPDTEDEIS
PDKVILCL
XP_011532096.1
>XP_011532096.1 CRBN [organism = Homo sapiens]
[GeneID = 51185][isoform = x4]
SEQ ID NO: 7
MRLQHLLKMIFRIQQAKVQILPECVLPSTMSAVQLESLNKCQIFP
SKPVSREDQCSYKWWQKYQKRKFHCANLTSWPRWLYSLYDAETLM
DRIKKQLREWDENLKDDSLPSNPIDESYRVAACLPIDDVLRIQLL
KIGSAIQRLRCELDIMNKCTSLCCKQCQETEITTKNEIFSLSLCG
PMAAYVNPHGYVHETLTVYKACNLNLIGRPSTEHSWFPGYAWTVA
QCKICASHIGWKFTATKKDMSPQKFWGLTRSALLPTIPDTEDEIS
PDKVILCL
XP_024309319.1
>XP_024309319.1 CRBN [organism = Homo sapiens]
[GeneID = 51185][isoform = X3]
SEQ ID NO: 8
MAGEGDQQDAAHNMGNHLPLLPAESEEEDEMEVEDQDSKEAKKPN
IINFDTSLPTSHTYLGADMEEFHGRTLHDDDSCQVIPVLPQVMMI
LIPGQTLPLQLFHPQEVSMVRNLIQ
KDRTFAVLAYSNVQEREAQFGTTAEIYAYREEQDFGIEIVKVKAI
GRQRFKVLELRTQSDGIQQAKVQILPECVLPSTMSAVQLESLNKC
QIFPSKPVSREDQCSYKWWQKYQKRKFHCANLTSWPRWLYSLYDA
ETLMDRIKKQLREWDENLKDDSLPSNPIVYFPLL
(VHL)
>sp|P40337|VHL HUMAN von Hippel-Lindau
disease tumor suppressor OS = Homo
sapiens OX = 9606 GN = VHL PE = 1 SV = 2
SEQ ID NO: 9
MPRRAENWDEAEVGAEEAGVEEYGPEEDGGEESGAEESGPEESGP
EELGAEEEMEAGRPRPVLRSVNSREPSQVIFCNRSPRVVLPVWLN
FDGEPQPYPTLPPGTGRRIHSYRGHLWLFRDAGTHDGLLVNQTEL
FVPSLNVDGQPIFANITLPVYTLKERCLQVVRSLVKPENYRRLDI
VRSLYEDLEDHPNVQKDLERLTQERIAHQRMGD
(NAIP; BIRC1)
>sp|Q13075|BIRC1 HUMAN Baculoviral IAP
repeat-containing protein 1 OS = Homo
sapiens OX = 9606 GN = NAIP PE = 1 SV = 3
SEQ ID NO: 10
MATQQKASDERISQFDHNLLPELSALLGLDAVQLAKELEEEEQKE
RAKMQKGYNSQMRSEAKRLKTFVTYEPYSSWIPQEMAAAGFYFTG
VKSGIQCFCCSLILFGAGLTRLPIEDHKRFHPDCGFLLNKDVGNI
AKYDIRVKNLKSRLRGGKMRYQEEEARLASFRNWPFYVQGISPCV
LSEAGFVFTGKQDTVQCFSCGGCLGNWEEGDDPWKEHAKWFPKCE
FLRSKKSSEEITQYIQSYKGFVDITGEHFVNSWVQRELPMASAYC
NDSIFAYEELRLDSFKDWPRESAVGVAALAKAGLFYTGIKDIVQC
FSCGGCLEKWQEGDDPLDDHTRCFPNCPFLQNMKSSAEVTPDLQS
RGELCELLETTSESNLEDSIAVGPIVPEMAQGEAQWFQEAKNLNE
QLRAAYTSASFRHMSLLDISSDLATDHLLGCDLSIASKHISKPVQ
EPLVLPEVFGNLNSVMCVEGEAGSGKTVLLKKIAFLWASGCCPLL
NRFQLVFYLSLSSTRPDEGLASIICDQLLEKEGSVTEMCVRNIIQ
QLKNQVLFLLDDYKEICSIPQVIGKLIQKNHLSRTCLLIAVRTNR
ARDIRRYLETILEIKAFPFYNTVCILRKLFSHNMTRLRKFMVYFG
KNQSLQKIQKTPLFVAAICAHWFQYPFDPSFDDVAVFKSYMERLS
LRNKATAEILKATVSSCGELALKGFFSCCFEFNDDDLAEAGVDED
EDLTMCLMSKFTAQRLRPFYRFLSPAFQEFLAGMRLIELLDSDRQ
EHQDLGLYHLKQINSPMMTVSAYNNFLNYVSSLPSTKAGPKIVSH
LLHLVDNKESLENISENDDYLKHQPEISLQMQLLRGLWQICPQAY
FSMVSEHLLVLALKTAYQSNTVAACSPFVLQFLQGRTLTLGALNL
QYFFDHPESLSLLRSIHFPIRGNKTSPRAHFSVLETCFDKSQVPT
IDQDYASAFEPMNEWERNLAEKEDNVKSYMDMQRRASPDLSTGYW
KLSPKQYKIPCLEVDVNDIDVVGQDMLEILMTVFSASQRIELHLN
HSRGFIESIRPALELSKASVTKCSISKLELSAAEQELLLTLPSLE
SLEVSGTIQSQDQIFPNLDKFLCLKELSVDLEGNINVFSVIPEEF
PNFHHMEKLLIQISAEYDPSKLVKLIQNSPNLHVFHLKCNFFSDF
GSLMTMLVSCKKLTEIKFSDSFFQAVPFVASLPNFISLKILNLEG
QQFPDEETSEKFAYILGSLSNLEELILPTGDGIYRVAKLIIQQCQ
QLHCLRVLSFFKTLNDDSVVEIAKVAISGGFQKLENLKLSINHKI
TEEGYRNFFQALDNMPNLQELDISRHFTECIKAQATTVKSLSQCV
LRLPRLIRLNMLSWLLDADDIALLNVMKERHPQSKYLTILQKWIL
PFSPIIQK
cIAP1 (BIRC2)
>sp|Q13490|BIRC2 HUMAN Baculoviral IAP
repeat-containing protein 2 OS = Homo
sapiens OX = 9606 GN = BIRC2 PE = 1 SV = 2
SEQ ID NO: 11
MHKTASQRLFPGPSYQNIKSIMEDSTILSDWTNSNKQKMKYDFSC
ELYRMSTYSTFPAGVPVSERSLARAGFYYTGVNDKVKCFCCGLML
DNWKLGDSPIQKHKQLYPSCSFIQNLVSASLGSTSKNTSPMRNSF
AHSLSPTLEHSSLFSGSYSSLSPNPLNSRAVEDISSSRTNPYSYA
MSTEEARFLTYHMWPLTFLSPSELARAGFYYIGPGDRVACFACGG
KLSNWEPKDDAMSEHRRHFPNCPFLENSLETLRFSISNLSMQTHA
ARMRTFMYWPSSVPVQPEQLASAGFYYVGRNDDVKCFCCDGGLRC
WESGDDPWVEHAKWFPRCEFLIRMKGQEFVDEIQGRYPHLLEQLL
STSDTTGEENADPPIIHFGPGESSSEDAVMMNTPVVKSALEMGEN
RDLVKQTVQSKILTTGENYKTVNDIVSALLNAEDEKREEEKEKQA
EEMASDDLSLIRKNRMALFQQLTCVLPILDNLLKANVINKQEHDI
IKQKTQIPLQARELIDTILVKGNAAANIFKNCLKEIDSTLYKNLF
VDKNMKYIPTEDVSGLSLEEQLRRLQEERTCKVCMDKEVSVVFIP
CGHLVVCQECAPSLRKCPICRGIIKGTVRTFLS
cIAP2 (BIRC3)
>sp|Q13489|BIRC3 HUMAN Baculoviral IAP
repeat-containing protein 3 OS = Homo
sapiens OX = 9606 GN = BIRC3 PE = 1 SV = 2
SEQ ID NO: 12
MNIVENSIFLSNLMKSANTFELKYDLSCELYRMSTYSTFPAGVPV
SERSLARAGFYYTGVNDKVKCFCCGLMLDNWKRGDSPTEKHKKLY
PSCRFVQSLNSVNNLEATSQPTFPSSVTNSTHSLLPGTENSGYFR
GSYSNSPSNPVNSRANQDESALMRSSYHCAMNNENARLLTFQTWP
LTFLSPTDLAKAGFYYIGPGDRVACFACGGKLSNWEPKDNAMSEH
LRHFPKCPFIENQLQDTSRYTVSNLSMQTHAARFKTFFNWPSSVL
VNPEQLASAGFYYVGNSDDVKCFCCDGGLRCWESGDDPWVQHAKW
FPRCEYLIRIKGQEFIRQVQASYPHLLEQLLSTSDSPGDENAESS
IIHFEPGEDHSEDAIMMNTPVINAAVEMGFSRSLVKQTVQRKILA
TGENYRLVNDLVLDLLNAEDEIREEERERATEEKESNDLLLIRKN
RMALFQHLTCVIPILDSLLTAGIINEQEHDVIKQKTQTSLQAREL
IDTILVKGNIAATVERNSLQEAEAVLYEHLFVQQDIKYIPTEDVS
DLPVEEQLRRLQEERTCKVCMDKEVSIVFIPCGHLVVCKDCAPSL
RKCPICRSTIKGTVRTELS
(XIAP; BIRC4)
>sp|P98170|XIAP HUMAN E3 ubiquitin-protein
ligase XIAP OS = Homo sapiens
OX = 9606 GN = XIAP PE = 1 SV = 2
SEQ ID NO: 13
MTFNSFEGSKTCVPADINKEEEFVEEFNRLKTFANFPSGSPVSAS
TLARAGFLYTGEGDTVRCFSCHAAVDRWQYGDSAVGRHRKVSPNC
RFINGFYLENSATQSTNSGIQNGQYKVENYLGSRDHFALDRPSET
HADYLLRTGQVVDISDTIYPRNPAMYSEEARLKSFQNWPDYAHLT
PRELASAGLYYTGIGDQVQCFCCGGKLKNWEPCDRAWSEHRRHFP
NCFFVLGRNLNIRSESDAVSSDRNFPNSTNLPRNPSMADYEARIF
TFGTWIYSVNKEQLARAGFYALGEGDKVKCFHCGGGLTDWKPSED
PWEQHAKWYPGCKYLLEQKGQEYINNIHLTHSLEECLVRTTEKTP
SLTRRIDDTIFQNPMVQEAIRMGFSFKDIKKIMEEKIQISGSNYK
SLEVLVADLVNAQKDSMQDESSQTSLQKEISTEEQLRRLQEEKLC
KICMDRNIAIVFVPCGHLVTCKQCAEAVDKCPMCYTVITFKQKIF
MS
(Survivin; BIRC5),
>sp|015392|BIRC5 HUMAN Baculoviral IAP
repeat-containing protein 5 OS = Homo
sapiens OX = 9606 GN = BIRC5 PE = 1 SV = 3
SEQ ID NO: 14
MGAPTLPPAWQPFLKDHRISTFKNWPFLEGCACTPERMAEAGFIH
CPTENEPDLAQCFFCFKELEGWEPDDDPIEEHKKHSSGCAFLSVK
KQFEELTLGEFLKLDRERAKNKIAKETNNKKKEFEETAKKVRRAI
EQLAAMD
(BRUCE; BIRC6)
>sp|Q9NR09|BIRC6 HUMAN Baculoviral IAP
repeat-containing protein 6 OS = Homo
sapiens OX = 9606 GN = BIRC6 PE = 1 SV = 2
SEQ ID NO: 15
MVTGGGAAPPGTVTEPLPSVIVLSAGRKMAAAAAAASGPGCSSAA
GAGAAGVSEWLVLRDGCMHCDADGLHSLSYHPALNAILAVTSRGT
IKVIDGTSGATLQASALSAKPGGQVKCQYISAVDKVIFVDDYAVG
CRKDLNGILLLDTALQTPVSKQDDVVQLELPVTEAQQLLSACLEK
VDISSTEGYDLFITQLKDGLKNTSHETAANHKVAKWATVTFHLPH
HVLKSIASAIVNELKKINQNVAALPVASSVMDRLSYLLPSARPEL
GVGPGRSVDRSLMYSEANRRETFTSWPHVGYRWAQPDPMAQAGFY
HQPASSGDDRAMCFTCSVCLVCWEPTDEPWSEHERHSPNCPFVKG
EHTQNVPLSVTLATSPAQFPCTDGTDRISCFGSGSCPHFLAAATK
RGKICIWDVSKLMKVHLKFEINAYDPAIVQQLILSGDPSSGVDSR
RPTLAWLEDSSSCSDIPKLEGDSDDLLEDSDSEEHSRSDSVTGHT
SQKEAMEVSLDITALSILQQPEKLQWEIVANVLEDTVKDLEELGA
NPCLTNSKSEKTKEKHQEQHNIPFPCLLAGGLLTYKSPATSPISS
NSHRSLDGLSRTQGESISEQGSTDNESCTNSELNSPLVRRTLPVL
LLYSIKESDEKAGKIFSQMNNIMSKSLHDDGFTVPQIIEMELDSQ
EQLLLQDPPVTYIQQFADAAANLTSPDSEKWNSVFPKPGTLVQCL
RLPKFAEEENLCIDSITPCADGIHLLVGLRTCPVESLSAINQVEA
LNNLNKLNSALCNRRKGELESNLAVVNGANISVIQHESPADVQTP
LIIQPEQRNVSGGYLVLYKMNYATRIVTLEEEPIKIQHIKDPQDT
ITSLILLPPDILDNREDDCEEPIEDMQLTSKNGFEREKTSDISTL
GHLVITTQGGYVKILDLSNFEILAKVEPPKKEGTEEQDTFVSVIY
CSGTDRLCACTKGGELHFLQIGGTCDDIDEADILVDGSLSKGIEP
SSEGSKPLSNPSSPGISGVDLLVDQPFTLEILTSLVELTRFETLT
PRESATVPPCWVEVQQEQQQRRHPQHLHQQHHGDAAQHTRTWKLQ
TDSNSWDEHVFELVLPKACMVGHVDFKFVLNSNITNIPQIQVTLL
KNKAPGLGKVNALNIEVEQNGKPSLVDLNEEMQHMDVEESQCLRL
CPFLEDHKEDILCGPVWLASGLDLSGHAGMLTLTSPKLVKGMAGG
KYRSFLIHVKAVNERGTEEICNGGMRPVVRLPSLKHQSNKGYSLA
SLLAKVAAGKEKSSNVKNENTSGTRKSENLRGCDLLQEVSVTIRR
FKKTSISKERVQRCAMLQFSEFHEKLVNTLCRKTDDGQITEHAQS
LVLDTLCWLAGVHSNGPGSSKEGNENLLSKTRKFLSDIVRVCFFE
AGRSIAHKCARFLALCISNGKCDPCQPAFGPVLLKALLDNMSFLP
AATTGGSVYWYFVLLNYVKDEDLAGCSTACASLLTAVSRQLQDRL
TPMEALLQTRYGLYSSPFDPVLFDLEMSGSSCKNVYNSSIGVQSD
EIDLSDVLSGNGKVSSCTAAEGSFTSLTGLLEVEPLHFTCVSTSD
GTRIERDDAMSSFGVTPAVGGLSSGTVGEASTALSSAAQVALQSL
SHAMASAEQQLQVLQEKQQQLLKLQQQKAKLEAKLHQTTAAAAAA
ASAVGPVHNSVPSNPVAAPGFFIHPSDVIPPTPKTTPLFMTPPLT
PPNEAVSVVINAELAQLFPGSVIDPPAVNLAAHNKNSNKSRMNPL
GSGLALAISHASHFLQPPPHQSIIIERMHSGARRFVTLDFGRPIL
LTDVLIPTCGDLASLSIDIWTLGEEVDGRRLVVATDISTHSLILH
DLIPPPVCREMKITVIGRYGSTNARAKIPLGFYYGHTYILPWESE
LKLMHDPLKGEGESANQPEIDQHLAMMVALQEDIQCRYNLACHRL
ETLLQSIDLPPLNSANNAQYFLRKPDKAVEEDSRVFSAYQDCIQL
QLQLNLAHNAVQRLKVALGASRKMLSETSNPEDLIQTSSTEQLRT
IIRYLLDTLLSLLHASNGHSVPAVLQSTFHAQACEELFKHLCISG
TPKIRLHTGLLLVQLCGGERWWGQFLSNVLQELYNSEQLLIFPQD
RVEMLLSCIGQRSLSNSGVLESLLNLLDNLLSPLQPQLPMHRRTE
GVLDIPMISWVVMLVSRLLDYVATVEDEAAAAKKPLNGNQWSFIN
NNLHTQSLNRSSKGSSSLDRLYSRKIRKQLVHHKQQLNLLKAKQK
ALVEQMEKEKIQSNKGSSYKLLVEQAKLKQATSKHFKDLIRLRRT
AEWSRSNLDTEVTTAKESPEIEPLPFTLAHERCISVVQKLVLFLL
SMDFTCHADLLLFVCKVLARIANATRPTIHLCEIVNEPQLERLLL
LLVGTDENRGDISWGGAWAQYSLTCMLQDILAGELLAPVAAEAME
EGTVGDDVGATAGDSDDSLQQSSVQLLETIDEPLTHDITGAPPLS
SLEKDKEIDLELLQDLMEVDIDPLDIDLEKDPLAAKVFKPISSTW
YDYWGADYGTYNYNPYIGGLGIPVAKPPANTEKNGSQTVSVSVSQ
ALDARLEVGLEQQAELMLKMMSTLEADSILQALTNTSPTLSQSPT
GTDDSLLGGLQAANQTSQLIIQLSSVPMLNVCFNKLFSMLQVHHV
QLESLLQLWLTLSLNSSSTGNKENGADIFLYNANRIPVISLNQAS
ITSFLTVLAWYPNTLLRTWCLVLHSLTLMTNMQLNSGSSSAIGTQ
ESTAHLLVSDPNLIHVLVKFLSGTSPHGTNQHSPQVGPTATQAMQ
EFLTRLQVHLSSTCPQIFSEFLLKLIHILSTERGAFQTGQGPLDA
QVKLLEFTLEQNFEVVSVSTISAVIESVTFLVHHYITCSDKVMSR
SGSDSSVGARACFGGLFANLIRPGDAKAVCGEMTRDQLMFDLLKL
VNILVQLPLSGNREYSARVSVTTNTTDSVSDEEKVSGGKDGNGSS
TSVQGSPAYVADLVLANQQIMSQILSALGLCNSSAMAMIIGASGL
HLTKHENFHGGLDAISVGDGLFTILTTLSKKASTVHMMLQPILTY
MACGYMGRQGSLATCQLSEPLLWFILRVLDTSDALKAFHDMGGVQ
LICNNMVTSTRAIVNTARSMVSTIMKFLDSGPNKAVDSTLKTRIL
ASEPDNAEGIHNFAPLGTITSSSPTAQPAEVLLQATPPHRRARSA
AWSYIFLPEEAWCDLTIHLPAAVLLKEIHIQPHLASLATCPSSVS
VEVSADGVNMLPLSTPVVTSGLTYIKIQLVKAEVASAVCLRLHRP
RDASTLGLSQIKLLGLTAFGTTSSATVNNPFLPSEDQVSKTSIGW
LRLLHHCLTHISDLEGMMASAAAPTANLLQTCAALLMSPYCGMHS
PNIEVVLVKIGLQSTRIGLKLIDILLRNCAASGSDPTDLNSPLLF
GRLNGLSSDSTIDILYQLGTTQDPGTKDRIQALLKWVSDSARVAA
MKRSGRMNYMCPNSSTVEYGLLMPSPSHLHCVAAILWHSYELLVE
YDLPALLDQELFELLENWSMSLPCNMVLKKAVDSLLCSMCHVHPN
YFSLLMGWMGITPPPVQCHHRLSMTDDSKKQDLSSSLTDDSKNAQ
APLALTESHLATLASSSQSPEAIKQLLDSGLPSLLVRSLASFCFS
HISSSESIAQSIDISQDKLRRHHVPQQCNKMPITADLVAPILRFL
TEVGNSHIMKDWLGGSEVNPLWTALLFLLCHSGSTSGSHNLGAQQ
TSARSASLSSAATTGLTTQQRTAIENATVAFFLQCISCHPNNQKL
MAQVLCELFQTSPQRGNLPTSGNISGFIRRLFLQLMLEDEKVTMF
LQSPCPLYKGRINATSHVIQHPMYGAGHKFRTLHLPVSTTLSDVL
DRVSDTPSITAKLISEQKDDKEKKNHEEKEKVKAENGFQDNYSVV
VASGLKSQSKRAVSATPPRPPSRRGRTIPDKIGSTSGAEAANKII
TVPVFHLFHKLLAGQPLPAEMTLAQLLTLLYDRKLPQGYRSIDLT
VKLGSRVITDPSLSKTDSYKRLHPEKDHGDLLASCPEDEALTPGD
ECMDGILDESLLETCPIQSPLQVFAGMGGLALIAERLPMLYPEVI
QQVSAPVVTSTTQEKPKDSDQFEWVTIEQSGELVYEAPETVAAEP
PPIKSAVQTMSPIPAHSLAAFGLFLRLPGYAEVLLKERKHAQCLL
RLVLGVTDDGEGSHILQSPSANVLPTLPFHVLRSLFSTTPLTTDD
GVLLRRMALEIGALHLILVCLSALSHHSPRVPNSSVNQTEPQVSS
SHNPTSTEEQQLYWAKGTGFGTGSTASGWDVEQALTKQRLEEEHV
TCLLQVLASYINPVSSAVNGEAQSSHETRGQNSNALPSVLLELLS
QSCLIPAMSSYLRNDSVLDMARHVPLYRALLELLRAIASCAAMVP
LLLPLSTENGEEEEEQSECQTSVGTLLAKMKTCVDTYTNRLRSKR
ENVKTGVKPDASDQEPEGLTLLVPDIQKTAEIVYAATTSLRQANQ
EKKLGEYSKKAAMKPKPLSVLKSLEEKYVAVMKKLQFDTFEMVSE
DEDGKLGFKVNYHYMSQVKNANDANSAARARRLAQEAVTLSTSLP
LSSSSSVFVRCDEERLDIMKVLITGPADTPYANGCFEFDVYFPQD
YPSSPPLVNLETTGGHSVRENPNLYNDGKVCLSILNTWHGRPEEK
WNPQTSSFLQVLVSVQSLILVAEPYFNEPGYERSRGTPSGTQSSR
EYDGNIRQATVKWAMLEQIRNPSPCFKEVIHKHFYLKRVEIMAQC
EEWIADIQQYSSDKRVGRTMSHHAAALKRHTAQLREELLKLPCPE
GLDPDTDDAPEVCRATTGAEETLMHDQVKPSSSKELPSDFQL
(ML-IAP; BIRC7)
>sp|Q96CA5|BIRC7 HUMAN Baculoviral IAP
repeat-containing protein 7 OS = Homo
sapiens OX = 9606 GN = BIRC7 PE = 1 SV = 2
SEQ ID NO: 16
MGPKDSAKCLHRGPQPSHWAAGDGPTQERCGPRSLGSPVLGLDTC
RAWDHVDGQILGQLRPLTEEEEEEGAGATLSRGPAFPGMGSEELR
LASFYDWPLTAEVPPELLAAAGFFHTGHQDKVRCFFCYGGLQSWK
RGDDPWTEHAKWFPSCQFLLRSKGRDFVHSVQETHSQLLGSWDPW
EEPEDAAPVAPSVPASGYPELPTPRREVQSESAQEPGGVSPAEAQ
RAWWVLEPPGARDVEAQLRRLQEERTCKVCLDRAVSIVFVPCGHL
VCAECAPGLQLCPICRAPVRSRVRTFLS
(ILP2; BIRC8)
>sp|Q96P09|BIRC8 HUMAN Baculoviral IAP
repeat-containing protein 8 OS = Homo
sapiens OX = 9606 GN = BIRC8 PE = 1 SV = 2
SEQ ID NO: 17
MTGYEARLITFGTWMYSVNKEQLARAGFYAIGQEDKVQCFHCGGG
LANWKPKEDPWEQHAKWYPGCKYLLEEKGHEYINNIHLTRSLEGA
LVQTTKKTPSLTKRISDTIFPNPMLQEAIRMGFDFKDVKKIMEER
IQTSGSNYKTLEVLVADLVSAQKDTTENELNQTSLQREISPEEPL
RRLQEEKLCKICMDRHIAVVFIPCGHLVTCKQCAEAVDRCPMCSA
VIDFKQRVEMS
(KEAP1)
>sp|Q14145|KEAP1 HUMAN Kelch-like ECH-
associated protein 1 OS = Homo sapiens
OX = 9606 GN = KEAP1 PE = 1 SV = 2
SEQ ID NO: 18
MQPDPRPSGAGACCRFLPLQSQCPEGAGDAVMYASTECKAEVTPS
QHGNRTFSYTLEDHTKQAFGIMNELRLSQQLCDVTLQVKYQDAPA
AQFMAHKVVLASSSPVFKAMFTNGLREQGMEVVSIEGIHPKVMER
LIEFAYTASISMGEKCVLHVMNGAVMYQIDSVVRACSDFLVQQLD
PSNAIGIANFAEQIGCVELHQRAREYIYMHFGEVAKQEEFFNLSH
CQLVTLISRDDLNVRCESEVFHACINWVKYDCEQRRFYVQALLRA
VRCHSLTPNFLQMQLQKCEILQSDSRCKDYLVKIFEELTLHKPTQ
VMPCRAPKVGRLIYTAGGYFRQSLSYLEAYNPSDGTWLRLADLQV
PRSGLAGCVVGGLLYAVGGRNNSPDGNTDSSALDCYNPMTNQWSP
CAPMSVPRNRIGVGVIDGHIYAVGGSHGCIHHNSVERYEPERDEW
HLVAPMLTRRIGVGVAVLNRLLYAVGGFDGTNRLNSAECYYPERN
EWRMITAMNTIRSGAGVCVLHNCIYAAGGYDGQDQLNSVERYDVE
TETWTFVAPMKHRRSALGITVHQGRIYVLGGYDGHTFLDSVECYD
PDTDTWSEVTRMTSGRSGVGVAVTMEPCRKQIDQQNCTC
(DCAF15)
>sp|Q66K64|DCA15 HUMAN DDB1- and CUL4-
associated factor 15 OS = Homo sapiens
OX = 9606 GN = DCAF15 PE = 1 SV = 1
SEQ ID NO: 19
MAPSSKSERNSGAGSGGGGPGGAGGKRAAGRRREHVLKQLERVKI
SGQLSPRLFRKLPPRVCVSLKNIVDEDFLYAGHIFLGFSKCGRYV
LSYTSSSGDDDESFYIYHLYWWEFNVHSKLKLVRQVRLFQDEEIY
SDLYLTVCEWPSDASKVIVFGFNTRSANGMLMNMMMMSDENHRDI
YVSTVAVPPPGRCAACQDASRAHPGDPNAQCLRHGFMLHTKYQVV
YPFPTFQPAFQLKKDQVVLLNTSYSLVACAVSVHSAGDRSFCQIL
YDHSTCPLAPASPPEPQSPELPPALPSFCPEAAPARSSGSPEPSP
AIAKAKEFVADIFRRAKEAKGGVPEEARPALCPGPSGSRCRAHSE
PLALCGETAPRDSPPASEAPASEPGYVNYTKLYYVLESGEGTEPE
DELEDDKISLPFVVTDLRGRNLRPMRERTAVQGQYLTVEQLTLDF
EYVINEVIRHDATWGHQFCSFSDYDIVILEVCPETNQVLINIGLL
LLAFPSPTEEGQLRPKTYHTSLKVAWDLNTGIFETVSVGDLTEVK
GQTSGSVWSSYRKSCVDMVMKWLVPESSGRYVNRMTNEALHKGCS
LKVLADSERYTWIVL
(RNF4)
>sp|P78317|RNF4 HUMAN E3 ubiquitin-
protein ligase RNF4 OS = Homo sapiens
OX = 9606 GN = RNF4 PE = 1 SV = 1
SEQ ID NO: 20
MSTRKRRGGAINSRQAQKRTREATSTPEISLEAEPIELVETAGDE
IVDLTCESLEPVVVDLTHNDSVVIVDERRRPRRNARRLPQDHADS
CVVSSDDEELSRDRDVYVTTHTPRNARDEGATGLRPSGTVSCPIC
MDGYSEIVQNGRLIVSTECGHVFCSQCLRDSLKNANTCPTCRKKI
NHKRYHPIYI
(RNF4)
>sp|P78317-2|RNF4 HUMAN Isoform 2 of E3
ubiquitin-protein ligase RNF4 OS = Homo
sapiens OX = 9606 GN = RNF4
SEQ ID NO: 21
MSTRKRRGGAINSRQAQKRTREATSTPEISLEAEPIELVETAGDE
IVDLTCESLEPVVVDLTHNDSVVIVDGPQVLSVVPSAWTDTQRSC
RMDVSSFPQNAAMSSVASASVIP
(RNF114)
>sp|Q9Y508|RN114 HUMAN E3 ubiquitin-
protein ligase RNF114 OS = Homo sapiens
OX = 9606 GN = RNF114 PE = 1 SV = 1
SEQ ID NO: 22
MAAQQRDCGGAAQLAGPAAEADPLGRFTCPVCLEVYEKPVQVPCG
HVFCSACLQECLKPKKPVCGVCRSALAPGVRAVELERQIESTETS
CHGCRKNFFLSKIRSHVATCSKYQNYIMEGVKATIKDASLQPRNV
PNRYTFPCPYCPEKNFDQEGLVEHCKLFHSTDTKSVVCPICASMP
WGDPNYRSANFREHIQRRHRFSYDTFVDYDVDEEDMMNQVLQRSI
IDQ
(RNF114)
>sp|Q9Y508-2|RN114 HUMAN Isoform 2 of E3
ubiquitin-protein ligase RNF114
OS = Homo sapiens OX = 9606 GN = RNF114
SEQ ID NO: 23
MAAQQRDCGGAAQLAGPAAEADPLGRFTCPVCLEVYEKPVQVPCG
HVFCSACLQECLKPKKPVCGVCRSALAPGVRAVELERQIESTETS
CHGCRKNFFLSKIRSHVATCSKYQNYIMEGVKATIKDASLQPRNV
PNRYTFPCPYCPEKNFDQEGLVEHCKLFHSTDTKSVVSEQSPCLL
SVSCYRASITY
(DCAF16)
>sp|Q9NXF7|DCA16 HUMAN DDB1- and CUL4-
associated factor 16 OS = Homo sapiens
OX = 9606 GN = DCAF16 PE = 1 SV = 1
SEQ ID NO: 24
MGPRNPSPDHLSESESEEEENISYLNESSGEEWDSSEEEDSMVPN
LSPLESLAWQVKCLLKYSTTWKPLNPNSWLYHAKLLDPSTPVHIL
REIGLRLSHCSHCVPKLEPIPEWPPLASCGVPPFQKPLTSPSRLS
RDHATLNGALQFATKQLSRTLSRATPIPEYLKQIPNSCVSGCCCG
WLTKTVKETTRTEPINTTYSYTDFQKAVNKLLTASL
(AHR)
>sp|P35869|AHR HUMAN Aryl hydrocarbon
receptor OS = Homo sapiens OX = 9606 GN = AHR
PE = 1 SV = 2
SEQ ID NO: 25
MNSSSANITYASRKRRKPVQKTVKPIPAEGIKSNPSKRHRDRINT
ELDRLASLLPFPQDVINKLDKLSVLRLSVSYLRAKSFFDVALKSS
PTERNGGQDNCRAANFREGLNLQEGEFLLQALNGFVLVVTTDALV
FYASSTIQDYLGFQQSDVIHQSVYELIHTEDRAEFQRQLHWALNP
SQCTESGQGIEEATGLPQTVVCYNPDQIPPENSPLMERCFICRLR
CLLDNSSGFLAMNFQGKLKYLHGQKKKGKDGSILPPQLALFAIAT
PLQPPSILEIRTKNFIFRTKHKLDFTPIGCDAKGRIVLGYTEAEL
CTRGSGYQFIHAADMLYCAESHIRMIKTGESGMIVFRLLTKNNRW
TWVQSNARLLYKNGRPDYIIVTQRPLTDEEGTEHLRKRNTKLPFM
FTTGEAVLYEATNPFPAIMDPLPLRTKNGTSGKDSATTSTLSKDS
LNPSSLLAAMMQQDESIYLYPASSTSSTAPFENNFFNESMNECRN
WQDNTAPMGNDTILKHEQIDQPQDVNSFAGGHPGLFQDSKNSDLY
SIMKNLGIDFEDIRHMQNEKFFRNDFSGEVDERDIDLTDEILTYV
QDSLSKSPFIPSDYQQQQSLALNSSCMVQEHLHLEQQQQHHQKQV
VVEPQQQLCQKMKHMQVNGMFENWNSNQFVPFNCPQQDPQQYNVF
TDLHGISQEFPYKSEMDSMPYTQNFISCNQPVLPQHSKCTELDYP
MGSFEPSPYPTTSSLEDFVTCLQLPENQKHGLNPQSAIITPQTCY
AGAVSMYQCQPEPQHTHVGQMQYNPVLPGQQAFLNKFQNGVLNET
YPAELNNINNTQTTTHLQPLHHPSEARPFPDLTSSGFL
(MDM2)
>sp|Q00987|MDM2 HUMAN E3 ubiquitin-
protein ligase Mdm2 OS = Homo sapiens
OX = 9606 GN = MDM2 PE = 1 SV = 1
SEQ ID NO: 26
MCNTNMSVPTDGAVTTSQIPASEQETLVRPKPLLLKLLKSVGAQK
DTYTMKEVLFYLGQYIMTKRLYDEKQQHIVYCSNDLLGDLFGVPS
FSVKEHRKIYTMIYRNLVVVNQQESSDSGTSVSENRCHLEGGSDQ
KDLVQELQEEKPSSSHLVSRPSTSSRRRAISETEENSDELSGERQ
RKRHKSDSISLSFDESLALCVIREICCERSSSSESTGTPSNPDLD
AGVSEHSGDWLDQDSVSDQFSVEFEVESLDSEDYSLSEEGQELSD
EDDEVYQVTVYQAGESDTDSFEEDPEISLADYWKCTSCNEMNPPL
PSHCNRCWALRENWLPEDKGKDKGEISEKAKLENSTQAEEGFDVP
DCKKTIVNDSRESCVEENDDKITQASQSQESEDYSQPSTSSSIIY
SSQEDVKEFEREETQDKEESVESSLPLNAIEPCVICQGRPKNGCI
VHGKTGHLMACFTCAKKLKKRNKPCPVCRQPIQMIVLTYFP
(UBR2)
>sp|Q8IWV8|UBR2 HUMAN E3 ubiquitin-
protein ligase UBR2 OS = Homo sapiens
OX = 9606 GN = UBR2 PE = 1 SV = 1
SEQ ID NO: 27
MASELEPEVQAIDRSLLECSAEEIAGKWLQATDLTREVYQHLAHY
VPKIYCRGPNPFPQKEDMLAQHVLLGPMEWYLCGEDPAFGFPKLE
QANKPSHLCGRVFKVGEPTYSCRDCAVDPTCVLCMECFLGSIHRD
HRYRMTTSGGGGFCDCGDTEAWKEGPYCQKHELNTSEIEEEEDPL
VHLSEDVIARTYNIFAITFRYAVEILTWEKESELPADLEMVEKSD
TYYCMLENDEVHTYEQVIYTLQKAVNCTQKEAIGFATTVDRDGRR
SVRYGDFQYCEQAKSVIVRNTSRQTKPLKVQVMHSSIVAHQNFGL
KLLSWLGSIIGYSDGLRRILCQVGLQEGPDGENSSLVDRLMLSDS
KLWKGARSVYHQLFMSSLLMDLKYKKLFAVRFAKNYQQLQRDFME
DDHERAVSVTALSVQFFTAPTLARMLITEENLMSIIIKTFMDHLR
HRDAQGRFQFERYTALQAFKFRRVQSLILDLKYVLISKPTEWSDE
LRQKFLEGFDAFLELLKCMQGMDPITRQVGQHIEMEPEWEAAFTL
QMKLTHVISMMQDWCASDEKVLIEAYKKCLAVLMQCHGGYTDGEQ
PITLSICGHSVETIRYCVSQEKVSIHLPVSRLLAGLHVLLSKSEV
AYKFPELLPLSELSPPMLIEHPLRCLVLCAQVHAGMWRRNGFSLV
NQIYYYHNVKCRREMFDKDVVMLQTGVSMMDPNHFLMIMLSRFEL
YQIFSTPDYGKRFSSEITHKDVVQQNNTLIEEMLYLIIMLVGERF
SPGVGQVNATDEIKREIIHQLSIKPMAHSELVKSLPEDENKETGM
ESVIEAVAHFKKPGLTGRGMYELKPECAKEFNLYFYHFSRAEQSK
AEEAQRKLKRQNREDTALPPPVLPPFCPLFASLVNILQSDVMLCI
MGTILQWAVEHNGYAWSESMLQRVLHLIGMALQEEKQHLENVTEE
HVVTFTFTQKISKPGEAPKNSPSILAMLETLQNAPYLEVHKDMIR
WILKTFNAVKKMRESSPTSPVAETEGTIMEESSRDKDKAERKRKA
EIARLRREKIMAQMSEMQRHFIDENKELFQQTLELDASTSAVLDH
SPVASDMTLTALGPAQTQVPEQRQFVTCILCQEEQEVKVESRAMV
LAAFVQRSTVLSKNRSKFIQDPEKYDPLFMHPDLSCGTHTSSCGH
IMHAHCWQRYFDSVQAKEQRRQQRLRLHTSYDVENGEFLCPLCEC
LSNTVIPLLLPPRNIFNNRLNFSDQPNLTQWIRTISQQIKALQFL
RKEESTPNNASTKNSENVDELQLPEGFRPDFRPKIPYSESIKEML
TTFGTATYKVGLKVHPNEEDPRVPIMCWGSCAYTIQSIERILSDE
DKPLFGPLPCRLDDCLRSLTRFAAAHWTVASVSVVQGHFCKLFAS
LVPNDSHEELPCILDIDMFHLLVGLVLAFPALQCQDFSGISLGTG
DLHIFHLVTMAHIIQILLTSCTEENGMDQENPPCEEESAVLALYK
TLHQYTGSALKEIPSGWHLWRSVRAGIMPFLKCSALFFHYLNGVP
SPPDIQVPGTSHFEHLCSYLSLPNNLICLFQENSEIMNSLIESWC
RNSEVKRYLEGERDAIRYPRESNKLINLPEDYSSLINQASNFSCP
KSGGDKSRAPTLCLVCGSLLCSQSYCCQTELEGEDVGACTAHTYS
CGSGVGIFLRVRECQVLFLAGKTKGCFYSPPYLDDYGETDQGLRR
GNPLHLCKERFKKIQKLWHQHSVTEEIGHAQEANQTLVGIDWQHL
(SPOP)
>sp|043791|SPOP HUMAN Speckle-type POZ
protein OS = Homo sapiens OX = 9606
GN = SPOP PE = 1 SV = 1
SEQ ID NO: 28
MSRVPSPPPPAEMSSGPVAESWCYTQIKVVKFSYMWTINNFSFCR
EEMGEVIKSSTESSGANDKLKWCLRVNPKGLDEESKDYLSLYLLL
VSCPKSEVRAKFKFSILNAKGEETKAMESQRAYRFVQGKDWGFKK
FIRRDFLLDEANGLLPDDKLTLFCEVSVVQDSVNISGQNTMNMVK
VPECRLADELGGLWENSRFTDCCLCVAGQEFQAHKAILAARSPVF
SAMFEHEMEESKKNRVEINDVEPEVFKEMMCFIYTGKAPNLDKMA
DDLLAAADKYALERLKVMCEDALCSNLSVENAAEILILADLHSAD
QLKTQAVDFINYHASDVLETSGWKSMVVSHPHLVAEAYRSLASAQ
CPFLGPPRKRLKQS
(KLHL3)
>sp|Q9UH77|KLHL3 HUMAN Kelch-like protein
3 OS = Homo sapiens OX = 9606 GN = KLHL3
PE = 1 SV = 2
SEQ ID NO: 29
MEGESVKLSSQTLIQAGDDEKNQRTITVNPAHMGKAFKVMNELRS
KQLLCDVMIVAEDVEIEAHRVVLAACSPYFCAMFTGDMSESKAKK
IEIKDVDGQTLSKLIDYIYTAEIEVTEENVQVLLPAASLLQLMDV
RQNCCDFLQSQLHPTNCLGIRAFADVHTCTDLLQQANAYAEQHFP
EVMLGEEFLSLSLDQVCSLISSDKLTVSSEEKVFEAVISWINYEK
ETRLEHMAKLMEHVRLPLLPRDYLVQTVEEEALIKNNNTCKDFLI
EAMKYHLLPLDQRLLIKNPRTKPRTPVSLPKVMIVVGGQAPKAIR
SVECYDFEEDRWDQIAELPSRRCRAGVVFMAGHVYAVGGFNGSLR
VRTVDVYDGVKDQWTSIASMQERRSTLGAAVLNDLLYAVGGFDGS
TGLASVEAYSYKTNEWFFVAPMNTRRSSVGVGVVEGKLYAVGGYD
GASRQCLSTVEQYNPATNEWIYVADMSTRRSGAGVGVLSGQLYAT
GGHDGPLVRKSVEVYDPGTNTWKQVADMNMCRRNAGVCAVNGLLY
VVGGDDGSCNLASVEYYNPVTDKWTLLPTNMSTGRSYAGVAVIHK
SL
(KLHL12)
>sp|Q53G59|KLH12 HUMAN Kelch-like protein
12 OS = Homo sapiens OX = 9606
GN = KLHL12 PE = 1 SV = 2
SEQ ID NO: 30
MGGIMAPKDIMTNTHAKSILNSMNSLRKSNTLCDVTLRVEQKDFP
AHRIVLAACSDYFCAMFTSELSEKGKPYVDIQGLTASTMEILLDF
VYTETVHVTVENVQELLPAACLLQLKGVKQACCEFLESQLDPSNC
LGIRDFAETHNCVDLMQAAEVFSQKHFPEVVQHEEFILLSQGEVE
KLIKCDEIQVDSEEPVFEAVINWVKHAKKEREESLPNLLQYVRMP
LLTPRYITDVIDAEPFIRCSLQCRDLVDEAKKFHLRPELRSQMQG
PRTRARLGANEVLLVVGGFGSQQSPIDVVEKYDPKTQEWSFLPSI
TRKRRYVASVSLHDRIYVIGGYDGRSRLSSVECLDYTADEDGVWY
SVAPMNVRRGLAGATTLGDMIYVSGGFDGSRRHTSMERYDPNIDQ
WSMLGDMQTAREGAGLVVASGVIYCLGGYDGLNILNSVEKYDPHT
GHWTNVTPMATKRSGAGVALLNDHIYVVGGFDGTAHLSSVEAYNI
RTDSWTTVTSMTTPRCYVGATVLRGRLYAIAGYDGNSLLSSIECY
DPIIDSWEVVTSMGTQRCDAGVCVLREK
(KLHL20)
>sp|Q9Y2M5|KLH20 HUMAN Kelch-like protein
20 OS = Homo sapiens OX = 9606
GN = KLHL20 PE = 1 SV = 4
SEQ ID NO: 31
MEGKPMRRCTNIRPGETGMDVTSRCTLGDPNKLPEGVPQPARMPY
ISDKHPRQTLEVINLLRKHRELCDVVLVVGAKKIYAHRVILSACS
PYFRAMFTGELAESRQTEVVIRDIDERAMELLIDFAYTSQITVEE
GNVQTLLPAACLLQLAEIQEACCEFLKRQLDPSNCLGIRAFADTH
SCRELLRIADKFTQHNFQEVMESEEFMLLPANQLIDIISSDELNV
RSEEQVENAVMAWVKYSIQERRPQLPQVLQHVRLPLLSPKFLVGT
VGSDPLIKSDEECRDLVDEAKNYLLLPQERPLMQGPRTRPRKPIR
CGEVLFAVGGWCSGDAISSVERYDPQTNEWRMVASMSKRRCGVGV
SVLDDLLYAVGGHDGSSYLNSVERYDPKTNQWSSDVAPTSTCRTS
VGVAVLGGFLYAVGGQDGVSCLNIVERYDPKENKWTRVASMSTRR
LGVAVAVLGGFLYAVGGSDGTSPLNTVERYNPQENRWHTIAPMGT
RRKHLGCAVYQDMIYAVGGRDDTTELSSAERYNPRTNQWSPVVAM
TSRRSGVGLAVVNGQLMAVGGFDGTTYLKTIEVFDPDANTWRLYG
GMNYRRLGGGVGVIKMTHCESHIW
(KLHDC2)
>sp|Q9Y2U9|KLDC2 HUMAN Kelch domain-
containing protein 2 OS = Homo sapiens
OX = 9606 GN = KLHDC2 PE = 1 SV = 1
SEQ ID NO: 32
MADGNEDLRADDLPGPAFESYESMELACPAERSGHVAVSDGRHMF
VWGGYKSNQVRGLYDFYLPREELWIYNMETGRWKKINTEGDVPPS
MSGSCAVCVDRVLYLFGGHHSRGNTNKFYMLDSRSTDRVLQWERI
DCQGIPPSSKDKLGVWVYKNKLIFFGGYGYLPEDKVLGTFEFDET
SFWNSSHPRGWNDHVHILDTETFTWSQPITTGKAPSPRAAHACAT
VGNRGFVFGGRYRDARMNDLHYLNLDTWEWNELIPQGICPVGRSW
HSLTPVSSDHLFLFGGFTTDKQPLSDAWTYCISKNEWIQFNHPYT
EKPRLWHTACASDEGEVIVEGGCANNLLVHHRAAHSNEILIFSVQ
PKSLVRLSLEAVICFKEMLANSWNCLPKHLLHSVNQRFGSNNTSG
S
(SPSB1)
>sp|Q96BD6|SPSB1 HUMAN SPRY domain-
containing SOCS box protein 1 OS = Homo
sapiens OX = 9606 GN = SPSB1 PE = 1 SV = 1
SEQ ID NO: 33
MGQKVTGGIKTVDMRDPTYRPLKQELQGLDYCKPTRLDLLLDMPP
VSYDVQLLHSWNNNDRSLNVFVKEDDKLIFHRHPVAQSTDAIRGK
VGYTRGLHVWQITWAMRQRGTHAVVGVATADAPLHSVGYTTLVGN
NHESWGWDLGRNRLYHDGKNQPSKTYPAFLEPDETFIVPDSELVA
LDMDDGTLSFIVDGQYMGVAFRGLKGKKLYPVVSAVWGHCEIRMR
YLNGLDPEPLPLMDLCRRSVRLALGRERLGEIHTLPLPASLKAYL
LYQ
(SPSB2)
>sp|Q99619|SPSB2 HUMAN SPRY domain-
containing SOCS box protein 2 OS = Homo
sapiens OX = 9606 GN = SPSB2 PE = 1 SV = 1
SEQ ID NO: 34
MGQTALAGGSSSTPTPQALYPDLSCPEGLEELLSAPPPDLGAQRR
HGWNPKDCSENIEVKEGGLYFERRPVAQSTDGARGKRGYSRGLHA
WEISWPLEQRGTHAVVGVATALAPLQTDHYAALLGSNSESWGWDI
GRGKLYHQSKGPGAPQYPAGTQGEQLEVPERLLVVLDMEEGTLGY
AIGGTYLGPAFRGLKGRTLYPAVSAVWGQCQVRIRYLGERRAEPH
SLLHLSRLCVRHNLGDTRLGQVSALPLPPAMKRYLLYQ
(SPSB4)
>sp|Q96A44|SPSB4 HUMAN SPRY domain
-containing SOCS box protein 4 OS = Homo
sapiens OX = 9606 GN = SPSB4 PE = 1 SV = 1
SEQ ID NO: 35
MGQKLSGSLKSVEVREPALRPAKRELRGAEPGRPARLDQLLDMPA
AGLAVQLRHAWNPEDRSLNVFVKDDDRLTFHRHPVAQSTDGIRGK
VGHARGLHAWQINWPARQRGTHAVVGVATARAPLHSVGYTALVGS
DAESWGWDLGRSRLYHDGKNQPGVAYPAFLGPDEAFALPDSLLVV
LDMDEGTLSFIVDGQYLGVAFRGLKGKKLYPVVSAVWGHCEVTMR
YINGLDPEPLPLMDLCRRSIRSALGRQRLQDISSLPLPQSLKNYL
QYQ
(SOCS2)
>sp|014508|SOCS2 HUMAN Suppressor of
cytokine signaling 2 OS = Homo sapiens
OX = 9606 GN = SOCS2 PE = 1 SV = 1
SEQ ID NO: 36
MTLRCLEPSGNGGEGTRSQWGTAGSAEEPSPQAARLAKALRELGQ
TGWYWGSMTVNEAKEKLKEAPEGTFLIRDSSHSDYLLTISVKTSA
GPTNLRIEYQDGKFRLDSIICVKSKLKQFDSVVHLIDYYVQMCKD
KRTGPEAPRNGTVHLYLTKPLYTSAPSLQHLCRLTINKCTGAIWG
LPLPTRLKDYLEEYKFQV
(SOCS6)
>sp|014544|SOCS6 HUMAN Suppressor of
cytokine signaling 6 OS = Homo sapiens
OX = 9606 GN = SOCS6 PE = 1 SV = 2
SEQ ID NO: 37
MKKISLKTLRKSFNLNKSKEETDFMVVQQPSLASDFGKDDSLFGS
CYGKDMASCDINGEDEKGGKNRSKSESLMGTLKRRLSAKQKSKGK
AGTPSGSSADEDTFSSSSAPIVEKDVRAQRPIRSTSLRSHHYSPA
PWPLRPTNSEETCIKMEVRVKALVHSSSPSPALNGVRKDFHDLQS
ETTCQEQANSLKSSASHNGDLHLHLDEHVPVVIGLMPQDYIQYTV
PLDEGMYPLEGSRSYCLDSSSPMEVSAVPPQVGGRAFPEDESQVD
QDLVVAPEIFVDQSVNGLLIGTTGVMLQSPRAGHDDVPPLSPLLP
PMQNNQIQRNFSGLTGTEAHVAESMRCHLNFDPNSAPGVARVYDS
VQSSGPMVVTSLTEELKKLAKQGWYWGPITRWEAEGKLANVPDGS
FLVRDSSDDRYLLSLSFRSHGKTLHTRIEHSNGRFSFYEQPDVEG
HTSIVDLIEHSIRDSENGAFCYSRSRLPGSATYPVRLTNPVSRFM
QVRSLQYLCRFVIRQYTRIDLIQKLPLPNKMKDYLQEKHY
(FBX04)
>sp|Q9UKT5|FBX4 HUMAN F-box only protein
4 OS = Homo sapiens OX = 9606 GN = FBXO4
PE = 1 SV = 2
SEQ ID NO: 38
MAGSEPRSGTNSPPPPESDWGRLEAAILSGWKTFWQSVSKERVAR
TTSREEVDEAASTLTRLPIDVQLYILSFLSPHDLCQLGSTNHYWN
ETVRDPILWRYFLLRDLPSWSSVDWKSLPDLEILKKPISEVTDGA
FFDYMAVYRMCCPYTRRASKSSRPMYGAVTSFLHSLIIQNEPRFA
MFGPGLEELNTSLVLSLMSSEELCPTAGLPQRQIDGIGSGVNFQL
NNQHKFNILILYSTTRKERDRAREEHTSAVNKMFSRHNEGDDQQG
SRYSVIPQIQKVCEVVDGFIYVANAEAHKRHEWQDEFSHIMAMTD
PAFGSSGRPLLVLSCISQGDVKRMPCFYLAHELHLNLLNHPWLVQ
DTEAETLTGELNGIEWILEEVESKRAR
(FBXO31)
>sp|Q5XUX0|FBX31 HUMAN F-box only protein
31 OS = Homo sapiens OX = 9606
GN = FBXO31 PE = 1 SV = 2
SEQ ID NO: 39
MAVCARLCGVGPSRGCRRRQQRRGPAETAAADSEPDTDPEEERIE
ASAGVGGGLCAGPSPPPPRCSLLELPPELLVEIFASLPGTDLPSL
AQVCTKFRRILHTDTIWRRRCREEYGVCENLRKLEITGVSCRDVY
AKLLHRYRHILGLWQPDIGPYGGLLNVVVDGLFIIGWMYLPPHDP
HVDDPMRFKPLFRIHLMERKAATVECMYGHKGPHHGHIQIVKKDE
FSTKCNQTDHHRMSGGRQEEFRTWLREEWGRTLEDIFHEHMQELI
LMKFIYTSQYDNCLTYRRIYLPPSRPDDLIKPGLFKGTYGSHGLE
IVMLSFHGRRARGTKITGDPNIPAGQQTVEIDLRHRIQLPDLENQ
RNFNELSRIVLEVRERVRQEQQEGGHEAGEGRGRQGPRESQPSPA
QPRAEAPSKGPDGTPGEDGGEPGDAVAAAEQPAQCGQGQPFVLPV
GVSSRNEDYPRTCRMCFYGTGLIAGHGFTSPERTPGVFILFDEDR
FGFVWLELKSFSLYSRVQATFRNADAPSPQAFDEMLKNIQSLTS
(BTRC)
>sp|Q9Y297|FBW1A HUMAN F-box/WD repeat-
containing protein 1A OS = Homo sapiens
OX = 9606 GN = BTRC PE = 1 SV = 1
SEQ ID NO: 40
MDPAEAVLQEKALKFMCSMPRSLWLGCSSLADSMPSLRCLYNPGT
GALTAFQNSSEREDCNNGEPPRKIIPEKNSLRQTYNSCARLCLNQ
ETVCLASTAMKTENCVAKTKLANGTSSMIVPKQRKLSASYEKEKE
LCVKYFEQWSESDQVEFVEHLISQMCHYQHGHINSYLKPMLQRDF
ITALPARGLDHIAENILSYLDAKSLCAAELVCKEWYRVTSDGMLW
KKLIERMVRTDSLWRGLAERRGWGQYLFKNKPPDGNAPPNSFYRA
LYPKIIQDIETIESNWRCGRHSLQRIHCRSETSKGVYCLQYDDQK
IVSGLRDNTIKIWDKNTLECKRILTGHTGSVLCLQYDERVIITGS
SDSTVRVWDVNTGEMLNTLIHHCEAVLHLRFNNGMMVTCSKDRSI
AVWDMASPTDITLRRVLVGHRAAVNVVDFDDKYIVSASGDRTIKV
WNTSTCEFVRTLNGHKRGIACLQYRDRLVVSGSSDNTIRLWDIEC
GACLRVLEGHEELVRCIRFDNKRIVSGAYDGKIKVWDLVAALDPR
APAGTLCLRTLVEHSGRVFRLQFDEFQIVSSSHDDTILIWDELND
PAAQAEPPRSPSRTYTYISR
(FBW7)
>sp|Q969H0|FBXW7 HUMAN F-box/WD repeat-
containing protein 7 OS = Homo sapiens
OX = 9606 GN = FBXW7 PE = 1 SV = 1
SEQ ID NO: 41
MNQELLSVGSKRRRTGGSLRGNPSSSQVDEEQMNRVVEEEQQQQL
RQQEEEHTARNGEVVGVEPRPGGQNDSQQGQLEENNNRFISVDED
SSGNQEEQEEDEEHAGEQDEEDEEEEEMDQESDDFDQSDDSSRED
EHTHTNSVTNSSSIVDLPVHQLSSPFYTKTTKMKRKLDHGSEVRS
FSLGKKPCKVSEYTSTTGLVPCSATPTTFGDLRAANGQGQQRRRI
TSVQPPTGLQEWLKMFQSWSGPEKLLALDELIDSCEPTQVKHMMQ
VIEPQFQRDFISLLPKELALYVLSFLEPKDLLQAAQTCRYWRILA
EDNLLWREKCKEEGIDEPLHIKRRKVIKPGFIHSPWKSAYIRQHR
IDTNWRRGELKSPKVLKGHDDHVITCLQFCGNRIVSGSDDNTLKV
WSAVTGKCLRTLVGHTGGVWSSQMRDNIIISGSTDRTLKVWNAET
GECIHTLYGHTSTVRCMHLHEKRVVSGSRDATLRVWDIETGQCLH
VLMGHVAAVRCVQYDGRRVVSGAYDFMVKVWDPETETCLHTLQGH
TNRVYSLQFDGIHVVSGSLDTSIRVWDVETGNCIHTLTGHQSLTS
GMELKDNILVSGNADSTVKIWDIKTGQCLQTLQGPNKHQSAVTCL
QFNKNFVITSSDDGTVKLWDLKTGEFIRNLVTLESGGSGGVVWRI
RASNTKLVCAVGSRNGTEETKLLVLDEDVDMK
(CDC20)
>sp|Q12834|CDC20 HUMAN Cell division cycle
protein 20 homolog OS = Homo sapiens
OX = 9606 GN = CDC20 PE = 1 SV = 2
SEQ ID NO: 42
MAQFAFESDLHSLLQLDAPIPNAPPARWQRKAKEAAGPAPSPMRA
ANRSHSAGRTPGRTPGKSSSKVQTTPSKPGGDRYIPHRSAAQMEV
ASFLLSKENQPENSQTPTKKEHQKAWALNLNGFDVEEAKILRLSG
KPQNAPEGYQNRLKVLYSQKATPGSSRKTCRYIPSLPDRILDAPE
IRNDYYLNLVDWSSGNVLAVALDNSVYLWSASSGDILQLLQMEQP
GEYISSVAWIKEGNYLAVGTSSAEVQLWDVQQQKRLRNMTSHSAR
VGSLSWNSYILSSGSRSGHIHHHDVRVAEHHVATLSGHSQEVCGL
RWAPDGRHLASGGNDNLVNVWPSAPGEGGWVPLQTFTQHQGAVKA
VAWCPWQSNVLATGGGTSDRHIRIWNVCSGACLSAVDAHSQVCSI
LWSPHYKELISGHGFAQNQLVIWKYPTMAKVAELKGHTSRVLSLT
MSPDGATVASAAADETLRLWRCFELDPARRREREKASAAKSSLIH
QGIR
(ITCH)
>sp|Q96J02|ITCH HUMAN E3 ubiquitin-protein
ligase Itchy homolog OS = Homo
sapiens OX = 9606 GN = ITCH PE = 1 SV = 2
SEQ ID NO: 43
MSDSGSQLGSMGSLTMKSQLQITVISAKLKENKKNWFGPSPYVEV
TVDGQSKKTEKCNNTNSPKWKQPLTVIVTPVSKLHFRVWSHQTLK
SDVLLGTAALDIYETLKSNNMKLEEVVVTLQLGGDKEPTETIGDL
SICLDGLQLESEVVTNGETTCSENGVSLCLPRLECNSAISAHCNL
CLPGLSDSPISASRVAGFTGASQNDDGSRSKDETRVSTNGSDDPE
DAGAGENRRVSGNNSPSLSNGGFKPSRPPRPSRPPPPTPRRPASV
NGSPSATSESDGSSTGSLPPTNTNTNTSEGATSGLIIPLTISGGS
GPRPLNPVTQAPLPPGWEQRVDQHGRVYYVDHVEKRTTWDRPEPL
PPGWERRVDNMGRIYYVDHFTRTTTWQRPTLESVRNYEQWQLQRS
QLQGAMQQFNQRFIYGNQDLFATSQSKEFDPLGPLPPGWEKRTDS
NGRVYFVNHNTRITQWEDPRSQGQLNEKPLPEGWEMRFTVDGIPY
FVDHNRRTTTYIDPRTGKSALDNGPQIAYVRDFKAKVQYFRFWCQ
QLAMPQHIKITVTRKTLFEDSFQQIMSFSPQDLRRRLWVIFPGEE
GLDYGGVAREWFFLLSHEVLNPMYCLFEYAGKDNYCLQINPASYI
NPDHLKYFRFIGRFIAMALFHGKFIDTGESLPFYKRILNKPVGLK
DLESIDPEFYNSLIWVKENNIEECDLEMYFSVDKEILGEIKSHDL
KPNGGNILVTEENKEEYIRMVAEWRLSRGVEEQTQAFFEGFNEIL
PQQYLQYFDAKELEVLLCGMQEIDLNDWQRHAIYRHYARTSKQIM
WFWQFVKEIDNEKRMRLLQFVTGTCRLPVGGFADLMGSNGPQKFC
IEKVGKENWLPRSHTCFNRLDLPPYKSYEQLKEKLLFAIEETEGF
GQE
(PML)
>sp|P29590|PML HUMAN Protein PML
OS = Homo sapiens OX = 9606 GN = PML PE = 1
SV = 3
SEQ ID NO: 44
MEPAPARSPRPQQDPARPQEPTMPPPETPSEGRQPSPSPSPTERA
PASEEEFQFLRCQQCQAEAKCPKLLPCLHTLCSGCLEASGMQCPI
CQAPWPLGADTPALDNVFFESLQRRLSVYRQIVDAQAVCTRCKES
ADFWCFECEQLLCAKCFEAHQWELKHEARPLAELRNQSVREFLDG
TRKTNNIFCSNPNHRTPTLTSIYCRGCSKPLCCSCALLDSSHSEL
KCDISAEIQQRQEELDAMTQALQEQDSAFGAVHAQMHAAVGQLGR
ARAETEELIRERVRQVVAHVRAQERELLEAVDARYQRDYEEMASR
LGRLDAVLQRIRTGSALVQRMKCYASDQEVLDMHGFLRQALCRLR
QEEPQSLQAAVRTDGFDEFKVRLQDLSSCITQGKDAAVSKKASPE
AASTPRDPIDVDLPEEAERVKAQVQALGLAEAQPMAVVQSVPGAH
PVPVYAFSIKGPSYGEDVSNTTTAQKRKCSQTQCPRKVIKMESEE
GKEARLARSSPEQPRPSTSKAVSPPHLDGPPSPRSPVIGSEVELP
NSNHVASGAGEAEERVVVISSSEDSDAENSSSRELDDSSSESSDL
QLEGPSTLRVLDENLADPQAEDRPLVFFDLKIDNETQKISQLAAV
NRESKFRVVIQPEAFFSIYSKAVSLEVGLQHFLSFLSSMRRPILA
CYKLWGPGLPNFFRALEDINRLWEFQEAISGFLAALPLIRERVPG
ASSFKLKNLAQTYLARNMSERSAMAAVLAMRDLCRLLEVSPGPQL
AQHVYPFSSLQCFASLQPLVQAAVLPRAEARLLALHNVSFMELLS
AHRRDRQGGLKKYSRYLSLQTTTLPPAQPAFNLQALGTYFEGLLE
GPALARAEGVSTPLAGRGLAERASQQS
(TRIM21)
>sp|P19474|RO52 HUMAN E3 ubiquitin-protein
ligase TRIM21 OS = Homo sapiens
OX = 9606 GN = TRIM21 PE = 1 SV = 1
SEQ ID NO: 45
MASAARLTMMWEEVTCPICLDPFVEPVSIECGHSFCQECISQVGK
GGGSVCPVCRQRFLLKNLRPNRQLANMVNNLKEISQEAREGTQGE
RCAVHGERLHLFCEKDGKALCWVCAQSRKHRDHAMVPLEEAAQEY
QEKLQVALGELRRKQELAEKLEVEIAIKRADWKKTVETQKSRIHA
EFVQQKNFLVEEEQRQLQELEKDEREQLRILGEKEAKLAQQSQAL
QELISELDRRCHSSALELLQEVIIVLERSESWNLKDLDITSPELR
SVCHVPGLKKMLRTCAVHITLDPDTANPWLILSEDRRQVRLGDTQ
QSIPGNEERFDSYPMVLGAQHFHSGKHYWEVDVTGKEAWDLGVCR
DSVRRKGHFLLSSKSGFWTIWLWNKQKYEAGTYPQTPLHLQVPPC
QVGIFLDYEAGMVSFYNITDHGSLIYSFSECAFTGPLRPFFSPGE
NDGGKNTAPLTLCPLNIGSQGSTDY
(TRIM24)
>sp|015164|TIF1A HUMAN Transcription
intermediary factor 1-alpha OS = Homo
sapiens OX = 9606 GN = TRIM24 PE = 1 SV = 3
SEQ ID NO: 46
MEVAVEKAVAAAAAASAAASGGPSAAPSGENEAESRQGPDSERGG
EAARLNLLDTCAVCHQNIQSRAPKLLPCLHSFCQRCLPAPQRYLM
LPAPMLGSAETPPPVPAPGSPVSGSSPFATQVGVIRCPVCSQECA
ERHIIDNFFVKDTTEVPSSTVEKSNQVCTSCEDNAEANGFCVECV
EWLCKTCIRAHQRVKFTKDHTVRQKEEVSPEAVGVTSQRPVFCPF
HKKEQLKLYCETCDKLTCRDCQLLEHKEHRYQFIEEAFQNQKVII
DTLITKLMEKTKYIKFTGNQIQNRIIEVNQNQKQVEQDIKVAIFT
LMVEINKKGKALLHQLESLAKDHRMKLMQQQQEVAGLSKQLEHVM
HFSKWAVSSGSSTALLYSKRLITYRLRHLLRARCDASPVTNNTIQ
FHCDPSFWAQNIINLGSLVIEDKESQPQMPKQNPVVEQNSQPPSG
LSSNQLSKFPTQISLAQLRLQHMQQQVMAQRQQVQRRPAPVGLPN
PRMQGPIQQPSISHQQPPPRLINFQNHSPKPNGPVLPPHPQQLRY
PPNQNIPRQAIKPNPLQMAFLAQQAIKQWQISSGQGTPSTTNSTS
STPSSPTITSAAGYDGKAFGSPMIDLSSPVGGSYNLPSLPDIDCS
STIMLDNIVRKDTNIDHGQPRPPSNRTVQSPNSSVPSPGLAGPVT
MTSVHPPIRSPSASSVGSRGSSGSSSKPAGADSTHKVPVVMLEPI
RIKQENSGPPENYDFPVVIVKQESDEESRPQNANYPRSILTSLLL
NSSQSSTSEETVLRSDAPDSTGDQPGLHQDNSSNGKSEWLDPSQK
SPLHVGETRKEDDPNEDWCAVCQNGGELLCCEKCPKVFHLSCHVP
TLTNFPSGEWICTFCRDLSKPEVEYDCDAPSHNSEKKKTEGLVKL
TPIDKRKCERLLLFLYCHEMSLAFQDPVPLTVPDYYKIIKNPMDL
STIKKRLQEDYSMYSKPEDFVADERLIFQNCAEFNEPDSEVANAG
IKLENYFEELLKNLYPEKRFPKPEFRNESEDNKFSDDSDDDFVQP
RKKRLKSIEERQLLK
(TRIM33)
>sp|Q9UPN9|TRI33 HUMAN E3 ubiquitin-
protein ligase TRIM33 OS = Homo sapiens
OX = 9606 GN = TRIM33 PE = 1 SV = 3
SEQ ID NO: 47
MAENKGGGEAESGGGGSGSAPVTAGAAGPAAQEAEPPLTAVLVEE
EEEEGGRAGAEGGAAGPDDGGVAAASSGSAQAASSPAASVGTGVA
GGAVSTPAPAPASAPAPGPSAGPPPGPPASLLDTCAVCQQSLQSR
REAEPKLLPCLHSFCLRCLPEPERQLSVPIPGGSNGDIQQVGVIR
CPVCRQECRQIDLVDNYFVKDTSEAPSSSDEKSEQVCTSCEDNAS
AVGFCVECGEWLCKTCIEAHQRVKFTKDHLIRKKEDVSESVGASG
QRPVFCPVHKQEQLKLFCETCDRLTCRDCQLLEHKEHRYQFLEEA
FQNQKGAIENLLAKLLEKKNYVHFAATQVQNRIKEVNETNKRVEQ
EIKVAIFTLINEINKKGKSLLQQLENVTKERQMKLLQQQNDITGL
SRQVKHVMNFTNWAIASGSSTALLYSKRLITFQLRHILKARCDPV
PAANGAIRFHCDPTFWAKNVVNLGNLVIESKPAPGYTPNVVVGQV
PPGTNHISKTPGQINLAQLRLQHMQQQVYAQKHQQLQQMRMQQPP
APVPTTTTTTQQHPRQAAPQMLQQQPPRLISVQTMQRGNMNCGAF
QAHQMRLAQNAARIPGIPRHSGPQYSMMQPHLQRQHSNPGHAGPF
PVVSVHNTTINPTSPTTATMANANRGPTSPSVTAIELIPSVTNPE
NLPSLPDIPPIQLEDAGSSSLDNLLSRYISGSHLPPQPTSTMNPS
PGPSALSPGSSGLSNSHTPVRPPSTSSTGSRGSCGSSGRTAEKTS
LSFKSDQVKVKQEPGTEDEICSFSGGVKQEKTEDGRRSACMLSSP
ESSLTPPLSTNLHLESELDALASLENHVKIEPADMNESCKQSGLS
SLVNGKSPIRSLMHRSARIGGDGNNKDDDPNEDWCAVCQNGGDLL
CCEKCPKVFHLTCHVPTLLSFPSGDWICTFCRDIGKPEVEYDCDN
LQHSKKGKTAQGLSPVDQRKCERLLLYLYCHELSIEFQEPVPASI
PNYYKIIKKPMDLSTVKKKLQKKHSQHYQIPDDFVADVRLIFKNC
ERFNEMMKVVQVYADTQEINLKADSEVAQAGKAVALYFEDKLTEI
YSDRTFAPLPEFEQEEDDGEVTEDSDEDFIQPRRKRLKSDERPVH
IK
(GID4)
>sp|Q8IVV7|GID4 HUMAN Glucose-induced
degradation protein 4 homolog OS = Homo
sapiens OX = 9606 GN = GID4 PE = 1 SV = 1
SEQ ID NO: 48
MCARGQVGRGTQLRTGRPCSQVPGSRWRPERLLRRQRAGGRPSRP
HPARARPGLSLPATLLGSRAAAAVPLPLPPALAPGDPAMPVRTEC
PPPAGASAASAASLIPPPPINTQQPGVATSLLYSGSKFRGHQKSK
GNSYDVEVVLQHVDTGNSYLCGYLKIKGLTEEYPTLTTFFEGEII
SKKHPFLTRKWDADEDVDRKHWGKFLAFYQYAKSFNSDDFDYEEL
KNGDYVFMRWKEQFLVPDHTIKDISGASFAGFYYICFQKSAASIE
GYYYHRSSEWYQSLNLTHVPEHSAPIYEFR
(DCAF11)
>sp|Q8TEB1|DCA11 HUMAN DDB1- and CUL4-
associated factor 11 OS = Homo sapiens
OX = 9606 GN = DCAF11 PE = 1 SV = 1
SEQ ID NO: 49
MGSRNSSSAGSGSGDPSEGLPRRGAGLRRSEEEEEEDEDVDLAQV
LAYLLRRGQVRLVQGGGAANLQFIQALLDSEEENDRAWDGRLGDR
YNPPVDATPDTRELEFNEIKTQVELATGQLGLRRAAQKHSFPRML
HQRERGLCHRGSFSLGEQSRVISHFLPNDLGFTDSYSQKAFCGIY
SKDGQIFMSACQDQTIRLYDCRYGRFRKFKSIKARDVGWSVLDVA
FTPDGNHFLYSSWSDYIHICNIYGEGDTHTALDLRPDERRFAVFS
IAVSSDGREVLGGANDGCLYVFDREQNRRTLQIESHEDDVNAVAF
ADISSQILFSGGDDAICKVWDRRTMREDDPKPVGALAGHQDGITE
IDSKGDARYLISNSKDQTIKLWDIRRESSREGMEASRQAATQQNW
DYRWQQVPKKAWRKLKLPGDSSLMTYRGHGVLHTLIRCRESPIHS
TGQQFIYSGCSTGKVVVYDLLSGHIVKKLTNHKACVRDVSWHPFE
EKIVSSSWDGNLRLWQYRQAEYFQDDMPESEECASAPAPVPQSST
PFSSPQ

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for generating a degron similarity score for one or more protein(s), the method comprising:
a) providing a first set of molecular surface features from a first set of one or more protein(s) comprising one or more known degron(s) of an E3 ligase substrate receptor and/or one or more predicted degron(s) of the E3 ligase substrate receptor;
b) providing a second set of molecular surface features from a second set of one or more protein(s); and
c) calculating a similarity score for the protein(s) of the second set by comparing the first and second sets of molecular surface features.

2. A method for identifying a predicted neosubstrate of an E3 ligase, the method comprising:
a) calculating a degron similarity score for one or more protein(s) according to the method of claim 1; and
b) based on the similarity score, identifying one or more of the protein(s) of the second set as a predicted neosubstrate(s) of the E3 ligase.

3. A method for identifying a putative neosubstrate of an E3 ligase, the method comprising:
a) identifying a predicted neosubstrate according to the method of claim 2;
b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and
c) if, based on said testing or having tested, the predicted neosubstrate is not determined to be a substrate of the E3 ligase, identifying the predicted neosubstrate as a putative neosubstrate of the E3 ligase.

4. A method for classifying protein(s) as substrate(s) and/or putative neosubstrate(s) of an E3 ligase, the method comprising:
a) calculating a degron similarity score for one or more protein(s) according to the method of claim 1;
b) based on the similarity score, identifying the protein(s) of the second set as a predicted neosubstrate of the E3 ligase or not; and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) i) if, based on said testing or having tested, the predicted neosubstrate is determined to be a substrate of the E3 ligase, classifying the predicted neosubstrate as a substrate of the E3 ligase; else
ii) if, based on said testing or having tested, the predicted neosubstrate is not determined to be a substrate of the E3 ligase, classifying the predicted neosubstrate as a putative neosubstrate of the E3 ligase,
thereby classifying protein(s) as substrate(s) and/or putative neosubstrate(s) of an E3 ligase.

5. A method for selecting putative neosubstrate(s) of an E3 ligase from a set of potential neosubstrates, the method comprising:
a) calculating a degron similarity score for one or more protein(s) according to the method of claim 1;
b) based on the similarity score, identifying a subset of the potential neosubstrates as predicted neosubstrate(s); and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates,
thereby selecting putative neosubstrate(s) of an E3 ligase from a set of potential neosubstrates.

6.-11. (canceled)

12. The method of claim 10, wherein the G-loop degron(s):
(i) comprise or consist of the amino acid sequence X¹-X²-X³-X⁴-G-X⁶, wherein: each of X¹, X², X³, X⁴, and X⁶ are independently selected from any one of the natural occurring amino acids; and G (i.e. X⁵) is glycine;
(ii) comprise or consists of the amino acid sequence X¹-X²-X³-X⁴-G-X⁶-X⁷, wherein: each of X¹, X², X³, X⁴, X⁶, and X⁷ are independently selected from any one of the natural occurring amino acids; and G (i.e. X⁵) is glycine;
(iii) comprise or consists of the amino acid sequence X¹-X²-X³-X⁴-G-X⁶-X⁷-X⁸; wherein: each of X¹, X², X³, X⁴, X⁶, X⁷, and X⁸ are independently selected from any one of the natural occurring amino acids; and G (i.e. X⁵) is glycine;
(iv) comprise or consists of the amino acid sequence X¹-X²-X³-X⁴-G-X⁶, wherein X¹ is selected from the group consisting of asparagine, aspartic acid, and cysteine; X² is selected from the group consisting of isoleucine, lysine, and asparagine; X³ is selected from the group consisting of threonine, lysine, and glutamine; X⁴ is selected from the group consisting of asparagine, serine, and cysteine; X⁵ is glycine; and X⁶ is selected from the group consisting of glutamic acid and glutamine;
(v) comprise or consists of the amino acid sequence X¹-X²-X³-X⁴-G-X⁶, wherein X¹ is asparagine; X² is isoleucine; X³ is threonine; X⁴ is asparagine; X⁵ is glycine; and X⁶ is glutamic acid;
(vi) comprise or consists of the amino acid sequence X¹-X²-X³-X⁴-G-X⁶, wherein X¹ is aspartic acid; X² is lysine; X³ is lysine; X⁴ is serine; X⁵ is glycine; and X⁶ is glutamic acid; and/or
(vii) comprise or consists of the amino acid sequence X¹-X²-X³-X⁴-G-X⁶, wherein X¹ is cysteine; X² is asparagine; X³ is glutamine; X⁴ is cysteine; X⁵ is glycine; and X⁶ is glutamine.

13.-16. (canceled)

17. The method of claim 1, wherein:
(i) the E3 ligase comprises the E3 ligase substrate receptor CRBN and the degron(s) are G-loop degron(s);
(ii) the E3 ligase comprises the E3 ligase substrate receptor BTRC and the degron(s) comprise or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid;
(iii) the E3 ligase comprises the E3 ligase substrate receptor KEAP1 and the degron(s) comprise or consists of the amino acid motif X¹-X²-X³-X⁴-X⁵-X⁶, wherein X¹ is selected from the group consisting of aspartic acid, asparagine, and serine; X² is any one of the naturally occurring amino acids; X³ is selected from the group consisting of aspartic acid, glutamic acid, and serine; X⁴ is selected from the group consisting of threonine, asparagine, and serine; X⁵ is glycine; and X⁶ is glutamic acid;
(iv) the E3 ligase comprises the E3 ligase substrate receptor KEAP1 and the degron(s) comprise or consists of the amino acid motif X¹-X²-X³-X⁴-X⁵-X⁶-X⁷-X⁸-X⁹, wherein X¹ is leucine; X² is any one of the naturally occurring amino acids; X³ is any one of the naturally occurring amino acids; X⁴ is glutamine; X⁵ is aspartic acid; X⁶ is any one of the naturally occurring amino acids; X⁷ is aspartic acid; X⁸ is leucine; and X⁹ is glycine;
(v) the E3 ligase comprises the E3 ligase substrate receptor KEAP1 and the degron(s) comprise or consists of the amino acid motif ETGE (SEQ ID NO: 1) and/or DLG;
(vi) the E3 ligase comprises the E3 ligase substrate receptor MDM2 and the degron(s) comprise or consists of the amino acid motif X¹-X²-X³-X⁴-X⁵-X⁶-X⁷-X⁸, wherein X¹ is phenylalanine; X² is any one of the naturally occurring amino acids; X3 is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine;
(vii) the E3 ligase comprises the E3 ligase substrate receptor MDM2 and the degron(s) comprise or consisting of the amino acid motif X¹-X²-X³-X⁴-X⁵-X⁶-X⁷-X⁸, wherein X¹ is phenylalanine; X² is any one of the naturally occurring amino acids; X³ is any one of the naturally occurring amino acids; X⁴ is any one of the naturally occurring amino acids; X⁵ is tryptophan; X⁶ is any one of the naturally occurring amino acids; X⁷ is any one of the naturally occurring amino acids; and X⁸ is selected from the group consisting of valine, isoleucine, and leucine forms an α-helix; or
(viii) the E3 ligase comprises the E3 ligase substrate receptor VHL and the degron(s) comprise or consists of the amino acid motif X¹-X²-X³-X⁴-X⁵-X⁶, wherein X¹ is leucine; X² is any naturally occurring amino acid; X³ is any naturally occurring amino acid; X⁴ is leucine; X⁵ is alanine; and X⁶ is proline or hydroxylated proline (e.g., 4(R)-L-hydroxyproline).

18. The method of claim 1, wherein the molecular surface features comprise geometric and/or chemical features, optionally wherein the geometric features are selected from the group consisting of shape index, distance-dependent curvature, geodesic polar coordinates, radial (angular) coordinates, and combinations thereof and/or wherein the chemical features are selected from the group consisting of hydropathy index, continuum electrostatics, location of free electrons, location of free proton donors, and combinations thereof.

19-20. (canceled)

21. The method of claim 1, wherein the similarity score is calculated using a geometric deep learning model, optionally a neural network, optionally wherein the neural network is trained on complementarity of E3 ligase surface(s) to known degron surface(s) or wherein the neural network is trained on similarity to known and/or predicted degron surface(s).

22.-30. (canceled)

31. A method for generating a degron complementarity score for one or more protein(s), the method comprising:
a) providing a first set of molecular surface features from a first set of one or more protein(s) comprising one or more E3 ligase substrate receptor proteins;
b) providing a second set of molecular surface features from a second set of one or more protein(s); and
c) calculating a complementarity score for the protein(s) of the second set by comparing the first and second sets of molecular surface features.

32. A method for identifying a predicted neosubstrate of an E3 ligase, the method comprising:
a) calculating a degron complementarity score for one or more protein(s) according to the method of claim 31; and
b) based on the complementarity score, identifying one or more of the protein(s) of the second set as a predicted neosubstrate(s) of the E3 ligase.

33. A method for identifying a putative neosubstrate of an E3 ligase, the method comprising:
a) identifying a predicted neosubstrate according to the method of claim 32;
b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and
c) if, based on said testing or having tested, the predicted neosubstrate is not determined to be a substrate of the E3 ligase, identifying the predicted neosubstrate as a putative neosubstrate of the E3 ligase.

34. A method for classifying protein(s) as substrate(s) and/or putative neosubstrate(s) of an E3 ligase, the method comprising:
a) calculating a degron complementarity score for one or more protein(s) according to the method of claim 31;
b) based on the complementarity score, identifying the protein(s) of the second set as a predicted neosubstrate of the E3 ligase or not; and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) i) if, based on said testing or having tested, the predicted neosubstrate is determined to be a substrate of the E3 ligase, classifying the predicted neosubstrate as a substrate of the E3 ligase; else
ii) if, based on said testing or having tested, the predicted neosubstrate is not determined to be a substrate of the E3 ligase, classifying the predicted neosubstrate as a putative neosubstrate of the E3 ligase,
thereby classifying protein(s) as substrate(s) and/or putative neosubstrate(s) of an E3 ligase.

35. A method for selecting putative neosubstrate(s) of an E3 ligase from a set of potential neosubstrates, the method comprising:
a) calculating a degron complementarity score for one or more protein(s) according to the method of claim 31;
b) based on the complementarity score, identifying a subset of the potential neosubstrates as predicted neosubstrate(s); and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates,
thereby selecting putative neosubstrate(s) of an E3 ligase from a set of potential neosubstrates.

36.-56. (canceled)

57. A method for generating a degron score for one or more protein(s), the method comprising:
a) providing a set of molecular surface features from a set of one or more protein(s); and
c) calculating a degron score for the protein(s) by comparing the molecular surface features to a reference set of molecular surface(s).

58. A method for identifying a predicted neosubstrate of an E3 ligase, the method comprising:
a) calculating a degron score for one or more protein(s) according to the method of claim 57; and
b) based on the degron score, identifying one or more of the protein(s) of the second set as a predicted neosubstrate(s) of the E3 ligase.

59. A method for identifying a putative neosubstrate of an E3 ligase, the method comprising:
a) identifying a predicted neosubstrate according to the method of claim 58;
b) for one or more of the predicted neosubstrate(s), testing or having tested the predicted neosubstrate in an E3 ligase substrate detection assay without a binding modulator of the E3 ligase to determine if the putative neosubstrate is a substrate of the E3 ligase; and
c) if, based on said testing or having tested, the predicted neosubstrate is not determined to be a substrate of the E3 ligase, identifying the predicted neosubstrate as a putative neosubstrate of the E3 ligase.

60. A method for classifying protein(s) as substrate(s) and/or putative neosubstrate(s) of an E3 ligase, the method comprising:
a) calculating a degron score for one or more protein(s) according to the method of claim 57;
b) based on the degron score, identifying the protein(s) of the second set as a predicted neosubstrate of the E3 ligase or not; and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) i) if, based on said testing or having tested, the predicted neosubstrate is determined to be a substrate of the E3 ligase, classifying the predicted neosubstrate as a substrate of the E3 ligase; else
ii) if, based on said testing or having tested, the predicted neosubstrate is not determined to be a substrate of the E3 ligase, classifying the predicted neosubstrate as a putative neosubstrate of the E3 ligase,
thereby classifying protein(s) as substrate(s) and/or putative neosubstrate(s) of an E3 ligase.

61. A method for selecting putative neosubstrate(s) of an E3 ligase from a set of potential neosubstrates, the method comprising:
a) calculating a degron score for one or more protein(s) according to the method of claim 57;
b) based on the degron score, identifying a subset of the potential neosubstrates as predicted neosubstrate(s); and
c) for one or more of the predicted neosubstrate(s), testing or having tested the predicted neosubstrate in an E3 ligase substrate detection assay without a binding modulator of the E3 ligase to determine if the predicted neosubstrate is substrate of the E3 ligase; and
d) if, based on said testing or having tested, the predicted neosubstrate is determined not to be a substrate of the E3 ligase, identifying the predicted neosubstrate as a putative neosubstrate of the E3 ligase and selecting it from the set of potential neosubstrates, thereby selecting putative neosubstrate(s) of an E3 ligase from a set of potential neosubstrates.

62.-83. (canceled)
